(19) United States
(12) Patent Application Publication
(10) Pub. No.: US 2011/0236953 A1
(43) Pub. Date: Sep. 29, 2011

(54) EMBRYONIC CEREBROSPINAL FLUID (e-CSF), PROTEINS FROM e-CSF, AND RELATED METHODS AND COMPOSITIONS

(75) Inventors: Christopher A. Walsh, Chestnut Hill, MA (US); Mauro Zappaterra, Jamaica Plain, MA (US); Maria Lehtinen, Brookline, MA (US)

(73) Assignee: Beth Israel Deaconness Medical Center, Boston, MA (US)

(21) Appl. No.: 12/672,009

(22) PCT Filed: Aug. 4, 2008
(86) PCT No.: PCT/US08/09405

§ 371 Date: May 24, 2011

Related U.S. Application Data
(60) Provisional application No. 60/963,211, filed on Aug. 3, 2007.

(51) Int. Cl. C12N 5/071; C12N 9/00; C12N 9/16; C12N 9/14; C12N 9/24; C12N 9/48; C12N 9/10; C12N 9/04; C12N 5/0797; C07K 14/65

(52) U.S. Cl. 435/190; 435/183; 435/196; 435/195; 435/200; 435/212; 435/193; 435/325; 530/350

(57) ABSTRACT

We have performed a proteomic analysis of embryonic cerebrospinal fluid (e-CSF) in human and rats. Based on this discovery, the invention features methods and compositions for cell culture including components of e-CSF or fragments thereof. Also provided are methods for extraction of e-CSF.

### STATEMENT AS TO FEDERALLY FUNDED RESEARCH

This invention was made with U.S. government support under grants HG00041, 2 RO1 NS032457, and P20 RR16462 awarded by National Institutes of Health. The Government has certain rights to this invention.

### BACKGROUND OF THE INVENTION

During the process of neurulation the neural groove forms and the neural folds fuse to form the neural tube. Once the neural tube is fused, the fluid within the lumen is considered cerebrospinal fluid, whereas before fusion is complete the neuroepithelium lining the inside of the neural tube is still in contact with amniotic fluid. During the early stages of neural tube growth and development, groups of specialized neuroepithelial cells lining the neural tube are believed to secrete fluid into the neural tube space in order to support growth and development of the embryo. As the neural tube continues to elongate and develop, specific highly vascularized epithelial cell types begin to invaginate at specific locations within the neural tube to form the specialized choroid plexus.

The choroid plexus is a highly vascularized epithelial cell structure that during development may be involved in the specific intracellular transfer of proteins into the CSF from the blood (Saunders et al., Cell Mol Neurobiol, 2000. 20:29-40). The choroid plexus develops in the lateral ventricles and in the third and fourth ventricles of the brain. In rats, the choroid plexus can be first identified as early as embryonic day 13 (E13) as a midline structure and by E15 it represents paired structures protruding into the lateral ventricles. In the human embryo the choroid plexus begins to develop in the lateral and fourth ventricle at Carnegie Stage (CS) 18, approximately 44 days post-ovulation. The first appearance of cerebral cortical neurons in the human embryo occurs at CS 21, shortly following the appearance of the choroid plexus and the production of CSF, and a similar temporal sequence is seen in mice and rats.

In adults, CSF has many functions, including an intermediary between blood and brain for the transport of nutrients and growth factors and as a fluid buffer for the brain to protect both the brain and the large vessels that supply blood to the brain (Chodobski et al., Microsc Res Tech, 2001. 52:65-82; Emerich et al., Bioessays, 2005. 27:262-74). It may also be involved in elimination of toxins and other metabolic byproducts (Emerich et al., Bioessays, 2005. 27:262-74; Miyan et al., Can J Physiol Pharmacol, 2003. 81:317-28). A mathematical analysis taking into account the pulsatile nature of CSF flow suggested that the CSF pulsations buffer the capillary bed from the effects of arterial pulsations that might otherwise prevent linear blood flow due to the mechanics of the brain being enclosed in the skull (Miyan et al., Can J Physiol Pharmacol, 2003. 81:317-28). CSF contains nerve growth factor (NGF), transforming growth factor alpha (TGF-alpha); levels of these proteins are altered in neurological and developmental disorders (Miyan et al., Can J Physiol Pharmacol, 2003. 81:317-28; Kasaian et al., Biofactors, 1989. 2:99-104; Massaro et al., Ital J Neurol Sci, 1994. 15:105-8; Patterson et al., Brain Res, 1993. 605:43-9; Van Setten et al., Int J Dev Neurosci, 1999. 17:131-4), but potential functions of these factors has not been demonstrated. Recently it was shown that the ciliary action of CSF in the lateral ventricle of adult rats creates a gradient of SLIT2 protein, a chemorepulsive factor for neuronal olfactory bulb migration, within the CSF (Sawamoto et al., Science, 2006. 311:629-32), suggesting that CSF factors might have instructive roles for developing neurons or neural progenitors.

Although the role of the CSF during embryogenesis is just starting to be studied, an important role has been suggested in brain development (Miyan et al., Can J Physiol Pharmacol, 2003. 81:317-28; Gato et al., Anat Rec A Discov Mol Cell Evol Biol, 2005. 284:475-84; Martin et al., Dev Biol, 2006. 297:402-16; Mashayekhi et al., Brain, 2002. 125:1859-74; Miyan et al., Cerebrospinal Fluid Res, 2006. 3:2; Owen-Lynch et al., Brain, 2003. 126:623-31). Miyan et al. have shown that rat cortical cells are viable and proliferate in e-CSF (Miyan et al., Cerebrospinal Fluid Res, 2006. 3:2). Other studies have tested discrete signaling factors that may regulate neurogenesis. Gato et al. and Martin et al. have studied the role of chick e-CSF in regulating survival, proliferation, and neurogenesis of neuroepithelial cells, and have identified FGF-2 in the chick CSF as a vital trophic factor (Gato et al., Anat Rec A Discov Mol Cell Evol Biol, 2005. 284:475-84; Martin et al., Dev Biol, 2006. 297:402-16). Intriguingly, in mutant animals, CSF factors that may inhibit proliferation have been suggested. In studies of the hydrocephalic Texas (H-Tx) rat, cell proliferation in the ventricular zone decreases, and although cell migration still occurs, there is a decrease in the number of migrating cells (Mashayekhi et al., Brain, 2002. 125:1859-74; Miyan et al., Cerebrospinal Fluid Res, 2006. 3:2). In addition, CSF from the lateral ventricles of affected H-Tx fetuses can completely inhibit in vitro proliferation of neuronal progenitors isolated from a normal fetus at 10% CSF addition to the media, suggesting that factors intrinsic to the CSF of the H-Tx fetuses are present that inhibit proliferation.

Prior to the present invention, the identification of such CSF factors with a developmental role has been impeded, as the components of the CSF were previously not known. While a first glimpse of the protein composition of e-CSF has been provided, a (Parada et al., Proteomics, 2006. 6:312-20; Parada et al., J Proteome Res, 2005. 4:2420-8), a complete analysis of the contents of e-CSF would be allow for identification of proteins important for neural developmental and differentiation.

### SUMMARY OF THE INVENTION

We have developed methods for isolating embryonic cerebrospinal fluid (e-CSF) and have identified the proteins found in rat and human e-CSF. On this basis, the invention features a method of isolating embryonic cerebrospinal fluid (e-CSF). As e-CSF is capable of supporting the culture of developing neural cells, the invention also features methods of culturing cells in the presence of various components of the e-CSF, as well as compositions including cells and e-CSF component(s).

In a first aspect, the invention features a composition including at least one (e.g., at least 2, 3, 4, 5, 6, 8, 10, 15, 25, 50, 75, or 100) component(s) of e-CSF (e.g., rat, mouse, or human). The composition may include the component or components at an enhanced level relative to the level in e-CSF and the composition is capable of supporting proliferation, maintenance, or differentiation of a cultured cell (e.g., a stem cell or progenitor cell such as neural cell). The component may be a polypeptide, or a functional fragment thereof (e.g., a soluble fragment). The polypeptide may be isolated, purified, or produced recombinantly. The component may be present at a level sufficient to enhance cell proliferation, maintenance, or differentiation, as compared to in the absence of the component. The component may be one that is not found in adult CSF.

The invention also features a cell culture composition including a cell (e.g., any described herein) and a composition of the first aspect.

The invention also features a kit including (a) a composition including at least one component of e-CSF, wherein the component is present at an enhanced level relative to naturally occurring e-CSF (e.g., the compositions described above); and (b) instructions for using (a) for cell culture.

In another aspect, the invention features a method of culturing a cell (e.g., a stem cell or a progenitor cell, such as a neural cell), including incubating the cell in culture media containing at least one isolated component of rat or human e-CSF (e.g., any of the compositions of the first aspect of the invention). The component may be a polypeptide, or a functional fragment thereof (e.g., a soluble fragment). The polypeptide may be isolated, purified, or produced recombinantly. In certain embodiments, the component is one which is not found in adult CSF.

In any of the above aspects, the e-CSF component may be one described in Tables 1-4.

In another aspect, the invention features a method of isolating embryonic cerebrospinal fluid (e-CSF) including (a) providing an embryo; (b) inserting a capillary needle into a ventricle of the central nervous system of the embryo such that the tip of the needle contacts CSF; and (c) extracting CSF from the embryo through the needle (e.g., a microcapillary pipette or syringe), thereby isolating e-CSF. The method may further include (d) removing intact contaminating cells (e.g., by filtration or centrifugation). Step (c) may be performed such that the needle tip does not contact the neuroepithelium during the extraction. The e-CSF may be removed from a lateral ventricle or from the third or fourth ventricle of the embryo, or a combination thereof. The method may further include storing the e-CSF at less than about 0-20° C. to about −80, −90, −100, −150° C.

By “isolated” is meant, with respect to a naturally occurring compound (e.g., a polypeptide), that the compound is at least partially free from the components (e.g., other polypeptides, nucleic acids, cell membranes) with which it naturally is found.

By “purified” is meant, with respect to a compound (e.g., a polypeptide), that the compound makes up at least 20% (e.g., at least 30%, 40%, 50%, 60%, 70%, 80%, 90%, 95%, or 99%) of the composition with which is it found.

By “enhanced level” of a component is meant that the component is present either a higher concentration (e.g., at least 10%, 25%, 50%, 100%, 250%, 500%, or 1000% greater) or at higher purity level (e.g., with 5%, 10%, 25%, 50%, or 75% less by mass other components, not including solvents or buffers) relative to the concentration or purity of the component in a control composition (e.g., a naturally occurring composition).

By “stem cell” is meant a self-renewing cell that is capable of differentiation into multiple mature cell types (e.g., a neuron, glial cell, or astrocyte).

By “progenitor cell” (e.g., neural progenitor cells) is meant a cell that is capable of forming at least one cell type has at least some capacity for self-renewal.

Other features and advantages of the invention will be apparent from the following Detailed Description, the drawings, and the claims.

### BRIEF DESCRIPTION OF THE DRAWINGS

FIGS. 1A-1C are a set of images showing extraction and SDS-PAGE analysis of human and rat embryonic CSF. FIG. 1A is an image of hematoxylin and eosin sagittal section of E14.5 rat showing CSF aspiration technique and the position of the syringe needle relative to surrounding tissues in the lateral ventricle (LV) and the 4^{th} ventricle (4^{th} V). The inset image of E14.5 rat embryo provides orientation. The arrow head is 4^{th} V, and the arrow is the mouth/chin. FIG. 1B shows CSF aspirated from the 4^{th} ventricle of a CS20 human embryo (CS20) and a CS19 human embryo (CS19) separated by size using SDS-PAGE on a 7.5% or 10% polyacrylamide gel respectively. For clarity, the CS20 sample shows 1/7^{th} of the sample used in the final analysis. FIG. 1C shows CSF aspirated from the lateral ventricles (LV) of E12.5, E14.5 and E17.5 rat. The arrow in all samples represents Apolipoprotein-B.

FIGS. 2A-2D are graphs showing classification and comparison of proteins based on subcellular localization. Graphic representation of the subcellular localization of proteins in CS 20 embryonic human CSF (FIG. 2A), embryonic rat CSF (FIG. 2B), and E16.5 mouse brain (FIG. 2C) is shown. The percentage of protein localization is calculated based on the total number of proteins localized to each space divided by the total number of proteins in the CSF that we were able to assign localization (human CSF-187 proteins, rat CSF-137 proteins, and mouse brain-179 proteins). Some proteins were localized to multiple compartments within the cell. FIG. 2D shows a comparison between human CSF, rat CSF, and mouse brain of the number of protein from each category based on localization.

FIG. 3 is a graph showing comparison of proteins based on molecular function. Proteins present in embryonic human CSF, embryonic rat CSF, and embryonic mouse brain were analyzed using the Panther gene ontology database and classified according to molecular function. The chart includes protein category name. Percentage is calculated from the number of proteins assigned to each category over total number of proteins analyzed.

FIG. 4 is a graph showing comparison of proteins based on biological process. Proteins present in embryonic human CSF, embryonic rat CSF, and embryonic mouse brain were analyzed using the Panther gene ontology database and classified according to the biological process with which the proteins are involved. The chart includes protein category name. Percentage is calculated from number of proteins assigned to each category over total number of proteins analyzed.

FIGS. 5A-5C are graphs showing classification of proteins based on molecular function. Proteins present in embryonic human CSF (FIG. 5A), embryonic rat CSF (FIG. 5B), and embryonic mouse brain (FIG. 5C) were analyzed using the Panther gene ontology database and classified according to molecular function. Each graph includes protein category name, number of proteins assigned to each category, and percentage of proteins assigned to each category. Proteins can be assigned to more than one category based on molecular function.

FIGS. 6A-6C are graphs showing classification of proteins based on biological process. Proteins present in embryonic human CSF (FIG. 6A), embryonic rat CSF (FIG. 6B), and embryonic mouse brain (FIG. 6C) were analyzed using the Panther gene ontology database and classified according to the biological process the proteins are involved with. Each graph includes protein category name, number of proteins assigned to each category, and percentage of proteins assigned to each category. Proteins can be assigned to more than one category based on biological process.

FIG. 7 is a graph showing sub-classification of regulatory molecules based on molecular function. Regulatory molecules present in the embryonic human CSF, rat CSF, and embryonic mouse brain were further sub-classified based on molecular function. Although in FIG. 3 the percentage of regulatory molecules found in CSF and mouse brain appears similar, further sub-classification shows a distinct similarity in protein classes between CSF samples and a distinct difference in protein classes between CSF and brain samples.

FIG. 8 is a graph showing sub-classification of protein metabolism and modification based on biological process. Proteins involved in protein metabolism and modification present in the embryonic human CSF, rat CSF, and embryonic mouse brain were further sub-classified based on biological process. Although in FIG. 4 the percentage of proteins involved in protein metabolism and modification found in CSF and mouse brain appeared similar, further sub-classification clearly shows a distinct similarity in protein classes between CSF samples and a distinct difference in protein classes between CSF and brain samples.

FIGS. 9A-9Z shows that embryonic CSF supports cortical explant viability and growth and E17 CSF stimulates proliferation of neural progenitor cells in cortical explants and in cell culture. FIG. 9A is a schematic diagram of cortical explant dissections; 3-D image of E16 rat brain with dark box depicting region of dissection for explant. Cross section image of rat brain depicts medial and lateral border of explant dissection. Crossed arrows designate orientation of explant (E) on membrane with orienting cut at medial-caudal side (L-lateral, M-medial, C-caudal, R-rostral). FIGS. 9B-9D are images showing tissue stained with Hoechst (blue), anti-PH3 (red), and anti-Tuj1 (green). (FIG. 9B) E17 rat cortex; (FIGS. 9C and 9D) E16 explants grown for 24 hours in 100% embryonic CSF (e-CSF) and 100% artificial CSF (ACSF), respectively. Explants grown in 100% embryonic CSF in vitro maintain tissue histology similar to embryo in vivo. FIGS. 9E-9G show tissue stained with anti-BrdU (blue), anti-PH3 (red), anti-Tuj1 (green). (FIG. 9E) E17 rat cortex labeled with BrdU, mother was administered a bolus of BrdU (60 mg/kg) 3 hours prior to removing embryos. (FIGS. 9F-9G) E16 explant grown for 24 hours in 100% embryonic CSF and ACSF respectively. Explants were administered BrdU (20 uM) 30 minutes prior to fixation. Explants grown in 100% embryonic CSF incorporated BrdU after 24 hours in vitro indicating cells undergoing DNA synthesis. Survival and proliferation of the explants grown with embryonic CSF are indicated by immunoreactivity for phospho-Histone H3 (PH3, a marker of cell division) along the ventricular surface, BrdU incorporation (marking proliferating cells at the time of BrdU exposure) in the ventricular zone, and Tuj1-positive-staining neurons in the developing cortical plate. FIGS. 9H-9M show E16 explants cultured in 100% E13 or E17 CSF for 24 hours, (FIGS. 9H and 9I) stained with anti-PH3 (red) and Hoechst (blue) (FIGS. 9J and 9K) stained with anti-Vimentin 4A4 (green) and Hoechst (blue), (FIGS. 9L and 9M) merged images of anti-PH3 (red), anti-Vimentin 4A4 (green) and Hoechst (blue). FIG. 9N shows quantification of total PH3-positive-staining cells per explant grown with E13 and E17 CSF. The number of PH3-positive-staining cells is represented as mean±SEM. The number of PH3-positive-staining cells was significantly increased in explants cultured with E17 CSF compared to E13 CSF (Mann-Whitney; E17: 44.1±1.43; E13: 25±4.2; p<0.05; n=4). FIG. 9O shows quantification of PH3-positive-staining cells along the ventricle per explant grown with E13 and E17 CSF. The number of PH3-positive-staining cells along the ventricle was significantly increased in explants cultured with E17 CSF compared to E13 CSF (Mann-Whitney; E17: 32.3±0.79; E13: 12.8±3.9; p<0.05; n=4). FIG. 9P shows quantification of Vimentin 4A4-positive-staining cells per explant grown with E13 and E17 CSF. The number of Vimentin 4A4-positive-staining cells was significantly increased in explants cultured with E17 CSF compared to E13 CSF (Mann-Whitney; E17: 45.9±6.6; E13: 13.9±2.2, p<0.05; n=3). FIGS. 9Q-9Y show single cells from dissociated primary neurospheres grown in: (FIGS. 9Q, 9T, and 9W) 20% ACSF, (FIGS. 9R, 9U, and 9X) 20% E14 CSF, (FIGS. 9S, 9V, and 9Y) 20% E17 CSF for 9 DIV and stained with anti-GLAST, Hoechst, and merged images, respectively. Primary dissociated spheres grown in E17 CSF proliferate and form spheres of slowing dividing GLAST positive cells. FIG. 9Z shows quantification of average number of spheres per cm² formed in the various conditions at 9 DIV.

FIGS. 10A-10F show that e-CSF supports cortical explant survival. FIGS. 10A, 10C, and 10E show E16 explants grown for 24 hours in 100% e-CSF, and FIGS. 10B, 10D, and 10F show 100% artificial CSF (ACSF) and stained for early apoptotic cell death marker Cleaved Caspase 3 (CC3). Explants grown in 100% embryonic CSF has decreased CC3 stain compared to explants grown in ACSF. The embryonic CSF supports tissue viability and survival.

FIGS. 11A-11I show neural stem cells grown in embryonic CSF maintain undifferentiated state. FIGS. 11A-11C show dissociated cells from primary neurospheres cultured in E17 CSF for 10 DIV. Cells maintain GLAST-positive neural progenitors when cultured in embryonic CSF. FIGS. 11D-11F show dissociated cells from primary neurospheres cultured in E17 CSF for 5 DIV and then supplemented with EGF and FGF. GLAST-positive-staining cells cultured in E17 CSF maintain responsiveness to EGF and FGF suggesting that stem cells cultured in CSF maintain undifferentiated and uncommitted state. FIGS. 11G-11I show dissociated cells from primary neurospheres cultured in EGF and FGF for 10 DIV.

FIGS. 12A-12G show embryonic CSF maintains GLAST-positive-staining stem cells for 44 DIV. FIG. 12A-12D show dissociated cells from primary neurospheres cultured in E17 CSF for 44 DIV. Cells maintain GLAST-positive neural progenitors when cultured in embryonic CSF for extended periods of time. FIG. 12E shows quantification of number of spheres per cm² when cultured for 10 DIV versus 44 DIV. FIG. 12F shows quantification of relative colony size of spheres cultured for 10 DIV versus 44 DIV. FIG. 12G shows quantification of circularity of spheres cultured for 10 DIV versus 44 DIV.

FIG. 13A-13C show dynamic changes in CSF protein concentration and composition during development. FIG. 13A is agGraph of total CSF protein concentration collected from rats at various stages in development. FIG. 13B is a silver stain of CSF from different ages in development, revealing a dynamic fluid with numerous changes in protein composition over time. FIG. 13C is a western blot analysis of specific proteins identified in the embryonic CSF. CSF collected from various ages during development and immunoblotted with antibodies to Albumin, Transferrin, FGF2, EC-SOD, Cathepsin B, Cystatin C, Amyloid Precursor Protein (sAPP).

FIGS. 14A-14F show that embryonic CSF activates IGF1R and p-AKT signaling and provides a source of insulin signaling to progenitor cells along the ventricle in the cortex. FIG. 14A shows Igf2 peptides recognized by LC-MS/MS in E17 CSF (red). FIG. 14B shows that Igf2 levels are detectable by western blot at E13 and then decrease into adulthood. FIG. 14C shows an in situ hybridization for IGF2 at E14. c′ and c″ are magnified images showing IGF2 levels highest in leptomeninges and blood vessels within the cortex, FIG. 14D shows an in situ hybridization for IGF2 at E17. d′ and d″ are magnified images showing IGF2 levels are highest in the choroid plexus (CP), leptomeninges, and blood vessels within the cortex, FIG. 14E 10× and (e′) 20× image of IHC analysis of Igf1R localization in the E17 developing rat brain reveals Igf1R localization along the apical surface of the ventricle. FIG. 14F shows lysates of cortical cells treated with ACSF, E17 CSF, or IGF2 for 5 minutes immunoblotted with antibodies to p-IGF1R, p-AKT, AKT, P-ERK1/2, and ERK1/2.

FIGS. 15A-15K show that Igf2 maintains and stimulates proliferation of neural progenitor cells. FIGS. 15A-15D show single cells dissociated from primary neurospheres grown in control media or control media plus IGF2 (20 ng/ml). Small secondary spheres cultured with Igf2 alone form after 10 DIV. IHC with anti-GLAST on secondary spheres after 10 DIV shows GLAST immunoreactivity, indicating maintenance of neural progenitor cell identity with IGF2 alone. FIGS. 15E-15G show E16 cortical explants cultured in control E17 CSF or E17 CSF with IGF2 neutralizing antibody (IGF2 NAb), stained with anti-Vimentin 4A4 (green) and Hoechst (blue). FIG. 15G shows quantification of Vimentin 4A4-positive-staining cells per explant grown with E17 control CSF or with IGF2 NAb. The number of Vimentin 4A4-positive-staining cells was significantly decreased in explants cultured with E17 CSF plus IGF2 NAb compared to control E17 CSF (Mann-Whitney; E17 control mean: 28.8±4.3; E17 Igf2 neutralizing antibody mean: 13.9±2.0; n=4, p<0.05). FIGS. 15H-15J show E16 cortical explants cultured with Neural Basal Media plus ACSF (control) or with supplemental IGF2 stained with anti-Vimentin 4A4 (green) and Hoechst (blue). FIG. 15J shows quantification of Vimentin 4A4-positive-staining cells per explant grown with control media or with supplemental IGF2. The number of Vimentin 4A4-positive-staining cells was increased in explants cultured with IGF2 supplementation compared with control (Mann-Whitney; Igf2 supplementation mean: 36.7±2.1; control mean: 20.4±4.46; n=8, p<0.05). FIG. 15K shows an overall model depicting factors released from the choroid plexus into the CSF can act over large distances to regulation progenitor cell survival, proliferation and maintenance. As an example, we illustrate IGF2 as a secreted factor that regulates the maintenance of progenitor cell fate.

### DETAILED DESCRIPTION

Here we undertake a systematic, detailed, and unbiased proteomic analysis of human e-CSF from Carnegie Stage 19-20 (approximately 48-51 days post ovulation). We also report an extensive proteome analysis of rat e-CSF from three different time points E12.5, E14.5, and E17.5 during cortical development and list all the proteins that are common among the three time points as well as those proteins that are different.

We report a list of the common proteins found between the human and rat e-CSF. Furthermore, using various gene ontology programs we categorize the proteins in the e-CSF and compare the subcellular localization, molecular function, and biological process of embryonic human and rat CSF. We find 130 proteins shared between the human and rat e-CSF and that there are many similarities in the categories of proteins found within the CSF based on molecular function and biological process. This systematic analysis of proteins common to many ages lays the groundwork for analysis of changing CSF components that may have more specific developmental roles.

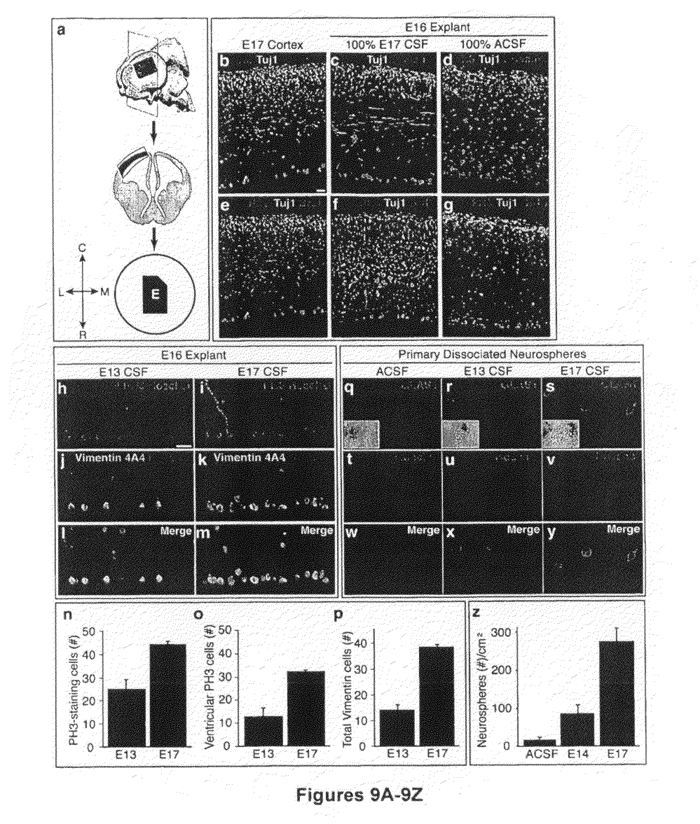

As described herein, rat embryonic CSF proteome is a complex and dynamic milieu of extracellular matrix proteins, intracellular proteins, and signaling factors (see also, Zappaterra et al., J Proteome Res 6, 3537-48 (2007)). However, prior to the present invention, the direct influence of embryonic CSF on cortical progenitor cells had been challenging to assess due to the difficulty of obtaining substantial amounts of CSF. In addition, we developed a cortical explant culture system in which embryonic cortex dissected from a consistent location of the lateral wall is placed on polycarbonate membranes and floated on embryonic CSF (FIG. 9A). This explant culturing technique enables variable pairings of cortical tissue and CSF, or e-CSF components to investigate the relationship between cortical progenitor cells and CSF-mediated signaling.

On the basis of this work, the present invention features methods for isolation of e-CSF, and methods of culturing cells (e.g., stem cells or progenitor cells such as neural stem cells) using one or more (e.g., 2, 3, 4, 5, 6, 8, or 10) components (e.g., a purified or recombinantly produced polypeptide) of e-CSF and compositions including cells with one or more such components.

Isolation of CSF from Human Embryos

In general, CSF can be isolated from any mammalian embryo using the methods described herein. Typically the embryos at the appropriate stage are collected, and the extra embryonic membranes and tissues are dissected away in a buffer solution (e.g., phosphate-buffer saline (PBS) or Hanks' Balanced Salt Solution (HBSS)). A capillary needle (e.g., a syringe or microcapillary pipette) is placed into a CNS ventricle (e.g., lateral, third, or fourth ventricle) and the CSF is withdrawn. To avoid contaminating cells, it is desirable that contact with either blood vessels or with the neuroepithelium be avoid. To ensure that the e-CSF sample is cell-free, the sample can treated to remove cells (e.g., by centrifugation or by filtration).

In one example, human embryos were collected through the joint MRC-Wellcome Trust Human Developmental Biology Resource at the University of Newcastle, Institute of Human Genetics. The embryos at CS 19-20 were placed in ice-cold sterile Phosphate Buffered Saline (PBS) solution and all extra-embryonic membranes and tissues were removed. The embryos were washed in sterile PBS and carefully placed on the dissection platform under the microscope. A Hamilton syringe was placed carefully into the fourth ventricle and the CSF was collected paying close attention not to make contact with the neuroepithelium lining of the fourth ventricle. The samples used for analysis had no microscopically visible contaminating neuroepithelial cells or red blood cells. Nonetheless, the CSF samples were centrifuged at 10,000 g at 4° C. for 10 minutes to remove any intact contaminating cells and then were frozen at −80° C. until further analysis.

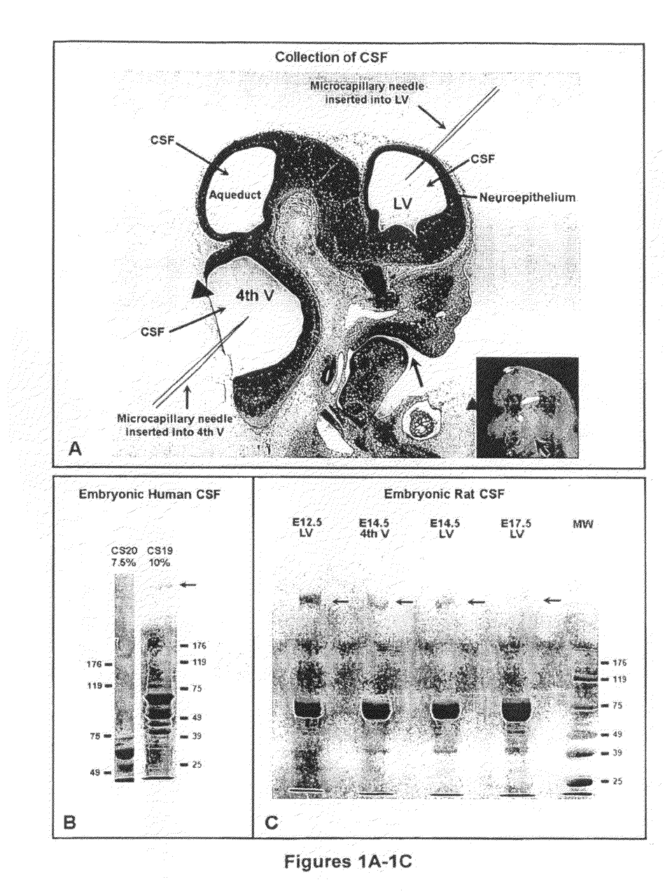

In another example, rat embryos (Sprague Dawley) at stage E12.5, E14.5 and E17.5 were removed from extra-embryonic membranes and tissues and placed in sterile Hanks Balanced Salt Solution (HBSS). Each embryo was handled individually and washed in HBSS, gently patted dry and placed on a microdissection tray. The CSF was carefully aspirated from each rat embryo under the microscope with a pulled tip glass microcapillary pipette (Drummond Scientific Company 20 μl). The needle was steadily held within the inside of the ventricle so as to prevent major contact with the neuroepithelial wall and the CSF was slowly aspirated. For E17.5, the embryo was placed on its back and the glass needle was inserted into the left lateral ventricle and then into the right lateral ventricle to collect the maximum amount of CSF from the lateral ventricles. For E12.5, the embryo was placed on its side and the glass needle was inserted directly into the lateral ventricle. Due to the patency of the neural tube at this stage, the CSF was collected from the developing lateral, third, and fourth ventricle. For E14.5, the embryo was also placed on its side and the glass needle was either inserted into the lateral ventricle or into the fourth ventricle and the CSF was collected from each location separately. FIG. 1A is a diagram depicting CSF isolation from E14.5 rat. CSF for each analysis was collected from two entire litters and pooled as one sample. To minimize protein degradation, CSF samples were kept at 4° C. during collection. CSF samples were centrifuged at 10,000 g at 4° C. for 10 minutes to remove any contaminating cells. The samples that we used for analysis had no visible sign of contaminating neuroepithelium cells or red blood cells as we could detect under the microscope. Samples were frozen at −80° C. until further analysis.

Cell Culture Using e-CSF Components

As we have shown that e-CSF stimulates proliferation and maintenance of neural stem cells in vitro and have, for the first time, identified many of the polypeptides found in e-CSF, it now becomes possible to uses these identified proteins in a cell culture system as proliferation, differentiation and maintenance, particularly with regard to stem cells and progenitors, especially those of neural origin. On this basis, the invention features methods of cell culture using e-CSF or one or more e-CSF components (e.g., polypeptides, either alone or as a supplement to standard cell culture media, and cell culture compositions including one or more components of e-CSF.

Any of the polypeptides identified in e-CSF may be used as a supplement in a cell culture (e.g., those described in Tables 1-4), or a combination of components from e-CSF (e.g., polypeptides) may be used for cell culture (e.g., for proliferating cells such as stem cells). Functional fragments (e.g., soluble fragments) of any of the proteins described herein) may be used in the invention. Soluble fragments are particularly useful for membrane bound proteins.

### Cell Types

The compositions and methods of the invention may employ any type of cultured cell known in the art. In particular embodiments, a proliferating cell, such as a stem cell or progenitor cell is used. Neural cells (e.g., neural stem cells and neural progenitor cells) can be used in the invention as well. Human and non-human mammal cells (e.g., rat or mouse) cells are used in certain embodiments. Stem cell lines are, for example, commercially available or can be obtained directly from laboratory animals such as mice or rats.

### Cell Media Compositions

In one embodiment, the cell media composition of the invention includes any medium known in the art supplemented with at least one (e.g., at least 2, 3, 4, 5, 6, 8, 10, 15, 25, 50) components identified in e-CSF (e.g., those described herein). Exemplary media types used for culturing neural stem cells include Neuralbasal Media (Invitrogen Corp.), Neural Stem Cell Commitment Media or Neural Stem Cell Growth Meida (AlphaGenix, Inc.), and NeuroCult NS-A Proliferation, Human; Kit or NeuroCult NS-A Differentiation Humam Kit (Stem Cell Technologies, Inc.). In another embodiment, the cell culture media is derived entirely from e-CSF components.

Determining Whether a Component of e-CSF Enhances Proliferation, Maintenance, or Differentiation of a Cell

Any component of e-CSF (e.g., those described herein) may be analyzed to determine its effect on a cell (e.g., a neuronal progenitor or stem cell described herein) in culture. Desirable components are those which result in maintenance of the cells or those that result in more rapid proliferation or differentiation of the cell. Assays to measure proliferation (e.g., using cell dyes or incorporation of a modified nucleotide such as BrdU) and differention (e.g., using differentiation markers known in the art) are well known by those of skill in the art.

The Role of e-CSF in Brain Development

Embryonic CSF plays a fundamental, dynamic role in defining an endogenous niche for the survival and proliferation of cortical neural progenitors. CSF alone supports the growth and proliferation of cortical explants in the absence of exogenous media or factors. CSF from different ages in development harbors distinct proliferative capacities for neural progenitor cells the characterization of the CSF proteome has identified several classes of proteins in the CSF established as essential regulators of proliferation and maintenance of neuronal progenitor cells; and we identified and characterized a novel role for IGF2 signaling in the embryonic CSF as a regulator of cortical progenitor cell proliferation.

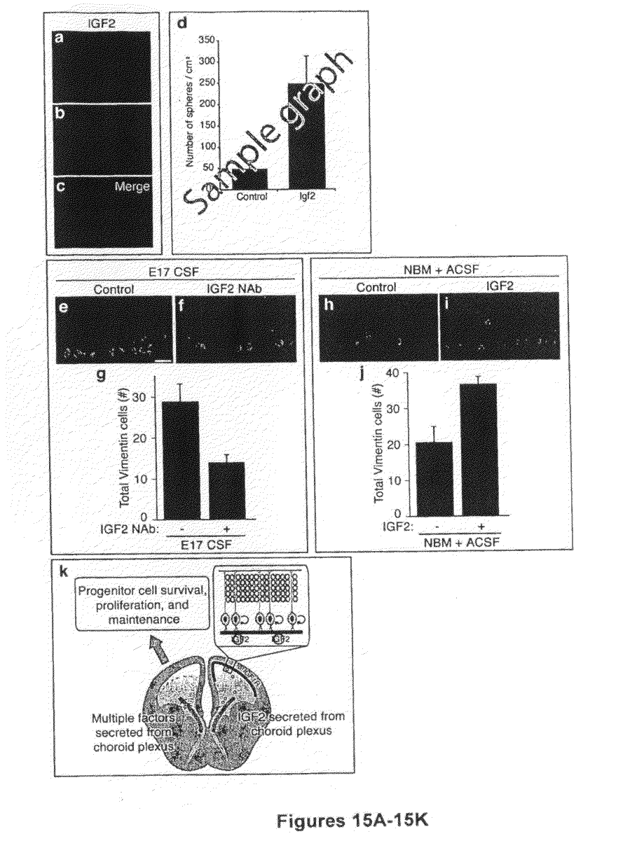

The CSF has been traditionally considered as a fluid cushion that bathes the central nervous system, acting as a passive sink for biomarkers of central nervous system function and pathology. Collectively, our study represents a paradigm shift in developmental neuroscience, suggesting that the embryonic CSF proteome as a dynamic milieu of growth-promoting signals for neural stem cells (FIG. 15K).

The CSF-choroid-plexus system is ideally suited to act as a rapid, spatially synchronized medium for triggering local and global changes in molecular signaling. Dynamism of factors such as IGF2 levels in the CSF is consistent with a role of the CSF as a vehicle for orchestrating cortical neurogenesis: IGF2 expression increases during development, is maximal during the peak of cortical neurogenesis in the rat brain (E17-E19), and declines as cortical neurogenesis nears completion around birth. Therefore, the appearance of signaling factors such as IGF2 stimulate the proliferation of cortical progenitors to maintain them in an uncommitted state through development. Igf2 and other molecules (Martin et al., Dev Biol 297, 402-16 (2006)) appear to be released in the CSF by choroid plexus, which appears in the lateral ventricles between E13 and E15. These signaling molecules, via the CSF must act widely on cortical precursors that, in the case of the embryonic human brain, may be centimeters away from the source of the factor. It is unclear whether there is a gradient of Igf2 in the embryonic CSF influences regional differences in proliferation across the cortical mantle (Bayer et al., Prog Neurobiol 29, 57-106 (1987)), as has been shown for Slit in the adult CSF (Sawamoto et al., Science 311, 629-32 (2006)), or whether ciliary movement or diffusion through a far smaller volume equilibrates Igf2 concentration in the embryonic ventricles. A fundamental aspect of neural differentiation may be the simple isolation of developing cells from this growth-promoting environment, by the withdrawal of the ventricular process (Cappello et al., Nat Neurosci 9, 1099-107 (2006)).

Our findings have several important implications. First, CSF in the embryo and the adult is a dynamic fluid that contacts a number of CNS precursors as well as differentiating neurons and glia. Second, CSF components can be dispersed over large areas and thus may be more significant and pervasive regulators of development, stem cell renewal, disease, neurodegeneration and behavior than previously thought. Third, since the CNS represents just one example of an epithelium that grows in relation to an extracellular fluid, our findings may generalize to other epithelia which are likely to develop using similar rules, with a major contributor to the “stem cell niche” being the fluid that bathes the epithelium (Bendall et al., Nature 448, 1015-21 (2007)), similar to the microenvironment that invests hematopoietic stem cells, of which Igf2 is also an essential component (Zhang et al., Blood 103, 2513-21 (2004); Orkin et al., Cell 132, 631-44 (2008)), as in the embryonic CSF. Finally, if a major component of the stem cell niche reflects secreted factors acting at large distances from their sources, a deeper understanding of the proteomic composition of extracellular fluids may provide unexpected ways to regulate stem cell behavior.

### In-Gel Digestion and Mass Spectrometry

To determine the protein contents of e-CSF, frozen CSF samples were thawed on ice. Sample buffer was added and the samples were boiled for 5 minutes and subjected to SDS-PAGE using either 10% or 7.5% polyacrylamide (37.5:1 acrylamide:bis-acrylamide) gels as indicated in FIG. 1B-1C. Each gel lane (which included the 4.2% polyacrylamide stacking gel) was cut into ten regions and each region was diced and subjected to in-gel digestion with sequencing grade modified trypsin (Promega, 6 ng/μl) in 50 mM ammonium bicarbonate overnight at 37° C. Peptides were extracted with 50% acetonitrile (MeCN), 2.5% formic acid (FA) and then dried. Peptides were then resuspended in 2.5% MeCN, 2.5% FA and loaded using an autosampler onto a microcapillary column packed with 12 cm of reverse phase MagicC18 material (5 μm, 200 Å, Michrom Bioresources, Inc.). Elution was performed with a 5-35% MeCN (0.1% FA) gradient over 60 minutes, after a 15 minute isocratic loading at 2.5% MeCN, 0.5% FA. Mass spectra were acquired in LTQ and LTQ-XL linear ion trap mass spectrometers (Termo Electron) over the entire 75 minutes using ten MS/MS scans following each survey scan. Raw data were searched against either the human or rat IPI forward and reverse concatenated databases using Sequest software requiring tryptic peptide matches with a 2 Da mass tolerance (Elias et al., Nat Methods, 2005. 2:667-75). Cysteine residues were required to have a static increase in 71.0 Da for acrylamide adduction and differential modification of 16.0 Da on methionine residues was permitted. The resultant top matches for all analyses of each gel lane were compiled. Each list was then filtered independently using a dCn2 score of 0.2 and Xcorr scores of 1.8, 2.0 and 2.5 for singly, doubly, and tripled charged ions respectively. Proteins on these filtered lists that had two or more peptides were retained. However keratin proteins were removed as they are known contaminants in most gel-based proteomic analyses. Based on the number of reverse database false-positives that were also retained following these filtering criteria, we estimate the following false-positive rates for the proteins in each sample: rat E12.5 lateral ventricle (LV), 0.45%; rat E14.5LV, 0.30%; rat E17.5LV, 0.50%; rat E14.5 4^{th} ventricle, <0.00%; and human CS 20, <0.00%. For the human CS 19 sample the estimated false-positive rate for proteins identified by more than three peptides is <0.00%. The dataset of proteins for the embryonic mouse brain was extracted from LC-MS/MS data collected from 16 strong cation exchange (SCX) fractions generated during our previous study of the forebrain and midbrain extracts of E16.5 mouse embryos (Ballif et al., Mol Cell Proteomics, 2004. 3:1093-101). We compiled the LC-MS/MS data from four SCX fractions in the middle of the SCX gradient (not enriched for phosphopeptides) from each of the four regions of the gel and the top 200 identified proteins were subjected to further analysis.

### Analysis of the Human Embryonic Proteome

Human CSF was collected from the fourth ventricle, as mentioned above, from two independent embryos at Carnegie Stage (CS) 19-20. From the first embryo (CS19) a total of 15 μl was collected, and from the second embryo (CS20) a total of 70 μl was collected. The CSF from these two independent samples was separated by I-D SDS-PAGE; FIG. 1B shows the Coomassie stained protein pattern of the CSF from CS20 and CS19 embryos run on 7.5% and 10% polyacrylamide gels, respectively. The two human e-CSF samples were analyzed separately. Table 1 shows the proteomic analysis of the CSF collected from the CS20 embryo and lists 188 proteins with 2 or more peptides identified. Using a number of protein analysis programs such as UniProt, Gene Ontology™ (GO), and the PANTHER (Protein Analysis Through Evolutionary Relationships) classification system we categorized the proteins found from the mass spectrometry data and list subcellular localization, protein function, tissue specificity, and relevant notes pertaining to each protein (Table 1) (Ballif et al., Mol Cell Proteomics, 2004. 3:1093-101). Analysis of the CSF from the CS19 human sample revealed 772 proteins with more than three peptides identified. The search results from this analysis suggested the presence of a number of non-CSF contaminants including 7 different mitochondrial specific precursor proteins such as the mitochondrial precursors for 4-aminobutyrate aminotransferase, fumarate hydratase, and isoform dut-M of deoxyuridine 5′-triphosphate nucleotidohydrolase, whereas no mitochondrial precursor proteins were identified in the rat CSF or in the CS20 human CSF sample. Therefore, the CS19 list was not further considered in the comparison to rat CSF. However, the proteins from this analysis are presented in Table 2 as this list is certainly enriched for human e-CSF proteins. The substantial differences between this sample and the other human and rat samples suggest that this sample contained multiple impurities, likely from lysed blood and/or neuroepithelial cells. Nonetheless, the differences highlight that the MS analysis is highly sensitive to contaminants, and that the absence of mitochondrial proteins in other samples indicates, that they are probably quite pure.

TABLE 1
Protein list of mass spectrometry analysis of Carnegie Stage 20 embryonic human CSF.

| Number | | | | | | | | | |
|---|---|---|---|---|---|---|---|---|---|
| of unique | Total | | | | | | | | |
| peptides | number | | | | | | | | |
| from | of | Accession | | | Subcellular | | | | Found in Rat |
| protein | peptides | Number | Name of Protein | MW | location | Function | Tissue Specificity | Notes | CSF |
| 206 | 584 | P04114 | APOLIPOPROTEIN B-100 | 515563 | Secreted | Lipid and fatty acid | Plasma | Apo B-100 functions as a recognition | Yes - all rat |
| | | | PRECURSOR | | | transport and | | signal for the cellular binding and | samples |
| | | | | | | metabolism | | internalization of LDL particles by the | |
| | | | | | | | | apoB/E receptor | |
| 43 | 79 | P02751 | ISOFORM 1 OF | 262607 | Secreted, | Cell adhesion, cell | Plasma | Integrin signaling pathway | Yes - all rat |
| | | | FIBRONECTIN | | extracellular | motility, wound | fibronectin made | | samples |
| | | | PRECURSOR | | space, | healing, and | by liver and | | |
| | | | | | extracellular | maintenance of cell | cellular | | |
| | | | | | matrix | shape | fibronectin made | | |
| | | | | | | | by fibroblasts, | | |
| | | | | | | | epithelial and | | |
| | | | | | | | other cell types is | | |
| | | | | | | | deposited in the | | |
| | | | | | | | extracellular | | |
| | | | | | | | matrix | | |
| 42 | 114 | P01023 | ALPHA-2- | 163278 | Secreted | Serine Protease | Plasma | Contrary to the rat protein, which is an | Yes - all rat |
| | | | MACROGLOBULIN | | | Inhibitor | | acute phase protein, this protein is | samples |
| | | | PRECURSOR | | | | | always present at high levels in | |
| | | | | | | | | circulation | |
| 27 | 63 | P02787 | SEROTRANSFERRIN | 77050 | Secreted | Transport, | Plasma | Transport of iron from sites of | Yes - all rat |
| | | | PRECURSOR | | | Transfer/Carrier | | absorption and heme degradation to | samples |
| | | | | | | | | those of storage and utilization. | |
| | | | | | | | | Serum transferrin may also play a role | |
| | | | | | | | | in stimulating cell proliferation | |
| 25 | 91 | Q5D0D7 | ALB (Albumin) PROTEIN | 71705 | Secreted | Transport, | Plasma | Serum albumin, the main plasma | Yes - all rat |
| | | | | | | Transfer/Carrier | | protein, has a good binding capacity | samples |
| | | | | | | | | for water, calcium, sodium, | |
| | | | | | | | | potassium, fatty acids, hormones, | |
| | | | | | | | | bilirubin and drugs. Its main function | |
| | | | | | | | | is the regulation of the colloidal | |
| | | | | | | | | osmotic pressure of blood | |
| 23 | 34 | P01024 | COMPLEMENT | 187306 | Secreted | Complement | Plasma | Plays a central role in the activation of | Yes - all rat |
| | | | COMPONENT 3 | | | mediated immunity | | the complement system | samples |
| | | | PRECURSOR | | | | | | |
| 21 | 87 | P02771 | ALPHA-FETOPROTEIN | 68678 | Secreted | Transport, | Plasma | Binds copper, nickel, and fatty acids | Yes - all rat |
| | | | PRECURSOR | | | Transfer/Carrier | | as well as, and bilirubin less well than, | samples |
| | | | | | | | | serum albumin | |
| 19 | 45 | P19823 | INTER-ALPHA-TRYPSIN | 106436 | Secreted | Serine Protease | Plasma, widely | May act as a carrier of hyaluronan in | Yes - present in |
| | | | INHIBITOR HEAVY CHAIN | | | Inhibitor | distributed | serum or as a binding protein | rat E14.5, |
| | | | H2 PRECURSOR | | | | | between hyaluronan and other matrix | E17.5LV |
| | | | | | | | | protein, including those on cell | |
| | | | | | | | | surfaces in tissues to regulate the | |
| | | | | | | | | localization, synthesis and | |
| | | | | | | | | degradation of hyaluronan which are | |
| | | | | | | | | essential to cells undergoing | |
| | | | | | | | | biological processes. | |
| 14 | 14 | P49327 | FATTY ACID SYNTHASE | 273400 | Cytoplasm | Lipid and fatty acid | Ubiquitous. | Involved in catalyzing the formation of | Yes - all rat |
| | | | | | | biosynthesis | Prominent | long chain fatty acids | samples |
| | | | | | | | expression in | | |
| | | | | | | | brain, lung, and | | |
| | | | | | | | liver | | |
| 14 | 15 | P24821 | ISOFORM 1 OF | 240866 | Secreted, | Cell adhesion, | Widely | Extracellular matrix protein implicated | Yes - all rat |
| | | | TENASCIN PRECURSOR | | extracellular | extracellular matrix | distributed | in guidance of migrating neurons as | samples |
| | | | | | space, | glycoprotein- | | well as axons during development, | |
| | | | | | extracellular | mediated signaling | | synaptic plasticity as well as neuronal | |
| | | | | | matrix | | | regeneration. Ligand for integrin | |
| | | | | | | | | receptors. | |
| 13 | 25 | P19827 | INTER-ALPHA-TRYPSIN | 101389 | Secreted | Protease Inhibitor | Plasma | See notes on INTER-ALPHA- | Similar to inter- |
| | | | INHIBITOR HEAVY CHAIN | | | | | TRYPSIN INHIBITOR HEAVY CHAIN | alpha-inhibitor |
| | | | H1 PRECURSOR | | | | | H2 PRECURSOR above. | H4 heavy chain |
| 12 | 17 | P01031 | COMPLEMENT C5 | 188331 | Secreted | Complement | Plasma | Activation of C5 by a C5 convertase | Yes - present in |
| | | | PRECURSOR | | | mediated immunity | | initiates the spontaneous assembly of | E17.5LV |
| | | | | | | | | the late complement components, C5-C9, | |
| | | | | | | | | into the membrane attack | |
| | | | | | | | | complex. | |
| 12 | 19 | P02671 | ISOFORM 1 OF | 94973 | Secreted | Blood clotting | Plasma | Monomers polymerize into fibrin and | Yes - all rat |
| | | | FIBRINOGEN ALPHA | | | | | also acts as a cofactor in platelet | samples |
| | | | CHAIN PRECURSOR | | | | | aggregation. | |
| 11 | 27 | P02679 | ISOFORM GAMMA-B OF | 51512 | Secreted | Blood clotting | Plasma | Monomers polymerize into fibrin and | Yes - all rat |
| | | | FIBRINOGEN GAMMA | | | | | also acts as a cofactor in platelet | samples |
| | | | CHAIN PRECURSOR | | | | | aggregation. | |
| 11 | 12 | P12111 | ALPHA 3 TYPE VI | 343669 | Secreted, | Cell adhesion - Cell | Widley | Collagen VI acts as a cell-binding | Yes - present in |
| | | | COLLAGEN ISOFORM 1 | | extracellular | structure | distributed, | protein | rat E14.5, |
| | | | PRECURSOR | | space, | | expressed in | | E17.5LV |
| | | | | | extracellular | | muscles | | |
| | | | | | matrix | | | | |
| 10 | 19 | P02647 | APOLIPOPROTEIN A-I | 30778 | Secreted | Lipid and fatty acid | Major protein of | Participates in the reverse transport of | Yes - all rat |
| | | | PRECURSOR | | | transport and | plasma HDL, | cholesterol from tissues to the liver for | samples |
| | | | | | | metabolism | also found in | excretion by promoting cholesterol | |
| | | | | | | | chylomicrons. | efflux from tissues. | |
| 10 | 13 | P06396 | ISOFORM 1 OF | 85698 | Secreted | Actin remodeling - | Plasma | Calcium-regulated, actin-modulating | Yes - all rat |
| | | | GELSOLIN PRECURSOR | | | Cell structure | | protein. It can promote the assembly | samples |
| | | | | | | | | of monomers into filaments as well as | |
| | | | | | | | | sever filaments already formed. May | |
| | | | | | | | | be involved in myelination. | |
| 10 | 16 | P05155 | PLASMA PROTEASE C1 | 55154 | Secreted | Serine Protease | Plasma | May regulate complement activation, | Yes - all rat |
| | | | INHIBITOR PRECURSOR | | | Inhibitor | | blood coagulation, fibrinolysis and the | samples |
| | | | | | | | | generation of kinins. | |
| 10 | 12 | P0C0L4 | COMPLEMENT C4-A | 192771 | Secreted | Complement | Plasma | Inflammatory response | Yes - all rat |
| | | | PRECURSOR | | | mediated immunity | | | samples |
| 10 | 10 | Q13822 | ISOFORM 1 OF | 99004 | Membrane | Hydrolase | Predominantly | Localized in secretory epithelial cells | Yes - all rat |
| | | | ECTONUCLEOTIDE | | | | expressed in | in the brain and the eye including | samples |
| | | | PYROPHOSPHATASE/ | | | | brain, placenta, | choroid plexus epithelial cells, ciliary | |
| | | | PHOSPHODIESTERASE 2 | | | | ovary, and small | epithelial cells, iris pigment epithelial | |
| | | | | | | | intestine. | cells, and retinal pigment cells. Has a | |
| | | | | | | | | potent tumor cell motility-stimulating | |
| | | | | | | | | activity. | |
| 9 | 16 | P02675 | FIBRINOGEN BETA | 55928 | Secreted | Blood clotting | Plasma | Monomers polymerize into fibrin and | Yes - all rat |
| | | | CHAIN PRECURSOR | | | | | also acts as a cofactor in platelet | samples |
| | | | | | | | | aggregation. | |
| 9 | 22 | P02766 | TRANSTHYRETIN | 15887 | Secreted | Hormone transport | Most abundant in | Thyroid hormone-binding protein. | Yes - all rat |
| | | | PRECURSOR | | | | the choroid | Probably transports thyroxine from the | samples |
| | | | | | | | plexus. Also | bloodstream to the brain. | |
| | | | | | | | present in the | | |
| | | | | | | | liver | | |
| 8 | 17 | P01009 | ALPHA-1-ANTITRYPSIN | 46737 | Secreted | Serine Protease | Plasma | Belongs to the serpin family | Yes - all rat |
| | | | PRECURSOR | | | Inhibitor | | | samples |
| 8 | 21 | P02760 | AMBP PROTEIN | 38999 | Secreted | Serine Protease | Plasma, urine, | It appears not only as a free monomer | Yes - all rat |
| | | | PRECURSOR | | | Inhibitor | and | but also in complexes with IgA and | samples |
| | | | | | | | cerebrospinal | albumin. | |
| | | | | | | | fluid | | |
| 8 | 11 | P23471 | ISOFORM SHORT OF | 163444 | Membrane | Cell surface | Central nervous | May be involved in the regulation of | Yes - all rat |
| | | | RECEPTOR-TYPE | | | receptor mediated | system | specific developmental processes in | samples |
| | | | TYROSINE-PROTEIN | | | signal transduction, | | the CNS | |
| | | | PHOSPHATASE ZETA | | | transmembrane | | | |
| | | | PRECURSOR | | | receptor protein | | | |
| | | | | | | tyrosine | | | |
| | | | | | | phosphatase activity | | | |
| 8 | 8 | P08238 | HYPOTHETICAL | 84843 | Cytoplasm | Chaperone, protein | Ubiquitous | Belongs to the heat shock protein 90 | Yes - all rat |
| | | | PROTEIN | | | folding, stress | | family | samples |
| | | | DKFZP761K0511 - heat | | | response | | | |
| | | | shock 90 kDa protein 1, | | | | | | |
| | | | beta | | | | | | |
| 8 | 15 | Q9P173 | PRO2275 - Serpin | 13097 | Secreted | Serine Protease | Plasma | Belongs to the serpin family | |
| | | | peptidase inhibitor, clade A | | | Inhibitor - Blood | | | |
| | | | (alpha-1 antiproteinase, | | | coagulation | | | |
| | | | antitrypsin), member 1 | | | | | | |
| 7 | 9 | P00450 | CERULOPLASMIN | 122205 | Secreted | Transport, | Plasma | It is involved in iron transport across | Yes - similar to |
| | | | PRECURSOR | | | Transfer/Carrier, | | the cell membrane, and metal ion | GPI anchored |
| | | | | | | Oxidoreductase | | oxidoreductase activity | ceruloplamsin |
| | | | | | | | | | present in all rat |
| | | | | | | | | | samples |
| 7 | 8 | N/A | 45 KDA PROTEIN - | 44847 | Secreted | Transport, Transfer | Plasma, widely | Involved in phospholipid transfer in | Yes - all rat |
| | | | Homologous to | | | Carries | distributed | the serum. | samples |
| | | | Phospholipid transfer | | | | | | |
| | | | protein | | | | | | |
| 7 | 10 | P19022 | CADHERIN-2 | 99851 | Membrane | Cell adhesion | Widely | May be involved in neuronal tissue | Yes - neural |
| | | | PRECURSOR (Neuronal | | | | distributed | recognition | cadherin |
| | | | cadherin) | | | | | | precursor |
| | | | | | | | | | present in all rat |
| | | | | | | | | | samples |
| 7 | 7 | P78527 | ISOFORM 1 OF DNA- | 469089 | Nucleus | Serine/threonine- | Ubiquitous | Molecular sensor for DNA damage | |
| | | | DEPENDENT PROTEIN | | | protein kinase | | | |
| | | | KINASE CATALYTIC | | | | | | |
| | | | SUBUNIT | | | | | | |
| 7 | 7 | Q5CAQ7 | HEAT SHOCK PROTEIN | 98113 | Cytoplasm | Molecular | Ubiquitous | Belongs to the heat shock protein 90 | Yes - HSP 90 |
| | | | HSP 90-ALPHA 2 | | | chaperone, protein | | family | family present in |
| | | | | | | folding, stress | | | all rat samples |
| | | | | | | response | | | |
| 7 | 7 | P35580 | MYOSIN-10 | 228939 | Intracellular | Cell structure, cell | Brain | Actin binding motor protein | Yes - all rat |
| | | | | | | motility | | | samples |
| 7 | 16 | P20742 | PREGNANCY ZONE | 163836 | Secreted | Protease Inhibitor | Plasma | Belongs to the protease inhibitor I39 | Yes - alpha-1- |
| | | | PROTEIN PRECURSOR | | | | | (alpha-2-macroglobulin) family | macroglobulin |
| | | | | | | | | | present in all rat |
| | | | | | | | | | samples |
| 6 | 10 | P02649 | APOLIPOPROTEIN E | 36154 | Secreted | Lipid and fatty acid | Plasma | Mediates the binding, internalization, | Yes - all rat |
| | | | PRECURSOR | | | transport and | | and catabolism of lipoprotein | samples |
| | | | | | | metabolism | | particles. | |
| 6 | 6 | P46821 | MICROTUBULE- | 270620 | Intracellular | Cell structure | Brain | The function of brain MAPS is | Yes - present in |
| | | | ASSOCIATED PROTEIN | | | | | unknown. Phosphorylated MAP1B | rat E12.5LV, |
| | | | 1B | | | | | may play a role in the cytoskeletal | E17.5LV |
| | | | | | | | | changes that accompany neurite | |
| | | | | | | | | extension. | |
| 6 | 6 | P06681 | COMPLEMENT C2 | 83268 | Secreted | Serine protease, | Plasma | Belongs to the peptidase S1 family. | Yes - all rat |
| | | | PRECURSOR | | | complement- | | | samples |
| | | | (FRAGMENT) | | | mediated immunity | | | |
| 6 | 6 | P11047 | LAMININ GAMMA-1 | 177607 | Secreted, | Extracellular matrix | Basement | Binds to cells and is thought to | Yes - present in |
| | | | CHAIN PRECURSOR | | extracellular | linker protein- | membranes | mediate the attachment, migration | rat E12.5LV, |
| | | | (Laminin B2 chain) | | space, | mediated signaling | | and organization of cells into tissues | E14.5 |
| | | | | | extracellular | | | during embryonic development by | |
| | | | | | matrix | | | interacting with other extracellular | |
| | | | | | | | | matrix components. | |
| 6 | 7 | P01019 | ANGIOTENSINOGEN | 53154 | Secreted | Serine Protease | Plasma | Belongs to the serpin family. Helps | Yes - all rat |
| | | | PRECURSOR | | | Inhibitor | | regulate volume and mineral balance | samples |
| | | | | | | | | of body fluids. | |
| 6 | 7 | P33151 | CADHERIN-5 | 87516 | Membrane | Cell adhesion | Endothelial | Plays a role in endothelial adhesion. | Yes - all rat |
| | | | PRECURSOR | | | | tissues and brain | | samples |
| 6 | 7 | P98160 | BASEMENT MEMBRANE- | 468825 | Secreted, | Cell adhesion | Widely | Integral component of basement | Yes - all rat |
| | | | SPECIFIC HEPARAN | | extracellular | | distributed in | membranes. | samples |
| | | | SULFATE | | space, | | various tissues | | |
| | | | PROTEOGLYCAN CORE | | extracellular | | | | |
| | | | PROTEIN PRECURSOR | | matrix | | | | |
| | | | (Perlecan) | | | | | | |
| 6 | 10 | O00468 | AGRIN PRECURSOR | 214889 | Secreted, | Cell adhesion | Embryonic | Component of the basal lamina that | Yes - all rat |
| | | | | | extracellular | mediated signaling | nervous system | binds to laminin. | samples |
| | | | | | space, | | and muscle. | | |
| | | | | | extracellular | | | | |
| | | | | | matrix | | | | |
| 5 | 7 | Q08380 | GALECTIN-3-BINDING | 65331 | Secreted, | Intergrin-mediated | Ubiquitous. | May stimulate host defense against | |
| | | | PROTEIN PRECURSOR | | extracellular | cell adhesion | Detected in body | viruses and tumor cells. | |
| | | | | | space, | | fluids such as | | |
| | | | | | extracellular | | semen, milk, | | |
| | | | | | matrix | | serum, tears, | | |
| | | | | | | | saliva and urine. | | |
| 5 | 5 | Q14624 | ISOFORM 2 OF INTER- | 101242 | Secreted | Protease Inhibitor | Plasma | May be involved in acute phase | Yes - all rat |
| | | | ALPHA-TRYPSIN | | | | | reactions. | samples |
| | | | INHIBITOR HEAVY CHAIN | | | | | | |
| | | | H4 PRECURSOR | | | | | | |
| 5 | 5 | P35579 | MYOSIN-9 | 226401 | Intracellular | Cytokinesis, cell | In the kidney, | Non-muscle myosin involved in a | Yes - present in |
| | | | | | | shape, and | expressed in the | number of cellular functions. | 12.5LV, |
| | | | | | | specialized | glomeruli. Also | | E14.5LV, |
| | | | | | | functions such as | expressed in | | E17.5LV |
| | | | | | | secretion and | leukocytes. | | |
| | | | | | | capping | | | |
| 5 | 7 | P04217 | ALPHA-1B- | 54273 | Secreted | Function unknown | Plasma | Immunoglobulin domain glycoprotein | Yes - present in |
| | | | GLYCOPROTEIN | | | | | | E12.5LV, E14.5 |
| | | | PRECURSOR | | | | | | |
| 5 | 5 | P13611 | ISOFORM V0 OF | 372820 | Secreted, | Extracellular matrix | Brain | Also known as chondroitin sulfate | Yes - all rat |
| | | | VERSICAN CORE | | extracellular | protein-mediated | | proteoglycan core protein 2 | samples |
| | | | PROTEIN PRECURSOR | | space, | signaling, cell | | | |
| | | | | | extracellular | adhesion, cell | | | |
| | | | | | matrix | motility | | | |
| 5 | 7 | P23142 | ISOFORM B OF FIBULIN-1 | 77186 | Secreted, | Cell adhesion, cell | Widely | Incorporated into fibronectin- | Yes - all rat |
| | | | PRECURSOR | | extracellular | motility | expressed during | containing matrix fibers. May play a | samples |
| | | | | | space, | | embryonic | role in cell adhesion and migration | |
| | | | | | extracellular | | development. | along protein fibers within the | |
| | | | | | matrix | | | extracellular matrix. Could play a | |
| | | | | | | | | significant role in modulating the | |
| | | | | | | | | neurotrophic activities of APP, | |
| | | | | | | | | particularly soluble APP. | |
| 5 | 7 | P36955 | PIGMENT EPITHELIUM- | 46342 | Secreted | Serpin family | Retinal pigment | Pigment epithelium-derived factor, a | |
| | | | DERIVED FACTOR | | | member - | epithelial cells, | neurotrophic protein, is a member | |
| | | | PRECURSOR (PEDF) | | | neurotrophic | adult murine | serpin that has been shown to | |
| | | | | | | properties, | SVZ cells, and | promote the survival and/or | |
| | | | | | | antiangiogenic | blood plasma | differentiation of rat cerebellar granule | |
| | | | | | | | | neurons and human retinoblastoma | |
| | | | | | | | | cells in vitro. Also PEDF was shown | |
| | | | | | | | | to prevent the death and atrophy of | |
| | | | | | | | | spinal motor neurons in the | |
| | | | | | | | | developing neonatal mouse after | |
| | | | | | | | | axotomy. PEDF is also secreted by | |
| | | | | | | | | retinal pigment epithelial cells into the | |
| | | | | | | | | interphotoreceptor matrix, where it | |
| | | | | | | | | acts on photoreceptor cells. | |
| 5 | 6 | P55287 | ISOFORM 2 OF | 76541 | Membrane | Cell adhesion | Expressed | Also known as osteoblast-cadherin | Yes - all rat |
| | | | CADHERIN-11 | | | | mainly in brain | | samples |
| | | | PRECURSOR | | | | but also found in | | |
| | | | | | | | other tissues. | | |
| | | | | | | | Expressed in | | |
| | | | | | | | neuroblasts | | |
| 5 | 5 | P02008 | HEMOGLOBIN SUBUNIT | 15506 | Intracellular | Oxygen Transport, | Red blood cells | The zeta chain is an alpha-type chain | |
| | | | ZETA | | | Transfer - Carrier | | of mammalian embryonic hemoglobin, | |
| | | | | | | Protein | | synthesized primarily in the yolk sac. | |
| 5 | 5 | O00533 | ISOFORM 2 OF NEURAL | 136654 | Membrane | Extracellular matrix | Expressed in the | Plays a role in nervous system | |
| | | | CELL ADHESION | | | protein-mediated | fetal and adult | development. Plays important roles in | |
| | | | MOLECULE L1-LIKE | | | signaling, cell | brain | neurite outgrowth and neuronal | |
| | | | PROTEIN PRECURSOR | | | adhesion, cell | | survival. | |
| | | | | | | motility | | | |
| 5 | 6 | P48681 | NESTIN | 176706 | Intracellular | Cell structure | CNS stem cells. | Upon terminal neural differentiation, | Yes - present in |
| | | | | | | | | nestin is down-regulated and replaced | E12.5LV, |
| | | | | | | | | by neurofilaments. | E14.5LV, |
| | | | | | | | | | E17.5LV |
| 5 | 7 | P00751 | ISOFORM 1 OF | 85533 | Secreted | Serine Protease - | Plasma | Hydrolase, Peptidase | Yes - all rat |
| | | | COMPLEMENT FACTOR | | | Complement | | | samples |
| | | | B PRECURSOR | | | Mediated Immunity | | | |
| | | | (FRAGMENT) | | | | | | |
| 4 | 5 | Q00839 | ISOFORM SHORT OF | 88814 | Nucleus. Cell | DNA and RNA | Ubiquitous | Invovled in RNA splicing | Yes - all rat |
| | | | HETEROGENEOUS | | surface | binding and RNA | | | samples |
| | | | NUCLEAR | | | processing. | | | |
| | | | RIBONUCLEOPROTEIN U | | | | | | |
| 4 | 4 | P12277 | CREATINE KINASE B- | 42644 | Membrane | Kinase, energy | Ubiquitous | Plays a central role in tissues with | Yes - all rat |
| | | | TYPE | | | modulation | | variable energy demands. | samples |
| 4 | 4 | P34932 | HEAT SHOCK 70 KDA | 94300 | Intracellular - | Heat shock, protein | Ubiquitous | Heat shock protein 70 family | Yes - all rat |
| | | | PROTEIN 4 | | Cytoplasm | folding, stress | | | samples |
| | | | | | | response | | | |
| 4 | 4 | P13010 | ATP-DEPENDENT DNA | 82573 | Nucleus | Single stranded | Ubiquitous | Plays a role in chromosome | |
| | | | HELICASE 2 SUBUNIT 2 | | | ATP-dependent | | translocation. Involved in DNA | |
| | | | | | | DNA helicase | | nonhomologous end joining required | |
| | | | | | | activity | | for double-strand break repair and | |
| | | | | | | | | V(D)J recombination. | |
| 4 | 4 | Q9BQ02 | NCL (Nucleolin) PROTEIN | 51641 | Nucleus | Major nucleolar | Ubiquitous | It is thought to play a role in pre-rRNA | Yes - present in |
| | | | | | | protein of growing | | transcription and ribosome assembly | E12.5LV, |
| | | | | | | eukaryotic cells. | | | E17.5LV |
| | | | | | | Nucleotide binding | | | |
| 4 | 4 | P49321 | ISOFORM 1 OF NUCLEAR | 85238 | Intracellular - | Nuclear-cytoplasmic | Testis and | Required for DNA replication, normal | Yes - all rat |
| | | | AUTOANTIGENIC SPERM | | Nucleus, | shuttling | Sperm | cell cycle progression and cell | samples |
| | | | PROTEIN | | Cytoplasm | | | proliferation. | |
| 4 | 10 | P07477 | TRYPSIN PRECURSOR | 26558 | Secreted, | Serine protease | Ubiquitous | Belongs to the peptidase S1 family | |
| | | | (EC | | extracellular | | | | |
| | | | 3.4.21.4) > PIR1:TRPGTR | | space, | | | | |
| | | | trypsin (EC 3.4.21.4) | | extracellular | | | | |
| | | | | | matrix | | | | |
| 4 | 7 | Q7KZ97 | ANTITHROMBIN III | 52692 | Secreted | Serine Protease | Plasma | Regulates the blood coagulation | |
| | | | VARIANT | | | Inhibitor | | cascade. Belongs to the serpin family. | |
| 4 | 9 | P04004 | VITRONECTIN | 54306 | Secreted, | Cell adhesion | Plasma | Vitronectin is an abundant | |
| | | | PRECURSOR | | extracellular | | | glycoprotein found in blood plasma | |
| | | | | | space, | | | and the extracellular matrix. It | |
| | | | | | extracellular | | | regulates proteolysis initiated by | |
| | | | | | matrix | | | plasminogen activation. It is | |
| | | | | | | | | recognized by certain members of the | |
| | | | | | | | | integrin family and serves as a cell-to- | |
| | | | | | | | | substrate adhesion molecule. | |
| 4 | 4 | Q99435 | PROTEIN KINASE C- | 91346 | Secreted | Cell communication, | Widely | Regulation of growth and | Yes - all rat |
| | | | BINDING PROTEIN NELL2 | | | Cell adhesion, Cell | distributed | neurogenesis | samples |
| | | | PRECURSOR | | | structure | | | |
| 4 | 4 | P43146 | NETRIN RECEPTOR DCC | 158457 | Membrane | Ligand mediated | Axons of the | Axon guidance and neuronal growth | Yes - all rat |
| | | | PRECURSOR | | | signaling, cell | central and | cone growth | samples |
| | | | | | | adhesion | peripheral | | |
| | | | | | | | nervous system | | |
| | | | | | | | and in | | |
| | | | | | | | differentiated cell | | |
| | | | | | | | types of the | | |
| | | | | | | | intestine | | |
| 4 | 4 | Q02246 | CONTACTIN-2 | 113393 | Membrane; | Cell Adhesion - | Highly expressed | May play a role in the initial growth | Yes - all rat |
| | | | PRECURSOR | | (attached to the | Neurogenesis | in brain | and guidance of axons. Belongs to | samples |
| | | | | | neuronal | | | the immunoglobulin superfamily. | |
| | | | | | membrane by a | | | | |
| | | | | | GPI-anchor and | | | | |
| | | | | | is also released | | | | |
| | | | | | from neurons) | | | | |
| 4 | 5 | O75882 | ISOFORM 1 OF | 158537 | Membrane | Protease involved in | Secreted by | May regulate chemotactic activity of | Yes - all rat |
| | | | ATTRACTIN | | | cell clustering | activated T | chemokines. Has a critical role in | samples |
| | | | PRECURSOR | | | during inflammatory | lymphocytes. | normal myelination in the central | |
| | | | | | | response | Also expressed | nervous system. | |
| | | | | | | | in peripheral | | |
| | | | | | | | blood leukocytes. | | |
| | | | | | | | spleen, lymph | | |
| | | | | | | | node, tonsil, | | |
| | | | | | | | bone marrow | | |
| | | | | | | | and fetal liver. | | |
| 4 | 6 | Q96PQ8 | FACTOR VII ACTIVE SITE | 75553 | Secreted | Serine Protease - | Plasma | Involved in blood coagulation, | |
| | | | MUTANT | | | Blood clotting | | activated by Factor Xa | |
| | | | IMMUNOCONJUGATE | | | | | | |
| 4 | 5 | O00391 | QUIESCIN Q6 ISOFORM A | 82578 | Membrane | Oxidase | Widely | May contribute to disulfide bond | Yes - all rat |
| | | | | | | | distributed, | formation in a variety of secreted | samples |
| | | | | | | | expressed in | proteins. Induced in quiescent cells. | |
| | | | | | | | heart, placenta, | | |
| | | | | | | | lung, liver, | | |
| | | | | | | | skeletal muscle, | | |
| | | | | | | | pancreas and | | |
| | | | | | | | very weakly in | | |
| | | | | | | | brain and kidney | | |
| 4 | 7 | P02461 | COLLAGEN ALPHA-1(III) | 138555 | Secreted, | Cell adhesion - Cell | Widely | Present in most soft connective tissue | Yes - all rat |
| | | | CHAIN PRECURSOR | | extracellular | structure | distributed, | | samples |
| | | | | | space, | | highly expressed | | |
| | | | | | extracellular | | in skin, lungs, | | |
| | | | | | matrix | | intestinal walls, | | |
| | | | | | | | and blood | | |
| | | | | | | | vessels | | |
| 4 | 6 | Q4KKU6 | DESMOGLEIN 2 | 122294 | Membrane | Cell adhesion | Widley | Component of desmosomes | |
| | | | | | | | distributed | | |
| 4 | 4 | Q92945 | FAR UPSTREAM | 72709 | Intracellular - | RNA binding, RNA | Detected in | | Yes - present in |
| | | | ELEMENT-BINDING | | Nucleus, | processing and may | neural and non- | | E12.5LV, |
| | | | PROTEIN 2 | | Cytoplasm | play a role in mRNA | neural cell lines. | | E14.5LV, |
| | | | | | | trafficking | | | E17.5LV |
| 4 | 7 | P05546 | HEPARIN COFACTOR 2 | 60178 | Secreted | Thrombin inhibitor | Expressed | Belongs to the serpin family. | Yes - present in |
| | | | PRECURSOR | | | involved in blood | predominantly in | | E14.5 4thV, |
| | | | | | | clotting - Serine | liver. | | E17.5LV |
| | | | | | | Protease Inhibitor | | | |
| 4 | 4 | Q5HY53 | FILAMIN A, ALPHA | 280018 | Intracellular | Cell structure, cell | Ubiquitous | Actin binding protein | Yes - all rat |
| | | | | | | motility | | | samples |
| 4 | 4 | Q86VP6 | ISOFORM 1 OF CULLIN- | 136376 | Nucleus | Transcriptional | Widely | Down-regulates ubiquitination of | Yes - all rat |
| | | | ASSOCIATED NEDD8- | | | Enhancer | distributed, | target proteins. | samples |
| | | | DISSOCIATED PROTEIN 1 | | | | highly expressed | | |
| | | | | | | | in lung | | |
| | | | | | | | fibroblasts | | |
| 4 | 5 | Q92859 | ISOFORM 1 OF | 159959 | Membrane | Cell adhesion | Widely | May be involved as a regulatory | Yes - all rat |
| | | | NEOGENIN PRECURSOR | | | | expressed | protein in the transition of | samples |
| | | | | | | | | undifferentiated proliferating cells to | |
| | | | | | | | | their differentiated state. Belongs to | |
| | | | | | | | | the immunoglobulin superfamily. | |
| 4 | 4 | P00747 | PLASMINOGEN | 90569 | Secreted | Protease | Present in | Plasmin dissolves the fibrin of blood | Yes - all rat |
| | | | PRECURSOR | | | | plasma and | clots and acts as a proteolytic factor in | samples |
| | | | | | | | many other | a variety of other processes including | |
| | | | | | | | extracellular | embryonic development, tissue | |
| | | | | | | | fluids | remodeling, tumor invasion, and | |
| | | | | | | | | inflammation. | |
| 3 | 8 | P02753 | PLASMA RETINOL- | 23010 | Secreted | Vitamin/Co-factor | Plasma | Delivers retinol from the liver stores to | Yes - all rat |
| | | | BINDING PROTEIN | | | transport | | the peripheral tissues. In plasma, the | samples |
| | | | PRECURSOR | | | | | RBP-retinol complex interacts with | |
| | | | | | | | | transthyretin, this prevents its loss by | |
| | | | | | | | | filtration through the kidney glomeruli. | |
| 3 | 4 | P06727 | APOLIPOPROTEIN A-IV | 45399 | Secreted | Lipid and fatty acid | Plasma | May have a role in chylomicrons and | Yes - all rat |
| | | | PRECURSOR | | | transport and | | VLDL secretion and catabolism. | samples |
| | | | | | | metabolism | | Apoa-IV is a major component of HDL | |
| | | | | | | | | and chylomicrons. | |
| 3 | 3 | O14980 | EXPORTIN-1 | 123386 | Intracellular - | Nuclear export of | Ubiquitous | Mediates nuclear export signal | Yes - present in |
| | | | | | Nucleus, | cellular proteins and | | dependent protein transport | E12.5LV, |
| | | | | | Cytoplasm | RNAs | | | E14.5LV, |
| | | | | | | | | | E17.5LV |
| 3 | 3 | P20908 | COLLAGEN ALPHA-1(V) | 183560 | Secreted, | Cell adhesion - Cell | Ubiquitous | Component of connective tissue | Yes - all rat |
| | | | CHAIN PRECURSOR | | extracellular | structure | | | samples |
| | | | | | space, | | | | |
| | | | | | extracellular | | | | |
| | | | | | matrix | | | | |
| 3 | 3 | P07942 | LAMININ BETA-1 CHAIN | 198066 | Secreted, | Extracellular matrix | Widely | Is thought to mediate the attachment, | Yes - all rat |
| | | | PRECURSOR | | extracellular | linker protein- | distributed in | migration and organization of cells | samples |
| | | | | | space, | mediated signaling | basement | into tissues during embryonic | |
| | | | | | extracellular | | membranes | development by interacting with other | |
| | | | | | matrix | | | extracellular matrix components. | |
| 3 | 3 | Q14974 | IMPORTIN BETA-1 | 97170 | Intracellular - | Nuclear protein | Ubiquitous | Import of proteins with nuclear | Yes - present in |
| | | | SUBUNIT | | Nucleus, | import | | localization signal | E12.5LV, |
| | | | | | Cytoplasm | | | | E14.5LV, |
| | | | | | | | | | E17.5LV |
| 3 | 3 | P23528 | COFILIN-1 | 18371 | Intracellular - | Cell structure - | Widely | Controls actin polymerization and | Yes - present in |
| | | | | | Nucleus, | cytoskeleton | distributed in | depolymerization. A major | E12.5LV, |
| | | | | | Cytoplasm | | various tissues | component of intranuclear and | E14.5LV, |
| | | | | | | | | cytoplasmic actin rods. | E17.5LV |
| 3 | 3 | P29966 | MYRISTOYLATED | 31423 | Intracellular | Actin binding, actin | Ubiquitous | MARCKS is the most prominent | |
| | | | ALANINE-RICH C-KINASE | | | cytoskeleton, cell | | cellular substrate for protein kinase C. | |
| | | | SUBSTRATE | | | structure | | MARCKS is a filamentous (F) actin | |
| | | | | | | | | cross-linking protein. | |
| 3 | 3 | Q5JRR8 | UBIQUITIN-ACTIVATING | 117849 | Intracellular | Protein modification | Ubiquitous | Involved with ubiquitin conjugation by | Yes - all rat |
| | | | ENZYME E1 | | | | | activating ubiquitin | samples |
| 3 | 3 | P09874 | POLY [ADP-RIBOSE] | 112953 | Nucleus | DNA repair | Ubiquitous | Involved in the base excision repair | Yes - present in |
| | | | POLYMERASE 1 | | | | | pathway | E12.5LV, |
| | | | | | | | | | E17.5LV |
| 3 | 3 | P55072 | TRANSITIONAL | 89191 | Intracellular - | Protein targeting | Ubiquitous | Necessary for the fragmentation of | Yes - all rat |
| | | | ENDOPLASMIC | | Nucleus, | and localization, | | Golgi stacks during mitosis and for | samples |
| | | | RETICULUM ATPASE | | Cytoplasm | intracellular protein | | their reassembly after mitosis. | |
| | | | | | | traffic | | Involved in the formation of the | |
| | | | | | | | | transitional endoplasmic reticulum. | |
| 3 | 3 | Q9NZ08 | TYPE 1 TUMOR | 107841 | Secreted | Protease | Ubiquitous | May play a role in the inactivation of | |
| | | | NECROSIS FACTOR | | | | | peptide hormones. May be involved in | |
| | | | RECEPTOR SHEDDING | | | | | the regulation of blood pressure. | |
| | | | AMINOPEPTIDASE | | | | | | |
| | | | REGULATOR ISOFORM A | | | | | | |
| 3 | 3 | P00736 | COMPLEMENT C1R | 80174 | Secreted | Protease - | Plasma | Classical pathway of the complement | |
| | | | SUBCOMPONENT | | | complement | | system | |
| | | | PRECURSOR | | | mediated immunity | | | |
| 3 | 3 | Q7Z7M0 | ISOFORM 1 OF | 254573 | Membrane | Cell adhesion, cell | Widely | Also known as Multiple EGF like | |
| | | | MULTIPLE EPIDERMAL | | | structure | distributed | domain protein 4 | |
| | | | GROWTH FACTOR-LIKE | | | | | | |
| | | | DOMAINS 8 | | | | | | |
| 3 | 3 | P04406 | GLYCERALDEHYDE-3- | 35922 | Cytoplasm | Glycolysis, | Ubiquitous | | Yes - present in |
| | | | PHOSPHATE | | | carbohydrate | | | E12.5LV, |
| | | | DEHYDROGENASE | | | degradation | | | E17.5LV |
| 3 | 3 | P22626 | ISOFORM B1 OF | 37430 | Nucleus | mRNA processing | Ubiquitous | Forms ribonucleosome complexes | Yes - present in |
| | | | HETEROGENEOUS | | | | | | E14.5LV |
| | | | NUCLEAR | | | | | | |
| | | | RIBONUCLEOPROTEINS | | | | | | |
| | | | A2/B1 | | | | | | |
| 3 | 3 | P10909 | CLUSTERIN | 52495 | Secreted | Function unclear; | Ubiquitous | Seems to be able to bind to cells, | Yes - present in |
| | | | PRECURSOR | | | has been implicated | | membranes and hydrophobic | E14.5, E17.5LV |
| | | | | | | in apoptosis | | proteins. Has been implicated in | |
| | | | | | | | | membrane lipid recycling, in apoptotic | |
| | | | | | | | | cell death, and as a stress-induced | |
| | | | | | | | | secreted chaperone protein. | |
| 3 | 4 | Q86XC7 | 127 KDA PROTEIN - RAN | 126522 | Intracellular - | Nuclear-cytoplasmic | Ubiquitous | Involved in the import of proteins with | Yes - one |
| | | | binding protein 5 | | Nucleus, | transport | | the nuclear localization signal. | isoform found in |
| | | | | | Cytoplasm | | | | E12.5LV, |
| | | | | | | | | | E14.5LV, |
| | | | | | | | | | E17.5LV, 2nd |
| | | | | | | | | | isoform found in |
| | | | | | | | | | E12.5LV, E14.5 |
| | | | | | | | | | 4thV, and |
| | | | | | | | | | E17.5LV |
| 3 | 4 | Q14568 | HEAT SHOCK PROTEIN | 35674 | Cytoplasm | Molecular | Ubiquitous | Heat shock protein family | Yes - all rat |
| | | | 86 (FRAGMENT) | | | chaperone, protein | | | samples |
| | | | | | | folding, stress | | | |
| | | | | | | response | | | |
| 3 | 5 | P02765 | ALPHA-2-HS- | 39325 | Secreted, | Cysteine protease | Synthesized in | Extracellular matrix glycoprotein | Yes - present in |
| | | | GLYCOPROTEIN | | extracellular | inhibitor | liver and | involved in skeletal development | E14.5, E17.5LV |
| | | | PRECURSOR | | space, | | concentrated in | | |
| | | | | | extracellular | | bone. Secreted | | |
| | | | | | matrix | | in plasma. | | |
| 3 | 3 | Q14403 | GAMMA-G GLOBIN | 16969 | Intracellular | Oxygen Transport, | Red blood cells | Belongs to the globin family | |
| | | | (FRAGMENT) | | | Transfer - Carrier | | | |
| | | | | | | Protein | | | |
| 3 | 3 | P00734 | PROTHROMBIN | 70037 | Secreted | Blood clotting | Expressed by the | Thrombin, converts fibrinogen to fibrin | Yes - present in |
| | | | PRECURSOR | | | | liver and | and activates factors V, VII, VIII, XIII, | E14.5 4thV, |
| | | | (FRAGMENT) | | | | secreted in | and, in complex with thrombomodulin. | E17.5LV |
| | | | | | | | plasma | | |
| 3 | 3 | Q16706 | ALPHA-MANNOSIDASE 2 | 131084 | Golgi apparatus | Glycosidase | Ubiquitous | Involved in N-glycosylation | Yes - all rat |
| | | | | | | | | | samples |
| 3 | 3 | Q9Y6N7 | ISOFORM 1 OF | 180930 | Membrane | Axon guidance | Widely | Receptor for SLIT1 and SLIT2 which | Yes - present in |
| | | | ROUNDABOUT | | | receptor | expressed | are thought to act as molecular | E14.5 4thV |
| | | | HOMOLOG 1 | | | | | guidance cue in cellular migration, | |
| | | | PRECURSOR | | | | | including axonal navigation at the | |
| | | | | | | | | ventral midline of the neural tube and | |
| | | | | | | | | projection of axons to different | |
| | | | | | | | | regions during neuronal development. | |
| 3 | 3 | Q15063 | ISOFORM 1 OF | 93314 | Secreted, | Cell adhesion, cell | Widely | Binds to heparin. Induces cell | Yes - present in |
| | | | PERIOSTIN PRECURSOR | | extracellular | structure, cell | expressed with | attachment and spreading and plays a | E14.5 |
| | | | | | space, | motility | highest levels in | role in cell adhesion | |
| | | | | | extracellular | | aorta, stomach, | | |
| | | | | | matrix | | lower | | |
| | | | | | | | gastrointestinal | | |
| | | | | | | | tract, placenta, | | |
| | | | | | | | uterus and | | |
| | | | | | | | breast. | | |
| 3 | 3 | P22792 | CARBOXYPEPTIDASE N | 60615 | Secreted | Enzyme regulator, | Plasma | Binds to catalytic subunit, stabilizes it | |
| | | | SUBUNIT 2 PRECURSOR | | | protein stabilization | | and keeps it in circulation | |
| 3 | 3 | P55786 | PUROMYCIN-SENSITIVE | 103276 | Intracellular - | Protease | Widely | Involved in proteolytic events | Yes - all rat |
| | | | AMINOPEPTIDASE | | Nucleus, | | distributed | essential for cell growth and viability. | samples |
| | | | | | Cytoplasm | | | May act as regulator of neuropeptide | |
| | | | | | | | | activity. | |
| 3 | 3 | P62736 | ACTIN, AORTIC SMOOTH | 42009 | Cytoplasm | Cell motility | Widely | Actins are highly conserved proteins | |
| | | | MUSCLE | | | | distributed | that are involved in various types of | |
| | | | | | | | | cell motility and are ubiquitously | |
| | | | | | | | | expressed in all eukaryotic cells | |
| 3 | 3 | P02452 | COLLAGEN ALPHA-1(I) | 138883 | Secreted, | Cell adhesion - Cell | Forms the fibrils | Component of connective tissue | Yes - all rat |
| | | | CHAIN PRECURSOR | | extracellular | structure | of tendon, | | samples |
| | | | | | space, | | ligaments and | | |
| | | | | | extracellular | | bones | | |
| | | | | | matrix | | | | |
| 3 | 3 | Q16819 | MEPRIN A SUBUNIT | 84368 | Membrane | Protease | Widely | Cell surface endopeptidase | |
| | | | ALPHA PRECURSOR | | | | distributed, | | |
| | | | | | | | highly expressed | | |
| | | | | | | | in gut | | |
| 3 | 13 | P23471 | PROTEIN TYROSINE | 254587 | Membrane | Transmembrane | Specifically | May be involved in the regulation of | |
| | | | PHOSPHATASE, | | | receptor protein | expressed in the | specific developmental processes in | |
| | | | RECEPTOR-TYPE, ZETA1 | | | | central nervous | the CNS | |
| | | | PRECURSOR | | | | system | | |
| 3 | 4 | O95373 | 120 KDA PROTEIN - | 119702 | Intracellular - | Nuclear-cytoplasmic | Ubiquitous | Involved in the import of proteins with | Yes - present in |
| | | | Importin 7 | | Nucleus, | transport | | the nuclear localization signal. | E12.5LV, |
| | | | | | Cytoplasm | | | | E17.5LV |
| 3 | 3 | Q9HBB3 | DNA-BINDING PROTEIN | 32891 | Intracellular | Protein biosynthesis - | Ubiquitous | | |
| | | | TAXREB107 | | | Ribonucleoprotein | | | |
| 3 | 3 | Q13263 | TRIPARTITE MOTIF- | 88550 | Nucleus | Transcriptional | Ubiquitous | Acts as a transcriptional corepressor | Yes - present in |
| | | | CONTAINING 28 | | | Regulation | | | E12.5LV, |
| | | | PROTEIN | | | | | | E14.5LV |
| 3 | 3 | P51693 | AMYLOID-LIKE PROTEIN | 72176 | Cell membrane, | May play a role in | Cerebral cortex | Can regulate neurite outgrowth | Yes - present in |
| | | | 1 PRECURSOR | | Cytoplasm | postsynaptic | | through binding to components of the | E14.5 4thV, |
| | | | | | | function | | extracellular matrix. | E17.5LV |
| 3 | 4 | P20849 | ISOFORM 1 OF | 91855 | Secreted, | Extracellular matrix | Structural | Component of connective tissue | |
| | | | COLLAGEN ALPHA-1(IX) | | extracellular | structural | component of | | |
| | | | CHAIN PRECURSOR | | space, | constituent | hyaline cartilage | | |
| | | | | | extracellular | | and vitreous of | | |
| | | | | | matrix | | the eye | | |
| 3 | 3 | Q14112 | NIDOGEN-2 PRECURSOR | 151395 | Secreted, | Cell adhesion | Heart, placenta | Cell adhesion glycoprotein which is | Yes - all rat |
| | | | | | extracellular | | and bone | widely distributed in basement | samples |
| | | | | | space, | | | membranes | |
| | | | | | extracellular | | | | |
| | | | | | matrix | | | | |
| 3 | 3 | Q6EMK4 | VASORIN PRECURSOR | 71713 | Membrane | Inhibitor of TGF- | Aorta, kidney, | Modulates the arterial response to | |
| | | | | | | beta signaling | placenta, brain, | injury | |
| | | | | | | | heart, liver, lung | | |
| | | | | | | | and skeletal | | |
| | | | | | | | muscle. | | |
| 3 | 3 | P11142 | ISOFORM 1 OF HEAT | 70898 | Cytoplasm | Chaperone | Ubiquitous | Belongs to the heat shock protein 70 | Yes - present in |
| | | | SHOCK COGNATE 71 KDA | | | | | family | E12.5LV, |
| | | | PROTEIN | | | | | | E14.5LV, |
| | | | | | | | | | E17.5LV |
| 3 | 3 | Q92823 | ISOFORM 1 OF | 144074 | Membrane | Cell adhesion, | Widely | Belongs to the immunoglobulin super | Yes - all rat |
| | | | NEURONAL CELL | | | involved in neuron- | distributed, | family | samples |
| | | | ADHESION MOLECULE | | | neuron adhesion | highly expressed | | |
| | | | PRECURSOR | | | | in brain | | |
| 2 | 2 | P30086 | PHOSPHATIDYLETHANOLAMINE- | 20926 | Cytoplasm | Serine Protease | Ubiquitous | Serine protease inhibitor which | Yes - present in |
| | | | BINDING | | | Inhibitor | | inhibits thrombin, neuropsin and | E17.5LV |
| | | | PROTEIN 1 | | | | | chymotrypsin. | |
| 2 | 4 | Q6PIH6 | IGKV1-5 (Immunoglobulin | 26234 | Membrane | MHC class I protein | Unknown | Antigen processing and presentation | |
| | | | kappa variable 1-5) | | | complex | | of peptides | |
| | | | PROTEIN | | | | | | |
| 2 | 2 | P08697 | ALPHA-2-ANTIPLASMIN | 55064 | Secreted | Serine Protease | Plasma | Belongs to the serpin family | Yes - all rat |
| | | | PRECURSOR | | | Inhibitor - Blood | | | samples |
| | | | | | | coagulation | | | |
| 2 | 2 | Q02487 | ISOFORM 2A OF | 99962 | Membrane | Cell adhesion | Expressed in | Component of intercellular | |
| | | | DESMOCOLLIN-2 | | | | epithelia, | desmosome junctions. | |
| | | | PRECURSOR | | | | myocardium and | | |
| | | | | | | | lymph nodes | | |
| 2 | 2 | P49736 | DNA REPLICATION | 101896 | Nucleus | DNA binding, cell | Ubiquitous | Acts as a factor that allows the DNA | Yes - present in |
| | | | LICENSING FACTOR | | | cycle progression | | to undergo a single round of | E12.5LV, |
| | | | MCM2 | | | | | replication per cell cycle. Required for | E17.5LV |
| | | | | | | | | the entry in S phase and for cell | |
| | | | | | | | | division | |
| 2 | 2 | P26022 | PENTRAXIN-RELATED | 42020 | Secreted | Inflammatory | Widely | Plays a role in the regulation of innate | |
| | | | PROTEIN PTX3 | | | response | distributed, | resistance to pathogens, and | |
| | | | PRECURSOR | | | | highly expressed | inflammatory reactions. | |
| | | | | | | | in connective | | |
| | | | | | | | tissue | | |
| 2 | 2 | O60462 | ISOFORM A22 OF | 104831 | Membrane | Vascular endothelial | Widely | High affinity receptor for semaphorins | Yes - present in |
| | | | NEUROPILIN-2 | | | growth factor | distributed | 3C, 3F, VEGF-165 and VEGF-145 | E14.5 4thV |
| | | | PRECURSOR | | | receptor activity - | | isoforms of VEGF, and the PLGF-2 | |
| | | | | | | involved in axon | | isoform of PGF | |
| | | | | | | guidance | | | |
| 2 | 4 | P51884 | LUMICAN PRECURSOR | 38429 | Secreted, | ECM structural | Widely | Binds to laminin | Yes - all rat |
| | | | | | extracellular | component | distributed, | | samples |
| | | | | | space, | | highly expressed | | |
| | | | | | extracellular | | in cornea | | |
| | | | | | matrix | | | | |
| 2 | 2 | P01042 | ISOFORM HMW OF | 71945 | Secreted, | Cysteine protease | Plasma | Bradykinin is released from kininogen | Yes - all rat |
| | | | KININOGEN-1 | | extracellular | inhibitor | | by plasma kallikrein. Plays an | samples |
| | | | PRECURSOR | | space, | | | important role in blood coagulation. | |
| | | | | | extracellular | | | | |
| | | | | | matrix | | | | |
| 2 | 2 | P55060 | ISOFORM 1 OF | 110417 | Intracellular - | Nuclear-cytoplasmic | Ubiquitous | Export receptor for importin-alpha. | |
| | | | EXPORTIN-2 | | Nucleus, | shuttling | | | |
| | | | | | Cytoplasm | | | | |
| 2 | 2 | P06748 | ISOFORM 2 OF | 29465 | Nucleus, | rRNA metabolism | Ubiquitous | Associated with nucleolar | |
| | | | NUCLEOPHOSMIN | | nucleolus | | | ribonucleoprotein structures and bind | |
| | | | | | | | | single-stranded nucleic acids. It may | |
| | | | | | | | | function in the assembly and/or | |
| | | | | | | | | transport of ribosome | |
| 2 | 2 | Q5UE57 | ALCADEIN BETA | 107033 | Membrane | Cell adhesion | Widely | Calcium ion binding | |
| | | | | | | | distributed, | | |
| | | | | | | | expressed highly | | |
| | | | | | | | in brain | | |
| 2 | 2 | P27348 | 14-3-3 PROTEIN THETA | 27764 | Cytoplasm | Chaperone, signal | Widely | Adapter protein implicated in the | Yes - present in |
| | | | | | | transduction | distributed, | regulation of a large spectrum of both | E12.5LV, |
| | | | | | | | expressed highly | general and specialized signaling | E14.5LV, |
| | | | | | | | in brain, heart | pathways. | E17.5LV |
| | | | | | | | and pancreas | | |
| 2 | 2 | P36578 | 60S RIBOSOMAL | 47566 | Cytoplasm | Protein biosynthesis | Ubiqutous | Cytosolic large ribosomal subunit | Yes - present in |
| | | | PROTEIN L4 | | | | | | E12.5LV, |
| | | | | | | | | | E14.5LV |
| 2 | 2 | P08253 | 72 KDA TYPE IV | 73882 | Secreted, | Hydrolase, | Fibroblasts | Cleaves gelatin and collagen | |
| | | | COLLAGENASE | | extracellular | proteolysis | | | |
| | | | PRECURSOR | | space, | | | | |
| | | | | | extracellular | | | | |
| | | | | | matrix | | | | |
| 2 | 2 | | HYPOTHETICAL | 42003 | Unknown | Unknown | Unknown | Unknown | |
| | | | PROTEIN LOC345651 | | | | | | |
| 2 | 2 | P08572 | COLLAGEN ALPHA-2(IV) | 167535 | Secreted, | Extracellular matrix | Widely | Type IV collagen is the major | Yes - present in |
| | | | CHAIN PRECURSOR | | extracellular | structural | distributed | structural component of glomerular | E12.5LV, E14.5 |
| | | | | | space, | constituent | | basement membranes, forming a | |
| | | | | | extracellular | | | meshwork together with laminins, | |
| | | | | | matrix | | | proteoglycans and entactin/nidogen. | |
| 2 | 2 | P52701 | ISOFORM GTBP-ALT OF | 120563 | Nucleus | DNA mismatch | Ubiquitous | Restores repair of base-base and | |
| | | | DNA MISMATCH REPAIR | | | repair | | single-nucleotide insertion-deletion | |
| | | | PROTEIN MSH6 | | | | | mismatches. | |
| 2 | 2 | P14625 | ENDOPLASMIN | 92469 | Endoplasmic | Chaperone, protein | Widely | Molecular chaperone that functions in | Yes - all rat |
| | | | PRECURSOR | | reticulum | folding | distributed | the processing and transport of | samples |
| | | | | | | | | secreted proteins | (TUMOR |
| | | | | | | | | | REJECTION |
| | | | | | | | | | ANTIGEN |
| | | | | | | | | | GP96) |
| 2 | 2 | P05067 | ISOFORM APP770 OF | 86943 | Membrane. | Cell surface | Expressed in all | During neuronal differentiation, the | Yes - present in |
| | | | AMYLOID BETA A4 | | After alpha- | receptor involved in | fetal tissues | Thr-743 phosphorylated form is | E12.5LV, E14.5 |
| | | | PROTEIN PRECURSOR | | secretase | neurite growth, | examined with | located mainly in growth cones, | 4thV, E17.5LV |
| | | | (FRAGMENT) | | cleavage, | neuronal adhesion | highest levels in | moderately in neurites and sparingly | |
| | | | | | soluble APP is | and axonogenesis. | brain, kidney, | in the cell body. Defects in APP are a | |
| | | | | | released into | | heart and | cause of autosomal dominant | |
| | | | | | the extracellular | | spleen. | Alzheimer disease. | |
| | | | | | space and the | | | | |
| | | | | | C-terminal is | | | | |
| | | | | | internalized to | | | | |
| | | | | | endosomes and | | | | |
| | | | | | lysosomes. | | | | |
| 2 | 2 | P09651 | HETEROGENEOUS | 38747 | Intracellular - | Component of | Ubiquitous | Involved in the packaging of pre- | Yes - present in |
| | | | NUCLEAR | | Nucleus, | ribonucleosomes. | | mRNA into hnRNP particles, transport | E12.5LV |
| | | | RIBONUCLEOPROTEIN | | Cytoplasm | Nuclear mRNA | | of poly(A) mRNA from the nucleus to | |
| | | | A1 ISOFORM B | | | splicing | | the cytoplasm and may modulate | |
| | | | | | | | | splice site selection. | |
| 2 | 2 | P61978 | ISOFORM 1 OF | 50976 | Intracellular - | RNA processing | Ubiquitous | One of the major pre-mRNA-binding | |
| | | | HETEROGENEOUS | | Nucleus, | | | proteins. Binds tenaciously to poly(C) | |
| | | | NUCLEAR | | Cytoplasm | | | sequences. | |
| | | | RIBONUCLEOPROTEIN K | | | | | | |
| 2 | 2 | P84077 | ADP-RIBOSYLATION | 20566 | Membrane - | Protein trafficking | Ubiquitous | Modulates vesicle budding and | |
| | | | FACTOR 1 | | Golgi | | | uncoating within the Golgi complex. | |
| 2 | 2 | P01034 | CYSTATIN C | 15799 | Secreted, | Cysteine protease | Widely | Important regulator of cysteine | Yes - all rat |
| | | | PRECURSOR | | extracellular | inhibitor | distributed, | proteases in a number of physiologic | samples |
| | | | | | space, | | highly expressed | functions | |
| | | | | | extracellular | | in epididymis, | | |
| | | | | | matrix | | vas deferens, | | |
| | | | | | | | brain, thymus, | | |
| | | | | | | | and ovary | | |
| 2 | 2 | P68371 | TUBULIN BETA-2C CHAIN | 49831 | Intracellular | Cell structure, cell | Widely | Tubulin is the major constituent of | Yes - all rat |
| | | | | | | mobility, | distributed | microtubules | samples |
| | | | | | | chromosome | | | |
| | | | | | | segregation, | | | |
| | | | | | | intracellular protein | | | |
| | | | | | | traffic | | | |
| 2 | 2 | P08123 | COLLAGEN ALPHA-2(I) | 129412 | Secreted, | Cell adhesion - Cell | Forms the fibrils | Involved in skeletal development | Yes - all rat |
| | | | CHAIN PRECURSOR | | extracellular | structure | of tendon, | | samples |
| | | | | | space, | | ligaments and | | |
| | | | | | extracellular | | bones | | |
| | | | | | matrix | | | | |
| 2 | 4 | P01842 | IGLC1 PROTEIN | 24793 | Membrane | Immune response | Ubiquitous | MHC class I protein complex antigen | |
| | | | | | | antigen binding | | processing and presentation. | |
| 2 | 2 | P62424 | 60S RIBOSOMAL | 29864 | Cytoplasm | Protein biosynthesis | Ubiquitous | Cytosolic large ribosomal subunit | Yes - present in |
| | | | PROTEIN L7A | | | | | | E12.5LV, |
| | | | | | | | | | E17.5LV |
| 2 | 2 | P24043 | LAMININ ALPHA 2 | 343905 | Secreted, | Extracellular matrix | Placenta, | Laminin is thought to mediate the | |
| | | | SUBUNIT PRECURSOR | | extracellular | linker protein- | striated muscle, | attachment, migration and | |
| | | | | | space, | mediated signaling | peripheral nerve, | organization of cells into tissues | |
| | | | | | extracellular | | cardiac muscle, | during embryonic development by | |
| | | | | | matrix | | pancreas, lung, | interacting with other extracellular | |
| | | | | | | | spleen, kidney, | matrix components. | |
| | | | | | | | adrenal gland, | | |
| | | | | | | | skin, testis, | | |
| | | | | | | | meninges, | | |
| | | | | | | | choroid plexus, | | |
| | | | | | | | and some other | | |
| | | | | | | | regions of the | | |
| | | | | | | | brain | | |
| 2 | 3 | P04196 | HISTIDINE-RICH | 59578 | Secreted | Function unknown | Expressed by the | It binds heme, dyes and divalent | Yes - present in |
| | | | GLYCOPROTEIN | | | | liver and | metal ions. It can inhibit rosette | E12.5LV, |
| | | | PRECURSOR | | | | secreted in | formation and is known to interact | E14.5LV |
| | | | | | | | plasma | with heparin, thrombospondin, and | |
| | | | | | | | | the lysine-binding site of plasminogen. | |
| | | | | | | | | On the basis of its homology with | |
| | | | | | | | | HMW kininogen, the His-rich region of | |
| | | | | | | | | this protein may mediate the contact | |
| | | | | | | | | activation phase of intrinsic blood | |
| | | | | | | | | coagulation cascade. | |
| 2 | 2 | P62826 | GTP-BINDING NUCLEAR | 24292 | Intracellular - | Nuclear-cytoplasmic | Ubiquitous | Required for the import of protein into | Yes - all rat |
| | | | PROTEIN RAN | | Nucleus, | shuttling | | the nucleus and also for RNA export. | samples |
| | | | | | Cytoplasm | | | | |
| 2 | 2 | P69905 | HEMOGLOBIN SUBUNIT | 15126 | Intracellular | Oxygen Transport, | Red blood cells | Involved in oxygen transport from the | Yes - present in |
| | | | ALPHA | | | Transfer - Carrier | | lung to the various peripheral tissues | E12.5LV, |
| | | | | | | Protein | | | E17.5LV |
| 2 | 2 | P68871 | HEMOGLOBIN SUBUNIT | 15867 | Intracellular | Oxygen Transport, | Red blood cells | Involved in oxygen transport from the | Yes - present in |
| | | | BETA | | | Transfer - Carrier | | lung to the various peripheral tissues | E17.5LV |
| | | | | | | Protein | | | |
| 2 | 2 | P08603 | ISOFORM 1 OF | 139070 | Secreted | Complement | Expressed by the | Involved in the regulation of | Yes - all rat |
| | | | COMPLEMENT FACTOR | | | activation | liver and | complement activation | samples |
| | | | H PRECURSOR | | | | secreted in | | |
| | | | | | | | plasma | | |
| 2 | 2 | P53618 | COATOMER SUBUNIT | 107139 | Cytoplasm. | Vesicle mediated | Ubiquitous | Coatomer complex is required for | Yes - present in |
| | | | BETA | | Golgi apparatus | transport | | budding from Golgi membranes, and | E12.5LV, |
| | | | | | | | | is essential for the retrograde Golgi- | E17.5LV |
| | | | | | | | | to-ER transport of dilysine-tagged | |
| | | | | | | | | proteins. | |
| 2 | 2 | P53675 | ISOFORM 1 OF | 187030 | Cytoplasmic | Receptor-mediated | Ubiquitous | Involved in the formation of clathrin | Yes - all rat |
| | | | CLATHRIN HEAVY CHAIN 2 | | vesicle; | endocytosis | | coated vesicles during vesicle | samples |
| | | | | | cytoplasmic | | | endocytosis | |
| | | | | | vesicle | | | | |
| | | | | | membrane | | | | |
| 2 | 2 | P09871 | COMPLEMENT C1S | 76684 | Secreted | Serine protease | Plasma | Immune response - Complement | Yes - present in |
| | | | SUBCOMPONENT | | | | | activation | E17.5LV |
| | | | PRECURSOR | | | | | | |
| 2 | 2 | Q8NBJ4 | GOLGI | 46273 | Golgi apparatus | Function unknown | Widely | Cellular response protein to viral | |
| | | | PHOSPHOPROTEIN 2 | | | | expressed | infection | |
| 2 | 2 | P13639 | ELONGATION FACTOR 2 | 95207 | Cytoplasm | Protein biosynthesis | Ubiquitous | Promotes translocation of protein | Yes - all rat |
| | | | | | | | | chain from A site to P site on | samples |
| | | | | | | | | ribosome | |
| 2 | 2 | P68366 | TUBULIN ALPHA-1 CHAIN | 49924 | Intracellular | Cell structure, cell | Widely | Tubulin is the major constituent of | Yes - all rat |
| | | | | | | mobility, intracellular | distributed | microtubules | samples |
| | | | | | | protein traffic | | | |
| 2 | 2 | Unknown | 22 KDA PROTEIN | 22066 | Unknown | Unknown | Unknown | Unknown | |
| 2 | 2 | P45974 | ISOFORM LONG OF | 95786 | Intracellular - | Protease | Ubiquitous | Cleaves linear and branched | Yes - present in |
| | | | UBIQUITIN CARBOXYL- | | Lysosome | | | multiubiquitin polymers with a marked | E14.5LV, |
| | | | TERMINAL HYDROLASE 5 | | | | | preference for branched polymers. | E17.5LV |
| 2 | 2 | Q14008 | CYTOSKELETON- | 225509 | Intracellular - | Centrosome | Skeletal muscle, | Plays a major role in organizing | |
| | | | ASSOCIATED PROTEIN 5 | | centrosome | organization and | brain, heart, | spindle poles | |
| | | | | | | biogenesis, and | placenta, lung, | | |
| | | | | | | establishment and | liver, kidney and | | |
| | | | | | | maintenance of | pancreas | | |
| | | | | | | microtubule | | | |
| | | | | | | cytoskeleton polarity | | | |
| 2 | 7 | Q8IUK7 | ALB PROTEIN | 45160 | Secreted | Transport, | Plasma | Serum albumin, the main protein of | Yes - all rat |
| | | | | | | Transfer/Carrier | | plasma, has a good binding capacity | samples |
| | | | | | | | | for water, Ca(2+), Na(+), K(+), fatty | |
| | | | | | | | | acids, hormones, bilirubin and drugs. | |
| | | | | | | | | Its main function is the regulation of | |
| | | | | | | | | the colloidal osmotic pressure of | |
| | | | | | | | | blood. | |
| 2 | 3 | P55285 | ISOFORM 1 OF | 88309 | Membrane | Cell adhesion | Highly expressed | Also known as Kidney cadherin | Yes - all rat |
| | | | CADHERIN-6 | | | | in ovary, brain, | | samples |
| | | | PRECURSOR | | | | cerebellum, and | | |
| | | | | | | | kidney | | |
| 2 | 2 | P23246 | ISOFORM LONG OF | 76149 | Nucleus | mRNA processing | Ubiquitous | DNA- and RNA binding protein, | |
| | | | SPLICING FACTOR, | | | | | involved in several nuclear processes. | |
| | | | PROLINE- AND | | | | | Essential pre-mRNA splicing factor | |
| | | | GLUTAMINE-RICH | | | | | required early in spliceosome | |
| | | | | | | | | formation. | |
| 2 | 2 | P04275 | VON WILLEBRAND | 309299 | Secreted | Cell adhesion - | Plasma | Important in the maintenance of | Yes - all rat |
| | | | FACTOR PRECURSOR | | | Blood clotting | | hemostasis, it promotes adhesion of | samples |
| | | | | | | | | platelets to the sites of vascular injury. | |
| | | | | | | | | Also acts as a chaperone for | |
| | | | | | | | | coagulation factor VIII, delivering it to | |
| | | | | | | | | the site of injury, stabilizing its | |
| | | | | | | | | heterodimeric structure and protecting | |
| | | | | | | | | it from premature clearance from | |
| | | | | | | | | plasma. | |
| 2 | 3 | P01011 | ISOFORM 1 OF ALPHA-1- | 47651 | Secreted | Serine protease | Plasma | Belongs to the serpin family | |
| | | | ANTICHYMOTRYPSIN | | | inhibitor | | | |
| | | | PRECURSOR | | | | | | |
| 2 | 2 | P09211 | GLUTATHIONE S- | 23225 | Cytoplasm | Transferase | Ubiquitous | Involved in detoxifing compounds by | Yes - present in |
| | | | TRANSFERASE P | | | | | linking glutathione to hydrophobic | E12.5LV |
| | | | | | | | | substances | |
| 2 | 2 | P33991 | DNA REPLICATION | 96558 | Nucleus | Involved in the | Ubiquitous | Involved in chromatin binding | |
| | | | LICENSING FACTOR | | | control of DNA | | | |
| | | | MCM4 | | | replication | | | |
| 2 | 8 | P23142 | ISOFORM C OF FIBULIN- | 74462 | Secreted, | Cell adhesion | Widely | Involved in a number of various ECM | Yes - all rat |
| | | | 1 PRECURSOR | | extracellular | mediated signaling | expressed during | related functions during development | samples |
| | | | | | space, | | embryonic | including cell adhesion, migration, | |
| | | | | | extracellular | | development | and ecm architecture. Binds to laminin | |
| | | | | | matrix | | | and nidogen (both found in the CSF). | |
| 2 | 3 | Q60847 | ISOFORM LONG OF | 333194 | Secreted, | Cell adhesion - Cell | Widely | Component of connective tissue | |
| | | | COLLAGEN ALPHA-1(XII) | | extracellular | structure | distributed | | |
| | | | CHAIN PRECURSOR | | space, | | | | |
| | | | | | extracellular | | | | |
| | | | | | matrix | | | | |
| 2 | 3 | Q24JP5 | TRANSMEMBRANE | 110110 | Membrane | Integral to | Unknown | Unknown | |
| | | | PROTEIN 132A ISOFORM B | | | membrane | | | |
| 2 | 2 | Q16610 | EXTRACELLULAR | 60674 | Secreted, | Signal transduction | Widely | Positive regulation of I-kappaB | |
| | | | MATRIX PROTEIN 1 | | extracellular | activity | distributed | kinase/NF-kappaB cascade | |
| | | | PRECURSOR | | space, | | | | |
| | | | | | extracellular | | | | |
| | | | | | matrix | | | | |
| 2 | 2 | P13671 | COMPLEMENT | 105752 | Secreted | Immune response - | Plasma | Involved in the formation of the lytic | Yes - present in |
| | | | COMPONENT C6 | | | complement | | complex, which inserts into plasma | E17.5LV |
| | | | PRECURSOR | | | pathway | | membranes and causes cells to lyse. | |
| 2 | 2 | P10586 | RECEPTOR-TYPE | 211845 | Membrane | Transmembrane | Widely | Signaling molecule invovled in cell | Yes-LAR |
| | | | TYROSINE-PROTEIN | | | receptor protein | distributed | growth and differentiation | RECEPTOR- |
| | | | PHOSPHATASE F | | | involved in cell | | | LINKED |
| | | | PRECURSOR | | | adhesion | | | TYROSINE |
| | | | | | | | | | PHOSPHATASE |
| | | | | | | | | | present in all rat |
| | | | | | | | | | samples |
| 2 | 2 | Q9HCU4 | CADHERIN EGF LAG | 317453 | Membrane | G-protein coupled | Highest | May have an important role in cell/cell | Yes - present in |
| | | | SEVEN-PASS G-TYPE | | | receptor protein | expression in | signaling during nervous system | E12.5LV, |
| | | | RECEPTOR 2 | | | signaling pathway | brain and testis | formation | E14.5LV |
| | | | PRECURSOR | | | | | | |
| 2 | 2 | P62917 | 60S RIBOSOMAL | 27893 | Cytoplasm | Protein Biosynthesis | Ubiquitous | Ribosomal structural protein | Yes - present in |
| | | | PROTEIN L8 | | | | | | E12.5LV, E14.5 |
| | | | | | | | | | 4thV |
| 2 | 2 | P33176 | KINESIN HEAVY CHAIN | 109685 | Intracellular | Microtubule motor | Ubiquitous | Microtubute-dependent motor | Yes - present in |
| | | | | | | activity | | required for normal distribution of | E17.5LV |
| | | | | | | | | mitochondria and lysosomes | |
| 2 | 2 | P62258 | 14-3-3 PROTEIN EPSILON | 29174 | Cytoplasm | Chaperone, signal | Ubiquitous | Adapter protein implicated in the | Yes - all rat |
| | | | | | | transduction - | | regulation of a large spectrum of both | samples |
| | | | | | | intracellular | | general and specialized signaling | |
| | | | | | | signaling cascade | | pathway | |
| 2 | 4 | Q8NF17 | FLJ00385 PROTEIN | 56111 | Membrane | Unknown | Unknown | IgG like protein | |
| | | | (FRAGMENT) | | | | | | |
| 2 | 2 | P43243 | MATRIN-3 | 94623 | Nucleus | Function unclear | Ubiquitous | May play a role in trascription, nuclear | Yes - present in |
| | | | | | | | | structure, or nuclear retention of | E12.5LV, |
| | | | | | | | | defective RNAs | E17.5LV |
| 2 | 2 | P15924 | ISOFORM DPI OF | 331776 | Membrane | Cell adhesion, cell | Isoform DPI is a | Major high molecular weight protein of | Yes - present in |
| | | | DESMOPLAKIN | | | junctions | constituent of all | desmosomes | E12.5LV, |
| | | | | | | | desmosomes | | E17.5LV |
| 2 | 2 | P19652 | ALPHA-1-ACID | 23603 | Secreted | Immune response - | Plasma | Appears to function in modulating the | |
| | | | GLYCOPROTEIN 2 | | | acute phase | | activity of the immune system during | |
| | | | PRECURSOR | | | | | the acute-phase reaction | |
| 2 | 2 | P60842 | EUKARYOTIC INITIATION | 46154 | Intracellular | Protein biosynthesis | Ubiquitous | Required for mRNA binding to | |
| | | | FACTOR 4A-I | | | | | ribosome | |
| 2 | 2 | P00338 | LACTATE | 36689 | Cytoplasm | Anaerobic glycolysis | Widely | Catalyzes the conversion of lactate | Yes - present in |
| | | | DEHYDROGENASE A | | | | distributed, | and NAD to pyruvate and NADH | E12.5LV, E14.5 |
| | | | | | | | highly expressed | | |
| | | | | | | | in muscle tissue | | |
| 2 | 2 | Q6UX71 | ISOFORM 1 OF PLEXIN | 59583 | Membrane | Cell surface | Endothelial cells | May play a role in tumor angiogenesis | |
| | | | DOMAIN-CONTAINING | | | endothelial marker | | | |
| | | | PROTEIN 2 PRECURSOR | | | | | | |
| 2 | 2 | P30041 | PEROXIREDOXIN-6 | 24904 | Cytoplasm | Oxidoreductase, | Ubiquitous | Involved in redox regulation of the cell | |
| | | | | | | Peroxidase | | in response to oxidative stress. | |
| 2 | 2 | O14594 | NEUROCAN CORE | 142973 | Secreted, | Extracellular matrix | Brain | May modulate neuronal adhesion and | Yes - present in |
| | | | PROTEIN PRECURSOR | | extracellular | protein-mediated | | neurite growth during development by | E14.5 4thV, |
| | | | | | space, | signaling, cell | | binding to neural cell adhesion | E17.5LV |
| | | | | | extracellular | adhesion, cell | | molecules. | |
| | | | | | matrix | motility | | | |
| 2 | 2 | Q12860 | ISOFORM 1 OF | 113320 | Membrane | Cell adhesion | Widely | Mediates cell surface interactions | Yes - present in |
| | | | CONTACTIN-1 | | | | distributed, | during nervous system development. | E17.5LV |
| | | | PRECURSOR | | | | highly expressed | Involved in the formation of axon-glial | |
| | | | | | | | in brain | junctions in myelinated peripheral | |
| | | | | | | | | nerves and in the signaling between | |
| | | | | | | | | axons and myelinating glial cells. | |
| | | | | | | | | Participates in oligodendrocytes | |
| | | | | | | | | generation by acting as a ligand of | |
| | | | | | | | | NOTCH1. | |
| 2 | 2 | Q8TF66 | LEUCINE-RICH REPEAT- | 64396 | Membrane | Unknown | Brain and | Unknown | |
| | | | CONTAINING PROTEIN | | | | placenta | | |
| | | | 15 PRECURSOR | | | | | | |

TABLE 2
Protein list from mass spectrometry CS19 human CSF.

| Number | | |
|---|---|---|
| of unique | Total | |
| peptides | number | |
| from | of | |
| protein | peptides | Protein matches for CS19 human CSF |
| 194 | 571 | Homo sapiens (Human) APOLIPOPROTEIN B-100 PRECURSOR. [MASS = 515563 |
| 120 | 176 | Homo sapiens (Human) DYNEIN HEAVY CHAIN, CYTOSOLIC. [MASS = 532408 |
| 68 | 168 | Homo sapiens (Human) MICROTUBULE-ASSOCIATED PROTEIN 1B. [MASS = 270620 |
| 53 | 58 | Homo sapiens (Human) MYOSIN-10. [MASS = 228939 |
| 52 | 66 | Homo sapiens (Human) FILAMIN A, ALPHA. [MASS = 280018 |
| 48 | 54 | Homo sapiens (Human) ISOFORM 1 OF SPECTRIN ALPHA CHAIN, BRAIN. [MASS = 284539 |
| 39 | 72 | Homo sapiens (Human) FATTY ACID SYNTHASE. [MASS = 273400 |
| 36 | 73 | Homo sapiens (Human) VIMENTIN. [MASS = 53520 |
| 36 | 36 | Homo sapiens (Human) ISOFORM LONG OF SPECTRIN BETA CHAIN, BRAIN 1. [MASS = 274631 |
| 36 | 72 | Homo sapiens (Human) NESTIN. [MASS = 176706 |
| 35 | 172 | Homo sapiens (Human) ALB PROTEIN. [MASS = 71705 |
| 34 | 127 | Homo sapiens (Human) SEROTRANSFERRIN PRECURSOR. [MASS = 77050 |
| 31 | 41 | Homo sapiens (Human) Heat shock 70 kDa protein 5. [MASS = 72422 |
| 31 | 45 | Homo sapiens (Human) ISOFORM 1 OF FIBRONECTIN PRECURSOR. [MASS = 262607 |
| 30 | 36 | Homo sapiens (Human) MOESIN. [MASS = 67689 |
| 29 | 84 | Homo sapiens (Human) HYPOTHETICAL PROTEIN DKFZP761K0511. [MASS = 84843 |
| 28 | 58 | Homo sapiens (Human) ISOFORM 1 OF HEAT SHOCK COGNATE 71 KDA PROTEIN. [MASS = 70898 |
| 27 | 46 | Homo sapiens (Human) ELONGATION FACTOR 2. [MASS = 95207 |
| 27 | 30 | Homo sapiens (Human) PRE-MRNA-PROCESSING-SPLICING FACTOR 8. [MASS = 273600 |
| 26 | 44 | Homo sapiens (Human) APOLIPOPROTEIN A-IV PRECURSOR. [MASS = 45399 |
| 26 | 102 | Homo sapiens (Human) GLYCERALDEHYDE-3-PHOSPHATE DEHYDROGENASE. [MASS = 35922 |
| 26 | 29 | Homo sapiens (Human) ISOFORM 1 OF DNA-DEPENDENT PROTEIN KINASE CATALYTIC |
| | | SUBUNIT. [MASS = 469089 |
| 25 | 109 | Homo sapiens (Human) TUBULIN BETA-2C CHAIN. [MASS = 49831 |
| 25 | 41 | Homo sapiens (Human) CLATHRIN HEAVY CHAIN 1. [MASS = 191615 |
| 24 | 27 | Homo sapiens (Human) MYOSIN-9. [MASS = 226401 |
| 23 | 30 | Homo sapiens (Human) T-COMPLEX PROTEIN 1 SUBUNIT BETA. [MASS = 57357 |
| 23 | 31 | Homo sapiens (Human) ENDOPLASMIN PRECURSOR. [MASS = 92469 |
| 23 | 35 | Homo sapiens (Human) ISOFORM 1 OF CULLIN-ASSOCIATED NEDD8-DISSOCIATED PROTEIN 1. |
| | | [MASS = 136376 |
| 23 | 23 | Homo sapiens (Human) ISOFORM 2 OF MICROTUBULE-ACTIN CROSSLINKING FACTOR 1, |
| | | ISOFORMS 1/2/3/5. [MASS = 620418 |
| 23 | 28 | Homo sapiens (Human) ISOFORM 2 OF HECT, UBA AND WWE DOMAIN-CONTAINING |
| | | PROTEIN 1. [MASS = 480198 |
| 22 | 77 | Homo sapiens (Human) CREATINE KINASE B-TYPE. [MASS = 42644 |
| 22 | 26 | Homo sapiens (Human) KINESIN HEAVY CHAIN. [MASS = 109685 |
| 22 | 34 | Homo sapiens (Human) IMPORTIN BETA-1 SUBUNIT. [MASS = 97170 |
| 22 | 31 | Homo sapiens (Human) DNA REPLICATION LICENSING FACTOR MCM2. [MASS = 101896 |
| 22 | 24 | Homo sapiens (Human) STRUCTURAL MAINTENANCE OF CHROMOSOME 3. [MASS = 141542 |
| 22 | 42 | Homo sapiens (Human) APOLIPOPROTEIN A-I PRECURSOR. [MASS = 30778 |
| 21 | 44 | Homo sapiens (Human) ISOFORM M1 OF PYRUVATE KINASE ISOZYMES M1/M2. [MASS = 57931 |
| 21 | 35 | Homo sapiens (Human) DPYSL3 PROTEIN. [MASS = 73910 |
| 21 | 34 | Homo sapiens (Human) RAN BINDING PROTEIN 5. [MASS = 125545 |
| 21 | 35 | Homo sapiens (Human) TRANSITIONAL ENDOPLASMIC RETICULUM ATPASE. [MASS = 89191 |
| 21 | 167 | Homo sapiens (Human) ALPHA-FETOPROTEIN PRECURSOR. [MASS = 68678 |
| 20 | 26 | Homo sapiens (Human) DIHYDROPYRIMIDINASE-RELATED PROTEIN 1. [MASS = 62184 |
| 20 | 27 | Homo sapiens (Human) ATP-DEPENDENT RNA HELICASE A. [MASS = 140881 |
| 20 | 30 | Homo sapiens (Human) MATRIN-3. [MASS = 94623 |
| 19 | 35 | Homo sapiens (Human) RAB GDP DISSOCIATION INHIBITOR ALPHA [MASS = 50583 |
| 19 | 24 | Homo sapiens (Human) APOLIPOPROTEIN E PRECURSOR. [MASS = 36154 |
| 19 | 20 | Homo sapiens (Human) C-1-TETRAHYDROFOLATE SYNTHASE, CYTOPLASMIC. [MASS = 101428 |
| 19 | 29 | Homo sapiens (Human) ISOFORM LONG OF SPLICING FACTOR, PROLINE- AND GLUTAMINE-RICH. |
| | | [MASS = 76149 |
| 19 | 35 | Homo sapiens (Human) ATP-DEPENDENT DNA HELICASE 2 SUBUNIT 2. [MASS = 82573 |
| 19 | 23 | Homo sapiens (Human) ISOFORM 1 OF MICROTUBULE-ASSOCIATED PROTEIN 2. [MASS = 199539 |
| 19 | 22 | Homo sapiens (Human) PROTEIN DISULFIDE-ISOMERASE A4 PRECURSOR. [MASS = 72932 |
| 18 | 38 | Homo sapiens (Human) FIBRINOGEN BETA CHAIN PRECURSOR. [MASS = 55928 |
| 18 | 23 | Homo sapiens (Human) PHOSPHOGLYCERATE KINASE 1. [MASS = 44483 |
| 18 | 18 | Homo sapiens (Human) DNA REPLICATION LICENSING FACTOR MCM6. [MASS = 92889 |
| 18 | 37 | Homo sapiens (Human) ISOFORM 1 OF NUCLEAR AUTOANTIGENIC SPERM PROTEIN. |
| | | [MASS = 85238 |
| 18 | 38 | Homo sapiens (Human) TRIPARTITE MOTIF-CONTAINING 28 PROTEIN. [MASS = 88550 |
| 18 | 20 | Homo sapiens (Human) ISOFORM 1 OF GENERAL TRANSCRIPTION FACTOR II-I. [MASS = 112416 |
| 18 | 35 | Homo sapiens (Human) NCL PROTEIN. [MASS = 51641 |
| 18 | 31 | Homo sapiens (Human) INTER-ALPHA-TRYPSIN INHIBITOR HEAVY CHAIN H2 PRECURSOR. |
| | | [MASS = 106436 |
| 18 | 29 | Homo sapiens (Human) ISOFORM 5 OF INTERLEUKIN ENHANCER-BINDING FACTOR 3. |
| | | [MASS = 74607 |
| 17 | 30 | Homo sapiens (Human) HEAT SHOCK 70 KDA PROTEIN 4. [MASS = 94300 |
| 17 | 19 | Homo sapiens (Human) LUPUS LA PROTEIN. [MASS = 46837 |
| 17 | 89 | Homo sapiens (Human) TUBULIN ALPHA-1 CHAIN. [MASS = 49924 |
| 17 | 24 | Homo sapiens (Human) POLY [ADP-RIBOSE] POLYMERASE 1. [MASS = 112953 |
| 17 | 18 | Homo sapiens (Human) LEUCYL-TRNA SYNTHETASE, CYTOPLASMIC. [MASS = 134466 |
| 17 | 21 | Homo sapiens (Human) PUROMYCIN-SENSITIVE AMINOPEPTIDASE. [MASS = 103276 |
| 17 | 17 | Homo sapiens (Human) ISOFORM 1 OF STRUCTURAL MAINTENANCE OF CHROMOSOME |
| | | 2-LIKE 1 PROTEIN. [MASS = 135781 |
| 16 | 126 | Homo sapiens (Human) ALPHA-1-ANTITRYPSIN PRECURSOR. [MASS = 46737 |
| 16 | 19 | Homo sapiens (Human) EUKARYOTIC INITIATION FACTOR 4A-I. [MASS = 46154 |
| 16 | 20 | Homo sapiens (Human) T-COMPLEX PROTEIN 1 SUBUNIT ETA. [MASS = 59367 |
| 16 | 21 | Homo sapiens (Human) ISOCITRATE DEHYDROGENASE (NADP) CYTOPLASMIC. [MASS = 46659 |
| 16 | 17 | Homo sapiens (Human) KINESIN HEAVY CHAIN ISOFORM 5C. [MASS = 109495 |
| 16 | 35 | Homo sapiens (Human) HETEROGENEOUS NUCLEAR RIBONUCLEOPROTEIN U. [MASS = 88814 |
| 16 | 18 | Homo sapiens (Human) SPLICING FACTOR 3B SUBUNIT 1. [MASS = 145815 |
| 16 | 18 | Homo sapiens (Human) 26S PROTEASOME NON-ATPASE REGULATORY SUBUNIT 2. |
| | | [MASS = 100200 |
| 16 | 17 | Homo sapiens (Human) CHAPERONIN CONTAINING TCP1, SUBUNIT 8. [MASS = 59779 |
| 15 | 17 | Homo sapiens (Human) ATP-CITRATE SYNTHASE. [MASS = 120825 |
| 15 | 53 | Homo sapiens (Human) ISOFORM GAMMA-B OF FIBRINOGEN GAMMA CHAIN |
| | | PRECURSOR. [MASS = 51512 |
| 15 | 18 | Homo sapiens (Human) ISOFORM 1 OF EXPORTIN-2. [MASS = 110417 |
| 15 | 22 | Homo sapiens (Human) HETEROGENEOUS NUCLEAR RIBONUCLEOPROTEIN |
| | | M ISOFORM A. [MASS = 77516 |
| 15 | 22 | Homo sapiens (Human) ISOFORM 2 OF FAR UPSTREAM ELEMENT-BINDING |
| | | PROTEIN 1. [MASS = 68605 |
| 15 | 18 | Homo sapiens (Human) SPLICING FACTOR 3 SUBUNIT 1. [MASS = 88886 |
| 15 | 43 | Homo sapiens (Human) HEAT SHOCK PROTEIN HSP 90-ALPHA 2. [MASS = 98113 |
| 15 | 23 | Homo sapiens (Human) 14-3-3 PROTEIN EPSILON. [MASS = 29174 |
| 15 | 15 | Homo sapiens (Human) ISOFORM 1 OF VINCULIN. [MASS = 116591 |
| 15 | 18 | Homo sapiens (Human) ALANYL-TRNA SYNTHETASE. [MASS = 106801 |
| 14 | 18 | Homo sapiens (Human) DNA REPLICATION LICENSING FACTOR MCM4. [MASS = 96558 |
| 14 | 14 | Homo sapiens (Human) ISOFORM 1 OF SPECTRIN BETA CHAIN, BRAIN 2. [MASS = 271295 |
| 14 | 21 | Homo sapiens (Human) FAR UPSTREAM ELEMENT-BINDING PROTEIN 2. [MASS = 72709 |
| 14 | 14 | Homo sapiens (Human) PROTEIN DISULFIDE-ISOMERASE A3 PRECURSOR. [MASS = 56782 |
| 14 | 19 | Homo sapiens (Human) RAB GDP DISSOCIATION INHIBITOR BETA. [MASS = 50663 |
| 14 | 21 | Homo sapiens (Human) DEAH (ASP-GLU-ALA-HIS) BOX POLYPEPTIDE 15. [MASS = 92829 |
| 14 | 22 | Homo sapiens (Human) LACTATE DEHYDROGENASE A. [MASS = 36689 |
| 14 | 22 | Homo sapiens (Human) EUKARYOTIC TRANSLATION INITIATION FACTOR 3 SUBUNIT 10. |
| | | [MASS = 166569 |
| 14 | 21 | Homo sapiens (Human) ISOFORM BETA OF HEAT-SHOCK PROTEIN 105 KDA. [MASS = 92116 |
| 14 | 25 | Homo sapiens (Human) ISOFORM 1 OF POLYADENYLATE-BINDING PROTEIN 1. |
| | | [MASS = 70671 |
| 13 | 19 | Homo sapiens (Human) 6-PHOSPHOGLUCONATE DEHYDROGENASE, |
| | | DECARBOXYLATING. [MASS = 53009 |
| 13 | 30 | Homo sapiens (Human) ISOFORM 1 OF HETEROGENEOUS NUCLEAR |
| | | RIBONUCLEOPROTEIN K. [MASS = 50976 |
| 13 | 14 | Homo sapiens (Human) DREBRIN. [MASS = 71294 |
| 13 | 14 | Homo sapiens (Human) EXPORTIN-1. [MASS = 123386 |
| 13 | 13 | Homo sapiens (Human) SMARCA4 ISOFORM 2. [MASS = 188149 |
| 13 | 15 | Homo sapiens (Human) HYDROXYMETHYLGLUTARYL-COA SYNTHASE, |
| | | CYTOPLASMIC. [MASS = 57294 |
| 13 | 16 | Homo sapiens (Human) RIBONUCLEOSIDE-DIPHOSPHATE REDUCTASE LARGE |
| | | SUBUNIT. [MASS = 90070 |
| 13 | 28 | Homo sapiens (Human) HNRPA2B1 PROTEIN. [MASS = 28412 |
| 13 | 45 | Homo sapiens (Human) ENOLASE 1. [MASS = 47169 |
| 13 | 15 | Homo sapiens (Human) ALPHA-ACTININ-1. [MASS = 103058 |
| 13 | 29 | Homo sapiens (Human) DIHYDROPYRIMIDINASE-LIKE 2. [MASS = 67017 |
| 13 | 15 | Homo sapiens (Human) DIHYDROPYRIMIDINASE-RELATED PROTEIN 4. [MASS = 61878 |
| 13 | 20 | Homo sapiens (Human) INSULIN-LIKE GROWTH FACTOR 2 MRNA BINDING |
| | | PROTEIN 1. [MASS = 63481 |
| 13 | 17 | Homo sapiens (Human) ISOFORM 2 OF NEUTRAL ALPHA-GLUCOSIDASE AB |
| | | PRECURSOR. [MASS = 109438 |
| 13 | 13 | Homo sapiens (Human) ISOFORM P150 OF DYNACTIN-1. [MASS = 141695 |
| 13 | 14 | Homo sapiens (Human) SPLICING FACTOR 3B SUBUNIT 2. [MASS = 100228 |
| 13 | 25 | Homo sapiens (Human) HEMOGLOBIN SUBUNIT EPSILON. [MASS = 16072 |
| 13 | 17 | Homo sapiens (Human) DNA REPLICATION LICENSING FACTOR MCM5. [MASS = 82286 |
| 13 | 18 | Homo sapiens (Human) FASCIN. [MASS = 54399 |
| 13 | 15 | Homo sapiens (Human) GLUTAMYL-PROLYL TRNA SYNTHETASE. [MASS = 170591 |
| 13 | 14 | Homo sapiens (Human) DNA REPLICATION LICENSING FACTOR MCM3. [MASS = 90981 |
| 12 | 25 | Homo sapiens (Human) UBIQUITIN-ACTIVATING ENZYME E1. [MASS = 117849 |
| 12 | 68 | Homo sapiens (Human) TRANSTHYRETIN PRECURSOR. [MASS = 15887 |
| 12 | 18 | Homo sapiens (Human) SPLICING FACTOR 3B SUBUNIT 3. [MASS = 135592 |
| 12 | 13 | Homo sapiens (Human) PHOSPHOFRUCTOKINASE, MUSCLE. [MASS = 85183 |
| 12 | 41 | Homo sapiens (Human) TUBULIN BETA-3 CHAIN. [MASS = 50433 |
| 12 | 12 | Homo sapiens (Human) ALPHA-ACTININ-4. [MASS = 104854 |
| 12 | 15 | Homo sapiens (Human) ISOFORM 1 OF GELSOLIN PRECURSOR. [MASS = 85698 |
| 12 | 14 | Homo sapiens (Human) MULTIFUNCTIONAL PROTEIN ADE2. [MASS = 49679 |
| 12 | 15 | Homo sapiens (Human) DIHYDROPYRIMIDINASE-RELATED PROTEIN 5. [MASS = 61421 |
| 12 | 22 | Homo sapiens (Human) ANNEXIN A5. [MASS = 35806 |
| 12 | 25 | Homo sapiens (Human) L-LACTATE DEHYDROGENASE B CHAIN. [MASS = 36507 |
| 12 | 16 | Homo sapiens (Human) CALNEXIN PRECURSOR. [MASS = 67568 |
| 12 | 23 | Homo sapiens (Human) ISOFORM 1 OF FIBRINOGEN ALPHA CHAIN PRECURSOR. |
| | | [MASS = 94973 |
| 12 | 18 | Homo sapiens (Human) NON-POU DOMAIN-CONTAINING OCTAMER-BINDING PROTEIN. |
| | | [MASS = 54232 |
| 12 | 13 | Homo sapiens (Human) 116 KDA U5 SMALL NUCLEAR RIBONUCLEOPROTEIN |
| | | COMPONENT. [MASS = 109436 |
| 12 | 15 | Homo sapiens (Human) STRESS-INDUCED-PHOSPHOPROTEIN 1. [MASS = 62639 |
| 12 | 12 | Homo sapiens (Human) COATOMER SUBUNIT ALPHA. [MASS = 138332 |
| 11 | 14 | Homo sapiens (Human) T-COMPLEX PROTEIN 1 SUBUNIT DELTA. [MASS = 57793 |
| 11 | 17 | Homo sapiens (Human) ISOFORM 1 OF HETEROGENEOUS NUCLEAR |
| | | RIBONUCLEOPROTEIN D0. [MASS = 38434 |
| 11 | 12 | Homo sapiens (Human) CDNA FLJ45525 FIS, CLONE BRTHA2026311, HIGHLY SIMILAR |
| | | TO PROTEIN DISULFIDE ISOMERASE A6. [MASS = 53929 |
| 11 | 12 | Homo sapiens (Human) ATP-DEPENDENT RNA HELICASE DDX3X. [MASS = 73112 |
| 11 | 13 | Homo sapiens (Human) 26S PROTEASE REGULATORY SUBUNIT 6A. [MASS = 49204 |
| 11 | 15 | Homo sapiens (Human) GLYCOGEN PHOSPHORYLASE, BRAIN FORM. [MASS = 96565 |
| 11 | 12 | Homo sapiens (Human) PROTEASOME 26S NON-ATPASE SUBUNIT 11 VARIANT |
| | | (FRAGMENT). [MASS = 47535 |
| 11 | 11 | Homo sapiens (Human) DNA DAMAGE-BINDING PROTEIN 1. [MASS = 126968 |
| 11 | 11 | Homo sapiens (Human) RAS GTPASE-ACTIVATING-LIKE PROTEIN IQGAP1. |
| | | [MASS = 189252 |
| 11 | 15 | Homo sapiens (Human) STAPHYLOCOCCAL NUCLEASE DOMAIN-CONTAINING |
| | | PROTEIN 1. [MASS = 101997 |
| 11 | 23 | Homo sapiens (Human) UBIQUITIN-ACTIVATING ENZYME E1. [MASS = 56852 |
| 11 | 12 | Homo sapiens (Human) ISOFORM 2 OF PROTEIN KIAA1967. [MASS = 103030 |
| 11 | 11 | Homo sapiens (Human) ISOFORM 2 OF MICROTUBULE-ASSOCIATED PROTEIN 4. |
| | | [MASS = 102906 |
| 11 | 14 | Homo sapiens (Human) ISOFORM GTBP-ALT OF DNA MISMATCH REPAIR PROTEIN |
| | | MSH6. [MASS = 120563 |
| 11 | 14 | Homo sapiens (Human) 150 KDA OXYGEN-REGULATED PROTEIN PRECURSOR. |
| | | [MASS = 111335 |
| 11 | 11 | Homo sapiens (Human) TALDO1 PROTEIN. [MASS = 35329 |
| 11 | 24 | Homo sapiens (Human) STATHMIN. [MASS = 17171 |
| 11 | 11 | Homo sapiens (Human) ISOFORM 1 OF CHROMODOMAIN HELICASE-DNA-BINDING |
| | | PROTEIN 4. [MASS = 217991 |
| 10 | 11 | Homo sapiens (Human) RUVB-LIKE 2. [MASS = 51025 |
| 10 | 14 | Homo sapiens (Human) PLASMA PROTEASE C1 INHIBITOR PRECURSOR. [MASS = 55154 |
| 10 | 34 | Homo sapiens (Human) HEAT SHOCK PROTEIN HSP 90-ALPHA 2. [MASS = 98113 |
| 10 | 10 | Homo sapiens (Human) PIGMENT EPITHELIUM-DERIVED FACTOR PRECURSOR. |
| | | [MASS = 46342 |
| 10 | 11 | Homo sapiens (Human) ISOFORM LONG OF UBIQUITIN CARBOXYL-TERMINAL |
| | | HYDROLASE 5. [MASS = 95786 |
| 10 | 47 | Homo sapiens (Human) PRO2275. [MASS = 13097 |
| 10 | 12 | Homo sapiens (Human) T-COMPLEX PROTEIN 1 SUBUNIT ZETA. [MASS = 57893 |
| 10 | 36 | Homo sapiens (Human) MYRISTOYLATED ALANINE-RICH C-KINASE SUBSTRATE. |
| | | [MASS = 31423 |
| 10 | 10 | Homo sapiens (Human) TALIN-1. [MASS = 269767 |
| 10 | 11 | Homo sapiens (Human) VACUOLAR PROTEIN SORTING 35. [MASS = 91707 |
| 10 | 12 | Homo sapiens (Human) T-COMPLEX PROTEIN 1 SUBUNIT EPSILON. [MASS = 59671 |
| 10 | 11 | Homo sapiens (Human) ISOFORM 3 OF POLYPYRIMIDINE TRACT-BINDING PROTEIN 2. |
| | | [MASS = 58084 |
| 10 | 10 | Homo sapiens (Human) ISOFORM 1 OF REGULATOR OF NONSENSE TRANSCRIPTS 1. |
| | | [MASS = 124345 |
| 10 | 10 | Homo sapiens (Human) UBIQUITIN-LIKE 1-ACTIVATING ENZYME E1B. [MASS = 71224 |
| 10 | 16 | Homo sapiens (Human) ISOFORM 1 OF CLATHRIN HEAVY CHAIN 2. [MASS = 187030 |
| 10 | 12 | Homo sapiens (Human) SEPTIN-7. [MASS = 50809 |
| 10 | 14 | Homo sapiens (Human) ADENYLYL CYCLASE-ASSOCIATED PROTEIN 1. [MASS = 51542 |
| 10 | 10 | Homo sapiens (Human) VALYL-TRNA SYNTHETASE. [MASS = 140476 |
| 10 | 11 | Homo sapiens (Human) DNA MISMATCH REPAIR PROTEIN MSH2. [MASS = 104743 |
| 10 | 28 | Homo sapiens (Human) HETEROGENEOUS NUCLEAR RIBONUCLEOPROTEIN R. |
| | | [MASS = 70943 |
| 10 | 19 | Homo sapiens (Human) FRUCTOSE-BISPHOSPHATE ALDOLASE A. [MASS = 39289 |
| 10 | 15 | Homo sapiens (Human) THREONYL-TRNA SYNTHETASE, CYTOPLASMIC. [MASS = 83435 |
| 10 | 11 | Homo sapiens (Human) STRESS-70 PROTEIN, MITOCHONDRIAL PRECURSOR. |
| | | [MASS = 73680 |
| 10 | 11 | Homo sapiens (Human) ELONGATION FACTOR 1-DELTA. [MASS = 30991 |
| 10 | 11 | Homo sapiens (Human) CYTOSKELETON-ASSOCIATED PROTEIN 5. [MASS = 225509 |
| 10 | 11 | Homo sapiens (Human) CALRETICULIN PRECURSOR. [MASS = 48142 |
| 10 | 13 | Homo sapiens (Human) PHOSPHORIBOSYLFORMYLGLYCINAMIDINE SYNTHASE. |
| | | [MASS = 144664 |
| 10 | 11 | Homo sapiens (Human) ISOFORM 4 OF TUBULIN-SPECIFIC CHAPERONE D. |
| | | [MASS = 138597 |
| 10 | 13 | Homo sapiens (Human) ISOFORM B OF ARSENITE-RESISTANCE PROTEIN 2. |
| | | [MASS = 100276 |
| 10 | 10 | Homo sapiens (Human) ISOFORM 2 OF STRUCTURAL MAINTENANCE OF |
| | | CHROMOSOMES 4-LIKE 1 PROTEIN. [MASS = 140278 |
| 10 | 13 | Homo sapiens (Human) ISOFORM 1 OF RETICULON-4. [MASS = 130102 |
| 10 | 45 | Homo sapiens (Human) HEMOGLOBIN SUBUNIT ZETA. [MASS = 15506 |
| 10 | 11 | Homo sapiens (Human) HSPC117 PROTEIN. [MASS = 55210 |
| 10 | 10 | Homo sapiens (Human) ISOFORM 1 OF SQUAMOUS CELL CARCINOMA ANTIGEN |
| | | RECOGNIZED BY T-CELLS 3. [MASS = 109935 |
| 10 | 14 | Homo sapiens (Human) 40S RIBOSOMAL PROTEIN S4, X ISOFORM. [MASS = 29467 |
| 10 | 10 | Homo sapiens (Human) TRANSKETOLASE. [MASS = 67878 |
| 9 | 13 | Homo sapiens (Human) ISOFORM 2 OF NUCLEOPHOSMIN. [MASS = 29465 |
| 9 | 10 | Homo sapiens (Human) UNCHARACTERIZED PROTEIN C20ORF77. [MASS = 36900 |
| 9 | 9 | Homo sapiens (Human) T-COMPLEX PROTEIN 1 SUBUNIT ALPHA. [MASS = 60344 |
| 9 | 10 | Homo sapiens (Human) INTERLEUKIN ENHANCER-BINDING FACTOR 2. [MASS = 43062 |
| 9 | 10 | Homo sapiens (Human) 26S PROTEASE REGULATORY SUBUNIT 8. [MASS = 45626 |
| 9 | 11 | Homo sapiens (Human) ISOFORM 1 OF DNA REPLICATION LICENSING FACTOR |
| | | MCM7. [MASS = 81308 |
| 9 | 19 | Homo sapiens (Human) GAMMA-ENOLASE. [MASS = 47137 |
| 9 | 11 | Homo sapiens (Human) HYPOTHETICAL PROTEIN DKFZP451D234. [MASS = 109187 |
| 9 | 23 | Homo sapiens (Human) TUBULIN, BETA 2. [MASS = 49907 |
| 9 | 11 | Homo sapiens (Human) ISOFORM 1 OF PROTEIN ARGININE |
| | | N-METHYLTRANSFERASE 1. [MASS = 41486 |
| 9 | 12 | Homo sapiens (Human) VILLIN 2. [MASS = 69413 |
| 9 | 11 | Homo sapiens (Human) RADIXIN. [MASS = 68564 |
| 9 | 11 | Homo sapiens (Human) ISOFORM 2 OF SWI/SNF-RELATED MATRIX-ASSOCIATED |
| | | ACTIN-DEPENDENT |
| | | REGULATOR OF CHROMATIN SUBFAMILY C MEMBER 2. [MASS = 124841 |
| 9 | 9 | Homo sapiens (Human) ASPARTYL-TRNA SYNTHETASE. [MASS = 57136 |
| 9 | 12 | Homo sapiens (Human) D-3-PHOSPHOGLYCERATE DEHYDROGENASE. [MASS = 56519 |
| 9 | 9 | Homo sapiens (Human) CAD PROTEIN. [MASS = 242984 |
| 9 | 9 | Homo sapiens (Human) CTP SYNTHASE 1. [MASS = 66690 |
| 9 | 9 | Homo sapiens (Human) SERINE-THREONINE KINASE RECEPTOR-ASSOCIATED |
| | | PROTEIN. [MASS = 38438 |
| 9 | 11 | Homo sapiens (Human) ADENOSYLHOMOCYSTEINASE. [MASS = 47585 |
| 9 | 11 | Homo sapiens (Human) ELONGATION FACTOR 1-GAMMA. [MASS = 49988 |
| 9 | 20 | Homo sapiens (Human) CDNA FLJ45706 FIS, CLONE FEBRA2028457, HIGHLY SIMILAR |
| | | TO NUCLEOLIN. [MASS = 65962 |
| 9 | 10 | Homo sapiens (Human) CHAPERONIN CONTAINING TCP1, SUBUNIT 3 ISOFORM B. |
| | | [MASS = 60463 |
| 9 | 12 | Homo sapiens (Human) ENO1P PROTEIN. [MASS = 42342 |
| 9 | 9 | Homo sapiens (Human) PREDICTED: SIMILAR TO HETEROGENEOUS NUCLEAR |
| | | RIBONUCLEOPROTEIN U. [MASS = 101752 |
| 9 | 11 | Homo sapiens (Human) ISOFORM SHORT OF RECEPTOR-TYPE TYROSINE-PROTEIN |
| | | PHOSPHATASE ZETA PRECURSOR. [MASS = 163444 |
| 9 | 11 | Homo sapiens (Human) 26S PROTEASE REGULATORY SUBUNIT 7. [MASS = 48503 |
| 9 | 10 | Homo sapiens (Human) PROTEIN DISULFIDE-ISOMERASE PRECURSOR. [MASS = 57116 |
| 9 | 11 | Homo sapiens (Human) FACT COMPLEX SUBUNIT SPT16. [MASS = 119914 |
| 9 | 14 | Homo sapiens (Human) LUMICAN PRECURSOR. [MASS = 38429 |
| 9 | 10 | Homo sapiens (Human) PROLIFERATION-ASSOCIATED PROTEIN 2G4. [MASS = 43656 |
| 9 | 10 | Homo sapiens (Human) IARS PROTEIN. [MASS = 120627 |
| 9 | 10 | Homo sapiens (Human) U5 SMALL NUCLEAR RIBONUCLEOPROTEIN 200 KDA |
| | | HELICASE. [MASS = 244508 |
| 9 | 9 | Homo sapiens (Human) UBIQUITIN CARBOXYL-TERMINAL HYDROLASE 7. |
| | | [MASS = 128272 |
| 9 | 10 | Homo sapiens (Human) INTER-ALPHA-TRYPSIN INHIBITOR HEAVY CHAIN |
| | | H1 PRECURSOR. [MASS = 101389 |
| 9 | 12 | Homo sapiens (Human) ATP-DEPENDENT RNA HELICASE DDX1. [MASS = 82432 |
| 9 | 13 | Homo sapiens (Human) ISOFORM 1 OF PROBABLE ATP-DEPENDENT RNA |
| | | HELICASE DDX17. [MASS = 72371 |
| 9 | 10 | Homo sapiens (Human) RNA-BINDING PROTEIN 12. [MASS = 97395 |
| 9 | 9 | Homo sapiens (Human) TRANSPORTIN 1. [MASS = 102355 |
| 9 | 9 | Homo sapiens (Human) GTP-BINDING NUCLEAR PROTEIN RAN. [MASS = 24292 |
| 9 | 9 | Homo sapiens (Human) 40S RIBOSOMAL PROTEIN S18. [MASS = 17719 |
| 9 | 30 | Homo sapiens (Human) ACTIN, AORTIC SMOOTH MUSCLE [MASS = 42009 |
| 9 | 10 | Homo sapiens (Human) EUKARYOTIC TRANSLATION INITIATION FACTOR 4 |
| | | GAMMA 2. [MASS = 102362 |
| 9 | 12 | Homo sapiens (Human) SPLICING FACTOR, ARGININE/SERINE-RICH 1. [MASS = 27745 |
| 9 | 9 | Homo sapiens (Human) PEROXIREDOXIN-1. [MASS = 22110 |
| 8 | 10 | Homo sapiens (Human) GLUTATHIONE S-TRANSFERASE P. [MASS = 23225 |
| 8 | 31 | Homo sapiens (Human) ACTIN, CYTOPLASMIC 1. [MASS = 41737 |
| 8 | 9 | Homo sapiens (Human) HEAT SHOCK 70 KDA PROTEIN 1L. [MASS = 70375 |
| 8 | 12 | Homo sapiens (Human) COFILIN-1. [MASS = 18371 |
| 8 | 13 | Homo sapiens (Human) ANGIOTENSINOGEN PRECURSOR. [MASS = 53154 |
| 8 | 10 | Hoino sapiens (Human) GLUCOSE-6-PHOSPHATE ISOMERASE. [MASS = 63016 |
| 8 | 51 | Homo sapiens (Human) TUBULIN BETA-2 CHAIN. [MASS = 49671 |
| 8 | 12 | Homo sapiens (Human) ISOFORM 1 OF CYTOSOLIC ACYL COENZYME A THIOESTER |
| | | HYDROLASE. [MASS = 41796 |
| 8 | 10 | Homo sapiens (Human) HEAT SHOCK 70 KDA PROTEIN 1. [MASS = 70052 |
| 8 | 9 | Homo sapiens (Human) 26S PROTEASOME NON-ATPASE REGULATORY SUBUNIT 12. |
| | | [MASS = 52773 |
| 8 | 23 | Homo sapiens (Human) GAMMA-G GLOBIN (FRAGMENT). [MASS = 16969 |
| 8 | 9 | Homo sapiens (Human) 14-3-3 PROTEIN THETA. WASS = 27764 |
| 8 | 8 | Homo sapiens (Human) MALATE DEHYDROGENASE, MITOCHONDRIAL PRECURSOR. |
| | | [MASS = 35531 |
| 8 | 9 | Homo sapiens (Human) A-KINASE ANCHOR PROTEIN 12 ISOFORM 2. [MASS = 181690 |
| 8 | 8 | Homo sapiens (Human) LIVER PHOSPHOFRUCTOKINASE ISOFORM A. [MASS = 90577 |
| 8 | 14 | Homo sapiens (Human) 14-3-3 PROTEIN ZETA/DELTA. [MASS = 27745 |
| 8 | 10 | Homo sapiens (Human) ATP-DEPENDENT RNA HELICASE A. [MASS = 140881 |
| 8 | 15 | Homo sapiens (Human) ISOFORM C1 OF HETEROGENEOUS NUCLEAR |
| | | RIBONUCLEOPROTEINS C1/C2. [MASS = 32338 |
| 8 | 8 | Homo sapiens (Human) NUCLEOSIDE DIPHOSPHATE KINASE A. [MASS = 17149 |
| 8 | 8 | Homo sapiens (Human) TRIPEPTIDYL-PEPTIDASE 2. [MASS = 138219 |
| 8 | 11 | Homo sapiens (Human) RCTPI1 (FRAGMENT). [MASS = 26943 |
| 8 | 11 | Homo sapiens (Human) ISOFORM 1 OF POLYPYRIMIDINE TRACT-BINDING PROTEIN 1. |
| | | [MASS = 57221 |
| 8 | 11 | Homo sapiens (Human) CELLULAR RETINOIC ACID-BINDING PROTEIN 1. [MASS = 15434 |
| 8 | 8 | Homo sapiens (Human) MALATE DEHYDROGENASE, CYTOPLASMIC. [MASS = 36295 |
| 8 | 10 | Homo sapiens (Human) ESTERASE D. [MASS = 31463 |
| 8 | 15 | Homo sapiens (Human) ISOFORM 1 OF HETEROGENEOUS NUCLEAR |
| | | RIBONUCLEOPROTEIN Q. [MASS = 69603 |
| 8 | 12 | Homo sapiens (Human) ISOFORM SHORT OF RNA-BINDING PROTEIN FUS. [MASS = 53355 |
| 8 | 9 | Homo sapiens (Human) F-ACTIN CAPPING PROTEIN ALPHA-1 SUBUNIT. [MASS = 32792 |
| 8 | 10 | Homo sapiens (Human) PROBABLE ATP-DEPENDENT RNA HELICASE DDX48. [MASS = 46740 |
| 8 | 10 | Homo sapiens (Human) INORGANIC PYROPHOSPHATASE. [MASS = 32660 |
| 8 | 21 | Homo sapiens (Human) HETEROGENEOUS NUCLEAR RIBONUCLEOPROTEIN A0. [MASS = 30841 |
| 8 | 9 | Homo sapiens (Human) ISOFORM 1 OF GLUCOSAMINE--FRUCTOSE-6-PHOSPHATE |
| | | AMINOTRANSFERASE [ISOMERIZING] 1. [MASS = 78806 |
| 8 | 10 | Homo sapiens (Human) ELAV. [MASS = 42417 |
| 8 | 8 | Homo sapiens (Human) CDNA FLJ33352 FIS, CLONE BRACE2005087, WEAKLY SIMILAR |
| | | TO PRE-MRNA SPLICING HELICASE BRR2. [MASS = 71472 |
| 8 | 8 | Homo sapiens (Human) PREDICTED: SIMILAR TO ATP-DEPENDENT DNA HELICASE II, |
| | | 70 KDA SUBUNIT (LUPUS KU AUTOANTIGEN PROTEIN P70) (KU70) (70 KDA SUBUNIT OF KU |
| | | ANTIGEN) (THYROID-LUPUS AUTOANTIGEN) (TLAA) (CTC BOX BINDING FACTOR 75 KDA |
| | | SUBUNIT) (CTCBF) (CTC75) ISOFORM 1. [MASS = 54430 |
| 8 | 11 | Homo sapiens (Human) IMPORTIN-7. [MASS = 119517 |
| 8 | 8 | Homo sapiens (Human) ISOFORM 1 OF CULLIN-3. [MASS = 88930 |
| 8 | 8 | Homo sapiens (Human) 26S PROTEASOME NON-ATPASE REGULATORY SUBUNIT 6. [MASS = 45531 |
| 8 | 9 | Homo sapiens (Human) PROTEIN PHOSPHATASE 2C ISOFORM GAMMA. [MASS = 59272 |
| 8 | 24 | Homo sapiens (Human) HYPOTHETICAL PROTEIN LOC345651. [MASS = 42003 |
| 8 | 9 | Homo sapiens (Human) 26S PROTEASOME NON-ATPASE REGULATORY SUBUNIT 3. [MASS = 60978 |
| 8 | 10 | Homo sapiens (Human) ISOFORM 1 OF EUKARYOTIC TRANSLATION INITIATION FACTOR 3 |
| | | SUBUNIT 9. [MASS = 92492 |
| 8 | 9 | Homo sapiens (Human) 60S ACIDIC RIBOSOMAL PROTEIN P0. [MASS = 34274 |
| 8 | 8 | Homo sapiens (Human) FACT COMPLEX SUBUNIT SSRP1. [MASS = 81075 |
| 8 | 12 | Homo sapiens (Human) NUCLEOSOME ASSEMBLY PROTEIN 1-LIKE 4. [MASS = 43011 |
| 8 | 12 | Homo sapiens (Human) ISOFORM BETA-2 OF DNA TOPOISOMERASE 2-BETA. [MASS = 183267 |
| 8 | 9 | Homo sapiens (Human) GARS PROTEIN. [MASS = 84648 |
| 8 | 8 | Homo sapiens (Human) STRUCTURAL MAINTENANCE OF CHROMOSOME 1-LIKE 1 PROTEIN. |
| | | [MASS = 143233 |
| 8 | 8 | Homo sapiens (Human) DNA-DIRECTED RNA POLYMERASE II 140 KDA POLYPEPTIDE. |
| | | [MASS = 133897 |
| 8 | 24 | Homo sapiens (Human) NESTIN. [MASS = 177439 |
| 8 | 8 | Homo sapiens (Human) PREDICTED: SIMILAR TO PEPTIDYLPROLYL ISOMERASE |
| | | A ISOFORM 1. [MASS = 24517 |
| 8 | 11 | Homo sapiens (Human) HETEROGENEOUS NUCLEAR RIBONUCLEOPROTEIN D-LIKE. [MASS = 46438 |
| 8 | 10 | Homo sapiens (Human) ISOFORM 2C OF CYTOPLASMIC DYNEIN 1 INTERMEDIATE CHAIN 2. |
| | | [MASS = 68426 |
| 7 | 7 | Homo sapiens (Human) CSNK2A1 PROTEIN. [MASS = 45909 |
| 7 | 7 | Homo sapiens (Human) PEROXIREDOXIN-6. [MASS = 24904 |
| 7 | 9 | Homo sapiens (Human) HIGH MOBILITY GROUP PROTEIN 1-LIKE 10. [MASS = 24218 |
| 7 | 10 | Homo sapiens (Human) CALMODULIN. [MASS = 16706 |
| 7 | 9 | Homo sapiens (Human) NUCLEOSOME ASSEMBLY PROTEIN 1-LIKE 1. [MASS = 45374 |
| 7 | 10 | Homo sapiens (Human) IMPORTIN-9. [MASS = 115832 |
| 7 | 13 | Homo sapiens (Human) ISOFORM 1 OF PROTEIN SET. [MASS = 33489 |
| 7 | 11 | Homo sapiens (Human) HETEROGENEOUS NUCLEAR RIBONUCLEOPROTEIN H1. [MASS = 49229 |
| 7 | 9 | Homo sapiens (Human) SEPTIN-2. [MASS = 41487 |
| 7 | 7 | Homo sapiens (Human) ATAXIN-10. [MASS = 53489 |
| 7 | 7 | Homo sapiens (Human) COATOMER SUBUNIT BETA. [MASS = 107139 |
| 7 | 9 | Homo sapiens (Human) ISOFORM 2 OF DNA REPLICATION LICENSING FACTOR MCM7. |
| | | [MASS = 44649 |
| 7 | 7 | Homo sapiens (Human) IRON-RESPONSIVE ELEMENT-BINDING PROTEIN 1. [MASS = 98399 |
| 7 | 10 | Homo sapiens (Human) TYROSINE 3-MONOOXYGENASE/TRYPTOPHAN 5-MONOOXYGENASE |
| | | ACTIVATION PROTEIN, BETA POLYPEPTIDE. [MASS = 28082 |
| 7 | 9 | Homo sapiens (Human) 26S PROTEASE REGULATORY SUBUNIT S10B. [MASS = 44173 |
| 7 | 8 | Homo sapiens (Human) HYPOTHETICAL PROTEIN DKFZP781K0743. [MASS = 105850 |
| 7 | 8 | Homo sapiens (Human) UBIQUITIN CARBOXYL-TERMINAL HYDROLASE ISOZYME L1. |
| | | [MASS = 24824 |
| 7 | 9 | Homo sapiens (Human) ISOFORM 1 OF HETEROGENEOUS NUCLEAR RIBONUCLEOPROTEIN |
| | | U-LIKE PROTEIN 1. [MASS = 95739 |
| 7 | 8 | Homo sapiens (Human) TUBULIN-TYROSINE LIGASE-LIKE PROTEIN 12. [MASS = 74404 |
| 7 | 7 | Homo sapiens (Human) ISOFORM 2 OF NUCLEAR MITOTIC APPARATUS PROTEIN 1. |
| | | [MASS = 236531 |
| 7 | 8 | Homo sapiens (Human) ALPHA ISOFORM OF REGULATORY SUBUNIT A, PROTEIN |
| | | PHOSPHATASE 2. [MASS = 65309 |
| 7 | 8 | Homo sapiens (Human) ISOFORM 4 OF HETEROGENEOUS NUCLEAR |
| | | RIBONUCLEOPROTEIN A/B. [MASS = 31233 |
| 7 | 8 | Homo sapiens (Human) CCR4-NOT TRANSCRIPTION COMPLEX, SUBUNIT 1 ISOFORM A. |
| | | [MASS = 266939 |
| 7 | 8 | Homo sapiens (Human) PROFILIN-1. [MASS = 14923 |
| 7 | 11 | Homo sapiens (Human) PROLIFERATING CELL NUCLEAR ANTIGEN. [MASS = 28769 |
| 7 | 7 | Homo sapiens (Human) METHIONYL-TRNA SYNTHETASE. [MASS = 101116 |
| 7 | 8 | Homo sapiens (Human) UBIQUITIN-LIKE 1-ACTIVATING ENZYME E1A. [MASS = 38450 |
| 7 | 8 | Homo sapiens (Human) ALCOHOL DEHYDROGENASE. [MASS = 36442 |
| 7 | 7 | Homo sapiens ,(Human) ADP-RIBOSYLATION FACTOR 1. [MASS = 20566 |
| 7 | 9 | Homo sapiens (Human) THIOREDOXIN-LIKE PROTEIN 1. [MASS = 32120 |
| 7 | 11 | Homo sapiens (Human) ISOFORM 1 OF 26S PROTEASOME NON-ATPASE REGULATORY |
| | | SUBUNIT 1. [MASS = 105836 |
| 7 | 7 | Homo sapiens (Human) EUKARYOTIC TRANSLATION INITIATION FACTOR 3 SUBUNIT 8. |
| | | [MASS = 105344 |
| 7 | 7 | Homo sapiens (Human) VESICLE-FUSING ATPASE. [MASS = 82654 |
| 7 | 8 | Homo sapiens (Human) Complement component 3 precursor. [MASS = 187306 |
| 7 | 7 | Homo sapiens (Human) EUKARYOTIC TRANSLATION INITIATION FACTOR 3 SUBUNIT 6. |
| | | [MASS = 52221 |
| 7 | 8 | Homo sapiens (Human) ATP-DEPENDENT DNA HELICASE 2 SUBUNIT 1. [MASS = 69712 |
| 7 | 7 | Homo sapiens (Human) ISOFORM 1 OF HOST CELL FACTOR. [MASS = 208842 |
| 7 | 7 | Homo sapiens (Human) METASTASIS-ASSOCIATED PROTEIN MTA2. [MASS = 75023 |
| 7 | 7 | Homo sapiens (Human) HETEROGENEOUS NUCLEAR RIBONUCLEOPROTEIN L ISOFORM |
| | | A. [MASS = 64133 |
| 7 | 8 | Homo sapiens (Human) 26S PROTEASE REGULATORY SUBUNIT 4. [MASS = 49185 |
| 7 | 8 | Homo sapiens (Human) UDP-GLUCOSE CERAMIDE GLUCOSYLTRANSFERASE-LIKE 1 |
| | | ISOFORM 1. [MASS = 177190 |
| 7 | 7 | Homo sapiens (Human) EUKARYOTIC TRANSLATION INITIATION FACTOR 5B. [MASS = 138800 |
| 7 | 7 | Homo sapiens (Human) ISOFORM V0 OF VERSICAN CORE PROTEIN PRECURSOR. [MASS = 372820 |
| 7 | 26 | Homo sapiens (Human) TUBULIN BETA-1 CHAIN. [MASS = 50327 |
| 7 | 7 | Homo sapiens (Human) ISOFORM 1 OF FILAMIN-B. [MASS = 278195 |
| 7 | 7 | Homo sapiens (Human) CONTACTIN-2 PRECURSOR. [MASS = 113393 |
| 7 | 7 | Homo sapiens (Human) UBIQUITIN SPECIFIC PROTEASE 9, X-LINKED ISOFORM 4. [MASS = 290497 |
| 6 | 11 | Homo sapiens (Human) ISOFORM 1 OF HETEROGENEOUS NUCLEAR RIBONUCLEOPROTEIN |
| | | A3. [MASS = 39595 |
| 6 | 11 | Homo sapiens (Human) HEMOGLOBIN SUBUNIT ALPHA: [MASS = 15126 |
| 6 | 8 | Homo sapiens (Human) ASTROCYTIC PHOSPHOPROTEIN PEA-15. [MASS = 15040 |
| 6 | 6 | Homo sapiens (Human) 26S PROTEASOME NON-ATPASE REGULATORY SUBUNIT 7. [MASS = 37025 |
| 6 | 7 | Homo sapiens (Human) PROTEASOME SUBUNIT BETA TYPE 1. [MASS = 26489 |
| 6 | 6 | Homo sapiens (Human) ALPHA-CENTRACTIN. [MASS = 42614 |
| 6 | 10 | Homo sapiens (Human) ISOFORM 1 OF PLASMINOGEN ACTIVATOR INHIBITOR 1 |
| | | RNA-BINDING PROTEIN. [MASS = 44965 |
| 6 | 7 | Homo sapiens (Human) CALPONIN-3. [MASS = 36414 |
| 6 | 7 | Homo sapiens (Human) PROTEASOME SUBUNIT ALPHA TYPE 2. [MASS = 25767 |
| 6 | 8 | Homo sapiens (Human) PHOSPHATIDYLETHANOLAMINE-BINDING. PROTEIN 1. [MASS = 20926 |
| 6 | 6 | Homo sapiens (Human) METHIONINE ADENOSYLTRANSFERASE II, BETA ISOFORM 1. |
| | | [MASS = 37552 |
| 6 | 12 | Homo sapiens (Human) VITRONECTIN PRECURSOR. [MASS = 54306 |
| 6 | 7 | Homo sapiens (Human) PHOSPHORIBOSYL PYROPHOSPHATE SYNTHETASE-ASSOCIATED |
| | | PROTEIN 2. [MASS = 40926 |
| 6 | 6 | Homo sapiens (Human) CONDENSIN COMPLEX SUBUNIT 3. [MASS = 114334 |
| 6 | 10 | Homo sapiens (Human) PEROXIREDOXIN-2. [MASS = 21761 |
| 6 | 12 | Homo sapiens (Human) HETEROGENEOUS NUCLEAR RIBONUCLEOPROTEIN A1 |
| | | ISOFORM B. [MASS = 38747 |
| 6 | 8 | Homo sapiens (Human) RHO GDP-DISSOCIATION INHIBITOR 1. [MASS = 23076 |
| 6 | 6 | Homo sapiens (Human) ISOFORM 1 OF DIPEPTIDYL-PEPTIDASE 3. [MASS = 82589 |
| 6 | 6 | Homo sapiens (Human) ISOFORM 1 OF ACTIN-LIKE PROTEIN 6A. [MASS = 47461 |
| 6 | 6 | Homo sapiens (Human) ISOFORM 2 OF NSFL1 COFACTOR P47. [MASS = 37325 |
| 6 | 6 | Homo sapiens (Human) EUKARYOTIC TRANSLATION INITIATION FACTOR 4 GAMMA, |
| | | 1 ISOFORM 2. [MASS = 166589 |
| 6 | 53 | Homo sapiens (Human) ELONGATION FACTOR 1-ALPHA 2. [MASS = 50470 |
| 6 | 8 | Homo sapiens (Human) HYPOTHETICAL PROTEIN DKFZP564E242. [MASS = 31424 |
| 6 | 6 | Homo sapiens (Human) 3-MERCAPTOPYRUVATE SULFURTRANSFERASE. [MASS = 33047 |
| 6 | 7 | Homo sapiens (Human) CLUSTERIN PRECURSOR. [MASS = 52495 |
| 6 | 6 | Homo sapiens (Human) REPLICATION PROTEIN A 70 KDA DNA-BINDING SUBUNIT. [MASS = 68138 |
| 6 | 7 | Homo sapiens (Human) DYNACTIN 2. [MASS = 44820 |
| 6 | 7 | Homo sapiens (Human) THIOREDOXIN-LIKE PROTEIN 2. [MASS = 37432 |
| 6 | 6 | Homo sapiens (Human) ISOFORM 2 OF CADHERIN-11 PRECURSOR. [MASS = 76541 |
| 6 | 9 | Homo sapiens (Human) S-ADENOSYLMETHIONINE SYNTHETASE ISOFORM TYPE-2. [MASS = 43661 |
| 6 | 7 | Homo sapiens (Human) ISOFORM 1 OF ELAV-LIKE PROTEIN 3. [MASS = 39547 |
| 6 | 6 | Homo sapiens (Human) SIMILAR TO ANNEXIN A2 ISOFORM 1. [MASS = 38659 |
| 6 | 8 | Homo sapiens (Human) ASPARTATE AMINOTRANSFERASE, CYTOPLASMIC. [MASS = 46116 |
| 6 | 7 | Homo sapiens (Human) NUCLEAR MIGRATION PROTEIN NUDC. [MASS = 38243 |
| 6 | 7 | Homo sapiens (Human) 40S RIBOSOMAL PROTEIN S19. [MASS = 15929 |
| 6 | 6 | Homo sapiens (Human) RNA BINDING PROTEIN (FRAGMENT). [MASS = 32550 |
| 6 | 6 | Homo sapiens (Human) ISOFORM 1 OF DNA. [MASS = 189566 |
| 6 | 9 | Homo sapiens (Human) ISOFORM 2 OF SERINE/THREONINE-PROTEIN KINASE DCAMKL1. |
| | | [MASS = 82224 |
| 6 | 6 | Homo sapiens (Human) PROTEIN TRANSPORT PROTEIN SEC23A. [MASS = 86147 |
| 6 | 10 | Homo sapiens (Human) 60S RIBOSOMAL PROTEIN L4. [MASS = 47566 |
| 6 | 7 | Homo sapiens (Human) PROTEASOME 26S NON-ATPASE SUBUNIT 13 ISOFORM 2. [MASS = 39871 |
| 6 | 6 | Homo sapiens (Human) ISOFORM 4 OF AFADIN. [MASS = 206804 |
| 6 | 16 | Homo sapiens (Human) DIHYDROPYRIMIDINASE-RELATED PROTEIN 2. [MASS = 62294 |
| 6 | 6 | Homo sapiens (Human) WUGSC:H_RG054D04.1 PROTEIN. [MASS = 29037 |
| 6 | 7 | Homo sapiens (Human) VACUOLAR ATP SYNTHASE CATALYTIC SUBUNIT A, UBIQUITOUS |
| | | ISOFORM. [MASS = 68304 |
| 6 | 6 | Homo sapiens (Human) MGEA5 PROTEIN. [MASS = 95331 |
| 6 | 7 | Homo sapiens (Human) GLUCOSIDASE 2 SUBUNIT BETA PRECURSOR. [MASS = 59296 |
| 6 | 6 | Homo sapiens (Human) CYSTEINYL-TRNA SYNTHETASE ISOFORM C.- [MASS = 94638 |
| 6 | 9 | Homo sapiens (Human) BIFUNCTIONAL PURINE BIOSYNTHESIS PROTEIN PURH. [MASS = 64616 |
| 6 | 6 | Homo sapiens (Human) HSC70-INTERACTING PROTEIN. [MASS = 41332 |
| 6 | 7 | Homo sapiens (Human) 40S RIBOSOMAL PROTEIN S3. [MASS = 26688 |
| 6 | 9 | Homo sapiens (Human) GALECTIN-3-BINDING PROTEIN PRECURSOR. [MASS = 65331 |
| 6 | 6 | Homo sapiens (Human) ISOFORM 1 OF COMPLEMENT FACTOR B PRECURSOR (FRAGMENT). |
| | | [MASS = 85533 |
| 6 | 6 | Homo sapiens (Human) ISOFORM 1 OF POLYADENYLATE-BINDING PROTEIN 4. [MASS = 70783 |
| 6 | 6 | Homo sapiens (Human) SPERMIDINE SYNTHASE. [MASS = 33825 |
| 6 | 7 | Homo sapiens (Human) PROTEIN DJ-1. [MASS = 19891 |
| 6 | 8 | Homo sapiens (Human) ISOFORM 1 OF HETEROGENEOUS NUCLEAR RIBONUCLEOPROTEIN |
| | | H3. [MASS = 36926 |
| 6 | 8 | Homo sapiens (Human) RAB1A, MEMBER RAS ONCOGENE FAMILY. [MASS = 22678 |
| 6 | 6 | Homo sapiens (Human) WD40 PROTEIN. [MASS = 35079 |
| 6 | 11 | Homo sapiens (Human) ISOFORM B1 OF HETEROGENEOUS NUCLEAR RIBONUCLEOPROTEINS |
| | | A2/B1. [MASS = 37430 |
| 6 | 7 | Homo sapiens (Human) RUVB-LIKE 1. [MASS = 50228 |
| 6 | 10 | Homo sapiens (Human) RNA BINDING MOTIF PROTEIN, X-LINKED-LIKE 1. [MASS = 42142 |
| 6 | 7 | Homo sapiens (Human) PHOSPHOGLYCERATE MUTASE 2. [MASS = 28635 |
| 6 | 7 | Homo sapiens (Human) DNA POLYMERASE DELTA CATALYTIC SUBUNIT. [MASS = 123631 |
| 6 | 6 | Homo sapiens (Human) SF3B3 PROTEIN. [MASS = 30210 |
| 6 | 6 | Homo sapiens (Human) RETINOBLASTOMA-ASSOCIATED FACTOR 600. [MASS = 185447 |
| 5 | 35 | TRYPSIN PRECURSOR (EC 3.4.21.4) > PIR1:TRPGTR trypsin (EC 3.4.21.4) |
| 5 | 8 | Homo sapiens (Human) TUBULIN BETA-4 CHAIN. [MASS = 49586 |
| 5 | 18 | Homo sapiens (Human) PROTHYMOSIN ALPHA. [MASS = 12203 |
| 5 | 6 | Homo sapiens (Human) 26S PROTEASOME NON-ATPASE REGULATORY SUBUNIT 14. [MASS = 34577 |
| 5 | 7 | Homo sapiens (Human) GLYOXYLATE REDUCTASE/HYDROXYPYRUVATE REDUCTASE. |
| | | [MASS = 35668 |
| 5 | 7 | Homo sapiens (Human) DNA-(APURINIC OR APYRIMIDINIC SITE) LYASE. [MASS = 35423 |
| 5 | 6 | Homo sapiens (Human) ISOFORM 2 OF GUANINE NUCLEOTIDE-BINDING PROTEIN G(I), |
| | | ALPHA-2 SUBUNIT. [MASS = 38473 |
| 5 | 5 | Homo sapiens (Human) ISOFORM 1 OF PROTEIN 4. 1. [MASS = 97017 |
| 5 | 7 | Homo sapiens (Human) ADP-SUGAR PYROPHOSPHATASE. [MASS = 24328 |
| 5 | 6 | Homo sapiens (Human) HISTONE H2B TYPE 2-E. [MASS = 13789 |
| 5 | 10 | Homo sapiens (Human) HETEROGENEOUS NUCLEAR RIBONUCLEOPROTEIN F. [MASS = 45672 |
| 5 | 9 | Homo sapiens (Human) PREDICTED: SIMILAR TO HETEROGENEOUS NUCLEAR |
| | | RIBONUCLEOPROTEIN A3 ISOFORM 1. [MASS = 31312 |
| 5 | 9 | Homo sapiens (Human) NG, NG-DIMETHYLARGININE DIMETHYLAMINOHYDROLASE 2. |
| | | [MASS = 29644 |
| 5 | 5 | Homo sapiens (Human) SERYL-TRNA SYNTHETASE. [MASS = 58646 |
| 5 | 5 | Homo sapiens (Human) NUCLEASE SENSITIVE ELEMENT-BINDING PROTEIN 1. |
| | | [MASS = 35793 |
| 5 | 10 | Homo sapiens (Human) ISOFORM 1 OF CLEAVAGE AND POLYADENYLATION SPECIFICITY |
| | | FACTOR 6. [MASS = 59210 |
| 5 | 7 | Homo sapiens (Human) POLY(RC)-BINDING PROTEIN 1. [MASS = 37498 |
| 5 | 7 | Homo sapiens (Human) ISOFORM B OF FIBULIN-1 PRECURSOR. [MASS = 77186 |
| 5 | 5 | Homo sapiens (Human) FK506-BINDING PROTEIN 3. [MASS = 25177 |
| 5 | 5 | Homo sapiens (Human) CTTN PROTEIN. [MASS = 70959 |
| 5 | 14 | Homo sapiens (Human) PREDICTED: SIMILAR TO HETEROGENEOUS NUCLEAR |
| | | RIBONUCLEOPROTEIN A1. [MASS = 32163 |
| 5 | 5 | Homo sapiens (Human) PRE-MRNA-SPLICING FACTOR 19. [MASS = 55181 |
| 5 | 7 | Homo sapiens (Human) 40S RIBOSOMAL PROTEIN S15. [MASS = 16909 |
| 5 | 6 | Homo sapiens (Human) GLUTAMINYL-TRNA SYNTHETASE. [MASS = 87799 |
| 5 | 5 | Homo sapiens (Human) PROTEIN RCC2. [MASS = 56085 |
| 5 | 8 | Homo sapiens (Human) DOUBLECORTEX\; LISSENCEPHALY, X-LINKED. [MASS = 49847 |
| 5 | 5 | Homo sapiens (Human) ISOFORM B OF MANNOSE-6-PHOSPHATE RECEPTOR-BINDING |
| | | PROTEIN 1. [MASS = 47047 |
| 5 | 6 | Homo sapiens (Human) COATOMER SUBUNIT BETA’. [MASS = 102356 |
| 5 | 7 | Homo sapiens (Human) HEPATOMA-DERIVED GROWTH FACTOR. [MASS = 26788 |
| 5 | 7 | Homo sapiens (Human) ELAV-LIKE PROTEIN 1. [MASS = 36092 |
| 5 | 5 | Homo sapiens (Human) PP856. [MASS = 43833 |
| 5 | 7 | Homo sapiens (Human) RAS-RELATED PROTEIN RAB-2A. [MASS = 23546 |
| 5 | 6 | Homo sapiens (Human) ISOFORM 2 OF PROTEIN ENABLED HOMOLOG. [MASS = 63924 |
| 5 | 7 | Homo sapiens (Human) ISOFORM 1 OF BETA-CATENIN. [MASS = 85497 |
| 5 | 5 | Homo sapiens (Human) AP-2 COMPLEX SUBUNIT ALPHA-2. [MASS = 104150 |
| 5 | 20 | Homo sapiens (Human) EEF1A1 PROTEIN. [MASS = 47869 |
| 5 | 7 | Homo sapiens (Human) 40S RIBOSOMAL PROTEIN S17. [MASS = 15419 |
| 5 | 6 | Homo sapiens (Human) 14-3-3 PROTEIN GAMMA. [MASS = 28171 |
| 5 | 5 | Homo sapiens (Human) RAS-GTPASE-ACTIVATING PROTEIN-BINDING PROTEIN 1. [MASS = 52164 |
| 5 | 11 | Homo sapiens (Human) POLY(RC)-BINDING PROTEIN 2 ISOFORM B. [MASS = 38222 |
| 5 | 5 | Homo sapiens (Human) EXPORTIN-T. [MASS = 109964 |
| 5 | 6 | Homo sapiens (Human) EUKARYOTIC TRANSLATION INITIATION FACTOR 2C 1. [MASS = 97214 |
| 5 | 5 | Homo sapiens (Human) PREDICTED: STRUCTURAL MAINTENANCE OF CHROMOSOMES |
| | | FLEXIBLE HINGE DOMAIN CONTAINING 1. [MASS = 220242 |
| 5 | 13 | Homo sapiens (Human) PREGNANCY ZONE PROTEIN PRECURSOR. [MASS = 163836 |
| 5 | 6 | Homo sapiens (Human) ISOFORM 1 OF LIM AND SH3 DOMAIN PROTEIN 1. [MASS = 29717 |
| 5 | 16 | Homo sapiens (Human) SIMILAR TO NESTIN. [MASS = 175922 |
| 5 | 5 | Homo sapiens (Human) ISOFORM 1 OF FILAMIN-C. [MASS = 291293 |
| 5 | 6 | Homo sapiens (Human) MICROTUBULE-ASSOCIATED PROTEIN RP/EB FAMILY MEMBER 1. |
| | | [MASS = 29868 |
| 5 | 5 | Homo sapiens (Human) 40S RIBOSOMAL PROTEIN S13. [MASS = 17091 |
| 5 | 5 | Homo sapiens (Human) MITOGEN-ACTIVATED PROTEIN KINASE 1. [MASS = 41259 |
| 5 | 5 | Homo sapiens (Human) UDP-GLUCOSE 6-DEHYDROGENASE. [MASS = 55024 |
| 5 | 9 | Homo sapiens (Human) PROBABLE ATP-DEPENDENT RNA HELICASE DDX5. [MASS = 69148 |
| 5 | 5 | Homo sapiens (Huirian) ISOFORM 1 OF SLIT-ROBO RHO GTPASE-ACTIVATING PROTEIN 3. |
| | | [MASS = 124504 |
| 5 | 5 | Homo sapiens (Human) HSP90 CO-CHAPERONE CDC37. [MASS = 44468 |
| 5 | 8 | Homo sapiens (Human) AMBP PROTEIN PRECURSOR. [MASS = 38999 |
| 5 | 5 | Homo sapiens (Human) CGI-150 PROTEIN. [MASS = 55012 |
| 5 | 6 | Homo sapiens (Human) PRE-MRNA-PROCESSING FACTOR 6 HOMOLOG. [MASS = 106925 |
| 5 | 5 | Homo sapiens (Human) TAR DNA-BINDING PROTEIN 43. [MASS = 44740 |
| 5 | 6 | Homo sapiens (Human) ISOFORM 1 OF KH DOMAIN-CONTAINING, RNA-BINDING, SIGNAL |
| | | TRANSDUCTION-ASSOCIATED PROTEIN 1. [MASS = 48227 |
| 5 | 5 | Homo sapiens (Human) HYPOTHETICAL PROTEIN DKFZP451P021. [MASS = 117896 |
| 5 | 7 | Homo sapiens (Human) KH-TYPE SPLICING REGULATORY PROTEIN. [MASS = 73115 |
| 5 | 5 | Homo sapiens (Human) ISOCITRATE DEHYDROGENASE [NADP], MITOCHONDRIAL |
| | | PRECURSOR. [MASS = 50909 |
| 5 | 5 | Homo sapiens (Human) 182 KDA TANKYRASE 1-BINDING PROTEIN. [MASS = 181816 |
| 5 | 5 | Homo sapiens (Human) CONDENSIN COMPLEX SUBUNIT 1. [MASS = 157169 |
| 5 | 7 | Homo sapiens (Human) ISOFORM 1 OF ACIDIC LEUCINE-RICH NUCLEAR PHOSPHOPROTEIN |
| | | 32 FAMILY MEMBER B. [MASS = 28788 |
| 5 | 5 | Homo sapiens (Human) SMALL NUCLEAR RIBONUCLEOPROTEIN SM D2. [MASS = 13527 |
| 5 | 5 | Homo sapiens (Human) FUSE-BINDING PROTEIN-INTERACTING REPRESSOR ISOFORM A. |
| | | [MASS = 59875 |
| 5 | 6 | Homo sapiens (Human) COP9 SIGNALOSOME COMPLEX SUBUNIT 6. [MASS = 36163 |
| 5 | 5 | Homo sapiens (Human) ACETYL-COA CARBOXYLASE 1. [MASS = 265040 |
| 5 | 5 | Homo sapiens (Human) ISOFORM 2 OF SUPPRESSOR OF G2 ALLELE OF SKP1 HOMOLOG. |
| | | [MASS = 37673 |
| 5 | 5 | Homo sapiens (Human) ISOFORM 5 OF DYNAMIN-1-LIKE PROTEIN. [MASS = 79123 |
| 5 | 5 | Homo sapiens (Human) 60S RIBOSOMAL PROTEIN L5. [MASS = 34231 |
| 5 | 6 | Homo sapiens (Human) PREDICTED: SIMILAR TO RIBOSOMAL PROTEIN L13 ISOFORM 1. |
| | | [MASS = 24280 |
| 5 | 5 | Homo sapiens (Human) INOSINE-5′-MONOPHOSPHATE DEHYDROGENASE 2. [MASS = 55805 |
| 5 | 5 | Homo sapiens (Human) ISOFORM 1 OF PHOSPHOSERINE AMINOTRANSFERASE. [MASS = 40423 |
| 5 | 9 | Homo sapiens (Human) HETEROGENEOUS NUCLEAR RIBONUCLEOPROTEIN C-LIKE 1. |
| | | [MASS = 32142 |
| 5 | 6 | Homo sapiens (Human) UBIQUITIN-CONJUGATING ENZYME E2 N. [MASS = 17138 |
| 5 | 5 | Homo sapiens (Human) PROTEIN KINASE C-BINDING PROTEIN NELL2 PRECURSOR. |
| | | [MASS = 91346 |
| 5 | 5 | Homo sapiens (Human) ISOFORM 1 OF DYNAMIN-2. [MASS = 98064 |
| 5 | 5 | Homo sapiens (Human) NEURONAL PROTEIN NP25. [MASS = 24893 |
| 5 | 5 | Homo sapiens (Human) SMALL GLUTAMINE-RICH TETRATRICOPEPTIDE |
| | | REPEAT-CONTAINING PROTEIN A. [MASS = 34063 |
| 4 | 4 | Homo sapiens (Human) PROLYL ENDOPEPTIDASE. [MASS = 80764 |
| 4 | 6 | Homo sapiens (Human) 60S ACIDIC RIBOSOMAL PROTEIN P2. [MASS = 11665 |
| 4 | 4 | Homo sapiens (Human) 40S RIBOSOMAL PROTEIN S7. [MASS = 22127 |
| 4 | 6 | Homo sapiens (Human) ISOFORM 1 OF DNA-BINDING PROTEIN A. [MASS = 40090 |
| 4 | 6 | Homo sapiens (Human) ISOFORM EWS-B OF RNA-BINDING PROTEIN EWS. [MASS = 61217 |
| 4 | 4 | Homo sapiens (Human) THYMIDYLATE SYNTHASE. [MASS = 31759 |
| 4 | 5 | Homo sapiens (Human) NASCENT POLYPEPTIDE-ASSOCIATED COMPLEX SUBUNIT |
| | | ALPHA. [MASS = 23384 |
| 4 | 5 | Homo sapiens (Human) HISTIDINE-RICH GLYCOPROTEIN PRECURSOR. [MASS = 59578 |
| 4 | 4 | Homo sapiens (Human) ISOFORM 1 OF PROTEASOME SUBUNIT ALPHA TYPE 7. [MASS = 27887 |
| 4 | 4 | Homo sapiens (Human) 1-PHOSPHATIDYLINOSITOL-4,5-BISPHOSPHATE PHOSPHODIESTERASE |
| | | GAMMA 1. [MASS = 148532 |
| 4 | 4 | Homo sapiens (Human) SPLICEOSOME RNA HELICASE BAT1. [MASS = 48991 |
| 4 | 5 | Homo sapiens (Human) PREDICTED: SIMILAR TO PHOSPHOGLYCERATE MUTASE 1 |
| | | (PHOSPHOGLYCERATE MUTASE ISOZYME B) (PGAM-B) (BPG-DEPENDENT PGAM 1) |
| | | ISOFORM 1. [MASS = 28850 |
| 4 | 4 | Homo sapiens (Human) CRK-LIKE PROTEIN. [MASS = 33777 |
| 4 | 5 | Homo sapiens (Human) RNA-BINDING PROTEIN MUSASHI HOMOLOG 1. [MASS = 39125 |
| 4 | 4 | Homo sapiens (Human) FLAP ENDONUCLEASE 1. [MASS = 42593 |
| 4 | 5 | Homo sapiens (Human) ISOFORM C OF FIBULIN-1 PRECURSOR: [MASS = 74462 |
| 4 | 39 | Homo sapiens (Human) TUBA6 PROTEIN. [MASS = 37021 |
| 4 | 5 | Homo sapiens (Human) BA395L14.12. [MASS = 28403 |
| 4 | 4 | Homo sapiens (Human) ISOFORM SHORT OF TATA-BINDING PROTEIN-ASSOCIATED |
| | | FACTOR 2N. [MASS = 61558 |
| 4 | 5 | Homo sapiens (Human) LUNG-CANCER ONCOGENE 7. [MASS = 37889 |
| 4 | 4 | Homo sapiens (Human) DNAJ HOMOLOG SUBFAMILY A MEMBER 1. [MASS = 44868 |
| 4 | 5 | Homo sapiens (Human) CLASS III ALCOHOL DEHYDROGENASE 5 CHI SUBUNIT. [MASS = 41601 |
| 4 | 5 | Homo sapiens (Human) DYNC1H1 PROTEIN. [MASS = 22182 |
| 4 | 4 | Homo sapiens (Human) ISOFORM 2 OF SERINE/THREONINE-PROTEIN KINASE PAK 1. [MASS = 61632 |
| 4 | 5 | Homo sapiens (Human) 60S RIBOSOMAL PROTEIN L21. [MASS = 18434 |
| 4 | 7 | Homo sapiens (Human) HETEROGENEOUS NUCLEAR RIBONUCLEOPROTEIN U ISOFORM A |
| | | [MASS = 90584 |
| 4 | 4 | Homo sapiens (Human) HISTONE H4. [MASS = 11236 |
| 4 | 4 | Homo sapiens (Human) SCC-112 PROTEIN. [MASS = 150830 |
| 4 | 4 | Homo sapiens (Human) WW DOMAIN-BINDING PROTEIN 11. [MASS = 69998 |
| 4 | 5 | Homo sapiens (Human) ISOFORM 2 OF PUTATIVE GTP-BINDING PROTEIN PTD004. [MASS = 27584 |
| 4 | 4 | Homo sapiens (Human) PLATELET-ACTIVATING FACTOR ACETYLHYDROLASE, ISOFORM IB, |
| | | ALPHA SUBUNIT. [MASS = 46638 |
| 4 | 4 | Homo sapiens (Human) ISOFORM II OF UBIQUITIN-PROTEIN LIGASE E3A. [MASS = 100646 |
| 4 | 4 | Homo sapiens (Human) BRAIN ACID SOLUBLE PROTEIN 1. [MASS = 22562 |
| 4 | 7 | Homo sapiens (Human) ACTIN-LIKE PROTEIN 2. [MASS = 44761 |
| 4 | 5 | Homo sapiens (Human) EUKARYOTIC TRANSLATION INITIATION FACTOR 5. [MASS = 49223 |
| 4 | 4 | Homo sapiens (Human) 14-3-3 PROTEIN ETA. [MASS = 28088 |
| 4 | 4 | Homo sapiens (Human) EUKARYOTIC TRANSLATION INITIATION FACTOR 2 SUBUNIT 1. |
| | | [MASS = 35981 |
| 4 | 5 | Homo sapiens (Human) ISOFORM DELTA-1 OF SERINE/THREONINE-PROTEIN PHOSPHATASE 2A |
| | | 56 KDA REGULATORY SUBUNIT DELTA ISOFORM. [MASS = 69992 |
| 4 | 4 | Homo sapiens (Human) UBIQUITIN CARBOXYL-TERMINAL HYDROLASE 10. [MASS = 87692 |
| 4 | 5 | Homo sapiens (Human) PDCD6IP PROTEIN. [MASS = 96818 |
| 4 | 4 | Homo sapiens (Human) PROTEIN FAM98B. [MASS = 37191 |
| 4 | 5 | Homo sapiens (Human) ISOFORM 1 OF 40S RIBOSOMAL PROTEIN S24. [MASS = 15423 |
| 4 | 5 | Homo sapiens (Human) ADENYLOSUCCINATE SYNTHETASE ISOZYME 2. [MASS = 50097 |
| 4 | 4 | Homo sapiens (Human) ISOFORM 1 OF MICROTUBULE-ASSOCIATED PROTEIN RP/EB FAMILY |
| | | MEMBER 2. [MASS = 37031 |
| 4 | 4 | Homo sapiens (Human) EXPORTIN-7. [MASS = 123776 |
| 4 | 4 | Homo sapiens (Human) PHOSPHOLIPASE A-2-ACTIVATING PROTEIN. [MASS = 87157 |
| 4 | 6 | Homo sapiens (Human) ISOFORM 1 OF PHOSPHOLIPID TRANSFER PROTEIN PRECURSOR. |
| | | [MASS = 54739 |
| 4 | 4 | Homo sapiens (Human) CERULOPLASMIN PRECURSOR. [MASS = 122205 |
| 4 | 5 | Homo sapiens (Human) RAS-RELATED PROTEIN RAB-7. [MASS = 23490 |
| 4 | 7 | Homo sapiens (Human) SERINE/THREONINE-PROTEIN PHOSPHATASE 2A CATALYTIC SUBUNIT |
| | | ALPHA ISOFORM. [MASS = 35594 |
| 4 | 4 | Homo sapiens (Human) ISOFORM 1 OF DOUBLE-STRAND BREAK REPAIR PROTEIN MRE11A. |
| | | [MASS = 80593 |
| 4 | 6 | Homo sapiens (Human) ADENYLATE KINASE ISOENZYME 1. [MASS = 21635 |
| 4 | 5 | Homo sapiens (Human) GPI-ANCHORED PROTEIN P137. [MASS = 72752 |
| 4 | 6 | Homo sapiens (Human) ALPHA-2-ANTIPLASMIN PRECURSOR. [MASS = 55064 |
| 4 | 5 | Homo sapiens (Human) PLASMA RETINOL-BINDING PROTEIN PRECURSOR. [MASS = 23010 |
| 4 | 4 | Homo sapiens (Human) ISOFORM 4 OF SAPS DOMAIN FAMILY MEMBER 3. [MASS = 88952 |
| 4 | 5 | Homo sapiens (Human) AP-1 COMPLEX SUBUNIT MU-1. [MASS = 48456 |
| 4 | 4 | Homo sapiens (Human) ALPHA-SOLUBLE NSF ATTACHMENT PROTEIN. [MASS = 33247 |
| 4 | 4 | Homo sapiens (Human) EUKARYOTIC TRANSLATION INITIATION FACTOR 4 GAMMA, 1 |
| | | ISOFORM 4. [MASS = 154805 |
| 4 | 4 | Homo sapiens (Human) PROTEASOME SUBUNIT ALPHA TYPE 6. [MASS = 27399 |
| 4 | 4 | Homo sapiens (Human) QUINONE OXIDOREDUCTASE. [MASS = 35207 |
| 4 | 4 | Homo sapiens (Human) SPLICING FACTOR 3A SUBUNIT 3. [MASS = 58849 |
| 4 | 4 | Homo sapiens (Human) GMP SYNTHASE. [MASS = 76715 |
| 4 | 4 | Homo sapiens (Human) ISOFORM 2 OF NMDA RECEPTOR-REGULATED PROTEIN 1. [MASS = 61602 |
| 4 | 4 | Homo sapiens (Human) EUKARYOTIC TRANSLATION INITIATION FACTOR 3 SUBUNIT 2. |
| | | [MASS = 36502 |
| 4 | 5 | Homo sapiens (Human) PROTEIN C14ORF166. [MASS = 28068 |
| 4 | 7 | Homo sapiens (Human) FARNESYL DIPHOSPHATE SYNTHASE. [MASS = 48275 |
| 4 | 5 | Homo sapiens (Human) COATOMER SUBUNIT GAMMA-2. [MASS = 97622 |
| 4 | 4 | Homo sapiens (Human) FIBRILLARIN. [MASS = 33784 |
| 4 | 4 | Homo sapiens (Human) NUCLEAR CAP-BINDING PROTEIN SUBUNIT 1. [MASS = 91839 |
| 4 | 4 | Homo sapiens (Human) PROTEASOME SUBUNIT BETA TYPE 4 PRECURSOR. [MASS = 29192 |
| 4 | 4 | Homo sapiens (Human) CALCIUM-BINDING PROTEIN 39. [MASS = 39869 |
| 4 | 4 | Homo sapiens (Human) PHENYLALANYL-TRNA SYNTHETASE BETA CHAIN. [MASS = 66130 |
| 4 | 4 | Homo sapiens (Human) PROTEIN FAM49B. [MASS = 36748 |
| 4 | 4 | Homo sapiens (Human) 47 KDA HEAT SHOCK PROTEIN PRECURSOR. [MASS = 46267 |
| 4 | 5 | Homo sapiens (Human) PEPTIDYLPROLYL ISOMERASE B PRECURSOR. [MASS = 23743 |
| 4 | 4 | Homo sapiens (Human) ISOFORM 1 OF EXPORTIN-5. [MASS = 136311 |
| 4 | 4 | Homo sapiens (Human) CADHERIN-2 PRECURSOR. [MASS = 99851 |
| 4 | 4 | Homo sapiens (Human) ISOFORM 3 OF DREBRIN-LIKE PROTEIN. [MASS = 49042 |
| 4 | 5 | Homo sapiens (Human) 16 KDA PROTEIN. [MASS = 16122 |
| 4 | 4 | Homo sapiens (Human) PROBABLE ATP-DEPENDENT RNA HELICASE DDX23. [MASS = 95647 |
| 4 | 4 | Homo sapiens (Human) CORONIN-1C. [MASS = 53249 |
| 4 | 4 | Homo sapiens (Human) SIGNAL RECOGNITION PARTICLE 14 KDA PROTEIN. [MASS = 14544 |
| 4 | 4 | Homo sapiens (Human) TROPOMYOSIN 1 ALPHA CHAIN ISOFORM 2. [MASS = 32678 |
| 4 | 4 | Homo sapiens (Human) VON HIPPEL-LINDAU BINDING PROTEIN 1. [MASS = 26535 |
| 4 | 7 | Homo sapiens (Human) ACETYL-COA ACETYLTRANSFERASE, CYTOSOLIC. [MASS = 41351 |
| 4 | 5 | Homo sapiens (Human) 60S RIBOSOMAL PROTEIN L19. [MASS = 23466 |
| 4 | 8 | Homo sapiens (Human) 40S RIBOSOMAL PROTEIN S2. [MASS = 31324 |
| 4 | 5 | Homo sapiens (Human) ISOFORM 1 OF MELANOMA-ASSOCIATED ANTIGEN D2. [MASS = 64954 |
| 4 | 7 | Homo sapiens (Human) 60S RIBOSOMAL PROTEIN L3. [MASS = 45978 |
| 4 | 18 | Homo sapiens (Human) UBIQUITIN AND RIBOSOMAL PROTEIN S27A PRECURSOR. |
| | | [MASS = 17965 |
| 4 | 4 | Homo sapiens (Human) PREDICTED: SIMILAR TO RAN-SPECIFIC GTPASE-ACTIVATING |
| | | PROTEIN. [MASS = 35024 |
| 4 | 5 | Homo sapiens (Human) SEPTIN 9. [MASS = 63633 |
| 4 | 4 | Homo sapiens (Human) SPERMATID PERINUCLEAR RNA-BINDING PROTEIN. [MASS = 73765 |
| 4 | 5 | Homo sapiens (Human) PROBABLE ATP-DEPENDENT RNA HELICASE DDX46. [MASS = 117461 |
| 4 | 4 | Homo sapiens (Human) RAS-RELATED PROTEIN RAB-14. [MASS = 23766 |
| 4 | 6 | Homo sapiens (Human) MARCKS-RELATED PROTEIN. [MASS = 19398 |
| 4 | 4 | Homo sapiens (Human) GCN1-LIKE PROTEIN 1. [MASS = 292930 |
| 4 | 4 | Homo sapiens (Human) KINESIN LIGHT CHAIN 1 ISOFORM 2. [MASS = 65310 |
| 4 | 4 | Homo sapiens (Human) ISOFORM 2 OF AT-RICH INTERACTIVE DOMAIN-CONTAINING |
| | | PROTEIN 1A. [MASS = 218335 |
| 4 | 5 | Homo sapiens (Human) TROPOMYOSIN 4. [MASS = 28522 |
| 4 | 4 | Homo sapiens (Human) EARLY ENDOSOME ANTIGEN 1. [MASS = 162466 |
| 4 | 6 | Homo sapiens (Human) RETICULOCALBIN-1 PRECURSOR. [MASS = 38890 |
| 4 | 4 | Homo sapiens (Human) ISOFORM 1 OF APOPTOSIS INHIBITOR 5. [MASS = 57561 |
| 4 | 4 | Homo sapiens (Human) ISOFORM 1 OF FOCAL ADHESION KINASE 1. [MASS = 119233 |
| 4 | 7 | Homo sapiens (Human) TUBULIN-SPECIFIC CHAPERONE B. [MASS = 27326 |
| 4 | 4 | Homo sapiens (Human) HSPC121. [MASS = 44423 |
| 4 | 6 | Homo sapiens (Human) SEPTIN-11. [MASS = 49267 |
| 4 | 6 | Homo sapiens (Human) NEUROCAN CORE PROTEIN PRECURSOR. [MASS = 142973 |
| 4 | 4 | Homo sapiens (Human) LYSYL-TRNA SYNTHETASE. [MASS = 68048 |
| 4 | 5 | Homo sapiens (Human) 6-PHOSPHOGLUCONOLACTONASE. [MASS = 27547 |
| 4 | 7 | Homo sapiens (Human) SPLICING FACTOR, ARGININE/SERINE-RICH 2. [MASS = 25345 |
| 4 | 4 | Homo sapiens (Human) 60S RIBOSOMAL PROTEIN L7A. [MASS = 29864 |
| 4 | 6 | Homo sapiens (Human) GUANINE NUCLEOTIDE-BINDING PROTEIN G(I)/G(S)/G(T) SUBUNIT |
| | | BETA 2. [MASS = 37200 |
| 4 | 4 | Homo sapiens (Human) ISOFORM LONG OF 60 KDA SS-A/RO RIBONUCLEOPROTEIN. |
| | | [MASS = 60671 |
| 4 | 4 | Homo sapiens (Human) SUPERKILLER VIRALICIDIC ACTIVITY 2-LIKE 2. [MASS = 117805 |
| 4 | 5 | Homo sapiens (Human) ISOFORM LONG OF TRIFUNCTIONAL PURINE BIOSYNTHETIC |
| | | PROTEIN ADENOSINE-3. [MASS = 107767 |
| 4 | 4 | Homo sapiens (Human) HEPARIN COFACTOR 2 PRECURSOR. [MASS = 60178 |
| 4 | 5 | Homo sapiens (Human) APOLIPOPROTEIN A-II PRECURSOR. [MASS = 11175 |
| 4 | 4 | Homo sapiens (Human) MYOSIN-11. [MASS = 227339 |
| 4 | 7 | Homo sapiens (Human) HISTONE H1.2. [MASS = 21234 |
| 4 | 8 | Homo sapiens (Human) HETEROGENEOUS NUCLEAR RIBONUCLEOPROTEIN G. [MASS = 42332 |
| 4 | 5 | Homo sapiens (Human) GLUCOSAMINE-6-PHOSPHATE ISOMERASE. [MASS = 32669 |
| 4 | 4 | Homo sapiens (Human) DNA LIGASE 1. [MASS = 101736 |
| 4 | 4 | Homo sapiens (Human) 60S RIBOSOMAL PROTEIN L18A. [MASS = 20762 |
| 4 | 5 | Homo sapiens (Human) CLEAVAGE AND POLYADENYLATION SPECIFICITY FACTOR 73 KDA |
| | | SUBUNIT. [MASS = 77486 |
| 3 | 3 | Homo sapiens (Human) PROTEASOME ACTIVATOR COMPLEX SUBUNIT 1. [MASS = 28723 |
| 3 | 3 | Homo sapiens (Human) ACYLAMINO-ACID-RELEASING ENZYME. [MASS = 81225 |
| 3 | 5 | Homo sapiens (Human) 60 KDA HEAT SHOCK PROTEIN, MITOCHONDRIAL PRECURSOR. |
| | | [MASS = 61055 |
| 3 | 3 | Homo sapiens (Human) THO COMPLEX SUBUNIT 4. [MASS = 27558 |
| 3 | 3 | Homo sapiens (Human) SMALL NUCLEAR RIBONUCLEOPROTEIN SM D1. [MASS = 13282 |
| 3 | 3 | Homo sapiens (Human) FRUCTOSE-BISPHOSPHATE ALDOLASE C. [MASS = 39325 |
| 3 | 14 | Homo sapiens (Human) ALPHA-2-HS-GLYCOPROTEIN PRECURSOR. [MASS = 39325 |
| 3 | 4 | Homo sapiens (Human) IMPORTIN ALPHA-4 SUBUNIT. [MASS = 57887 |
| 3 | 5 | Homo sapiens (Human) GTP BINDING PROTEIN 1. [MASS = 72454 |
| 3 | 3 | Homo sapiens (Human) ISOFORM 1 OF DAZ-ASSOCIATED PROTEIN 1. [MASS = 43383 |
| 3 | 3 | Homo sapiens (Human) VACUOLAR PROTEIN SORTING 26A. [MASS = 38170 |
| 3 | 3 | Homo sapiens (Human) CYTOPLASMIC FMR1 INTERACTING PROTEIN 1 ISOFORM A. |
| | | [MASS = 145182 |
| 3 | 3 | Homo sapiens (Human) ATP SYNTHASE SUBUNIT ALPHA, MITOCHONDRIAL |
| | | PRECURSOR. [MASS = 59751 |
| 3 | 4 | Homo sapiens (Human) ACONITATE HYDRATASE, MITOCHONDRIAL PRECURSOR. |
| | | [MASS = 85425 |
| 3 | 6 | Homo sapiens (Human) LUNG CANCER ONCOGENE 7. [MASS = 37889 |
| 3 | 4 | Homo sapiens (Human) F-ACTIN CAPPING PROTEIN ALPHA-2 SUBUNIT. [MASS = 32818 |
| 3 | 4 | Homo sapiens (Human) ISOFORM DUT-M OF DEOXYURIDINE 5′-TRIPHOSPHATE |
| | | NUCLEOTIDOHYDROLASE, MITOCHONDRIAL PRECURSOR. [MASS = 26706 |
| 3 | 6 | Homo sapiens (Human) ISOFORM 1 OF ALPHA-ADDUCIN. [MASS = 80955 |
| 3 | 3 | Homo sapiens (Human) ISOFORM 1 OF PROTEIN PHOSPHATASE 1 REGULATORY |
| | | SUBUNIT 7. [MASS = 41564 |
| 3 | 3 | Homo sapiens (Human) BM-010. [MASS = 36153 |
| 3 | 3 | Homo sapiens (Human) PROTEIN TYROSINE PHOSPHATASE,-RECEPTOR-TYPE, ZETA1 |
| | | PRECURSOR. [MASS = 254587 |
| 3 | 3 | Homo sapiens (Human) ISOFORM 3 OF UDP-N-ACETYLGLUCOSAMINE-PEPTIDE N- |
| | | ACETYLGLUCOSAMINYLTRANSFERASE 110 KDA SUBUNIT. [MASS = 116925 |
| 3 | 3 | Homo sapiens (Human) HIV TAT SPECIFIC FACTOR 1. [MASS = 85853 |
| 3 | 4 | Homo sapiens (Human) UV EXCISION REPAIR PROTEIN RAD23 HOMOLOG B. |
| | | [MASS = 43171 |
| 3 | 3 | Homo sapiens (Human) PNAS-125. [MASS = 23755 |
| 3 | 5 | Homo sapiens (Human) IGKV1-5 PROTEIN. [MASS = 26234 |
| 3 | 19 | Homo sapiens (Human) HEMOGLOBIN SUBUNIT GAMMA-1. [MASS = 16009 |
| 3 | 3 | Homo sapiens (Human) EXOSOME COMPLEX EXONUCLEASE RRP42. [MASS = 31835 |
| 3 | 3 | Homo sapiens (Human) ISOFORM 2 OF TRANSCRIPTION FACTOR BTF3. [MASS = 17699 |
| 3 | 4 | Homo sapiens (Human) HISTONE-BINDING PROTEIN RBBP4. [MASS = 47525 |
| 3 | 3 | Homo sapiens (Human) SERINE/THREONINE-PROTEIN KINASE MRCK BETA. |
| | | [MASS = 194315 |
| 3 | 3 | Homo sapiens (Human) HYPOTHETICAL PROTEIN DKFZP68610180 (FRAGMENT). |
| | | [MASS = 28810 |
| 3 | 4 | Homo sapiens (Human) THYMOPOIETIN ISOFORM BETA. [MASS = 50670 |
| 3 | 3 | Homo sapiens (Human) DNA-DIRECTED RNA POLYMERASE II LARGEST SUBUNIT. |
| | | [MASS = 217206 |
| 3 | 5 | Homo sapiens (Human) FACTOR VII ACTIVE SITE MUTANT IMMUNOCONJUGATE. |
| | | [MASS = 75553 |
| 3 | 3 | Homo sapiens (Human) TUBULIN-SPECIFIC CHAPERONE A. [MASS = 12724 |
| 3 | 3 | Homo sapiens (Human) TRYPTOPHANYL-TRNA SYNTHETASE. [MASS = 53165 |
| 3 | 3 | Homo sapiens (Human) ISOFORM 1 OF SYMPLEKIN. [MASS = 141148 |
| 3 | 3 | Homo sapiens (Human) SERINE/THREONINE-PROTEIN PHOSPHATASE 4 CATALYTIC |
| | | SUBUNIT. [MASS = 35080 |
| 3 | 3 | Homo sapiens (Human) ANTITHROMBIN III VARIANT. [MASS = 52692 |
| 3 | 7 | Homo sapiens (Human) UBIQUITIN-ACTIVATING ENZYME E1. [MASS = 25052 |
| 3 | 3 | Homo sapiens (Human) PREDICTED: SIMILAR TO CHLORIDE INTRACELLULAR CHANNEL |
| | | PROTEIN 4. [MASS = 17863 |
| 3 | 3 | Homo sapiens (Human) ISOFORM 1 OF TRANSCRIPTION ELONGATION FACTOR SPT5. |
| | | [MASS = 121000 |
| 3 | 4 | Homo sapiens (Human) EUKARYOTIC INITIATION FACTOR 5A ISOFORM I VARIANT A. |
| | | [MASS = 20170 |
| 3 | 3 | Homo sapiens (Human) ISOFORM 3 OF DNA REPAIR PROTEIN RAD50. [MASS = 138432 |
| 3 | 3 | Homo sapiens (Human) COP9 SIGNALOSOME COMPLEX SUBUNIT 5. [MASS = 37448 |
| 3 | 3 | Homo sapiens (Human) HYPOTHETICAL PROTEIN DKFZP547J2313. [MASS = 18829 |
| 3 | 4 | Homo sapiens (Human) CORTICOSTEROID-BINDING GLOBULIN PRECURSOR. [MASS = 45141 |
| 3 | 4 | Homo sapiens (Human) PROFILIN 2 ISOFORM A. [MASS = 15046 |
| 3 | 4 | Homo sapiens (Human) ISOFORM GAMMA-1 OF SERINE/THREONINE-PROTEIN PHOSPHATASE |
| | | PP1-GAMMA CATALYTIC SUBUNIT. [MASS = 36984 |
| 3 | 4 | Homo sapiens (Human) ISOFORM 1 OF UBIQUITIN-PROTEIN LIGASE BRE1B. [MASS = 113678 |
| 3 | 3 | Homo sapiens (Human) ISOFORM 1 OF RAS GTPASE-ACTIVATING PROTEIN 1. [MASS = 116403 |
| 3 | 5 | Homo sapiens (Human) ACTIN-RELATED PROTEIN 2/3 COMPLEX SUBUNIT 1A. [MASS = 41569 |
| 3 | 3 | Homo sapiens (Human) CADHERIN-5 PRECURSOR. [MASS = 87516 |
| 3 | 3 | Homo sapiens (Human) 40S RIBOSOMAL PROTEIN S14. [MASS = 16142 |
| 3 | 3 | Homo sapiens (Human) LETHAL GIANT LARVAE HOMOLOG 1. [MASS = 115388 |
| 3 | 3 | Homo sapiens (Human) LEUCINE ZIPPER TRANSCRIPTION FACTOR-LIKE 1. [MASS = 34592 |
| 3 | 4 | Homo sapiens (Human) MEPRIN A SUBUNIT ALPHA PRECURSOR. [MASS = 84368 |
| 3 | 4 | Homo sapiens (Human) WD REPEAT PROTEIN 61. [MASS = 33581 |
| 3 | 3 | Homo sapiens (Human) TRIPARTITE MOTIF-CONTAINING PROTEIN 2. [MASS = 81530 |
| 3 | 4 | Homo sapiens (Human) ISOFORM 1 OF ATP-DEPENDENT RNA HELICASE DDX19B. |
| | | [MASS = 53927 |
| 3 | 3 | Homo sapiens (Human) SORTING NEXIN 1 ISOFORM C. [MASS = 53304 |
| 3 | 3 | Homo sapiens (Human) HISTONE ACETYLTRANSFERASE TYPE B CATALYTIC SUBUNIT. |
| | | [MASS = 49513 |
| 3 | 3 | Homo sapiens (Human) SWI/SNF-RELATED MATRIX-ASSOCIATED ACTIN-DEPENDENT |
| | | REGULATOR OF CHROMATIN SUBFAMILY A MEMBER 5. [MASS = 121905 |
| 3 | 3 | Homo sapiens (Human) HEAT SHOCK 70 KDA PROTEIN 4L. [MASS = 94486 |
| 3 | 4 | Homo sapiens (Human) AFLATOXIN B1 ALDEHYDE REDUCTASE MEMBER 2. [MASS = 39589 |
| 3 | 4 | Homo sapiens (Human) D-DOPACHROME DECARBOXYLASE. [MASS = 12581 |
| 3 | 3 | Homo sapiens (Human) THIMET OLIGOPEPTIDASE. [MASS = 78709 |
| 3 | 3 | Homo sapiens (Human) 60S RIBOSOMAL PROTEIN L38. [MASS = 8087 |
| 3 | 3 | Homo sapiens (Human) ALDEHYDE DEHYDROGENASE 16 FAMILY, MEMBER A1. |
| | | [MASS = 85127 |
| 3 | 3 | Homo sapiens (Human) CYTOCHROME B5 REDUCTASE ISOFORM 1. [MASS = 34235 |
| 3 | 3 | Homo sapiens (Human) NETRIN RECEPTOR DCC PRECURSOR. [MASS = 158457 |
| 3 | 4 | Homo sapiens (Human) ISOPENTENYL-DIPHOSPHATE DELTA ISOMERASE. [MASS = 32485 |
| 3 | 4 | Homo sapiens (Human) PHOSPHATIDYLINOSITOL TRANSFER PROTEIN, BETA. |
| | | [MASS = 31540 |
| 3 | 3 | Homo sapiens (Human) HIGH MOBILITY GROUP PROTEIN B2. [MASS = 23903 |
| 3 | 5 | Homo sapiens (Human) TUMOR PROTEIN, TRANSLATIONALLY-CONTROLLED 1. |
| | | [MASS = 21526 |
| 3 | 3 | Homo sapiens (Human) BLEOMYCIN HYDROLASE. [MASS = 52562 |
| 3 | 9 | Homo sapiens (Human) ALPHA-ENOLASE, LUNG SPECIFIC. [MASS = 49477 |
| 3 | 4 | Homo sapiens (Human) 60S RIBOSOMAL PROTEIN L12. [MASS = 17819 |
| 3 | 3 | Homo sapiens (Human) CELL DIVISION CYCLE 5-LIKE PROTEIN. [MASS = 92251 |
| 3 | 3 | Homo sapiens (Human) ISOFORM 2 OF UBIQUITIN CARBOXYL-TERMINAL |
| | | HYDROLASE 47. [MASS = 147180 |
| 3 | 3 | Homo sapiens (Human) COMPLEMENT C5 PRECURSOR. [MASS = 188331 |
| 3 | 3 | Homo sapiens (Human) ISOFORM 1 OF CYTOPLASMIC LINKER PROTEIN 2. [MASS = 115837 |
| 3 | 3 | Homo sapiens (Human) THIOREDOXIN REDUCTASE 1, CYTOPLASMIC PRECURSOR. |
| | | [MASS = 54707 |
| 3 | 4 | Homo sapiens (Human) HISTONE H1X. [MASS = 22487 |
| 3 | 3 | Homo sapiens (Human) ISOFORM GTBP-N OF DNA MISMATCH REPAIR PROTEIN |
| | | MSH6. NASS = 152786 |
| 3 | 3 | Homo sapiens (Human) BILIVERDIN REDUCTASE A PRECURSOR. [MASS = 33428 |
| 3 | 3 | Homo sapiens (Human) LAMINA-ASSOCIATED POLYPEPTIDE 2 ISOFORM ALPHA. |
| | | [MASS = 75361 |
| 3 | 3 | Homo sapiens (Human) SYNAPTIC VESICLE MEMBRANE PROTEIN VAT-1 |
| | | HOMOLOG. [MASS = 41920 |
| 3 | 3 | Homo sapiens (Human) 60S RIBOSOMAL PROTEIN L8. [MASS = 27893 |
| 3 | 3 | Homo sapiens (Human) PHYTANOYL-COA HYDROXYLASE INTERACTING |
| | | PROTEIN-LIKE. [MASS = 42486 |
| 3 | 3 | Homo sapiens (Human) CONDENSIN COMPLEX SUBUNIT 2. [MASS = 82535 |
| 3 | 3 | Homo sapiens (Human) HIGH-MOBILITY GROUP BOX 1. [MASS = 15185 |
| 3 | 3 | Homo sapiens (Human) SELENIDE, WATER DIKINASE 1. [MASS = 42911 |
| 3 | 5 | Homo sapiens (Human) APOLIPOPROTEIN M. [MASS = 21253 |
| 3 | 3 | Homo sapiens (Human) HYPOTHETICAL PROTEIN DKFZP686M09245. [MASS = 61598 |
| 3 | 3 | Homo sapiens (Human.) ISOFORM 1 OF EXOSOME COMPLEX EXONUCLEASE RRP44. |
| | | [MASS = 109003 |
| 3 | 3 | Homo sapiens (Human) 60S RIBOSOMAL PROTEIN L10A. [MASS = 24700 |
| 3 | 3 | Homo sapiens (Human) ECHINODERM MICROTUBULE-ASSOCIATED.PROTEIN-LIKE 4. |
| | | [MASS = 108903 |
| 3 | 3 | Homo sapiens (Human) CALPAIN-1 CATALYTIC SUBUNIT. [MASS = 81890 |
| 3 | 4 | Homo sapiens (Human) 55 KDA PROTEIN. [MASS = 55183 |
| 3 | 3 | Homo sapiens (Human) ZYXIN. [MASS = 67285 |
| 3 | 5 | Homo sapiens (Human) DEVELOPMENTALLY-REGULATED GTP-BINDING PROTEIN 1. |
| | | [MASS = 40542 |
| 3 | 3 | Homo sapiens (Human) TYROSYL-TRNA SYNTHETASE, CYTOPLASMIC. [MASS = 59012 |
| 3 | 3 | Homo sapiens (Human) PEROXISOMAL MULTIFUNCTIONAL ENZYME TYPE 2. |
| | | [MASS = 79555 |
| 3 | 3 | Homo sapiens (Human) U4/U6.U5 TRI-SNRNP-ASSOCIATED PROTEIN 1. [MASS = 90255 |
| 3 | 3 | Homo sapiens (Human) 40S RIBOSOMAL PROTEIN S21. [MASS = 9111 |
| 3 | 3 | Homo sapiens (Human) DNAJ HOMOLOG SUBFAMILY C MEMBER 7. [MASS = 56441 |
| 3 | 4 | Homo sapiens (Human) CALSYNTENIN 1 ISOFORM 2. [MASS = 108643 |
| 3 | 3 | Homo sapiens (Human) TWINFILIN ISOFORM 1. [MASS = 43918 |
| 3 | 3 | Homo sapiens (Human) CENTROSOMAL PROTEIN 170 KDA ISOFORM ALPHA. |
| | | [MASS = 175436 |
| 3 | 3 | Homo sapiens (Human) ISOFORM 1 OF 26S PROTEASE REGULATORY SUBUNIT 6B. |
| | | [MASS = 47366 |
| 3 | 4 | Homo sapiens (Human) U1 SMALL NUCLEAR RIBONUCLEOPROTEIN A. [MASS = 31148 |
| 3 | 4 | Homo sapiens (Human) PEPTIDYL-PROLYL CIS-TRANS ISOMERASE A. [MASS = 17881 |
| 3 | 3 | Homo sapiens (Human) PEROXISOMAL MULTIFUNCTIONAL ENZYME TYPE 2. |
| | | [MASS = 79555 |
| 3 | 3 | Homo sapiens (Human) RAS-RELATED PROTEIN RAB-5C. [MASS = 23483 |
| 3 | 3 | Homo sapiens (Human) 40S RIBOSOMAL PROTEIN S8. [MASS = 24074 |
| 3 | 6 | Homo sapiens (Human) PERIPHERIN. [MASS = 53878 |
| 3 | 3 | Homo sapiens (Human) NUCLEOSIDE DIPHOSPHATE KINASE B. [MASS = 17298 |
| 3 | 5 | Homo sapiens (Human) SEC31L1 PROTEIN. [MASS = 121651 |
| 3 | 3 | Homo sapiens (Human) 40S RIBOSOMAL PROTEIN S16. [MASS = 16314 |
| 3 | 3 | Homo sapiens (Human) UROPORPHYRINOGEN DECARBOXYLASE. [MASS = 40787 |
| 3 | 3 | Homo sapiens (Human) 114 KDA PROTEIN. [MASS = 113977 |
| 3 | 4 | Homo sapiens (Human) COLLAGEN ALPHA-1(V) CHAIN PRECURSOR. [MASS = 183560 |
| 3 | 3 | Homo sapiens (Human) CYTOPLASMIC DYNEIN 1 LIGHT INTERMEDIATE CHAIN 2. |
| | | [MASS = 54099 |
| 3 | 3 | Homo sapiens (Human) CYTOSOLIC AMINOPEPTIDASE P. [MASS = 74798 |
| 3 | 3 | Homo sapiens (Human) ISOFORM 2 OF SPLICING FACTOR 1. [MASS = 68502 |
| 3 | 3 | Homo sapiens (Human) PREDICTED: SIMILAR TO BASIC LEUCINE ZIPPER AND W2 |
| | | DOMAINS 1. [MASS = 34090 |
| 3 | 3 | Homo sapiens (Human) ISOFORM 1 OF URIDINE 5′-MONOPHOSPHATE SYNTHASE. |
| | | [MASS = 52222 |
| 3 | 3 | Homo sapiens (Human) DEAD BOX POLYPEPTIDE 42 PROTEIN. [MASS = 102975 |
| 3 | 4 | Homo sapiens (Human) ISOFORM C OF NEURAL CELL ADHESION MOLECULE 1, 120 |
| | | KDA ISOFORM PRECURSOR. [MASS = 83985 |
| 3 | 3 | Homo sapiens (Human) COLD-INDUCIBLE RNA-BINDING PROTEIN. [MASS = 18648 |
| 3 | 4 | Homo sapiens (Human) 60S RIBOSOMAL PROTEIN L18. [MASS = 21503 |
| 3 | 3 | Homo sapiens (Human) ISOFORM 1 OF JMJC DOMAIN-CONTAINING.HISTONE |
| | | DEMETHYLATION PROTEIN 2B. [MASS = 191611 |
| 3 | 3 | Homo sapiens (Human) RCC1 PROTEIN. [MASS = 48146 |
| 3 | 3 | Homo sapiens (Human) LEUCINE-RICH REPEAT-CONTAINING PROTEIN 47. [MASS = 63473 |
| 3 | 3 | Homo sapiens (Human) ISOFORM 1 OF RNA-BINDING PROTEIN NOVA-1. [MASS = 52056 |
| 3 | 6 | Homo sapiens (Human) 40S RIBOSOMAL PROTEIN S10. [MASS = 18898 |
| 3 | 3 | Homo sapiens (Human) SPLICING FACTOR U2AF 65 KDA SUBUNIT. [MASS = 53501 |
| 3 | 3 | Homo sapiens (Human) ISOFORM SHORT OF PROTEASOME SUBUNIT ALPHA TYPE 1. |
| | | [MASS = 29556 |
| 3 | 3 | Homo sapiens (Human) ALPHA-1-ACID GLYCOPROTEIN 2 PRECURSOR. [MASS = 23603 |
| 3 | 4 | Homo sapiens (Human) 40S RIBOSOMAL PROTEIN S23. [MASS = 15676 |
| 3 | 3 | Homo sapiens (Human) DOLICHYL-DIPHOSPHOOLIGOSACCHARIDE--PROTEIN |
| | | GLYCOSYLTRANSFERASE 67 KDA SUBUNIT PRECURSOR. [MASS = 72778 |
| 3 | 3 | Homo sapiens (Human) HYPOTHETICAL PROTEIN LOC387104. [MASS = 103199 |
| 3 | 3 | Homo sapiens (Human) PROTEIN TRANSPORT PROTEIN SEC24C. [MASS = 118315 |
| 3 | 4 | Homo sapiens (Human) THIOREDOXIN. [MASS = 11606 |
| 3 | 3 | Homo sapiens (Human) CYTOSOLIC PURINE 5′-NUCLEOTIDASE. [MASS = 64970 |
| 3 | 6 | Homo sapiens (Human) PREDICTED: SIMILAR TO RIBOSOMAL PROTEIN S3A ISOFORM 1. |
| | | [MASS = 24821 |
| 3 | 4 | Homo sapiens (Human) ACIDIC LEUCINE-RICH NUCLEAR PHOSPHOPROTEIN 32 |
| | | FAMILY MEMBER A. [MASS = 28585 |
| 3 | 3 | Homo sapiens (Human) 40S RIBOSOMAL PROTEIN S25. [MASS = 13742 |
| 3 | 3 | Homo sapiens (Human) ADP-RIBOSYLATION FACTOR-LIKE PROTEIN 3. [MASS = 20456 |
| 3 | 4 | Homo sapiens (Human) CYSTATIN B. [MASS = 11140 |
| 3 | 3 | Homo sapiens (Human) PHOSPHOGLUCOMUTASE-2-LIKE 1. [MASS = 70456 |
| 3 | 3 | Homo sapiens (Human) 40S RIBOSOMAL PROTEIN S9. [MASS = 22460 |
| 3 | 3 | Homo sapiens (Human) ANKYRIN REPEAT AND FYVE DOMAIN CONTAINING 1 |
| | | ISOFORM 1. [MASS = 128486 |
| 3 | 3 | Homo sapiens (Human) ISOFORM LONG OF COLD SHOCK DOMAIN-CONTAINING |
| | | PROTEIN E1. [MASS = 88885 |
| 3 | 3 | Homo sapiens (Human) DEAD (ASP-GLU-ALA-ASP) BOX POLYPEPTIDE 39, ISOFORM 2. |
| | | [MASS = 35095 |
| 3 | 3 | Homo sapiens (Human) 60S RIBOSOMAL PROTEIN L23A. [MASS = 17695 |
| 3 | 4 | Homo sapiens (Human) ALPHA-INTERNEXIN. [MASS = 55391 |
| 3 | 3 | Homo sapiens (Human) ISOFORM 3 OF ANAMORSIN. [MASS = 32213 |
| 3 | 3 | Homo sapiens (Human) SPLICING FACTOR, ARGININE/SERINE-RICH 4. [MASS = 56678 |
| 3 | 3 | Homo sapiens (Human) DEBRANCHING ENZYME HOMOLOG 1. [MASS = 61555 |
| 3 | 3 | Homo sapiens (Human) ISOFORM 2 OF PROTEASOME SUBUNIT ALPHA TYPE 3. [MASS = 27516 |
| 3 | 3 | Homo sapiens (Human) MICROSOMAL TRIGLYCERIDE TRANSFER PROTEIN LARGE SUBUNIT |
| | | PRECURSOR. [MASS = 99351 |
| 3 | 4 | Homo sapiens (Human) ISOFORM B OF NEURONAL-SPECIFIC SEPTIN-3. [MASS = 40100 |
| 3 | 4 | Homo sapiens (Human) 60S RIBOSOMAL PROTEIN L28. [MASS = 15616 |
| 3 | 6 | Homo sapiens (Human) PREDICTED: SIMILAR TO HETEROGENEOUS NUCLEAR |
| | | RIBONUCLEOPROTEIN K ISOFORM A ISOFORM 2. [MASS = 24258 |

### Patent

### Attorney Docket No. 01948/144WO2

### Characterization of the Rat Embryonic Proteome

CSF was collected from the lateral ventricle of E12.5, E14.5, and E17.5 rat embryos and from the fourth ventricle of E14.5 rat embryos. CSF from two litters (approximately 20-24 rat embryos) was pooled for each time point and was separated by 1-D SDS-PAGE and the proteins were visualized with Coomassie blue stain. FIG. 1C shows the Coomassie stained protein pattern of CSF collected from all three time-points. Mass spectrometry analysis of the rat CSF was performed separately for E12.5, E14.5, E17.5 lateral ventricle, and E14.5 fourth ventricle and presented as Supplementary information table 4. There were 423 proteins identified in E12.5 LV CSF, 318 proteins in E14.5 LV, 249 proteins in E14.5 4^{th}V, and 382 proteins in E17.5 LV. There are 137 proteins common to E12.5, E14.5, and E17.5 rat CSF samples that are presented in Table 3, which includes the name of the protein, its molecular weight, subcellular localization, function, tissue specificity. Also included are relevant notes about each protein. Interestingly, there are 61 proteins identified in E12.5 LV, E14.5 LV, and E17.5 LV that were not identified in E14.5 4^{th}V and only 5 proteins identified in E12.5 LV, E14.5 4^{th}V, and E17.5 LV that were not identified in E14.5 LV. This does not appear to be simply due to an overall reduction in E14.5^{th}V protein concentration as similar numbers of peptides were identified for the proteins found in common with LV CSF samples. Instead, the difference suggests potential differences in the protein composition of CSF between the lateral and fourth ventricles, though further studies would be needed to confirm this and to assess its significance.

TABLE 3
Common proteins from mass spectrometry analysis of embryonic rat CSF isolated from E12.5 LV, E14.5
LV and 4^{th} V, and E17.5 LV. The number of peptides is listed from E14.5 4^{th} V.

| # of | | | | | | | | |
|---|---|---|---|---|---|---|---|---|
| unique | | | | | | | | |
| peptides | Total # | | | | | | | |
| from | of | Accession | | | Subcellular | | | |
| protein | peptides | Number | Name of Protein | MW | location | Function | Tissue specificity | Notes |
| 120 | 617 | Q7TMA5 | AA1064 - apolipoprotein B | 536024 | Secreted | Lipid and fatty acid | Plasma | Apo B-100 functions as a |
| | | | | | | transport and metabolism | | recognition signal for the |
| | | | | | | | | cellular binding and |
| | | | | | | | | internalization of LDL |
| | | | | | | | | particles by the apoB/E |
| | | | | | | | | receptor |
| 66 | 382 | Q4G047 | Apolipoprotein B - fragment | 165356 | Secreted | Lipid and fatty acid | Plasma | Fragment molecule |
| | | | | | | transport and metabolism | | |
| 37 | 135 | P04937 | SPLICE ISOFORM 1 OF FIBRONECTIN | 272511 | Secreted, | Cell adhesion mediated | Plasma | Integrin signaling |
| | | | PRECURSOR | | extracellular | signaling | fibronectin made | pathway |
| | | | | | space, | | by liver and | |
| | | | | | extracellular | | cellular | |
| | | | | | matrix | | fibronectin made | |
| | | | | | | | by fibroblasts, | |
| | | | | | | | epithelial and | |
| | | | | | | | other cell types is | |
| | | | | | | | deposited in the | |
| | | | | | | | extracellular | |
| | | | | | | | matrix | |
| 34 | 365 | P02773 | SPLICE ISOFORM 1 OF ALPHA-FETOPROTEIN | 68386 | Secreted | Transport/Transfer-Carrier | Plasma | Binds copper, nickel, and |
| | | | PRECURSOR | | | | | fatty acids as well as, and |
| | | | | | | | | bilirubin less well than, |
| | | | | | | | | serum albumin. |
| 31 | 120 | P02651 | APOLIPOPROTEIN A-IV PRECURSOR | 44456 | Secreted | Lipid and fatty acid | Plasma | May have a role in |
| | | | | | | transport and metabolism | | chylomicrons and VLDL |
| | | | | | | | | secretion and catabolism. |
| | | | | | | | | Apoa-IV is a major |
| | | | | | | | | component of HDL and |
| | | | | | | | | chylomicrons. |
| 28 | 53 | P06238 | ALPHA-2-MACROGLOBULIN PRECURSOR | 163701 | Secreted | Serine Protease Inhibitor | Plasma | Plays role in acute phase |
| | | | | | | | | response |
| 21 | 26 | Q9JL97 | GPI-ANCHORED CERULOPLASMIN | 123749 | Cell Membrane | Transport, Transfer/Carrier, | Brain | Metal ion binding |
| | | | | | | Oxidoreductase | | oxidoreductase activity |
| | | | | | | | | ion transport |
| 21 | 35 | P14046 | ALPHA-1-INHIBITOR 3 PRECURSOR | 163773 | Secreted | Serine Protease Inhibitor | Plasma | Belongs to the alpha-2- |
| | | | | | | | | macroglobulin family |
| | | | | | | | | involved in inflammtory |
| | | | | | | | | response |
| 20 | 96 | Q7TP24 | BA1-667 - Transferrin | 107448 | Secreted | Transport, Transfer/Carrier | Plasma | Iron ion homeostasis, |
| | | | | | | | | iron ion transport |
| 19 | 30 | P22063 | CONTACTIN-2 PRECURSOR | 113043 | Cell Membrane | Cell Adhesion - | In neural tissues | May play a role in the |
| | | | | | | Neurogenesis | in embryos, and | initial growth and |
| | | | | | | | in adult brain, | guidance of axons. |
| | | | | | | | spinal cord and | Belongs to the |
| | | | | | | | cerebellum. | immunoglobulin |
| | | | | | | | | superfamily. |
| 18 | 79 | P04639 | APOLIPOPROTEIN A-I PRECURSOR | 30088 | Secreted | Lipid and fatty acid | Major protein of | Participates in the |
| | | | | | | transport and metabolism | plasma HDL, also | reverse transport of |
| | | | | | | | found in | cholesterol from tissues |
| | | | | | | | chylomicrons. | to the liver for excretion |
| | | | | | | | | by promoting cholesterol |
| | | | | | | | | efflux from tissues and by |
| | | | | | | | | acting as a cofactor for |
| | | | | | | | | the lecithin cholesterol |
| | | | | | | | | acyltransferase (LCAT). |
| 18 | 97 | P02770 | SERUM ALBUMIN PRECURSOR | 68719 | Secreted | Transport, Transfer/Carrier | Plasma | The main protein of |
| | | | | | | | | plasma, has a good |
| | | | | | | | | binding capacity for |
| | | | | | | | | water, Ca(2+), Na(+), |
| | | | | | | | | K(+), fatty acids, |
| | | | | | | | | hormones, bilirubin and |
| | | | | | | | | drugs. Its main function is |
| | | | | | | | | the regulation of the |
| | | | | | | | | colloidal osmotic |
| | | | | | | | | pressure of blood. |
| 18 | 33 | P08460 | PREDICTED: NIDOGEN | 138365 | Secreted, | Cell - extracellular matrix | Wide distribution | Found in basement |
| | | | | | extracellular | adhesion | | membranes often |
| | | | | | space, | | | associated with laminin. |
| | | | | | extracellular | | | |
| | | | | | matrix | | | |
| 15 | 34 | P17475 | ALPHA-1-ANTIPROTEINASE PRECURSOR | 46136 | Secreted | Serine Protease Inhibitor | Plasma | Blood coagulation. The |
| | | | | | | | | primary target is elastase, |
| | | | | | | | | but also has a moderate |
| | | | | | | | | affinity for plasmin and |
| | | | | | | | | thrombin. |
| 15 | 17 | Q7TP05 | DA1-24 - Complement Factor B | 124379 | Secreted | Serine Protease - | Plasma | Hydrolase, Peptidase |
| | | | | | | Complement Mediated | | |
| | | | | | | Immunity | | |
| 14 | 23 | GeneID: 307618 | PREDICTED: SIMILAR TO CADHERIN-5 | 135230 | Cell junction, | Cell Adhesion | Endothelial | Cell - Cell interactions |
| | | | | | cell membrane | | tissues and brain | and juntions |
| 13 | 20 | Q80ZA3 | ALPHA-2 ANTIPLASMIN | 46465 | Secreted | Serine Protease Inhibitor | Plasma | Blood coagulation |
| 13 | 18 | P25304 | SPLICE ISOFORM 1 OF AGRIN PRECURSOR | 208646 | Secreted, | Cell adhesion mediated | Embryonic | Component of the basal |
| | | | | | extracellular | signaling | nervous system | lamina that binds to |
| | | | | | space, | | and muscle | laminin. |
| | | | | | extracellular | | | |
| | | | | | matrix | | | |
| 13 | 25 | GeneID: 313641 | PREDICTED: SIMILAR TO HEPARAN SULFATE | 377284 | Secreted, | Cell adhesion mediated | Widely | Integral component of |
| | | | PROTEOGLYCAN 2 (Perlecan) | | extracellular | signaling | distrubuted | basement membranes |
| | | | | | space, | | | |
| | | | | | extracellular | | | |
| | | | | | matrix | | | |
| 13 | 19 | O35802 | INTER-ALPHA-INHIBITOR H4 HEAVY CHAIN | 103755 | Secreted | Serine Protease Inhibitor | Plasma | Involved in acute phase |
| | | | | | | | | response |
| 12 | 14 | Q68FP1 | GELSOLIN | 86286 | Secreted and | Cell structure | Ubiquitous | Actin binding protein, |
| | | | | | Cytoplasm | | | may be involved in |
| | | | | | | | | myelination |
| 12 | 15 | Q5M7T5 | SERINE/CYSTEINE PROTEINASE INHIBITOR, | 52234 | Secreted | Protease inhibitor with a | Plasma | Belongs to the serpin |
| | | | CLADE C, MEMBER 1 | | | wide spectrum of protein | | family |
| | | | | | | targets | | |
| 12 | 22 | P01026 | COMPLEMENT C3 PRECURSOR | 186460 | Secreted | Complement mediated | Plasma | Plays a central role in the |
| | | | | | | immunity | | activation of the |
| | | | | | | | | complement system. |
| 12 | 24 | P02650 | APOLIPOPROTEIN E PRECURSOR | 35753 | Secreted | Lipid and fatty acid | Plasma | Mediates the binding, |
| | | | | | | transport and metabolism | | internalization, and |
| | | | | | | | | catabolism of lipoprotein |
| | | | | | | | | particles |
| 11 | 14 | Q62917 | LAR RECEPTOR-LINKED TYROSINE | 181130 | Cell Membrane | Cell adhesion mediated | Widely | May play a role in neurite |
| | | | PHOSPHATASE | | | signaling, cell structure and | distrubuted | outgrowth. |
| | | | | | | motility | | |
| 11 | 12 | Q63155 | DELETED IN COLORECTAL CANCER | 158142 | Membrane - | Ligand mediated signaling, | During | Netrin receptor activity, |
| | | | | | Intracellular | cell adhesion | development | transcription coactivator |
| | | | | | | | expressed highly | activity, axon guidance, |
| | | | | | | | in brain and | and apoptosis |
| | | | | | | | neural tube | |
| 11 | 12 | NM_199093 | SERINE PEPTIDASE INHIBITOR, CLADE G, | 55611 | Secreted | Protease inhibitor | Plasma | Involved in the regulation |
| | | | MEMBER 1 | | | | | of classical component |
| | | | | | | | | pathway |
| 11 | 17 | P31211 | CORTICOSTEROID-BINDING GLOBULIN | 44672 | Secreted | Transpost and Serine | Expressed by the | Major transport protein |
| | | | PRECURSOR | | | Protease Inhibitor | liver; secreted in | for glucocorticoids and |
| | | | | | | | plasma. | progestins in the blood |
| 11 | 12 | GeneID: 298941 | PREDICTED: SIMILAR TO LAMININ B1 | 228429 | Secreted, | Extracellular matrix linker | Widely distributed | Is thought to mediate the |
| | | | | | extracellular | protein-mediated signaling | in basement | attachment, migration |
| | | | | | space, | | membranes | and organization of cells |
| | | | | | extracellular | | | into tissues during |
| | | | | | matrix | | | embryonic development |
| | | | | | | | | by interacting with other |
| | | | | | | | | extracellular matrix |
| | | | | | | | | components. |
| 11 | 11 | P05197 | ELONGATION FACTOR 2 | 95153 | Cytoplasm - | Protein biosynthesis - | Ubiquitous | Promotes translocation of |
| | | | | | Intracellular | Translational elongation | | protein chain from A site |
| | | | | | | factor | | to P site on ribosome |
| 11 | 14 | P06399 | SPLICE ISOFORM 1 OF FIBRINOGEN ALPHA | 86686 | Secreted | Blood clotting | Plasma | Monomers polymerize |
| | | | CHAIN PRECURSOR | | | | | into fibrin and acts as a |
| | | | | | | | | cofactor in platelet |
| | | | | | | | | aggregation |
| 10 | 21 | Q9Z1Y3 | NEURAL-CADHERIN PRECURSOR | 99686 | Cell Membrane | Cell adhesion | Testis and | May be involved in |
| | | | | | | | neurons | neuronal tissue |
| | | | | | | | | recognition |
| 10 | 22 | GeneID: 315191 | PREDICTED: SIMILAR TO FIBULIN-1 | 78072 | Secreted, | Cell adhesion mediated | Detected in most | Incorporated into |
| | | | PRECURSOR | | extracellular | signaling | organs (brain, | fibronectin-containing |
| | | | | | space, | | heart, lung, | matrix fibers. May play a |
| | | | | | extracellular | | spleen, liver and | role in cell adhesion and |
| | | | | | matrix | | kidney). Neurons | migration along protein |
| | | | | | | | are the | fibers within the |
| | | | | | | | predominant | extracellular matrix |
| | | | | | | | source of | (ECM). Could be |
| | | | | | | | production in the | important for certain |
| | | | | | | | brain. Not | developmental processes |
| | | | | | | | expressed | and contribute to the |
| | | | | | | | significantly by | supramolecular |
| | | | | | | | astrocytes or | organization of ECM |
| | | | | | | | microglia. | architecture, in particular |
| | | | | | | | | to those of basement |
| | | | | | | | | membranes. |
| 10 | 11 | Q64610 | ECTONUCLEOTIDE | 101310 | Membrane | Hydrolase | Abundantly | Hydrolytically removes 5′- |
| | | | PYROPHOSPHATASE/PHOSPHODIESTERASE 2 | | | | expressed in | nucleotides successively |
| | | | | | | | cerebrum and | from the 3′-hydroxy |
| | | | | | | | cerebellum. | termini of 3′-hydroxy- |
| | | | | | | | Localized in | terminated |
| | | | | | | | secretory | oligonucleotides |
| | | | | | | | epithelial cells in | |
| | | | | | | | the brain and the | |
| | | | | | | | eye including | |
| | | | | | | | choroid plexus | |
| | | | | | | | epithelial cells, | |
| | | | | | | | ciliary epithelial | |
| | | | | | | | cells, iris pigment | |
| | | | | | | | epithelial cells, | |
| | | | | | | | and retinal | |
| | | | | | | | pigment cells. | |
| 10 | 21 | P02454 | COLLAGEN ALPHA-1(I) CHAIN PRECURSOR | 137886 | Secreted, | Cell adhesion - Cell | Forms the fibrils | Component of connective |
| | | | | | extracellular | structure | of tendon, | tissue |
| | | | | | space, | | ligaments and | |
| | | | | | extracellular | | bones | |
| | | | | | matrix | | | |
| 10 | 21 | P51886 | LUMICAN PRECURSOR | 38279 | Secreted, | Cell adhesion mediated | Widely distributed | Binds to laminin |
| | | | | | extracellular | signaling | | |
| | | | | | space, | | | |
| | | | | | extracellular | | | |
| | | | | | matrix | | | |
| 9 | 51 | P02767 | TRANSTHYRETIN PRECURSOR | 15720 | Secreted | Hormone transport | Most abundant in | Thyroid hormone-binding |
| | | | | | | | the choroid | protein. Probably |
| | | | | | | | plexus. Also | transports thyroxine from |
| | | | | | | | present in the | the bloodstream to the |
| | | | | | | | liver | brain |
| 9 | 13 | P00787 | CATHEPSIN B PRECURSOR | 37470 | Lysosome | Cysteine protease | Widely distributed | Participates in |
| | | | | | | | | intracellular degradation |
| | | | | | | | | and turnover of proteins. |
| | | | | | | | | Has been implicated in |
| | | | | | | | | tumor invasion and |
| | | | | | | | | metastasis |
| 9 | 13 | Q03626 | ALPHA(1)-INHIBITOR 3, VARIANT I | 165326 | Secreted | Serine Protease Inhibitor | Serum | Invovled in acute phase |
| | | | PRECURSOR | | | | | inflammatory response |
| 9 | 11 | GeneID: 116669 | PREDICTED: VON WILLEBRAND FACTOR | 308474 | Secreted, | Cell adhesion - Blood | Plasma | Major coagulation factor |
| | | | | | extracellular | clotting | | |
| | | | | | space, | | | |
| | | | | | extracellular | | | |
| | | | | | matrix | | | |
| 9 | 18 | Q5M7V3 | LOC367586 PROTEIN - Immunoglobulin Gamma | 50949 | Cell Membrane | B-cell and antibody- | Plasma | Associates non- |
| | | | heavy Chain | | | mediated immunity | | covalently with beta-2- |
| | | | | | | | | microglobulin, antigen |
| | | | | | | | | binding |
| 8 | 10 | Q91XW0 | HEAT SHOCK PROTEIN 86 | 84815 | Cytoplasm | Molecular chaperone, | Ubiquitous | |
| | | | | | | protein folding, stress | | |
| | | | | | | response | | |
| 8 | 8 | Q920H8 | HEPHAESTIN PRECURSOR | 129593 | Membrane | Oxidase | Expressed highly | May function as a |
| | | | | | | | in intestine, lung | ferroxidase for ferrous (II) |
| | | | | | | | and brain | to ferric ion (III) |
| | | | | | | | | conversion and in copper |
| | | | | | | | | transport and |
| | | | | | | | | homeostasis |
| 8 | 13 | P13941 | COLLAGEN ALPHA-1(III) CHAIN PRECURSOR | 138936 | Secreted, | Cell adhesion - Cell | Widely | Component of connective |
| | | | | | extracellular | structure | distributed, highly | tissue |
| | | | | | space, | | expressed in | |
| | | | | | extracellular | | colon and blood | |
| | | | | | matrix | | vessels | |
| 8 | 9 | P58751 | SPLICE ISOFORM 1 OF REELIN PRECURSOR | 387531 | Secreted, | Serine protease | Brain | Plays a role in layering of |
| | | | | | extracellular | | | neurons in the cerebral |
| | | | | | space, | | | cortex and cerebellum. |
| | | | | | extracellular | | | Regulates microtubule |
| | | | | | matrix | | | function in neurons and |
| | | | | | | | | neuronal migration. |
| | | | | | | | | Affects migration of |
| | | | | | | | | sympathetic |
| | | | | | | | | preganglionic neurons in |
| | | | | | | | | the spinal cord, where it |
| | | | | | | | | seems to act as a barrier |
| | | | | | | | | to neuronal migration |
| 8 | 20 | Q01177 | PLASMINOGEN PRECURSOR | 90536 | Secreted | Protease | Plasma | Plasmin dissolves the |
| | | | | | | | | fibrin of blood clots and |
| | | | | | | | | acts as a proteolytic |
| | | | | | | | | factor in a variety of other |
| | | | | | | | | processes including |
| | | | | | | | | embryonic development, |
| | | | | | | | | tissue remodeling, tumor |
| | | | | | | | | invasion, and |
| | | | | | | | | inflammation |
| 8 | 12 | O88752 | EPSILON 1 GLOBIN | 16105 | Secreted | Oxygen Transport, Transfer - | Blood | Involved in oxygen |
| | | | | | | Carrier Protein | | transport from the lung to |
| | | | | | | | | the various peripheral |
| | | | | | | | | tissues |
| 8 | 13 | P08934 | SPLICE ISOFORM HMW OF KININOGEN-1 | 70933 | Secreted | Cysteine protease inhibitor | Plasma | Bradykinin is released |
| | | | PRECURSOR | | | | | from kininogen by plasma |
| | | | | | | | | kallikrein |
| 8 | 19 | P02680 | SPLICE ISOFORM GAMMA-B OF FIBRINOGEN | 50633 | Secreted | Blood clotting | Plasma | Monomers polymerize |
| | | | GAMMA CHAIN PRECURSOR | | | | | into fibrin and also acts |
| | | | | | | | | as a cofactor in platelet |
| | | | | | | | | aggregation |
| 8 | 9 | P55280 | CADHERIN-6 PRECURSOR | 88341 | Cell Membrane | Cell adhesion | Highly expressed | Also known as Kidney |
| | | | | | | | in kidney and | cadherin |
| | | | | | | | brain | |
| 7 | 9 | P69897 | TUBULIN BETA-5 CHAIN | 49671 | Intracellular | Cell structure, cell mobility, | Ubiquitously | Tubulin is the major |
| | | | | | | intracellular protein traffic | expressed with | constituent of |
| | | | | | | | highest levels in | microtubules |
| | | | | | | | spleen, thymus | |
| | | | | | | | and immature | |
| | | | | | | | brain | |
| 7 | 8 | P01015 | ANGIOTENSINOGEN PRECURSOR | 51982 | Secreted | Serine Protease Inhibitor | Expressed by the | Helps regulate volume |
| | | | | | | | liver and secreted | and mineral balance of |
| | | | | | | | in plasma | body fluids |
| 7 | 8 | P24054 | SPARC-LIKE PROTEIN 1 PRECURSOR | 70634 | Secreted, | Matrix glycoprotein Sc1 | Expressed in | Function unknown, |
| | | | | | extracellular | | many types of | believed to bind calcium |
| | | | | | space, | | neurons in the | and play a role in brain |
| | | | | | extracellular | | brain | development |
| | | | | | matrix | | | |
| 7 | 7 | P97603 | 156 KDA PROTEIN - Neogenin precursor | 156144 | Membrane | Cell adhesion | Widely expressed | May be involved as a |
| | | | | | | | | regulatory protein in the |
| | | | | | | | | transition of |
| | | | | | | | | undifferentiated |
| | | | | | | | | proliferating cells to their |
| | | | | | | | | differentiated state. |
| | | | | | | | | Belongs to the |
| | | | | | | | | immunoglobulin |
| | | | | | | | | superfamily. |
| 7 | 9 | P68136 | ACTIN, ALPHA SKELETAL MUSCLE | 42051 | Cytoplasm | Cell structure, cell motility | Muscle | Actins are highly |
| | | | | | | | | conserved proteins that |
| | | | | | | | | are involved in various |
| | | | | | | | | types of cell motility and |
| | | | | | | | | are ubiquitously |
| | | | | | | | | expressed in all |
| | | | | | | | | eukaryotic cells. |
| 7 | 9 | P62630 | ELONGATION FACTOR 1-ALPHA 1 | 50114 | Cytoplasm | Protein biosynthesis, | Ubiquitous | Links tRNA to ribosome |
| | | | | | | translation regulation | | during protein synthesis |
| 7 | 7 | P46462 | TRANSITIONAL ENDOPLASMIC RETICULUM | 89534 | Intracellular | Protein targeting and | Ubiquitous | Necessary for the |
| | | | ATPASE | | | localization, intracellular | | fragmentation of Golgi |
| | | | | | | protein traffic | | stacks during mitosis and |
| | | | | | | | | for their reassembly after |
| | | | | | | | | mitosis. Involved in the |
| | | | | | | | | formation of the |
| | | | | | | | | transitional endoplasmic |
| | | | | | | | | reticulum (tER). |
| 7 | 11 | P02466 | COLLAGEN ALPHA-2(I) CHAIN PRECURSOR | 129564 | Secreted, | Cell adhesion - Cell | Forms the fibrils | Involved in skeletal |
| | | | | | extracellular | structure | of tendon, | development |
| | | | | | space, | | ligaments and | |
| | | | | | extracellular | | bones | |
| | | | | | matrix | | | |
| 7 | 7 | P28494 | ALPHA-MANNOSIDASE 2 | 131242 | Golgi apparatus - | Glycosidase | All tissues, mostly | Involved in N- |
| | | | | | Intracellular | | in adrenal and | glycosylation |
| | | | | | | | thymus | |
| 7 | 9 | Q99J86 | SPLICE ISOFORM 1 OF ATTRACTIN | 163296 | Cell membrane | Cell adhesion - Immune | Widely | Involved in the initial |
| | | | PRECURSOR | | | defense | distributed, highly | immune cell clustering |
| | | | | | | | expressed in | during Inflammatory |
| | | | | | | | colon and brain | response and may |
| | | | | | | | | regulate chemotactic |
| | | | | | | | | activity of chemokines. |
| | | | | | | | | Has a critical role in |
| | | | | | | | | normal myelination in the |
| | | | | | | | | central nervous system, |
| | | | | | | | | and enhancing cell |
| | | | | | | | | survival against oxidative |
| | | | | | | | | stress. |
| 6 | 9 | P68370 | TUBULIN ALPHA-1 CHAIN | 50136 | Intracellular | Cell structure, cell mobility, | Widely distributed | Tubulin is the major |
| | | | | | | chromosome segregation, | | constituent of |
| | | | | | | intracellular protein traffic | | microtubules. |
| 6 | 8 | P97686 | SPLICE ISOFORM 1 OF NEURONAL CELL | 133912 | Cell membrane | Cell adhesion | Widely | Ankyrin-binding protein |
| | | | ADHESION MOLECULE PRECURSOR | | | | distributed, highly | involved in neuron- |
| | | | | | | | expressed in | neuron adhesion. May |
| | | | | | | | brain | play a role in the |
| | | | | | | | | molecular assembly of |
| | | | | | | | | the nodes of Ranvier |
| 6 | 8 | Q91V15 | ALPHA-2-GLOBIN CHAIN | 15285 | Secreted | Oxygen Transport, Transfer - | Blood | Involved in oxygen |
| | | | | | | Carrier Protein | | transport from the lung to |
| | | | | | | | | the various peripheral |
| | | | | | | | | tissues |
| 6 | 6 | Q9WVR2 | ARCADLIN | 103800 | Cell Membrane | Cell adhesion | Brain | Neural activity-regulated |
| | | | | | | | | cadherin may be involved |
| | | | | | | | | in long term potentiation |
| 6 | 9 | Q62657 | TENASCIN (FRAGMENT) | 62473 | Secreted, | Cell adhesion | Widely | Negative regulation of |
| | | | | | extracellular | | distrubuted | cell adhesion |
| | | | | | space, | | | |
| | | | | | extracellular | | | |
| | | | | | matrix | | | |
| 6 | 12 | P04916 | PREDICTED: RETINOL BINDING PROTEIN 4, | 23220 | Secreted | Vitamin/Co-factor transport - | Plasma | Delivers retinol from the |
| | | | PLASMA | | | retinol binding, transporter | | liver stores to the |
| | | | | | | activity | | peripheral tissues. In |
| | | | | | | | | plasma, the RBP-retinol |
| | | | | | | | | complex interacts with |
| | | | | | | | | transthyretin, this |
| | | | | | | | | prevents its loss by |
| | | | | | | | | filtration through the |
| | | | | | | | | kidney glomeruli. |
| 6 | 10 | GeneID: 313717 | PREDICTED: CALSYNTENIN 1 | 109351 | Cell Membrane | Cell adhesion | Widely distributed | May modulate calcium- |
| | | | | | | | | mediated postsynaptic |
| | | | | | | | | signals |
| 6 | 8 | GeneID: 302248 | PREDICTED: NIDOGEN 2 | 173960 | Secreted, | Cell adhesion, Cell - | Widely distributed | Sulfated glycoprotein |
| | | | | | extracellular | extracellular matrix | in basement | widely distributed in |
| | | | | | space, | interaction | membranes | basement membranes |
| | | | | | extracellular | | | and tightly associated |
| | | | | | matrix, | | | with laminin. Also binds |
| | | | | | basement | | | to collagen IV and |
| | | | | | membrane | | | perlecan. It probably has |
| | | | | | | | | a role in cell-extracellular |
| | | | | | | | | matrix interactions |
| 6 | 7 | P11980 | PREDICTED: SIMILAR TO PYRUVATE KINASE | 57731 | Intracellular | Carbohydrate degradation, | Widely distributed | ATP + pyruvate = ADP + |
| | | | (EC 2.7.1.40) ISOZYME M2 - RAT | | | glycolysis | | phosphoenolpyruvate |
| 6 | 7 | Q63581 | T-KININOGEN | 47618 | Secreted | Cysteine protease inhibitor | Plasma | Plays a role in blood |
| | | | | | | | | clotting and regulation of |
| | | | | | | | | vasoconstriction and |
| | | | | | | | | dilation |
| 6 | 10 | GeneID: 84407 | PREDICTED: CADHERIN 11 | 88036 | Cell Membrane | Cell adhesion | Widely distributed | Involved in mesenchymal |
| | | | | | | | | tissue formation |
| 6 | 6 | Q66HD0 | PREDICTED: TUMOR REJECTION ANTIGEN | 74208 | Cytoplasm | Molecular chaperone, | Widely distributed | Highly homologous to |
| | | | GP96 | | | protein folding, stress | | endoplasmin precursor |
| | | | | | | response | | |
| 6 | 8 | GeneID: 290559 | PREDICTED: SIMILAR TO STABILIN-1 | 288663 | Membrane | Extracellular matrix | High levels found | Acts as a scavenger |
| | | | | | | structural protein, | in human spleen, | receptor for acetylated |
| | | | | | | extracellular matrix protein- | lymph node, liver | low density lipoprotein. |
| | | | | | | mediated signaling | and placenta | Binds to both Gram- |
| | | | | | | | | positive and Gram- |
| | | | | | | | | negative bacteria and |
| | | | | | | | | may play a role in |
| | | | | | | | | defense against bacterial |
| | | | | | | | | infection. When inhibited |
| | | | | | | | | in endothelial tube |
| | | | | | | | | formation assays, there is |
| | | | | | | | | a marked decrease in |
| | | | | | | | | cell-cell interactions, |
| | | | | | | | | suggesting a role in |
| | | | | | | | | angiogenesis |
| 6 | 7 | Q6MG73 | COMPLEMENT COMPONENT 2 | 83699 | Secreted | Serine protease, | Plasma | Belongs to the peptidase |
| | | | | | | complement-mediated | | S1 family |
| | | | | | | immunity | | |
| 6 | 9 | Q6IRS6 | FETUB PROTEIN | 43169 | Secreted | Cysteine protease inhibitor | Liver | Inhibits insulin receptor |
| | | | | | | | | tyrosine kinase activity. |
| 5 | 6 | P62963 | PROFILIN-1 | 14826 | Intracellular | Cell structure | Ubiquitous | At high concentrations, |
| | | | | | | | | profilin prevents the |
| | | | | | | | | polymerization of actin, |
| | | | | | | | | whereas it enhances it at |
| | | | | | | | | low concentrations |
| 5 | 5 | Q5U300 | HYPOTHETICAL PROTEIN LOC314432, Similar | 117788 | Intracellular | Proteolysis | Ubiquitous | Ubiquitin activating |
| | | | to ubiquitin-protein ligase (EC 6.3.2.19) E1 | | | | | enzyme activity |
| 5 | 12 | Q68FT8 | SERINE PEPTIDASE INHIBITOR, CLADE F, | 54893 | Secreted | Serine Protease Inhibitor | Plasma | Belongs to the serpin |
| | | | MEMBER 2 | | | | | family |
| 5 | 8 | Q9JI03 | COLLAGEN ALPHA-1(V) CHAIN PRECURSOR | 184610 | Secreted, | Extracellular matrix, | Ubiquitous | Component of connective |
| | | | | | extracellular | Structural protein | | tissue |
| | | | | | space, | | | |
| | | | | | extracellular | | | |
| | | | | | matrix | | | |
| 5 | 5 | P08649 | COMPLEMENT C4 PRECURSOR | 192163 | Secreted | Complement-mediated | Plasma | Inflammatory response |
| | | | | | | immunity | | |
| 5 | 7 | GeneID: 307351 | HYPOTHETICAL PROTEIN RGD1305887- | 50059 | Intracellular | Cell structure, cell mobility, | Ubiquitous | Tubulin is the major |
| | | | TUBULIN BETA CHAIN | | | chromosome segregation, | | constituent of |
| | | | | | | intracellular protein traffic | | microtubules |
| 5 | 6 | Q6IUU3 | QUIESCIN Q6 | 82412 | Isoform 1: | Oxidase | Widely | May contribute to |
| | | | | | Membrane; | | distributed, | disulfide bond formation |
| | | | | | Isoform 2: | | expressed in | in a variety of secreted |
| | | | | | Secreted | | heart, placenta, | proteins |
| | | | | | protein | | lung, liver, | |
| | | | | | | | skeletal muscle, | |
| | | | | | | | pancreas and | |
| | | | | | | | very weakly in | |
| | | | | | | | brain and kidney | |
| 5 | 6 | GeneID: 296371 | PREDICTED: SIMILAR TO PHOSPHOLIPID | 65430 | Secreted | Lipid and fatty acid | Plasma | Involved in phospholipid |
| | | | TRANSFER PROTEIN | | | transport | | transfer in the serum. |
| 5 | 5 | Q63041 | ALPHA-1-MACROGLOBULIN | 167125 | Secreted | Serine protease inhibitor | Plasma | Also known as |
| | | | | | | | | PREGNANCY ZONE |
| | | | | | | | | PROTEIN |
| 5 | 5 | GeneID: 21846 | PREDICTED: TYROSINE KINASE RECEPTOR 1 | 125210 | Cell Membrane | Receptor protein-tyrosine | Unknown | Unknown |
| | | | | | | kinase | | |
| 5 | 26 | Q08420 | EXTRACELLULAR SUPEROXIDE DISMUTASE | 26620 | Secreted, | Oxidoreductase - Immunity | Widely distributed | Destroys radicals which |
| | | | [CU - ZN] PRECURSOR | | extracellular | and Defense | | are normally produced |
| | | | | | space, | | | within the cells and which |
| | | | | | extracellular | | | are toxic to biological |
| | | | | | matrix | | | systems. |
| 5 | 11 | Q6AYQ9 | PEPTIDYLPROLYL ISOMERASE C | 23009 | Cytoplasm | Isomerase, Protein folding, | Widely | Involved in immunity and |
| | | | | | | Nuclear transport | distributed, highly | defense |
| | | | | | | | expressed in eye, | |
| | | | | | | | vascular tissue, | |
| | | | | | | | kidney and brain | |
| 5 | 6 | Q62918 | PROTEIN KINASE C-BINDING PROTEIN NELL2 | 91334 | Secreted | Cell communication, Cell | Widely distributed | Regulation of growth and |
| | | | | | | adhesion, Cell structure | | neurogenesis |
| 4 | 7 | GeneID: 114489 | PREDICTED: DYSTROGLYCAN 1 | 96706 | Membrane | Extracellular matrix protein- | Ubiquitous | Basement membrane |
| | | | | | | mediated signaling | | |
| 4 | 4 | O35217 | PREDICTED: SIMILAR TO HEPATIC MULTIPLE | 54619 | Endoplasmic | Nucleotide phosphatase | Widely expressed | Calcium mediated |
| | | | INOSITOL POLYPHOSPHATE PHOSPHATASE | | reticulum | | with highest | signaling involved in cell |
| | | | | | | | levels in kidney | proliferation and |
| | | | | | | | and liver | differentiation |
| 4 | 4 | Q91XN5 | PROMININ-1S1 SPLICE VARIANT | 96632 | Cell Membrane | Cellular component, integral | Hematopoeitc | May be involved in |
| | | | | | | to membrane | stem cells, retina, | membrane traffic. Has |
| | | | | | | | placenta, lung | been localized to |
| | | | | | | | brain | extracellular membrane |
| | | | | | | | | bound particles in the |
| | | | | | | | | CSF. |
| 4 | 5 | O88754 | EPSILON 3 GLOBIN | 16540 | Secreted | Oxygen Transport, Transfer - | Blood | Involved in oxygen |
| | | | | | | Carrier Protein | | transport from the lung to |
| | | | | | | | | the various peripheral |
| | | | | | | | | tissues |
| 4 | 9 | P14841 | CYSTATIN C PRECURSOR | 15437 | Secreted, | Cysteine protease inhibitor | Widely distributed | Regulation of |
| | | | | | extracellular | | with high | programmed cell death |
| | | | | | space, | | expression in | |
| | | | | | extracellular | | brain and muscle | |
| | | | | | matrix | | | |
| 4 | 4 | GeneID: 295027 | PREDICTED: SIMILAR TO PROTOCADHERIN | 123552 | Cell Membrane | Cell adhesion | Expressed in | May be involved in the |
| | | | 18 PRECURSOR | | | | brain | establishment and |
| | | | | | | | | maintenance of specific |
| | | | | | | | | neuronal connections in |
| | | | | | | | | the brain |
| 4 | 7 | Q9ERB4 | SPLICE ISOFORM V0 OF VERSICAN CORE | 300008 | Secreted, | Extracellular matrix protein- | Widely distributed | May be involved in axon |
| | | | PROTEIN PRECURSOR (FRAGMENT) | | extracellular | mediated signaling, cell | | regeneration and |
| | | | | | space, | adhesion, cell motility | | physiological response to |
| | | | | | extracellular | | | wounding |
| | | | | | matrix | | | |
| 4 | 5 | GeneID: 116782 | PROTOCADHERIN GAMMA SUBFAMILY C, 3 | 101038 | Cell Membrane | Cell adhesion | Widely | May play a role during |
| | | | | | | | distributed, highly | spermatogenesis |
| | | | | | | | expressed in | |
| | | | | | | | pineal gland | |
| 4 | 4 | O54861 | SORTILIN PRECURSOR | 91169 | Membranes; | Functions as a sorting | Highly expressed | Also known as |
| | | | | | localized | receptor, endocytosis, | in fat, brain, and | Neurotensin receptor 3 |
| | | | | | endosomes, | general intracellular vesicle | lung | |
| | | | | | golgi, | transport | | |
| | | | | | lysosomes and | | | |
| | | | | | nucleus | | | |
| 4 | 4 | O88600 | ISCHEMIA RESPONSIVE 94 KDA PROTEIN | 94057 | Intracellular, | Heat shock, protein folding, | Widely distributed | Heat shock 70 kDa |
| | | | | | Cytoplasm | stress response | | protein 4 |
| 4 | 4 | Q63002 | MANNOSE 6-PHOSPHATE/INSULIN-LIKE | 273608 | Cell Membrane | Insulin/IGF signaling | Widely distributed | Soluble receptor found in |
| | | | GROWTH FACTOR II RECEPTOR | | | pathway | | serum, amniotic fluid and |
| | | | | | | | | urine. |
| 4 | 5 | Q91YB6 | COMPLEMENT INHIBITORY FACTOR H | 140344 | Secreted | Complement-mediated | Plasma | Regulation of |
| | | | | | | immunity | | complement activation |
| 4 | 6 | Q62701 | TENASCIN (FRAGMENT) | 67815 | Secreted, | Cell adhesion, extracellular | Widely | Negative regulation of |
| | | | | | extracellular | matrix glycoprotein- | distrubuted | cell adhesion. Ligand for |
| | | | | | space, | mediated signaling | | integrin receptors. |
| | | | | | extracellular | | | |
| | | | | | matrix | | | |
| 4 | 4 | GeneID: 311416 | PREDICTED: SIMILAR TO VINCULIN | 116615 | Intracellular | Cell adhesion, cell structure | Unknown | Non-motor actin binding |
| | | | | | | | | protein |
| 3 | 3 | P63102 | 14-3-3 PROTEIN ZETA/DELTA | 27771 | Cytoplasm | Chaperone, signal | Highly expressed | Adapter protein |
| | | | | | | transduction | in brain | implicated in the |
| | | | | | | | | regulation of a large |
| | | | | | | | | spectrum of both general |
| | | | | | | | | and specialized signaling |
| | | | | | | | | pathway |
| 3 | 4 | P07335 | CREATINE KINASE B-TYPE | 42712 | Cytoplasm | Kinase, energy modulation | Brain specific | Phosphocreatine |
| | | | | | | | isoform | metabolism that may be |
| | | | | | | | | necessary for brain |
| | | | | | | | | development |
| 3 | 3 | P35704 | PEROXIREDOXIN-2 | 21652 | Cytoplasm | Oxidoreductase, | Widely | Antiadation and free |
| | | | | | | Peroxidase | distributed, highly | radical removal |
| | | | | | | | expressed in | |
| | | | | | | | bone marrow, | |
| | | | | | | | heart, brain, | |
| | | | | | | | kidney and | |
| | | | | | | | muscle | |
| 3 | 3 | GeneID: 366031 | PREDICTED: SIMILAR TO CRB2 PROTEIN | 138781 | Membrane | Membrane-bound signaling | Unknown | Immunity and defense, |
| | | | | | | molecule? | | mRNA transcription |
| | | | | | | | | regulation |
| 3 | 4 | P60711 | ACTIN, CYTOPLASMIC 1 | 41737 | Cytoplasm | Cell structure, cell mobility, | Ubiquitous | Actins are highly |
| | | | | | | intracellular protein traffic | | conserved proteins that |
| | | | | | | | | are involved in various |
| | | | | | | | | types of cell motility and |
| | | | | | | | | are ubiquitously |
| | | | | | | | | expressed in all |
| | | | | | | | | eukaryotic cells. |
| 3 | 4 | P35952 | LOW-DENSITY LIPOPROTEIN RECEPTOR | 96622 | Cell Membrane | Lipid and fatty acid | Plasma | Binds LDL, the major |
| | | | PRECURSOR | | | transport and metabolism | | cholesterol-carrying |
| | | | | | | | | lipoprotein of plasma, |
| | | | | | | | | and transports it into cells |
| | | | | | | | | by endocytosis |
| 3 | 3 | P12785 | FATTY ACID SYNTHASE | 272650 | Intracellular- | Lipid and fatty acid | Ubiquitously | Invovled in catalyzing the |
| | | | | | cytoplasm | biosynthesis | expressed | formation of long chain |
| | | | | | | | | fatty acids. |
| 3 | 4 | P10111 | PEPTIDYL-PROLYL CIS-TRANS ISOMERASE A | 17743 | Cytoplasm | Isomerase, Protein folding, | Widely | PPlases accelerate the |
| | | | | | | Nuclear transport | distributed, highly | folding of proteins |
| | | | | | | | expressed in | |
| | | | | | | | nerve ganglia | |
| 3 | 4 | P07151 | BETA-2-MICROGLOBULIN PRECURSOR | 13720 | Cell Membrane | Immune response, Major | Expressed on | Beta chain of major |
| | | | | | | histocompatibility complex | nucleated cells | histocompatibility |
| | | | | | | antigen | | complex class I |
| | | | | | | | | molecules |
| 3 | 6 | Q62632 | FOLLISTATIN-RELATED PROTEIN 1 | 34622 | Secreted | Select regulatory molecule - | Widely | Modulates action of some |
| | | | PRECURSOR | | | Homeostatis | distributed, highly | growth factors on cell |
| | | | | | | | expressed in | proliferation and |
| | | | | | | | vascular tissue | differentiation |
| | | | | | | | and nerve ganglia | |
| 3 | 3 | Q5M822 | SERINE (OR CYSTEINE) PROTEINASE | 44671 | Secreted | Serine Protease Inhibitor | Plasma | Belongs to the serpin |
| | | | INHIBITOR, CLADE A (ALPHA-1 | | | | | family |
| | | | ANTIPROTEINASE, ANTITRYPSIN), MEMBER 6 | | | | | |
| 3 | 6 | Q63555 | SP120 - Heterogeneous nuclear | 87748 | Intracellular | Transporter activity - RNA | Widely distributed | Nuclear scaffold protein |
| | | | ribonucleoprotein U | | | binding | | |
| 3 | 6 | Q6PED0 | RIBOSOMAL PROTEIN S27A | 17951 | Intracellular | Protein biosynthesis | Ubiquitous | Ribosomal structural |
| | | | | | | | | protein |
| 3 | 4 | Q62656 | SPLICE ISOFORM 2 OF RECEPTOR-TYPE | 164596 | Cell Membrane | Cell surface receptor | CNS | May be involved in the |
| | | | TYROSINE-PROTEIN PHOSPHATASE ZETA | | | mediated signal | | regulation of specific |
| | | | PRECURSOR | | | transduction | | developmental processes |
| | | | | | | | | in the CNS. |
| 3 | 3 | P31000 | VIMENTIN | 53602 | Intracellular | Cell structure | Widely | Found in various non- |
| | | | | | | | distributed, highly | epithelial cells, especially |
| | | | | | | | expressed in | mesenchymal cells |
| | | | | | | | nerve ganglia | |
| 3 | 3 | P97536 | CULLIN-ASSOCIATED NEDD8-DISSOCIATED | 136362 | Nucleus | Transcriptional Enhancer | Detected in heart, | Also negative regulator of |
| | | | PROTEIN 1 | | | | brain, spleen, | ubiquitin ligase complex |
| | | | | | | | liver, skeletal | |
| | | | | | | | muscle, kidney | |
| | | | | | | | and testis | |
| 3 | 3 | O70535 | LEUKEMIA INHIBITORY FACTOR RECEPTOR | 122394 | Isoform 1: Cell | Cytokine receptor - Signal | Secreted form | The soluble form inhibits |
| | | | PRECURSOR | | membrane; | transducing molecule | found in plasma. | the biological activity of |
| | | | | | Isoform 2: | | Membrane form | LIF by blocking its |
| | | | | | Secreted | | highly expressed | binding to receptors on |
| | | | | | | | in placenta, liver, | target cells |
| | | | | | | | kidney, heart, | |
| | | | | | | | lung and brain. | |
| 3 | 3 | P11442 | CLATHRIN HEAVY CHAIN | 191599 | Intracellular - | Ligand-mediated signaling, | Ubiquitously | Involved in the formation |
| | | | | | vesicle coat | Receptor mediated | expressed | of clathrin coated |
| | | | | | | endocytosis | | vesicles during vesicle |
| | | | | | | | | endocytosis |
| 2 | 3 | P01835 | IG KAPPA CHAIN C REGION, B ALLELE | 11601 | Secreted | Immunoglobulin; B-cell and | Lymphoreticular | Immunoglobulin family |
| | | | | | | antibody-mediated immunity | tissue | |
| 2 | 2 | Q5RKI1 | EUKARYOTIC TRANSLATION INITIATION | 46489 | Intracellular | Protein biosynthesis, | Ubiquitous | Required for mRNA |
| | | | FACTOR 4A2 | | | Translation Initiation factor | | binding to ribosome |
| 2 | 3 | P04276 | VITAMIN D-BINDING PROTEIN PRECURSOR | 53544 | Secreted | Transport | Found in plasma, | Binds and carries vitamin |
| | | | | | | | ascitic fluid, | D and also prevents actin |
| | | | | | | | cerebrospinal | polymerization |
| | | | | | | | fluid, and urine | |
| | | | | | | | and on the | |
| | | | | | | | surface of many | |
| | | | | | | | cell types. | |
| 2 | 3 | GeneID: 500303 | PREDICTED: SIMILAR TO ALPHA ENOLASE | 46489 | Cytoplasm, Cell | Glycolysis and plasminogen | Expressed in | Multifunctional enzyme |
| | | | | | membrane | activation | embryo and in | that plays a part in |
| | | | | | | | most adult | various processes such |
| | | | | | | | tissues, striated | as growth control, |
| | | | | | | | muscle, neurons | hypoxia tolerance and |
| | | | | | | | | allergic responses. May |
| | | | | | | | | also function in the |
| | | | | | | | | fibrinolytic system due to |
| | | | | | | | | its ability to serve as a |
| | | | | | | | | receptor and activator of |
| | | | | | | | | plasminogen on the cell |
| | | | | | | | | surface of several cell |
| | | | | | | | | types |
| 2 | 3 | Q9R0T4 | EPITHELIAL-CADHERIN PRECURSOR | 98715 | Cell Membrane, | Cell adhesion | Widely | May play a role in axonal |
| | | | | | Cell junction | | distributed, highly | growth and synapse |
| | | | | | | | expressed in the | formation |
| | | | | | | | colon | |
| 2 | 2 | Q6P502 | PREDICTED: similar to T-complex protein 1 | 59745 | Cytoplasm | Chaperone | Widely | Belongs to the TCP-1 |
| | | | subunit theta | | | | distributed, highly | chaperonin family |
| | | | | | | | expressed in | |
| | | | | | | | brain | |
| 2 | 2 | Q66HD3 | NUCLEAR AUTOANTIGENIC SPERM PROTEIN | 84200 | Intracellular | Transport | Widely expressed | Required for DNA |
| | | | | | | | | replication, normal cell |
| | | | | | | | | cycle progression and |
| | | | | | | | | cell proliferation |
| 2 | 2 | GeneID: 363155 | PREDICTED: SIMILAR TO PROGRAMMED | 75806 | Cytoplasm | Transmembrane receptor | Expressed in | DNA damage response, |
| | | | CELL DEATH 6 INTERACTING PROTEIN | | | regulatory/adaptor protein | astrocytes and | signal transduction |
| | | | | | | | glioma cells | resulting in induction of |
| | | | | | | | | apoptosis |
| 2 | 2 | P34058 | HEAT SHOCK PROTEIN HSP 90-BETA | 83185 | Cytoplasm | Chaperone, protein folding, | Ubiquitous | Belongs to the heat |
| | | | | | | stress response | | shock protein 90 family |
| 2 | 6 | Q71DI1 | DERMCIDIN | 11284 | Secreted | Neuronal survival, | Brain, skin | A maternal blood-borne |
| | | | | | | phosphatase activity | | factor promotes survival |
| | | | | | | | | of the developing |
| | | | | | | | | thalamus |
| 2 | 8 | Q64240 | AMBP PROTEIN PRECURSOR | 38851 | Secreted | Serine protease inhibitor | Plasma, urine, | It appears not only as a |
| | | | | | | | and cerebrospinal | free monomer but also in |
| | | | | | | | fluid | complexes with IgA and |
| | | | | | | | | albumin |
| 2 | 2 | Q8VID2 | PREDICTED: AMINOPEPTIDASE PUROMYCIN | 103344 | Cytoplasm | Metalloprotease - | Widely | Also known as |
| | | | SENSITIVE | | | Aminopeptidase activity | distributed, highly | METALLOPROTEASE |
| | | | | | | | detected in | MP100 |
| | | | | | | | hippocampus | |
| 2 | 2 | GeneID: 293860 | PREDICTED: SIMILAR TO FILAMIN A | 290169 | Intracellular | Cell structure, Cell motility | Ubiquitous | Non-motor actin binding |
| | | | | | | | | protein |
| 2 | 2 | Q9JLT0 | MYOSIN-10 | 228965 | Intracellular | Cell structure, cell motility | Brain | Actin binding motor |
| | | | | | | | | protein |
| 2 | 4 | P15865 | HISTONE H1.2 | 21856 | Nucleus | Chromatin packaging and | Ubiquitous | Necessary for |
| | | | | | | remodeling | | condensation of nuclear |
| | | | | | | | | DNA |
| 2 | 2 | Q8K586 | GTP-BINDING NUCLEAR PROTEIN RAN, | 24451 | Nucleus | Nucleocytoplasmic | Testis | Required for the import of |
| | | | TESTIS-SPECIFIC ISOFORM | | | transport | | protein into the nucleus |
| | | | | | | | | and also for RNA export: |
| 2 | 2 | P62260 | 14-3-3 PROTEIN EPSILON | 29174 | Cytoplasm | Chaperone, signal | Present at high | Adapter protein |
| | | | | | | transduction | levels in the | implicated in the |
| | | | | | | | pineal gland early | regulation of a large |
| | | | | | | | in development | spectrum of both general |
| | | | | | | | | and specialized signaling |
| | | | | | | | | pathway |

TABLE 4
Protein list from mass spectrometry of all individual samples of embryonic rat CSF collected from E12.5
lateral ventricle (LV), E14.5 lateral ventricle (LV), 4th ventricle (4thV), and E17.5 lateral ventricle (LV).

| # of | | | # of | | | # of | | | # of | | |
|---|---|---|---|---|---|---|---|---|---|---|---|
| unique | | | unique | | | unique | Total | | unique | | |
| peptides | | | peptides | | | peptides | number | | peptides | Total # | |
| from | Total # | | from | Total # | | from | of | Protein matches E14 | from | of | |
| protein | peptides | Protein matches E12.5 | protein | peptides | Protein matches E14 LV | protein | peptides | 4thV | protein | peptides | Protein matches E17.5 LV |
| 121 | 587 | Rattus norvegicus (Rat) | 107 | 437 | Rattus norvegicus (Rat) | 120 | 617 | Rattus norvegicus (Rat) | 111 | 414 | Rattus norvegicus (Rat) |
| | | AA1064-apolipoprotein B. | | | AA1064-apolipoprotein B. | | | AA1064-apolipoprotein B. | | | AA1064-apolipoprotein B. |
| | | [MASS = 536024 | | | [MASS = 536024 | | | [MASS = 536024 | | | [MASS = 536024 |
| 64 | 367 | Rattus norvegicus (Rat) | 62 | 293 | Rattus norvegicus (Rat) | 66 | 382 | Rattus norvegicus (Rat) | 58 | 226 | Rattus norvegicus (Rat) |
| | | Apolipoprotein B - | | | Apolipoprotein B - | | | Apolipoprotein B - | | | Apolipoprotein B - fragment. |
| | | fragment. [MASS = 165356 | | | fragment. [MASS = 165356 | | | fragment. [MASS = 165356 | | | [MASS = 165356 |
| 34 | 96 | Rattus norvegicus (Rat) | 34 | 73 | Rattus norvegicus (Rat) | 37 | 135 | Rattus norvegicus (Rat) | 45 | 61 | Rattus norvegicus (Rat) |
| | | SPLICE ISOFORM 1 OF | | | SPLICE ISOFORM 1 OF | | | SPLICE ISOFORM 1 OF | | | DYNEIN HEAVY CHAIN, |
| | | FIBRONECTIN | | | FIBRONECTIN | | | FIBRONECTIN | | | CYTOSOLIC. [MASS = 532252 |
| | | PRECURSOR. | | | PRECURSOR. | | | PRECURSOR. | | | |
| | | [MASS = 272511 | | | [MASS = 272511 | | | [MASS = 272511 | | | |
| 29 | 104 | Rattus norvegicus (Rat) | 32 | 90 | Rattus norvegicus (Rat) | 34 | 365 | Rattus norvegicus (Rat) | 40 | 55 | Rattus norvegicus (Rat) |
| | | APOLIPOPROTEIN A-IV | | | APOLIPOPROTEIN A-IV | | | SPLICE ISOFORM 1 OF | | | PREDICTED: SIMILAR TO |
| | | PRECURSOR. | | | PRECURSOR. | | | ALPHA-FETOPROTEIN | | | FILAMIN A. [MASS = 290169 |
| | | [MASS = 44456 | | | [MASS = 44456 | | | PRECURSOR. | | | |
| | | | | | | | | [MASS = 68386 | | | |
| 28 | 32 | Rattus norvegicus (Rat) | 29 | 290 | Rattus norvegicus (Rat) | 31 | 120 | Rattus norvegicus (Rat) | 39 | 44 | Rattus norvegicus (Rat) FATTY |
| | | PREDICTED: SIMILAR | | | SPLICE ISOFORM 1 OF | | | APOLIPOPROTEIN A-IV | | | ACID SYNTHASE. |
| | | TO FILAMIN A. | | | ALPHA-FETOPROTEIN | | | PRECURSOR. | | | [MASS = 272650 |
| | | [MASS = 290169 | | | PRECURSOR. | | | [MASS = 44456 | | | |
| | | | | | [MASS = 68386 | | | | | | |
| 27 | 227 | Rattus norvegicus (Rat) | 24 | 54 | Rattus norvegicus (Rat) | 28 | 53 | Rattus norvegicus (Rat) | 39 | 79 | Rattus norvegicus (Rat) |
| | | SPLICE ISOFORM 1 OF | | | ALPHA-2- | | | ALPHA-2- | | | SPLICE ISOFORM 1 OF |
| | | ALPHA-FETOPROTEIN | | | MACROGLOBULIN | | | MACROGLOBULIN | | | FIBRONECTIN PRECURSOR. |
| | | PRECURSOR. | | | PRECURSOR. | | | PRECURSOR. | | | [MASS = 272511 |
| | | [MASS = 68386 | | | [MASS = 163701 | | | [MASS = 163701 | | | |
| 26 | 62 | Rattus norvegicus (Rat) | 24 | 30 | Rattus norvegicus (Rat) | 21 | 26 | Rattus norvegicus (Rat) | 37 | 99 | Rattus norvegicus (Rat) |
| | | ALPHA-1-INHIBITOR 3 | | | GPI-ANCHORED | | | GPI-ANCHORED | | | ALPHA-2-MACROGLOBULIN |
| | | PRECURSOR. | | | CERULOPLASMIN. | | | CERULOPLASMIN. | | | PRECURSOR. [MASS = 163701 |
| | | [MASS = 163773 | | | [MASS = 123749 | | | [MASS = 123749 | | | |
| 26 | 32 | Rattus norvegicus (Rat) | 22 | 38 | Rattus norvegicus (Rat) | 21 | 35 | Rattus norvegicus (Rat) | 33 | 418 | Rattus norvegicus (Rat) |
| | | FATTY ACID SYNTHASE. | | | ALPHA-1-INHIBITOR 3 | | | ALPHA-1-INHIBITOR 3 | | | SPLICE ISOFORM 1 OF |
| | | [MASS = 272650 | | | PRECURSOR. | | | PRECURSOR. | | | ALPHA-FETOPROTEIN |
| | | | | | [MASS = 163773 | | | [MASS = 163773 | | | PRECURSOR. [MASS = 68386 |
| 22 | 33 | Rattus norvegicus (Rat) | 21 | 22 | Rattus norvegicus (Rat) | 20 | 96 | Rattus norvegicus (Rat) | 28 | 30 | Rattus norvegicus (Rat) |
| | | GPI-ANCHORED | | | FATTY ACID SYNTHASE. | | | BA1-667 - Transferrin. | | | MYOSIN-10. [MASS = 228965 |
| | | CERULOPLASMIN. | | | [MASS = 272650 | | | [MASS = 107448 | | | |
| | | [MASS = 123749 | | | | | | | | | |
| 20 | 123 | Rattus norvegicus (Rat) | 21 | 96 | Rattus norvegicus (Rat) | 19 | 30 | Rattus norvegicus (Rat) | 26 | 78 | Rattus norvegicus (Rat) |
| | | SERUM ALBUMIN | | | BA1-667 - Transferrin. | | | CONTACTIN-2 | | | APOLIPOPROTEIN A-IV |
| | | PRECURSOR. | | | [MASS = 107448 | | | PRECURSOR. | | | PRECURSOR. [MASS = 44456 |
| | | [MASS = 68719 | | | | | | [MASS = 113043 | | | |
| 19 | 20 | Rattus norvegicus (Rat) | 19 | 60 | Rattus norvegicus (Rat) | 18 | 79 | Rattus norvegicus (Rat) | 25 | 33 | Rattus norvegicus (Rat) |
| | | MYOSIN-10. | | | APOLIPOPROTEIN A-I | | | APOLIPOPROTEIN A-I | | | CONTACTIN-1 PRECURSOR. |
| | | [MASS = 228965 | | | PRECURSOR. | | | PRECURSOR. | | | [MASS = 113495 |
| | | | | | [MASS = 30088 | | | [MASS = 30088 | | | |
| 18 | 38 | Rattus norvegicus (Rat) | 19 | 112 | Rattus norvegicus (Rat) | 18 | 97 | Rattus norvegicus (Rat) | 23 | 160 | Rattus norvegicus (Rat) |
| | | HEAT SHOCK PROTEIN | | | SERUM ALBUMIN | | | SERUM ALBUMIN | | | SERUM ALBUMIN |
| | | 86. [MASS = 84815 | | | PRECURSOR. | | | PRECURSOR. | | | PRECURSOR. [MASS = 68719 |
| | | | | | [MASS = 68719 | | | [MASS = 68719 | | | |
| 18 | 27 | Rattus norvegicus (Rat) | 17 | 19 | Rattus norvegicus (Rat) | 18 | 33 | Rattus norvegicus (Rat) | 23 | 29 | Rattus norvegicus (Rat) |
| | | ELONGATION FACTOR | | | PREDICTED: NIDOGEN. | | | PREDICTED: NIDOGEN. | | | ALPHA-1-MACROGLOBULIN. |
| | | 2. [MASS = 95153 | | | [MASS = 138365 | | | [MASS = 138365 | | | [MASS = 167125 |
| 18 | 43 | Rattus norvegicus (Rat) | 15 | 38 | Rattus norvegicus (Rat) | 15 | 34 | Rattus norvegicus (Rat) | 20 | 21 | Rattus norvegicus (Rat) |
| | | PREDICTED: SIMILAR | | | ALPHA-1- | | | ALPHA-1- | | | MYOSIN-9. [MASS = 226207 |
| | | TO PYRUVATE KINASE | | | ANTIPROTEINASE | | | ANTIPROTEINASE | | | |
| | | (EC 2.7.1.40) ISOZYME | | | PRECURSOR. | | | PRECURSOR. | | | |
| | | M2 - RAT. [MASS = 57731 | | | [MASS = 46136 | | | [MASS = 46136 | | | |
| 17 | 32 | Rattus norvegicus (Rat) | 15 | 20 | Rattus norvegicus (Rat) | 15 | 17 | Rattus norvegicus (Rat) | 20 | 49 | Rattus norvegicus (Rat) |
| | | ALPHA-1- | | | CONTACTIN-2 | | | DA1-24-Complement | | | PREDICTED: SIMILAR TO |
| | | ANTIPROTEINASE | | | PRECURSOR. | | | Factor B. [MASS = 124379 | | | INTER-ALPHA-INHIBITOR H2 |
| | | PRECURSOR. | | | [MASS = 113043 | | | | | | CHAIN. [MASS = 105715 |
| | | [MASS = 46136 | | | | | | | | | |
| 17 | 25 | Rattus norvegicus (Rat) | 14 | 18 | Rattus norvegicus (Rat) | 14 | 23 | Rattus norvegicus (Rat) | 19 | 47 | Rattus norvegicus (Rat) |
| | | COMPLEMENT C3 | | | HEAT SHOCK PROTEIN | | | PREDICTED: SIMILAR | | | ALPHA-1-ANTIPROTEINASE |
| | | PRECURSOR. | | | 86. [MASS = 84815 | | | TO CADHERIN-5. | | | PRECURSOR. [MASS = 46136 |
| | | [MASS = 186460 | | | | | | [MASS = 135230 | | | |
| 17 | 63 | Rattus norvegicus (Rat) | 14 | 29 | Rattus norvegicus (Rat) | 13 | 20 | Rattus norvegicus (Rat) | 19 | 24 | Rattus norvegicus (Rat) |
| | | BA1-667 - Transferrin. | | | PREDICTED: SIMILAR | | | ALPHA-2 ANTIPLASMIN. | | | ELONGATION FACTOR 2. |
| | | [MASS = 107448 | | | TO PYRUVATE KINASE | | | [MASS = 46465 | | | [MASS = 95153 |
| | | | | | (EC 2.7.1.40) ISOZYME | | | | | | |
| | | | | | M2 - RAT. [MASS = 57731 | | | | | | |
| 16 | 56 | Rattus norvegicus (Rat) | 12 | 17 | Rattus norvegicus (Rat) | 13 | 18 | Rattus norvegicus (Rat) | 19 | 19 | Rattus norvegicus (Rat) |
| | | APOLIPOPROTEIN A-I | | | ALPHA-2 ANTIPLASMIN. | | | SPLICE ISOFORM 1 OF | | | PREDICTED: SIMILAR TO |
| | | PRECURSOR. | | | [MASS = 46465 | | | AGRIN PRECURSOR. | | | CAD PROTEIN. |
| | | [MASS = 30088 | | | | | | [MASS = 208646 | | | [MASS = 250725 |
| 16 | 28 | Rattus norvegicus (Rat) | 12 | 24 | Rattus norvegicus (Rat) | 13 | 25 | Rattus norvegicus (Rat) | 18 | 24 | Rattus norvegicus (Rat) |
| | | ALPHA-2- | | | NEURAL-CADHERIN | | | PREDICTED: SIMILAR | | | HYPOTHETICAL PROTEIN |
| | | MACROGLOBULIN | | | PRECURSOR. | | | TO HEPARAN SULFATE | | | LOC314432-Similar to |
| | | PRECURSOR. | | | [MASS = 99686 | | | PROTEOGLYCAN 2. | | | ubiquitin-protein ligase (EC |
| | | [MASS = 163701 | | | | | | [MASS = 377284 | | | 6.3.2.19) E1. [MASS = 117788 |
| 16 | 17 | Rattus norvegicus (Rat) | 12 | 22 | Rattus norvegicus (Rat) | 13 | 19 | Rattus norvegicus (Rat) | 18 | 102 | Rattus norvegicus (Rat) BA1- |
| | | CLATHRIN HEAVY | | | PREDICTED: SIMILAR | | | INTER-ALPHA- | | | 667 - Transferrin. |
| | | CHAIN. [MASS = 191599 | | | TO CADHERIN-5. | | | INHIBITOR H4 HEAVY | | | [MASS = 107448 |
| | | | | | [MASS = 135230 | | | CHAIN. [MASS = 103755 | | | |
| 14 | 14 | Rattus norvegicus (Rat) | 12 | 14 | Rattus norvegicus (Rat) | 12 | 14 | Rattus norvegicus (Rat) | 17 | 20 | Rattus norvegicus (Rat) DA1- |
| | | DYNEIN HEAVY CHAIN, | | | PREDICTED: SIMILAR | | | GELSOLIN. | | | 24-Complement Factor B. |
| | | CYTOSOLIC. | | | TO LAMININ B1. | | | [MASS = 86286 | | | [MASS = 124379 |
| | | [MASS = 532252 | | | [MASS = 228429 | | | | | | |
| 13 | 23 | Rattus norvegicus (Rat) | 11 | 12 | Rattus norvegicus (Rat) | 12 | 15 | Rattus norvegicus (Rat) | 17 | 34 | Rattus norvegicus (Rat) |
| | | RAT ALPHA(1)- | | | GELSOLIN. | | | SERINE/CYSTEINE | | | PREDICTED: MICROTUBULE- |
| | | INHIBITOR 3, VARIANT I | | | [MASS = 86286 | | | PROTEINASE | | | ASSOCIATED PROTEIN 1B. |
| | | PRECURSOR. | | | | | | INHIBITOR, CLADE C, | | | [MASS = 269643 |
| | | [MASS = 165326 | | | | | | MEMBER 1. | | | |
| | | | | | | | | [MASS = 52234 | | | |
| 13 | 16 | Rattus norvegicus (Rat) | 11 | 13 | Rattus norvegicus (Rat) | 12 | 22 | Rattus norvegicus (Rat) | 16 | 17 | Rattus norvegicus (Rat) |
| | | PREDICTED: NIDOGEN. | | | ELONGATION FACTOR | | | COMPLEMENT C3 | | | CONTACTIN-2 PRECURSOR. |
| | | [MASS = 138365 | | | 2. [MASS = 95153 | | | PRECURSOR. | | | [MASS = 113043 |
| | | | | | | | | [MASS = 186460 | | | |
| 13 | 13 | Rattus norvegicus (Rat) | 11 | 18 | Rattus norvegicus (Rat) | 12 | 24 | Rattus norvegicus (Rat) | 16 | 23 | Rattus norvegicus (Rat) HEAT |
| | | PREDICTED: SIMILAR | | | COMPLEMENT C3 | | | APOLIPOPROTEIN E | | | SHOCK PROTEIN 86. |
| | | TO CAD PROTEIN. | | | PRECURSOR. | | | PRECURSOR. | | | [MASS = 84815 |
| | | [MASS = 250725 | | | [MASS = 186460 | | | [MASS = 35753 | | | |
| 13 | 15 | Rattus norvegicus (Rat) | 11 | 13 | Rattus norvegicus (Rat) | 11 | 14 | Rattus norvegicus (Rat) | 16 | 17 | Rattus norvegicus (Rat) |
| | | VIMENTIN. [MASS = 53602 | | | TRANSITIONAL | | | LAR RECEPTOR- | | | CLATHRIN HEAVY CHAIN. |
| | | | | | ENDOPLASMIC | | | LINKED TYROSINE | | | [MASS = 191599 |
| | | | | | RETICULUM ATPASE. | | | PHOSPHATASE. | | | |
| | | | | | [MASS = 89534 | | | [MASS = 181130 | | | |
| 13 | 14 | Rattus norvegicus (Rat) | 11 | 11 | Rattus norvegicus (Rat) | 11 | 12 | Rattus norvegicus (Rat) | 15 | 25 | Rattus norvegicus (Rat) |
| | | PREDICTED: SIMILAR | | | NUCLEAR | | | DELETED IN | | | COMPLEMENT C3 |
| | | TO GCN1 GENERAL | | | AUTOANTIGENIC | | | COLORECTAL CANCER. | | | PRECURSOR. [MASS = 186460 |
| | | CONTROL OF AMINO- | | | SPERM PROTEIN. | | | [MASS = 158142 | | | |
| | | ACID SYNTHESIS 1- | | | [MASS = 84200 | | | | | | |
| | | LIKE 1. [MASS = 302942 | | | | | | | | | |
| 12 | 12 | Rattus norvegicus (Rat) | 11 | 18 | Rattus norvegicus (Rat) | 11 | 12 | Rattus norvegicus (Rat) | 15 | 15 | Rattus norvegicus (Rat) |
| | | HYPOTHETICAL | | | CORTICOSTEROID- | | | SERINE PEPTIDASE | | | PREDICTED: SIMILAR TO |
| | | PROTEIN LOC314432- | | | BINDING GLOBULIN | | | INHIBITOR, CLADE G, | | | GCN1 GENERAL CONTROL |
| | | Similar to ubiquitin-protein | | | PRECURSOR. | | | MEMBER 1. | | | OF AMINO-ACID SYNTHESIS |
| | | ligase (EC 6.3.2.19) E1. | | | [MASS = 44672 | | | [MASS = 55611 | | | 1-LIKE 1. [MASS = 302942 |
| | | [MASS = 117788 | | | | | | | | | |
| 12 | 17 | Rattus norvegicus (Rat) | 11 | 22 | Rattus norvegicus (Rat) | 11 | 17 | Rattus norvegicus (Rat) | 14 | 32 | Rattus norvegicus (Rat) |
| | | ATP-CITRATE | | | APOLIPOPROTEIN E | | | CORTICOSTEROID- | | | APOLIPOPROTEIN A-I |
| | | SYNTHASE. | | | PRECURSOR. | | | BINDING GLOBULIN | | | PRECURSOR. [MASS = 30088 |
| | | [MASS = 120781 | | | [MASS = 35753 | | | PRECURSOR. | | | |
| | | | | | | | | [MASS = 44672 | | | |
| 12 | 15 | Rattus norvegicus (Rat) | 10 | 47 | Rattus norvegicus (Rat) | 11 | 12 | Rattus norvegicus (Rat) | 14 | 46 | Rattus norvegicus (Rat) |
| | | TRANSITIONAL | | | TRANSTHYRETIN | | | PREDICTED: SIMILAR | | | CORTICOSTEROID-BINDING |
| | | ENDOPLASMIC | | | PRECURSOR. | | | TO LAMININ B1. | | | GLOBULIN PRECURSOR. |
| | | RETICULUM ATPASE. | | | [MASS = 15720 | | | [MASS = 228429 | | | [MASS = 44672 |
| | | [MASS = 89534 | | | | | | | | | |
| 11 | 14 | Rattus norvegicus (Rat) | 10 | 17 | Rattus norvegicus (Rat) | 11 | 11 | Rattus norvegicus (Rat) | 14 | 15 | Rattus norvegicus (Rat) |
| | | ALPHA-2 ANTIPLASMIN. | | | RAT ALPHA(1)- | | | ELONGATION FACTOR | | | CULLIN-ASSOCIATED |
| | | [MASS = 46465 | | | INHIBITOR 3, VARIANT I | | | 2. [MASS = 95153 | | | NEDD8-DISSOCIATED |
| | | | | | PRECURSOR. | | | | | | PROTEIN 1. [MASS = 136362 |
| | | | | | [MASS = 165326 | | | | | | |
| 11 | 12 | Rattus norvegicus (Rat) | 10 | 13 | Rattus norvegicus (Rat) | 11 | 14 | Rattus norvegicus (Rat) | 14 | 42 | Rattus norvegicus (Rat) |
| | | IMPORTIN BETA-1 | | | CONTRAPSIN-LIKE | | | SPLICE ISOFORM 1 OF | | | TUBULIN ALPHA-1 CHAIN. |
| | | SUBUNIT. [MASS = 97184 | | | PROTEASE INHIBITOR 6 | | | FIBRINOGEN ALPHA | | | [MASS = 50136 |
| | | | | | PRECURSOR. | | | CHAIN PRECURSOR. | | | |
| | | | | | [MASS = 46652 | | | [MASS = 86686 | | | |
| 11 | 14 | Rattus norvegicus (Rat) | 10 | 12 | Rattus norvegicus (Rat) | 11 | 16 | Rattus norvegicus (Rat) | 14 | 14 | Rattus norvegicus (Rat) GPI- |
| | | CULLIN-ASSOCIATED | | | PREDICTED: SIMILAR | | | PREDICTED: SIMILAR | | | ANCHORED |
| | | NEDD8-DISSOCIATED | | | TO KINESIN FAMILY | | | TO ALPHA 3 TYPE VI | | | CERULOPLASMIN. |
| | | PROTEIN 1. | | | MEMBER 23. | | | COLLAGEN ISOFORM 1 | | | [MASS = 123749 |
| | | [MASS = 136362 | | | [MASS = 108791 | | | PRECURSOR. | | | |
| | | | | | | | | [MASS = 369017 | | | |
| 11 | 40 | Rattus norvegicus (Rat) | 10 | 16 | Rattus norvegicus (Rat) | 10 | 21 | Rattus norvegicus (Rat) | 14 | 28 | Rattus norvegicus (Rat) |
| | | TUBULIN ALPHA-1 | | | COLLAGEN ALPHA-1(I) | | | NEURAL-CADHERIN | | | ECTONUCLEOTIDE |
| | | CHAIN. [MASS = 50136 | | | CHAIN PRECURSOR. | | | PRECURSOR. | | | PYROPHOSPHATASE/ |
| | | | | | [MASS = 137886 | | | [MASS = 99686 | | | PHOSPHODIESTERASE 2. |
| | | | | | | | | | | | [MASS = 101310 |
| 11 | 18 | Rattus norvegicus (Rat) | 10 | 10 | Rattus norvegicus (Rat) | 10 | 14 | Rattus norvegicus (Rat) | 13 | 18 | Rattus norvegicus (Rat) |
| | | PREDICTED: SIMILAR | | | CLATHRIN HEAVY | | | PREDICTED: LAMININ, | | | GELSOLIN. [MASS = 86286 |
| | | TO FIBULIN-1 | | | CHAIN. [MASS = 191599 | | | GAMMA 1. | | | |
| | | PRECURSOR. | | | | | | [MASS = 177387 | | | |
| | | [MASS = 78072 | | | | | | | | | |
| 11 | 18 | Rattus norvegicus (Rat) | 9 | 20 | Rattus norvegicus (Rat) | 10 | 22 | Rattus norvegicus (Rat) | 13 | 15 | Rattus norvegicus (Rat) |
| | | APOLIPOPROTEIN E | | | TUBULIN ALPHA-1 | | | PREDICTED: SIMILAR | | | VIMENTIN. [MASS = 53602 |
| | | PRECURSOR. | | | CHAIN. [MASS = 50136 | | | TO FIBULIN-1 | | | |
| | | [MASS = 35753 | | | | | | PRECURSOR. | | | |
| | | | | | | | | [MASS = 78072 | | | |
| 11 | 11 | Rattus norvegicus (Rat) | 9 | 9 | Rattus norvegicus (Rat) | 10 | 11 | Rattus norvegicus (Rat) | 12 | 21 | Rattus norvegicus (Rat) |
| | | Glutamyl-prolyl-tRNA | | | LAR RECEPTOR- | | | ECTONUCLEOTIDE | | | TRANSITIONAL |
| | | synthetase. | | | LINKED TYROSINE | | | PYROPHOSPHATASE/ | | | ENDOPLASMIC RETICULUM |
| | | [MASS = 170088 | | | PHOSPHATASE. | | | PHOSPHODIESTERASE | | | ATPASE. [MASS = 89534 |
| | | | | | [MASS = 181130 | | | 2. [MASS = 101310 | | | |
| 11 | 11 | Rattus norvegicus (Rat) | 9 | 11 | Rattus norvegicus (Rat) | 10 | 21 | Rattus norvegicus (Rat) | 12 | 12 | Rattus norvegicus (Rat) |
| | | MYOSIN-9. | | | IMPORTIN BETA-1 | | | COLLAGEN ALPHA-1(I) | | | COMPLEMENT COMPONENT |
| | | [MASS = 226207 | | | SUBUNIT. [MASS = 97184 | | | CHAIN PRECURSOR. | | | 2. [MASS = 83699 |
| | | | | | | | | [MASS = 137886 | | | |
| 11 | 15 | Rattus norvegicus (Rat) | 9 | 9 | Rattus norvegicus (Rat) | 10 | 21 | Rattus norvegicus (Rat) | 12 | 15 | Rattus norvegicus (Rat) |
| | | INTER-ALPHA- | | | PREDICTED: VON | | | LUMICAN PRECURSOR. | | | PREDICTED: SIMILAR TO |
| | | INHIBITOR H4 HEAVY | | | WILLEBRAND FACTOR. | | | [MASS = 38279 | | | DESMOPLAKIN ISOFORM II. |
| | | CHAIN. [MASS = 103755 | | | [MASS = 308474 | | | | | | [MASS = 264186 |
| 10 | 12 | Rattus norvegicus (Rat) | 9 | 15 | Rattus norvegicus (Rat) | 9 | 51 | Rattus norvegicus (Rat) | 12 | 15 | Rattus norvegicus (Rat) |
| | | GELSOLIN. | | | PREDICTED: SIMILAR | | | TRANSTHYRETIN | | | PREDICTED: SIMILAR TO |
| | | [MASS = 86286 | | | TO HEPARAN SULFATE | | | PRECURSOR. | | | ALPHA 3 TYPE VI COLLAGEN |
| | | | | | PROTEOGLYCAN 2. | | | [MASS = 15720 | | | ISOFORM 1 PRECURSOR. |
| | | | | | [MASS = 377284 | | | | | | [MASS = 369017 |
| 10 | 10 | Rattus norvegicus (Rat) | 9 | 9 | Rattus norvegicus (Rat) | 9 | 13 | Rattus norvegicus (Rat) | 11 | 11 | Rattus norvegicus (Rat) |
| | | NUCLEAR | | | PREDICTED: LAMININ, | | | CATHEPSIN B | | | PREDICTED: SIMILAR TO |
| | | AUTOANTIGENIC | | | GAMMA 1. | | | PRECURSOR. | | | COATOMER PROTEIN |
| | | SPERM PROTEIN. | | | [MASS = 177387 | | | [MASS = 37470 | | | COMPLEX SUBUNIT ALPHA. |
| | | [MASS = 84200 | | | | | | | | | [MASS = 138360 |
| 10 | 16 | Rattus norvegicus (Rat) | 9 | 20 | Rattus norvegicus (Rat) | 9 | 10 | Rattus norvegicus (Rat) | 11 | 19 | Rattus norvegicus (Rat) |
| | | SP120-Heterogeneous | | | LUMICAN PRECURSOR. | | | FIBRINOGEN BETA | | | CLUSTERIN PRECURSOR. |
| | | nuclear ribonucleoprotein | | | [MASS = 38279 | | | CHAIN PRECURSOR. | | | [MASS = 51375 |
| | | U. [MASS = 87748 | | | | | | [MASS = 54303 | | | |
| 10 | 11 | Rattus norvegicus (Rat) | 9 | 9 | Rattus norvegicus (Rat) | 9 | 13 | Rattus norvegicus (Rat) | 11 | 16 | Rattus norvegicus (Rat) |
| | | CONTACTIN-2 | | | Neogenin precursor. | | | RAT ALPHA(1)- | | | PREDICTED: SIMILAR TO |
| | | PRECURSOR. | | | [MASS = 156144 | | | INHIBITOR 3, VARIANT I | | | PYRUVATE KINASE (EC |
| | | [MASS = 113043 | | | | | | PRECURSOR. | | | 2.7.1.40) ISOZYME M2 - RAT. |
| | | | | | | | | [MASS = 165326 | | | [MASS = 57731 |
| 9 | 10 | Rattus norvegicus (Rat) | 9 | 16 | Rattus norvegicus (Rat) | 9 | 11 | Rattus norvegicus (Rat) | 11 | 15 | Rattus norvegicus (Rat) |
| | | PHOSPHOGLYCERATE | | | PREDICTED: SIMILAR | | | PREDICTED: VON | | | ALPHA-1-INHIBITOR 3 |
| | | KINASE 1. [MASS = 44423 | | | TO FIBULIN-1 | | | WILLEBRAND FACTOR. | | | PRECURSOR. [MASS = 163773 |
| | | | | | PRECURSOR. | | | [MASS = 308474 | | | |
| | | | | | [MASS = 78072 | | | | | | |
| 9 | 10 | Rattus norvegicus (Rat) | 9 | 10 | Rattus norvegicus (Rat) | 9 | 18 | Rattus norvegicus (Rat) | 11 | 11 | Rattus norvegicus (Rat) |
| | | HEAT SHOCK COGNATE | | | DA1-24-Complement | | | LOC367586 PROTEIN- | | | HEPHAESTIN PRECURSOR. |
| | | 71 KDA PROTEIN. | | | Factor B. [MASS = 124379 | | | Immunoglobulin Gamma | | | [MASS = 129593 |
| | | [MASS = 70871 | | | | | | heavy Chain. | | | |
| | | | | | | | | [MASS = 50949 | | | |
| 9 | 22 | Rattus norvegicus (Rat) | 9 | 12 | Rattus norvegicus (Rat) | 9 | 11 | Rattus norvegicus (Rat) | 11 | 36 | Rattus norvegicus (Rat) |
| | | TUBULIN BETA-5 CHAIN. | | | ACTIN, ALPHA | | | SPLICE ISOFORM LONG | | | LOC367586 PROTEIN- |
| | | [MASS = 49671 | | | SKELETAL MUSCLE. | | | OF HYALURONAN AND | | | Immunoglobulin Gamma heavy |
| | | | | | [MASS = 42051 | | | PROTEOGLYCAN LINK | | | Chain. [MASS = 50949 |
| | | | | | | | | PROTEIN 1 | | | |
| | | | | | | | | PRECURSOR. | | | |
| | | | | | | | | [MASS = 40262 | | | |
| 9 | 12 | Rattus norvegicus (Rat) | 9 | 13 | Rattus norvegicus (Rat) | 8 | 10 | Rattus norvegicus (Rat) | 11 | 19 | Rattus norvegicus (Rat) ACTIN, |
| | | CONTRAPSIN-LIKE | | | INTER-ALPHA- | | | HEAT SHOCK PROTEIN | | | ALPHA SKELETAL MUSCLE. |
| | | PROTEASE INHIBITOR 6 | | | INHIBITOR H4 HEAVY | | | 86. [MASS = 84815 | | | [MASS = 42051 |
| | | PRECURSOR. | | | CHAIN. [MASS = 103755 | | | | | | |
| | | [MASS = 46652 | | | | | | | | | |
| 9 | 19 | Rattus norvegicus (Rat) | 8 | 16 | Rattus norvegicus (Rat) | 8 | 8 | Rattus norvegicus (Rat) | 10 | 20 | Rattus norvegicus (Rat) |
| | | HYPOTHETICAL | | | TUBULIN BETA-5 CHAIN. | | | HEPHAESTIN | | | NEURAL-CADHERIN |
| | | PROTEIN RGD1305887- | | | [MASS = 49671 | | | PRECURSOR. | | | PRECURSOR. [MASS = 99686 |
| | | TUBULIN BETA CHAIN. | | | | | | [MASS = 129593 | | | |
| | | [MASS = 50059 | | | | | | | | | |
| 9 | 11 | Rattus norvegicus (Rat) | 8 | 14 | Rattus norvegicus (Rat) | 8 | 13 | Rattus norvegicus (Rat) | 10 | 105 | Rattus norvegicus (Rat) |
| | | PROMININ-1S1 SPLICE | | | CONTRAPSIN-LIKE | | | COLLAGEN ALPHA-1(III) | | | TRANSTHYRETIN |
| | | VARIANT. [MASS = 96632 | | | PROTEASE INHIBITOR 1 | | | CHAIN PRECURSOR. | | | PRECURSOR. [MASS = 15720 |
| | | | | | PRECURSOR. | | | [MASS = 138936 | | | |
| | | | | | [MASS = 46562 | | | | | | |
| 9 | 9 | Rattus norvegicus (Rat) | 8 | 14 | Rattus norvegicus (Rat) | 8 | 9 | Rattus norvegicus (Rat) | 10 | 11 | Rattus norvegicus (Rat) |
| | | IKAP. [MASS = 149171 | | | SERINE PEPTIDASE | | | SPLICE ISOFORM 1 OF | | | IMPORTIN BETA-1 SUBUNIT. |
| | | | | | INHIBITOR, CLADE G, | | | REELIN PRECURSOR. | | | [MASS = 97184 |
| | | | | | MEMBER 1. | | | [MASS = 387531 | | | |
| | | | | | [MASS = 55611 | | | | | | |
| 9 | 10 | Rattus norvegicus (Rat) | 8 | 9 | Rattus norvegicus (Rat) | 8 | 20 | Rattus norvegicus (Rat) | 10 | 10 | Rattus norvegicus (Rat) |
| | | PREDICTED: NEURAL | | | ISCHEMIA RESPONSIVE | | | PLASMINOGEN | | | PREDICTED: SIMILAR TO |
| | | PRECURSOR CELL | | | 94 KDA PROTEIN. | | | PRECURSOR. | | | CELLULAR APOPTOSIS |
| | | EXPRESSED, | | | [MASS = 94057 | | | [MASS = 90536 | | | SUSCEPTIBILITY PROTEIN. |
| | | DEVELOPMENTALLY | | | | | | | | | [MASS = 110214 |
| | | DOWN-REGULATED | | | | | | | | | |
| | | GENE 4A. | | | | | | | | | |
| | | [MASS = 112368 | | | | | | | | | |
| 9 | 18 | Rattus norvegicus (Rat) | 8 | 8 | Rattus norvegicus (Rat) | 8 | 12 | Rattus norvegicus (Rat) | 10 | 11 | Rattus norvegicus (Rat) |
| | | ACTIN, ALPHA | | | HYPOTHETICAL | | | EPSILON 1 GLOBIN. | | | NUCLEAR AUTOANTIGENIC |
| | | SKELETAL MUSCLE. | | | PROTEIN LOC314432- | | | [MASS = 16105 | | | SPERM PROTEIN. |
| | | [MASS = 42051 | | | Similar to ubiquitin-protein | | | | | | [MASS = 84200 |
| | | | | | ligase (EC 6.3.2.19) E1. | | | | | | |
| | | | | | [MASS = 117788 | | | | | | |
| 9 | 27 | Rattus norvegicus (Rat) | 8 | 10 | Rattus norvegicus (Rat) | 8 | 13 | Rattus norvegicus (Rat) | 10 | 11 | Rattus norvegicus (Rat) |
| | | ELONGATION FACTOR | | | HEPHAESTIN | | | SPLICE ISOFORM HMW | | | ISCHEMIA RESPONSIVE 94 KDA |
| | | 1-ALPHA 1. | | | PRECURSOR. | | | OF KININOGEN-1 | | | PROTEIN. [MASS = 94057 |
| | | [MASS = 50114 | | | [MASS = 129593 | | | PRECURSOR. | | | |
| | | | | | | | | [MASS = 70933 | | | |
| 9 | 12 | Rattus norvegicus (Rat) | 8 | 15 | Rattus norvegicus (Rat) | 8 | 19 | Rattus norvegicus (Rat) | 10 | 10 | Rattus norvegicus (Rat) |
| | | PREDICTED: SIMILAR | | | PLASMINOGEN | | | SPLICE ISOFORM | | | PREDICTED: SIMILAR TO |
| | | TO HEPARAN SULFATE | | | PRECURSOR. | | | GAMMA-B OF | | | RAS GTPASE-ACTIVATING- |
| | | PROTEOGLYCAN 2. | | | [MASS = 90536 | | | FIBRINOGEN GAMMA | | | LIKE PROTEIN IQGAP1. |
| | | [MASS = 377284 | | | | | | CHAIN PRECURSOR. | | | [MASS = 196522 |
| | | | | | | | | [MASS = 50633 | | | |
| 9 | 9 | Rattus norvegicus (Rat) | 8 | 8 | Rattus norvegicus (Rat) | 8 | 9 | Rattus norvegicus (Rat) | 10 | 11 | Rattus norvegicus (Rat) |
| | | SPLICE ISOFORM LONG | | | CADHERIN-6 | | | CADHERIN-6 | | | PREDICTED: SIMILAR TO |
| | | OF HYALURONAN AND | | | PRECURSOR. | | | PRECURSOR. | | | UBIQUITIN CARBOXYL- |
| | | PROTEOGLYCAN LINK | | | [MASS = 88341 | | | [MASS = 88341 | | | TERMINAL HYDROLASE 5. |
| | | PROTEIN 1 | | | | | | | | | [MASS = 95779 |
| | | PRECURSOR. | | | | | | | | | |
| | | [MASS = 40262 | | | | | | | | | |
| 8 | 32 | Rattus norvegicus (Rat) | 8 | 10 | Rattus norvegicus (Rat) | 7 | 9 | Rattus norvegicus (Rat) | 10 | 12 | Rattus norvegicus (Rat) |
| | | TRANSTHYRETIN | | | ECTONUCLEOTIDE | | | TUBULIN BETA-5 CHAIN. | | | PREDICTED: |
| | | PRECURSOR. | | | PYROPHOSPHATASE/ | | | [MASS = 49671 | | | AMINOPEPTIDASE |
| | | [MASS = 15720 | | | PHOSPHODIESTERASE | | | | | | PUROMYCIN SENSITIVE. |
| | | | | | 2. [MASS = 101310 | | | | | | [MASS = 103344 |
| 8 | 8 | Rattus norvegicus (Rat) | 8 | 10 | Rattus norvegicus (Rat) | 7 | 8 | Rattus norvegicus (Rat) | 10 | 10 | Rattus norvegicus (Rat) |
| | | ISCHEMIA RESPONSIVE | | | PROMININ-1S1 SPLICE | | | ANGIOTENSINOGEN | | | COATOMER SUBUNIT BETA. |
| | | 94 KDA PROTEIN. | | | VARIANT. [MASS = 96632 | | | PRECURSOR. | | | [MASS = 107011 |
| | | [MASS = 94057 | | | | | | [MASS = 51982 | | | |
| 8 | 12 | Rattus norvegicus (Rat) | 8 | 9 | Rattus norvegicus (Rat) | 7 | 8 | Rattus norvegicus (Rat) | 10 | 13 | Rattus norvegicus (Rat) |
| | | NEURAL-CADHERIN | | | SPLICE ISOFORM 1 OF | | | SPARC-LIKE PROTEIN 1 | | | SPLICE ISOFORM 1 OF |
| | | PRECURSOR. | | | AGRIN PRECURSOR. | | | PRECURSOR. | | | AGRIN PRECURSOR. |
| | | [MASS = 99686 | | | [MASS = 208646 | | | [MASS = 70634 | | | [MASS = 208646 |
| 8 | 15 | Rattus norvegicus (Rat) | 8 | 20 | Rattus norvegicus (Rat) | 7 | 7 | Rattus norvegicus (Rat) | 10 | 14 | Rattus norvegicus (Rat) |
| | | CONTRAPSIN-LIKE | | | LOC367586 PROTEIN- | | | Neogenin precursor. | | | PREDICTED: SIMILAR TO |
| | | PROTEASE INHIBITOR 3 | | | Immunoglobulin Gamma | | | [MASS = 156144 | | | GAMMA-FILAMIN. |
| | | PRECURSOR. | | | heavy Chain. | | | | | | [MASS = 290986 |
| | | [MASS = 46277 | | | [MASS = 50949 | | | | | | |
| 8 | 10 | Rattus norvegicus (Rat) | 8 | 10 | Rattus norvegicus (Rat) | 7 | 16 | Rattus norvegicus (Rat) | 10 | 10 | Rattus norvegicus (Rat) 284 KDA |
| | | LUMICAN PRECURSOR. | | | VIMENTIN. | | | CONTRAPSIN-LIKE | | | PROTEIN. |
| | | [MASS = 38279 | | | [MASS = 53602 | | | PROTEASE INHIBITOR 3 | | | [MASS = 284430 |
| | | | | | | | | PRECURSOR. | | | |
| | | | | | | | | [MASS = 46277 | | | |
| 8 | 8 | Rattus norvegicus (Rat) | 7 | 12 | Rattus norvegicus (Rat) | 7 | 9 | Rattus norvegicus (Rat) | 9 | 18 | Rattus norvegicus (Rat) |
| | | PREDICTED: MINI | | | CONTRAPSIN-LIKE | | | ACTIN, ALPHA | | | TUBULIN BETA-5 CHAIN. |
| | | CHROMOSOME | | | PROTEASE INHIBITOR 3 | | | SKELETAL MUSCLE. | | | [MASS = 49671 |
| | | MAINTENANCE | | | PRECURSOR. | | | [MASS = 42051 | | | |
| | | DEFICIENT 6. | | | [MASS = 46277 | | | | | | |
| | | [MASS = 92815 | | | | | | | | | |
| 8 | 11 | Rattus norvegicus (Rat) | 7 | 9 | Rattus norvegicus (Rat) | 7 | 9 | Rattus norvegicus (Rat) | 9 | 11 | Rattus norvegicus (Rat) |
| | | SERINE PEPTIDASE | | | PREDICTED: NIDOGEN | | | ELONGATION FACTOR | | | SERINE PEPTIDASE |
| | | INHIBITOR, CLADE G, | | | 2. [MASS = 173960 | | | 1-ALPHA 1. | | | INHIBITOR, CLADE G, |
| | | MEMBER 1. | | | | | | [MASS = 50114 | | | MEMBER 1. [MASS = 55611 |
| | | [MASS = 55611 | | | | | | | | | |
| 8 | 10 | Rattus norvegicus (Rat) | 7 | 8 | Rattus norvegicus (Rat) | 7 | 7 | Rattus norvegicus (Rat) | 9 | 10 | Rattus norvegicus (Rat) 170 KDA |
| | | PREDICTED: LAMININ, | | | SPLICE ISOFORM 1 OF | | | TRANSITIONAL | | | PROTEIN-Glutamyl-prolyl- |
| | | GAMMA 1. | | | REELIN PRECURSOR. | | | ENDOPLASMIC | | | tRNA synthetase. |
| | | [MASS = 177387 | | | [MASS = 387531 | | | RETICULUM ATPASE. | | | [MASS = 170088 |
| | | | | | | | | [MASS = 89534 | | | |
| 8 | 8 | Rattus norvegicus (Rat) | 7 | 7 | Rattus norvegicus (Rat) | 7 | 11 | Rattus norvegicus (Rat) | 9 | 10 | Rattus norvegicus (Rat) |
| | | PREDICTED: SIMILAR | | | PREDICTED: | | | COLLAGEN ALPHA-2(I) | | | PROTEASOME (PROSOME, |
| | | TO RNA HELICASE A. | | | TRANSFORMING | | | CHAIN PRECURSOR. | | | MACROPAIN) 26S SUBUNIT, |
| | | [MASS = 150362 | | | GROWTH FACTOR, | | | [MASS = 129564 | | | NON-ATPASE, 2. |
| | | | | | BETA INDUCED, 68 KDA. | | | | | | [MASS = 100188 |
| | | | | | [MASS = 74369 | | | | | | |
| 8 | 11 | Rattus norvegicus (Rat) | 7 | 10 | Rattus norvegicus (Rat) | 7 | 7 | Rattus norvegicus (Rat) | 9 | 10 | Rattus norvegicus (Rat) |
| | | PREDICTED: SIMILAR | | | SERINE/CYSTEINE | | | ALPHA-MANNOSIDASE | | | PREDICTED: SIMILAR TO |
| | | TO CADHERIN-5. | | | PROTEINASE | | | 2. [MASS = 131242 | | | RNA HELICASE A. |
| | | [MASS = 135230 | | | INHIBITOR, CLADE C, | | | | | | [MASS = 150362 |
| | | | | | MEMBER 1. | | | | | | |
| | | | | | [MASS = 52234 | | | | | | |
| 8 | 12 | Rattus norvegicus (Rat) | 7 | 7 | Rattus norvegicus (Rat) | 7 | 9 | Rattus norvegicus (Rat) | 9 | 9 | Rattus norvegicus (Rat) |
| | | EPSILON 1 GLOBIN. | | | IKAP. [MASS = 149171 | | | SPLICE ISOFORM 1 OF | | | DELETED IN COLORECTAL |
| | | [MASS = 16105 | | | | | | ATTRACTIN | | | CANCER. [MASS = 158142 |
| | | | | | | | | PRECURSOR. | | | |
| | | | | | | | | [MASS = 163296 | | | |
| 8 | 8 | Rattus norvegicus (Rat) | 7 | 8 | Rattus norvegicus (Rat) | 6 | 9 | Rattus norvegicus (Rat) | 9 | 10 | Rattus norvegicus (Rat) |
| | | PREDICTED: SIMILAR | | | DELETED IN | | | TUBULIN ALPHA-1 | | | ALPHA-MANNOSIDASE 2. |
| | | TO METHIONINE-TRNA | | | COLORECTAL CANCER. | | | CHAIN. [MASS = 50136 | | | [MASS = 131242 |
| | | SYNTHETASE. | | | [MASS = 158142 | | | | | | |
| | | [MASS = 101582 | | | | | | | | | |
| 8 | 8 | Rattus norvegicus (Rat) | 7 | 7 | Rattus norvegicus (Rat) | 6 | 6 | Rattus norvegicus (Rat) | 8 | 13 | Rattus norvegicus (Rat) |
| | | EXPORTIN-1. | | | COMPLEMENT C4 | | | CONTRAPSIN-LIKE | | | VITAMIN D-BINDING |
| | | [MASS = 123335 | | | PRECURSOR. | | | PROTEASE INHIBITOR 6 | | | PROTEIN PRECURSOR. |
| | | | | | [MASS = 192163 | | | PRECURSOR. | | | [MASS = 53544 |
| | | | | | | | | [MASS = 46652 | | | |
| 8 | 12 | Rattus norvegicus (Rat) | 7 | 13 | Rattus norvegicus (Rat) | 6 | 8 | Rattus norvegicus (Rat) | 8 | 11 | Rattus norvegicus (Rat) IRON- |
| | | LOC367586 PROTEIN- | | | HYPOTHETICAL | | | SPLICE ISOFORM 1 OF | | | RESPONSIVE ELEMENT- |
| | | Immunoglobulin Gamma | | | PROTEIN RGD1305887- | | | NEURONAL CELL | | | BINDING PROTEIN 1. |
| | | heavy Chain. | | | TUBULIN BETA CHAIN. | | | ADHESION MOLECULE | | | [MASS = 98128 |
| | | [MASS = 50949 | | | [MASS = 50059 | | | PRECURSOR. | | | |
| | | | | | | | | [MASS = 133912 | | | |
| 8 | 8 | Rattus norvegicus (Rat) | 7 | 12 | Rattus norvegicus (Rat) | 6 | 7 | Rattus norvegicus (Rat) | 8 | 8 | Rattus norvegicus (Rat) |
| | | PREDICTED: | | | SP120-Heterogeneous | | | PREDICTED: SIMILAR | | | ALPHA-2 ANTIPLASMIN. |
| | | AMINOPEPTIDASE | | | nuclear ribonucleoprotein | | | TO RIKEN CDNA | | | [MASS = 46465 |
| | | PUROMYCIN SENSITIVE. | | | U. [MASS = 87748 | | | B430218L07 GENE. | | | |
| | | [MASS = 103344 | | | | | | [MASS = 143906 | | | |
| 8 | 9 | Rattus norvegicus (Rat) | 7 | 7 | Rattus norvegicus (Rat) | 6 | 6 | Rattus norvegicus (Rat) | 8 | 13 | Rattus norvegicus (Rat) SP120- |
| | | PREDICTED: similar to T- | | | COLLAGEN ALPHA-2(I) | | | PREDICTED: | | | Heterogeneous nuclear |
| | | complex protein 1 subunit | | | CHAIN PRECURSOR. | | | TRANSFORMING | | | ribonucleoprotein U. |
| | | theta. [MASS = 59745 | | | [MASS = 129564 | | | GROWTH FACTOR, | | | [MASS = 87748 |
| | | | | | | | | BETA INDUCED, 68 KDA. | | | |
| | | | | | | | | [MASS = 74369 | | | |
| 8 | 8 | Rattus norvegicus (Rat) | 7 | 7 | Rattus norvegicus (Rat) | 6 | 8 | Rattus norvegicus (Rat) | 8 | 8 | Rattus norvegicus (Rat) |
| | | PREDICTED: SIMILAR | | | PREDICTED: similar to T- | | | ALPHA-2-GLOBIN | | | PREDICTED: MINI |
| | | TO FILAMIN B. | | | complex protein 1 subunit | | | CHAIN. [MASS = 15285 | | | CHROMOSOME |
| | | [MASS = 291469 | | | theta. [MASS = 59745 | | | | | | MAINTENANCE DEFICIENT 6. |
| | | | | | | | | | | | [MASS = 92815 |
| 8 | 8 | Rattus norvegicus (Rat) | 6 | 8 | Rattus norvegicus (Rat) | 6 | 6 | Rattus norvegicus (Rat) | 8 | 9 | Rattus norvegicus (Rat) |
| | | STAPHYLOCOCCAL | | | CATHEPSIN B | | | ARCADLIN. | | | PREDICTED: SIMILAR TO |
| | | NUCLEASE DOMAIN- | | | PRECURSOR. | | | [MASS = 103800 | | | FIBULIN-1 PRECURSOR. |
| | | CONTAINING PROTEIN | | | [MASS = 37470 | | | | | | [MASS = 78072 |
| | | 1. [MASS = 101952 | | | | | | | | | |
| 8 | 9 | Rattus norvegicus (Rat) | 6 | 8 | Rattus norvegicus (Rat) | 6 | 9 | Rattus norvegicus (Rat) | 8 | 8 | Rattus norvegicus (Rat) |
| | | SPLICE ISOFORM V0 OF | | | ANGIOTENSINOGEN | | | TENASCIN | | | STAPHYLOCOCCAL |
| | | VERSICAN CORE | | | PRECURSOR. | | | (FRAGMENT). | | | NUCLEASE DOMAIN- |
| | | PROTEIN PRECURSOR | | | [MASS = 51982 | | | [MASS = 62473 | | | CONTAINING PROTEIN 1. |
| | | (FRAGMENT). | | | | | | | | | [MASS = 101952 |
| | | [MASS = 300008 | | | | | | | | | |
| 8 | 14 | Rattus norvegicus (Rat) | 6 | 7 | Rattus norvegicus (Rat) | 6 | 12 | Rattus norvegicus (Rat) | 8 | 13 | Rattus norvegicus (Rat) |
| | | FETUB PROTEIN. | | | RAT T-KININOGEN. | | | PREDICTED: RETINOL | | | APOLIPOPROTEIN E |
| | | [MASS = 43169 | | | [MASS = 47618 | | | BINDING PROTEIN 4, | | | PRECURSOR. [MASS = 35753 |
| | | | | | | | | PLASMA. [MASS = 50139 | | | |
| 8 | 8 | Rattus norvegicus (Rat) | 6 | 6 | Rattus norvegicus (Rat) | 6 | 10 | Rattus norvegicus (Rat) | 8 | 8 | Rattus norvegicus (Rat) |
| | | DA1-24-Complement | | | TENASCIN | | | PREDICTED: | | | PREDICTED: BRAIN |
| | | Factor B. [MASS = 124379 | | | (FRAGMENT). | | | CALSYNTENIN 1. | | | GLYCOGEN |
| | | | | | [MASS = 62473 | | | [MASS = 109351 | | | PHOSPHORYLASE. |
| | | | | | | | | | | | [MASS = 96738 |
| 7 | 9 | Rattus norvegicus (Rat) | 6 | 8 | Rattus norvegicus (Rat) | 6 | 7 | Rattus norvegicus (Rat) | 8 | 23 | Rattus norvegicus (Rat) |
| | | RAT T-KININOGEN. | | | SPLICE ISOFORM 1 OF | | | SPLICE ISOFORM 1 OF | | | PLASMINOGEN |
| | | [MASS = 47618 | | | ATTRACTIN | | | ALPHA-1B- | | | PRECURSOR. [MASS = 90536 |
| | | | | | PRECURSOR. | | | GLYCOPROTEIN | | | |
| | | | | | [MASS = 163296 | | | PRECURSOR. | | | |
| | | | | | | | | [MASS = 56479 | | | |
| 7 | 9 | Rattus norvegicus (Rat) | 6 | 7 | Rattus norvegicus (Rat) | 6 | 8 | Rattus norvegicus (Rat) | 8 | 8 | Rattus norvegicus (Rat) IKAP, |
| | | SERINE/CYSTEINE | | | PREDICTED: SIMILAR | | | PREDICTED: NIDOGEN | | | [MASS = 149171 |
| | | PROTEINASE | | | TO RIKEN CDNA | | | 2. [MASS = 173960 | | | |
| | | INHIBITOR, CLADE C, | | | B430218L07 GENE. | | | | | | |
| | | MEMBER 1. | | | [MASS = 143906 | | | | | | |
| | | [MASS = 52234 | | | | | | | | | |
| 7 | 8 | Rattus norvegicus (Rat) | 6 | 7 | Rattus norvegicus (Rat) | 6 | 7 | Rattus norvegicus (Rat) | 8 | 9 | Rattus norvegicus (Rat) |
| | | CLIP-ASSOCIATING | | | PROFILIN-1. | | | PREDICTED: SIMILAR | | | MANNOSE 6- |
| | | PROTEIN 2. | | | [MASS = 14826 | | | TO PYRUVATE KINASE | | | PHOSPHATE/INSULIN-LIKE |
| | | [MASS = 140638 | | | | | | (EC 2.7.1.40) ISOZYME | | | GROWTH FACTOR II |
| | | | | | | | | M2 - RAT. [MASS = 57731 | | | RECEPTOR. [MASS = 273608 |
| 7 | 9 | Rattus norvegicus (Rat) | 6 | 9 | Rattus norvegicus (Rat) | 6 | 7 | Rattus norvegicus (Rat) | 7 | 11 | Rattus norvegicus (Rat) |
| | | RIBONUCLEOTIDE | | | PREDICTED: CADHERIN | | | RAT T-KININOGEN. | | | CREATINE KINASE B-TYPE. |
| | | REDUCTASE M1. | | | 11. [MASS = 88036 | | | [MASS = 47618 | | | [MASS = 42712 |
| | | [MASS = 90293 | | | | | | | | | |
| 7 | 7 | Rattus norvegicus (Rat) | 6 | 10 | Rattus norvegicus (Rat) | 6 | 10 | Rattus norvegicus (Rat) | 7 | 13 | Rattus norvegicus (Rat) |
| | | ECTONUCLEOTIDE | | | SPLICE ISOFORM | | | PREDICTED: CADHERIN | | | HYPOTHETICAL PROTEIN |
| | | PYROPHOSPHATASE/ | | | GAMMA-B OF | | | 11. [MASS = 88036 | | | RGD1305887-TUBULIN BETA |
| | | PHOSPHODIESTERASE 2. | | | FIBRINOGEN GAMMA | | | | | | CHAIN. [MASS = 50059 |
| | | [MASS = 101310 | | | CHAIN PRECURSOR. | | | | | | |
| | | | | | [MASS = 50633 | | | | | | |
| 7 | 8 | Rattus norvegicus (Rat) | 6 | 12 | Rattus norvegicus (Rat) | 6 | 6 | Rattus norvegicus (Rat) | 7 | 8 | Rattus norvegicus (Rat) |
| | | HEPHAESTIN | | | ELONGATION FACTOR | | | PREDICTED: TUMOR | | | PREDICTED: similar to |
| | | PRECURSOR. | | | 1-ALPHA 1. | | | REJECTION ANTIGEN | | | phosphoribosylformylglycinamidine |
| | | [MASS = 129593 | | | [MASS = 50114 | | | GP96. [MASS = 92771 | | | synthase. [MASS = 146178 |
| 7 | 7 | Rattus norvegicus (Rat) | 6 | 8 | Rattus norvegicus (Rat) | 6 | 8 | Rattus norvegicus (Rat) | 7 | 12 | Rattus norvegicus (Rat) ATP- |
| | | NONO/P54NRB | | | PREDICTED: SIMILAR | | | PREDICTED: SIMILAR | | | CITRATE SYNTHASE. |
| | | HOMOLOG. | | | TO CRB2 PROTEIN. | | | TO STABILIN-1. | | | [MASS = 120781 |
| | | [MASS = 75487 | | | [MASS = 138781 | | | [MASS = 288663 | | | |
| 7 | 14 | Rattus norvegicus (Rat) | 6 | 6 | Rattus norvegicus (Rat) | 6 | 7 | Rattus norvegicus (Rat) | 7 | 10 | Rattus norvegicus (Rat) |
| | | PREDICTED: | | | TRIPEPTIDYL- | | | PREDICTED: SIMILAR | | | ANGIOTENSINOGEN |
| | | MICROTUBULE- | | | PEPTIDASE 2. | | | TO HEMICENTIN 1. | | | PRECURSOR. [MASS = 51982 |
| | | ASSOCIATED PROTEIN | | | [MASS = 138162 | | | [MASS = 639647 | | | |
| | | 1B. [MASS = 269643 | | | | | | | | | |
| 7 | 7 | Rattus norvegicus (Rat) | 6 | 6 | Rattus norvegicus (Rat) | 6 | 7 | Rattus norvegicus (Rat) | 7 | 10 | Rattus norvegicus (Rat) |
| | | PREDICTED | | | PREDICTED: SIMILAR | | | COMPLEMENT | | | PREDICTED: SIMILAR TO |
| | | NUCLEOLIN-RELATED | | | TO CAD PROTEIN. | | | COMPONENT 2. | | | CADHERIN-5. [MASS = 135230 |
| | | PROTEIN NRP. | | | [MASS = 250725 | | | [MASS = 83699 | | | |
| | | [MASS = 57036 | | | | | | | | | |
| 7 | 8 | Rattus norvegicus (Rat) | 6 | 7 | Rattus norvegicus (Rat) | 6 | 9 | Rattus norvegicus (Rat) | 7 | 12 | Rattus norvegicus (Rat) |
| | | NESTIN. [MASS = 208797 | | | PREDICTED: SIMILAR | | | FETUB PROTEIN. | | | PREDICTED: SIMILAR TO |
| | | | | | TO RNA HELICASE A. | | | [MASS = 43169 | | | PHOSPHOLIPID TRANSFER |
| | | | | | [MASS = 150362 | | | | | | PROTEIN. [MASS = 65430 |
| 7 | 7 | Rattus norvegicus (Rat) | 5 | 8 | Rattus norvegicus (Rat) | 5 | 6 | Rattus norvegicus (Rat) | 7 | 7 | Rattus norvegicus (Rat) |
| | | SPLICE ISOFORM 1 OF | | | CREATINE KINASE B- | | | PROFILIN-1. | | | SEZ6B. [MASS = 105607 |
| | | AGRIN PRECURSOR. | | | TYPE. [MASS = 42712 | | | [MASS = 14826 | | | |
| | | [MASS = 208646 | | | | | | | | | |
| 7 | 8 | Rattus norvegicus (Rat) | 5 | 5 | Rattus norvegicus (Rat) | 5 | 9 | Rattus norvegicus (Rat) | 7 | 8 | Rattus norvegicus (Rat) |
| | | SPLICE ISOFORM 1 OF | | | PHOSPHOGLYCERATE | | | CONTRAPSIN-LIKE | | | DYNACTIN-1. [MASS = 141930 |
| | | CULLIN-ASSOCIATED | | | KINASE 1. [MASS = 44423 | | | PROTEASE INHIBITOR 1 | | | |
| | | NEDD8-DISSOCIATED | | | | | | PRECURSOR. | | | |
| | | PROTEIN 2. | | | | | | [MASS = 46562 | | | |
| | | [MASS = 139673 | | | | | | | | | |
| 6 | 10 | Rattus norvegicus (Rat) | 5 | 5 | Rattus norvegicus (Rat) | 5 | 5 | Rattus norvegicus (Rat) | 7 | 8 | Rattus norvegicus (Rat) INTER- |
| | | CREATINE KINASE B- | | | HEAT SHOCK COGNATE | | | PROPROTEIN | | | ALPHA-INHIBITOR H4 HEAVY |
| | | TYPE. [MASS = 42712 | | | 71 KDA PROTEIN. | | | CONVERTASE | | | CHAIN. [MASS = 103755 |
| | | | | | [MASS = 70871 | | | SUBTILISIN/KEXIN TYPE | | | |
| | | | | | | | | 9 PRECURSOR. | | | |
| | | | | | | | | [MASS = 74709 | | | |
| 6 | 6 | Rattus norvegicus (Rat) | 5 | 6 | Rattus norvegicus (Rat) | 5 | 5 | Rattus norvegicus (Rat) | 7 | 7 | Rattus norvegicus (Rat) |
| | | PREDICTED: SIMILAR | | | PREDICTED: SIMILAR | | | HYPOTHETICAL | | | SPECTRIN ALPHA CHAIN, |
| | | TO SPLICING FACTOR | | | TO PROGRAMMED | | | PROTEIN LOC314432- | | | BRAIN. [MASS = 284713 |
| | | 3B, SUBUNIT 3, 130 KDA. | | | CELL DEATH 6 | | | Similar to ubiquitin-protein | | | |
| | | [MASS = 174174 | | | INTERACTING | | | ligase (EC 6.3.2.19) E1. | | | |
| | | | | | PROTEIN. [MASS = 75806 | | | [MASS = 117788 | | | |
| 6 | 8 | Rattus norvegicus (Rat) | 5 | 5 | Rattus norvegicus (Rat) | 5 | 12 | Rattus norvegicus (Rat) | 7 | 10 | Rattus norvegicus (Rat) |
| | | GLUCOSE PHOSPHATE | | | FIBRINOGEN BETA | | | SERINE PEPTIDASE | | | ELONGATION FACTOR 1- |
| | | ISOMERASE. | | | CHAIN PRECURSOR. | | | INHIBITOR, CLADE F, | | | ALPHA 1. [MASS = 50114 |
| | | [MASS = 62827 | | | [MASS = 54303 | | | MEMBER 2. | | | |
| | | | | | | | | [MASS = 54893 | | | |
| 6 | 11 | Rattus norvegicus (Rat) | 5 | 5 | Rattus norvegicus (Rat) | 5 | 8 | Rattus norvegicus (Rat) | 7 | 7 | Rattus norvegicus (Rat) |
| | | CONTRAPSIN-LIKE | | | ATP-CITRATE | | | COLLAGEN ALPHA-1(V) | | | PREDICTED: SIMILAR TO |
| | | PROTEASE INHIBITOR 1 | | | SYNTHASE. | | | CHAIN PRECURSOR. | | | VERY LARGE G PROTEIN- |
| | | PRECURSOR. | | | [MASS = 120781 | | | [MASS = 184610 | | | COUPLED RECEPTOR 1. |
| | | [MASS = 46562 | | | | | | | | | [MASS = 413840 |
| 6 | 9 | Rattus norvegicus (Rat) | 5 | 5 | Rattus norvegicus (Rat) | 5 | 5 | Rattus norvegicus (Rat) | 7 | 7 | Rattus norvegicus (Rat) |
| | | PLASMINOGEN | | | SPLICE ISOFORM 1 OF | | | COMPLEMENT C4 | | | PROTEIN KINASE C-BINDING |
| | | PRECURSOR. | | | NEURONAL CELL | | | PRECURSOR. | | | PROTEIN NELL2. |
| | | [MASS = 90536 | | | ADHESION MOLECULE | | | [MASS = 192163 | | | [MASS = 91334 |
| | | | | | PRECURSOR. | | | | | | |
| | | | | | [MASS = 133912 | | | | | | |
| 6 | 7 | Rattus norvegicus (Rat) | 5 | 11 | Rattus norvegicus (Rat) | 5 | 7 | Rattus norvegicus (Rat) | 7 | 9 | Rattus norvegicus (Rat) |
| | | Inter-alpha trypsin | | | CYSTATIN C | | | GLUTATHIONE | | | COMPLEMENT INHIBITORY |
| | | inhibitor, heavy chain 3. | | | PRECURSOR. | | | PEROXIDASE 3 | | | FACTOR H. [MASS = 140344 |
| | | [MASS = 98968 | | | [MASS = 15437 | | | PRECURSOR. | | | |
| | | | | | | | | [MASS = 25393 | | | |
| 6 | 7 | Rattus norvegicus (Rat) | 5 | 7 | Rattus norvegicus (Rat) | 5 | 8 | Rattus norvegicus (Rat) | 7 | 8 | Rattus norvegicus (Rat) |
| | | LEUCYL-TRNA | | | PEROXIREDOXIN-2. | | | FIBRILLIN-2. | | | PREDICTED: SIMILAR TO |
| | | SYNTHETASE. | | | [MASS = 21652 | | | [MASS = 313374 | | | EUKARYOTIC TRANSLATION |
| | | [MASS = 134279 | | | | | | | | | INITIATION FACTOR 3, |
| | | | | | | | | | | | SUBUNIT 10 THETA, |
| | | | | | | | | | | | 150/170 KDA. [MASS = 192616 |
| 6 | 7 | Rattus norvegicus (Rat) | 5 | 10 | Rattus norvegicus (Rat) | 5 | 6 | Rattus norvegicus (Rat) | 6 | 7 | Rattus norvegicus (Rat) ACTIN, |
| | | ALPHA-2-GLOBIN | | | ACTIN, CYTOPLASMIC | | | SPLICE ISOFORM | | | CYTOPLASMIC 1. |
| | | CHAIN. [MASS = 15285 | | | 1. [MASS = 41737 | | | APP770 OF AMYLOID | | | [MASS = 41737 |
| | | | | | | | | BETA A4 PROTEIN | | | |
| | | | | | | | | PRECURSOR | | | |
| | | | | | | | | (FRAGMENT). | | | |
| | | | | | | | | [MASS = 86704 | | | |
| 6 | 6 | Rattus norvegicus (Rat) | 5 | 5 | Rattus norvegicus (Rat) | 5 | 7 | Rattus norvegicus (Rat) | 6 | 9 | Rattus norvegicus (Rat) |
| | | CC2-27. [MASS = 120523 | | | TRIOSEPHOSPHATE | | | HYPOTHETICAL | | | LUMICAN PRECURSOR. |
| | | | | | ISOMERASE. | | | PROTEIN RGD1305887- | | | [MASS = 38279 |
| | | | | | [MASS = 26790 | | | TUBULIN BETA CHAIN. | | | |
| | | | | | | | | [MASS = 50059 | | | |
| 6 | 6 | Rattus norvegicus (Rat) | 5 | 5 | Rattus norvegicus (Rat) | 5 | 6 | Rattus norvegicus (Rat) | 6 | 6 | Rattus norvegicus (Rat) |
| | | PREDICTED: TUMOR | | | LEUCYL-TRNA | | | QUIESCIN Q6. | | | PREDICTED C-1- |
| | | REJECTION ANTIGEN | | | SYNTHETASE. | | | [MASS = 82412 | | | TETRAHYDROFOLATE |
| | | GP96. [MASS = 92771 | | | [MASS = 134279 | | | | | | SYNTHASE, CYTOPLASMIC. |
| | | | | | | | | | | | [MASS = 100351 |
| 6 | 11 | Rattus norvegicus (Rat) | 5 | 8 | Rattus norvegicus (Rat) | 5 | 5 | Rattus norvegicus (Rat) | 6 | 6 | Rattus norvegicus (Rat) |
| | | PREDICTED: RETINOL | | | SPLICE ISOFORM V0 OF | | | Inter-alpha trypsin | | | VESICLE ASSOCIATED |
| | | BINDING PROTEIN 4, | | | VERSICAN CORE | | | inhibitor, heavy chain 3. | | | PROTEIN. [MASS = 135350 |
| | | PLASMA. [MASS = 50139 | | | PROTEIN PRECURSOR | | | [MASS = 98968 | | | |
| | | | | | (FRAGMENT). | | | | | | |
| | | | | | [MASS = 300008 | | | | | | |
| 6 | 7 | Rattus norvegicus (Rat) | 5 | 5 | Rattus norvegicus (Rat) | 5 | 6 | Rattus norvegicus (Rat) | 6 | 6 | Rattus norvegicus (Rat) HEAT |
| | | PREDICTED: | | | GAMMA-GLUTAMYL | | | PREDICTED: SIMILAR | | | SHOCK COGNATE 71 KDA |
| | | TRIPARTITE MOTIF | | | HYDROLASE | | | TO PHOSPHOLIPID | | | PROTEIN. [MASS = 70871 |
| | | PROTEIN 28. | | | PRECURSOR. | | | TRANSFER PROTEIN. | | | |
| | | [MASS = 108785 | | | [MASS = 35830 | | | [MASS = 65430 | | | |
| 6 | 6 | Rattus norvegicus (Rat) | 5 | 13 | Rattus norvegicus (Rat) | 5 | 7 | Rattus norvegicus (Rat) | 6 | 8 | Rattus norvegicus (Rat) HEAT |
| | | PREDICTED: SIMILAR | | | PREDICTED: RETINOL | | | PREDICTED: | | | SHOCK PROTEIN HSP 90- |
| | | TO CELLULAR | | | BINDING PROTEIN 4, | | | HYPOTHETICAL | | | BETA. [MASS = 83185 |
| | | APOPTOSIS | | | PLASMA. [MASS = 50139 | | | PROTEIN XP_344107. | | | |
| | | SUSCEPTIBILITY | | | | | | [MASS = 189275 | | | |
| | | PROTEIN. | | | | | | | | | |
| | | [MASS = 110214 | | | | | | | | | |
| 6 | 9 | Rattus norvegicus (Rat) | 5 | 5 | Rattus norvegicus (Rat) | 5 | 5 | Rattus norvegicus (Rat) | 6 | 6 | Rattus norvegicus (Rat) |
| | | COLLAGEN ALPHA-1(I) | | | PROTEIN KINASE C- | | | ALPHA-1- | | | MATRIN-3. [MASS = 94447 |
| | | CHAIN PRECURSOR. | | | BINDING PROTEIN | | | MACROGLOBULIN. | | | |
| | | [MASS = 137886 | | | NELL2. [MASS = 91334 | | | [MASS = 167125 | | | |
| 6 | 7 | Rattus norvegicus (Rat) | 5 | 5 | Rattus norvegicus (Rat) | 5 | 5 | Rattus norvegicus (Rat) | 6 | 6 | Rattus norvegicus (Rat) |
| | | ANGIOTENSINOGEN | | | PREDICTED: TYROSINE | | | PREDICTED: TYROSINE | | | SPLICE ISOFORM 1 OF |
| | | PRECURSOR. | | | KINASE RECEPTOR 1. | | | KINASE RECEPTOR 1. | | | REELIN PRECURSOR. |
| | | [MASS = 51982 | | | [MASS = 125210 | | | [MASS = 125210 | | | [MASS = 387531 |
| 6 | 12 | Rattus norvegicus (Rat) | 5 | 5 | Rattus norvegicus (Rat) | 5 | 26 | Rattus norvegicus (Rat) | 6 | 6 | Rattus norvegicus (Rat) |
| | | SPLICE ISOFORM 1 OF | | | CHAPERONIN | | | EXTRACELLULAR | | | PROMININ-1S1 SPLICE |
| | | ALPHA-1B- | | | CONTAINING TCP1, | | | SUPEROXIDE | | | VARIANT. [MASS = 96632 |
| | | GLYCOPROTEIN | | | SUBUNIT 2. | | | DISMUTASE [CU—ZN] | | | |
| | | PRECURSOR. | | | [MASS = 57458 | | | PRECURSOR. | | | |
| | | [MASS = 56479 | | | | | | [MASS = 26620 | | | |
| 6 | 6 | Rattus norvegicus (Rat) | 5 | 5 | Rattus norvegicus (Rat) | 5 | 11 | Rattus norvegicus (Rat) | 6 | 7 | Rattus norvegicus (Rat) |
| | | VALYL-TRNA | | | ALPHA-1- | | | PEPTIDYLPROLYL | | | CHLORIDE INTRACELLULAR |
| | | SYNTHETASE. | | | MACROGLOBULIN. | | | ISOMERASE C. | | | CHANNEL 6. [MASS = 64786 |
| | | [MASS = 141275 | | | [MASS32 167125 | | | [MASS = 23009 | | | |
| 6 | 7 | Rattus norvegicus (Rat) | 5 | 5 | Rattus norvegicus (Rat) | 5 | 6 | Rattus norvegicus (Rat) | 6 | 6 | Rattus norvegicus (Rat) |
| | | CHAPERONIN | | | Inter-alpha trypsin | | | PROTEIN KINASE C- | | | SPLICE ISOFORM 1 OF |
| | | CONTAINING TCP1, | | | inhibitor, heavy chain 3. | | | BINDING PROTEIN | | | NEURONAL CELL ADHESION |
| | | SUBUNIT 2. | | | [MASS = 98968 | | | NELL2. [MASS = 91334 | | | MOLECULE PRECURSOR. |
| | | [MASS = 57458 | | | | | | | | | [MASS = 133912 |
| 6 | 6 | Rattus norvegicus (Rat) | 5 | 6 | Rattus norvegicus (Rat) | 4 | 7 | Rattus norvegicus (Rat) | 6 | 6 | Rattus norvegicus (Rat) |
| | | CHAPERONIN | | | SPLICE ISOFORM LONG | | | PREDICTED: | | | VASCULAR CELL ADHESION |
| | | CONTAINING TCP1, | | | OF HYALURONAN AND | | | DYSTROGLYCAN 1. | | | PROTEIN 1 PRECURSOR. |
| | | SUBUNIT 5. | | | PROTEOGLYCAN LINK | | | [MASS = 96706 | | | [MASS = 81246 |
| | | [MASS = 59537 | | | PROTEIN 1 | | | | | | |
| | | | | | PRECURSOR. | | | | | | |
| | | | | | [MASS = 40262 | | | | | | |
| 6 | 9 | Rattus norvegicus (Rat) | 5 | 5 | Rattus norvegicus (Rat) | 4 | 4 | Rattus norvegicus (Rat) | 6 | 7 | Rattus norvegicus (Rat) |
| | | CORTICOSTEROID- | | | PREDICTED: | | | PREDICTED: SIMILAR | | | PROPROTEIN CONVERTASE |
| | | BINDING GLOBULIN | | | TRIPARTITE MOTIF | | | TO HEPATIC MULTIPLE | | | SUBTILISIN/KEXIN TYPE 9 |
| | | PRECURSOR. | | | PROTEIN 28. | | | INOSITOL | | | PRECURSOR. [MASS = 74709 |
| | | [MASS = 44672 | | | [MASS = 108785 | | | POLYPHOSPHATE | | | |
| | | | | | | | | PHOSPHATASE. | | | |
| | | | | | | | | [MASS = 54619 | | | |
| 6 | 8 | Rattus norvegicus (Rat) | 5 | 5 | Rattus norvegicus (Rat) | 4 | 4 | Rattus norvegicus (Rat) | 6 | 6 | Rattus norvegicus (Rat) MAMA. |
| | | 60S RIBOSOMAL | | | STAPHYLOCOCCAL | | | PROMININ-1S1 SPLICE | | | [MASS = 63772 |
| | | PROTEIN L18. | | | NUCLEASE DOMAIN- | | | VARIANT. [MASS = 96632 | | | |
| | | [MASS = 21527 | | | CONTAINING PROTEIN | | | | | | |
| | | | | | 1. [MASS = 101952 | | | | | | |
| 6 | 6 | Rattus norvegicus (Rat) | 5 | 5 | Rattus norvegicus (Rat) | 4 | 5 | Rattus norvegicus (Rat) | 6 | 6 | Rattus norvegicus (Rat) TLN |
| | | Neogenin precursor. | | | PREDICTED: MINI | | | EPSILON 3 GLOBIN. | | | PROTEIN. [MASS = 161978 |
| | | [MASS = 156144 | | | CHROMOSOME | | | [MASS = 16540 | | | |
| | | | | | MAINTENANCE | | | | | | |
| | | | | | DEFICIENT 6. | | | | | | |
| | | | | | [MASS = 92815 | | | | | | |
| 6 | 7 | Rattus norvegicus (Rat) | 5 | 5 | Rattus norvegicus (Rat) | 4 | 9 | Rattus norvegicus (Rat) | 6 | 8 | Rattus norvegicus (Rat) |
| | | TRIPEPTIDYL- | | | 170 KDA PROTEIN- | | | CYSTATIN C | | | NESTIN. [MASS = 208797 |
| | | PEPTIDASE 2. | | | Glutamyl-prolyl-tRNA | | | PRECURSOR. | | | |
| | | [MASS = 138162 | | | synthetase. | | | [MASS = 15437 | | | |
| | | | | | [MASS = 170088 | | | | | | |
| 6 | 7 | Rattus norvegicus (Rat) | 5 | 5 | Rattus norvegicus (Rat) | 4 | 4 | Rattus norvegicus (Rat) | 6 | 8 | Rattus norvegicus (Rat) |
| | | PREDICTED: SIMILAR | | | MYOSIN-10. | | | PREDICTED: SIMILAR | | | ALPHA-2-GLOBIN CHAIN. |
| | | TO LAMININ B1. | | | [MASS = 228965 | | | TO PTK7 PROTEIN | | | [MASS = 15285 |
| | | [MASS = 228429 | | | | | | TYROSINE KINASE 7. | | | |
| | | | | | | | | [MASS = 139818 | | | |
| 6 | 8 | Rattus norvegicus (Rat) | 5 | 5 | Rattus norvegicus (Rat) | 4 | 5 | Rattus norvegicus (Rat) | 6 | 12 | Rattus norvegicus (Rat) |
| | | MANNOSE 6- | | | ALPHA-MANNOSIDASE | | | GRP78 BINDING | | | PREDICTED: NEURAL |
| | | PHOSPHATE/INSULIN- | | | 2. [MASS = 131242 | | | PROTEIN. | | | PRECURSOR CELL |
| | | LIKE GROWTH FACTOR | | | | | | [MASS = 110574 | | | EXPRESSED, |
| | | II RECEPTOR. | | | | | | | | | DEVELOPMENTALLY DOWN- |
| | | [MASS = 273608 | | | | | | | | | REGULATED GENE 4A. |
| | | | | | | | | | | | [MASS = 112368 |
| 6 | 6 | Rattus norvegicus (Rat) | 5 | 5 | Rattus norvegicus (Rat) | 4 | 4 | Rattus norvegicus (Rat) | 6 | 7 | Rattus norvegicus (Rat) 14-3-3 |
| | | PREDICTED: SIMILAR | | | PREDICTED: NEURAL | | | PREDICTED: SIMILAR | | | PROTEIN EPSILON. |
| | | TO SMC2 PROTEIN. | | | PRECURSOR CELL | | | TO PROTOCADHERIN | | | [MASS = 29714 |
| | | [MASS = 134280 | | | EXPRESSED, | | | 18 PRECURSOR. | | | |
| | | | | | DEVELOPMENTALLY | | | [MASS = 123552 | | | |
| | | | | | DOWN-REGULATED | | | | | | |
| | | | | | GENE 4A. | | | | | | |
| | | | | | [MASS = 112368 | | | | | | |
| 6 | 6 | Rattus norvegicus (Rat) | 5 | 5 | Rattus norvegicus (Rat) | 4 | 4 | Rattus norvegicus (Rat) | 6 | 7 | Rattus norvegicus (Rat) |
| | | 14-3-3 PROTEIN | | | PREDICTED: SIMILAR | | | M-CADHERIN. | | | MICROTUBULE-ASSOCIATED |
| | | EPSILON. [MASS = 29174 | | | TO GCN1 GENERAL | | | [MASS = 85753 | | | PROTEIN 4. [MASS = 110301 |
| | | | | | CONTROL OF AMINO- | | | | | | |
| | | | | | ACID SYNTHESIS 1- | | | | | | |
| | | | | | LIKE 1. [MASS = 302942 | | | | | | |
| 6 | 6 | Rattus norvegicus (Rat) | 4 | 7 | Rattus norvegicus (Rat) | 4 | 16 | Rattus norvegicus (Rat) | 6 | 6 | Rattus norvegicus (Rat) FAR |
| | | PREDICTED: SIMILAR | | | PREDICTED: | | | PREDICTED: similar to | | | UPSTREAM ELEMENT- |
| | | TO COATOMER | | | DYSTROGLYCAN 1. | | | Fibulin-1 precursor. | | | BINDING PROTEIN 2. |
| | | PROTEIN COMPLEX | | | [MASS = 96706 | | | [MASS = 75381 | | | [MASS = 74226 |
| | | SUBUNIT ALPHA. | | | | | | | | | |
| | | [MASS = 138360 | | | | | | | | | |
| 6 | 6 | Rattus norvegicus (Rat) | 4 | 5 | Rattus norvegicus (Rat) | 4 | 4 | Rattus norvegicus (Rat) | 6 | 6 | Rattus norvegicus (Rat) |
| | | PREDICTED: SIMILAR | | | 14-3-3 PROTEIN | | | AFAMIN PRECURSOR. | | | JUNCTION PLAKOGLOBIN. |
| | | TO CHROMATIN- | | | ZETA/DELTA. | | | [MASS = 75381 | | | [MASS = 81801 |
| | | SPECIFIC | | | [MASS = 27771 | | | | | | |
| | | TRANSCRIPTION | | | | | | | | | |
| | | ELONGATION FACTOR, | | | | | | | | | |
| | | 140 KDA SUBUNIT. | | | | | | | | | |
| | | [MASS = 130435 | | | | | | | | | |
| 6 | 6 | Rattus norvegicus (Rat) | 4 | 4 | Rattus norvegicus (Rat) | 4 | 4 | Rattus norvegicus (Rat) | 6 | 6 | Rattus norvegicus (Rat) |
| | | PREDICTED: KINESIN | | | GLUCOSE PHOSPHATE | | | PREDICTED: SIMILAR | | | PREDICTED: similar to T- |
| | | FAMILY MEMBER 4. | | | ISOMERASE. | | | TO SEMA6A PROTEIN. | | | complex protein 1 subunit theta. |
| | | [MASS = 139682 | | | [MASS = 62827 | | | [MASS = 114583 | | | [MASS = 59745 |
| 5 | 8 | Rattus norvegicus (Rat) | 4 | 5 | Rattus norvegicus (Rat) | 4 | 7 | Rattus norvegicus (Rat) | 6 | 9 | Rattus norvegicus (Rat) |
| | | ACTIN, CYTOPLASMIC 1. | | | EPSILON 1 GLOBIN. | | | SPLICE ISOFORM V0 OF | | | SERINE PEPTIDASE |
| | | [MASS = 41737 | | | [MASS = 16105 | | | VERSICAN CORE | | | INHIBITOR, CLADE F, |
| | | | | | | | | PROTEIN PRECURSOR | | | MEMBER 2. [MASS = 54893 |
| | | | | | | | | (FRAGMENT). | | | |
| | | | | | | | | [MASS = 300008 | | | |
| 5 | 5 | Rattus norvegicus (Rat) | 4 | 4 | Rattus norvegicus (Rat) | 4 | 5 | Rattus norvegicus (Rat) | 6 | 6 | Rattus norvegicus (Rat) |
| | | PREDICTED similar to C- | | | SPLICE ISOFORM 1 OF | | | PROTOCADHERIN | | | PREDICTED: SIMILAR TO |
| | | 1-TETRAHYDROFOLATE | | | FIBRINOGEN ALPHA | | | GAMMA SUBFAMILY C, | | | ISOLEUCINE-TRNA |
| | | SYNTHASE, | | | CHAIN PRECURSOR. | | | 3. [MASS = 101038 | | | SYNTHETHASE. |
| | | CYTOPLASMIC. | | | [MASS = 86686 | | | | | | [MASS = 144169 |
| | | [MASS = 100351 | | | | | | | | | |
| 5 | 8 | Rattus norvegicus (Rat) | 4 | 4 | Rattus norvegicus (Rat) | 4 | 4 | Rattus norvegicus (Rat) | 6 | 6 | Rattus norvegicus (Rat) VALYL- |
| | | CATHEPSIN B | | | PREDICTED: similar to | | | SORTILIN PRECURSOR. | | | TRNA SYNTHETASE. |
| | | PRECURSOR. | | | Heterogeneous nuclear | | | [MASS = 91169 | | | [MASS = 141275 |
| | | [MASS = 37470 | | | ribonucleoproteins | | | | | | |
| | | | | | A2/B1. [MASS = 32468 | | | | | | |
| 5 | 6 | Rattus norvegicus (Rat) | 4 | 6 | Rattus norvegicus (Rat) | 4 | 4 | Rattus norvegicus (Rat) | 6 | 15 | Rattus norvegicus (Rat) |
| | | PROTEASOME | | | ALPHA-2-GLOBIN | | | ISCHEMIA RESPONSIVE | | | SPLICE ISOFORM HMW OF |
| | | (PROSOME, | | | CHAIN. [MASS = 15285 | | | 94 KDA PROTEIN. | | | KININOGEN-1 PRECURSOR. |
| | | MACROPAIN) 26S | | | | | | [MASS = 94057 | | | [MASS = 70933 |
| | | SUBUNIT, NON-ATPASE, | | | | | | | | | |
| | | 2. [MASS = 100188 | | | | | | | | | |
| 5 | 7 | Rattus norvegicus (Rat) | 4 | 4 | Rattus norvegicus (Rat) | 4 | 4 | Rattus norvegicus (Rat) | 5 | 6 | Rattus norvegicus (Rat) 14-3-3 |
| | | PREDICTED: CADHERIN | | | PREDICTED: SIMILAR | | | MANNOSE 6- | | | PROTEIN ZETA/DELTA. |
| | | 11. [MASS = 88036 | | | TO RAN BINDING | | | PHOSPHATE/INSULIN- | | | [MASS = 27771 |
| | | | | | PROTEIN 5. | | | LIKE GROWTH FACTOR | | | |
| | | | | | [MASS = 99947 | | | II RECEPTOR. | | | |
| | | | | | | | | [MASS = 273608 | | | |
| 5 | 6 | Rattus norvegicus (Rat) | 4 | 4 | Rattus norvegicus (Rat) | 4 | 4 | Rattus norvegicus (Rat) | 5 | 5 | Rattus norvegicus (Rat) |
| | | RAB GDP | | | PEPTIDYL-PROLYL CIS- | | | PROCOLLAGEN- | | | PREDICTED NUCLEOLIN- |
| | | DISSOCIATION | | | TRANS ISOMERASE A. | | | LYSINE,2- | | | RELATED PROTEIN NRP. |
| | | INHIBITOR BETA. | | | [MASS = 17743 | | | OXOGLUTARATE 5- | | | [MASS = 57036 |
| | | [MASS = 50685 | | | | | | DIOXYGENASE 3 | | | |
| | | | | | | | | PRECURSOR. | | | |
| | | | | | | | | [MASS = 85060 | | | |
| 5 | 5 | Rattus norvegicus (Rat) | 4 | 4 | Rattus norvegicus (Rat) L- | 4 | 5 | Rattus norvegicus (Rat) | 5 | 6 | Rattus norvegicus (Rat) |
| | | PREDICTED similar to | | | LACTATE | | | COMPLEMENT | | | PHOSPHOGLYCERATE |
| | | Nuclear autoantigenic | | | DEHYDROGENASE A | | | INHIBITORY FACTOR H. | | | KINASE 1. [MASS = 44423 |
| | | sperm protein. | | | CHAIN. [MASS = 36451 | | | [MASS = 140344 | | | |
| | | [MASS = 45764 | | | | | | | | | |
| 5 | 5 | Rattus norvegicus (Rat) | 4 | 8 | Rattus norvegicus (Rat) | 4 | 5 | Rattus norvegicus (Rat) | 5 | 7 | Rattus norvegicus (Rat) |
| | | PREDICTED: SIMILAR | | | PREDICTED: similar to | | | PREDICTED: SIMILAR | | | COLLAGEN TYPE A1(XI)7-8. |
| | | TO RIKEN CDNA | | | Fibulin-1 precursor. | | | TO LAMININ-2 ALPHA2 | | | [MASS = 45691 |
| | | B430218L07 GENE. | | | [MASS = 75381 | | | CHAIN PRECURSOR. | | | |
| | | [MASS = 143906 | | | | | | [MASS = 359007 | | | |
| 5 | 5 | Rattus norvegicus (Rat) | 4 | 4 | Rattus norvegicus (Rat) | 4 | 6 | Rattus norvegicus (Rat) | 5 | 6 | Rattus norvegicus (Rat) RAT T- |
| | | LAR RECEPTOR-LINKED | | | PROTEASOME | | | TENASCIN | | | KININOGEN. [MASS = 47618 |
| | | TYROSINE | | | (PROSOME, | | | (FRAGMENT). | | | |
| | | PHOSPHATASE. | | | MACROPAIN) 26S | | | [MASS = 67815 | | | |
| | | [MASS = 181130 | | | SUBUNIT, NON-ATPASE, | | | | | | |
| | | | | | 2. [MASS = 100188 | | | | | | |
| 5 | 7 | Rattus norvegicus (Rat) | 4 | 4 | Rattus norvegicus (Rat) | 4 | 6 | Rattus norvegicus (Rat) | 5 | 6 | Rattus norvegicus (Rat) |
| | | SPLICE ISOFORM | | | AFAMIN PRECURSOR. | | | FIBULIN-2 ISOFORM A. | | | PREDICTED: SIMILAR TO |
| | | GAMMA-B OF | | | [MASS = 69335 | | | [MASS = 126193 | | | IMPORTIN 9. [MASS = 131739 |
| | | FIBRINOGEN GAMMA | | | | | | | | | |
| | | CHAIN PRECURSOR. | | | | | | | | | |
| | | [MASS = 50633 | | | | | | | | | |
| 5 | 5 | Rattus norvegicus (Rat) | 4 | 5 | Rattus norvegicus (Rat) | 4 | 5 | Rattus norvegicus (Rat) | 5 | 6 | Rattus norvegicus (Rat) |
| | | ADENOSYLHOMO- | | | SPARC-LIKE PROTEIN 1 | | | PREDICTED: SIMILAR | | | COMPLEMENT C4 |
| | | CYSTEINASE. | | | PRECURSOR. | | | TO COLLAGEN ALPHA1 | | | PRECURSOR. [MASS = 192163 |
| | | [MASS = 47407 | | | [MASS = 70634 | | | TYPE VI-PRECURSOR. | | | |
| | | | | | | | | [MASS = 130760 | | | |
| 5 | 5 | Rattus norvegicus (Rat) | 4 | 10 | Rattus norvegicus (Rat) | 4 | 5 | Rattus norvegicus (Rat) | 5 | 6 | Rattus norvegicus (Rat) |
| | | SPLICE ISOFORM 1 OF | | | SERINE PEPTIDASE | | | PROTHROMBIN | | | NONO/P54NRB HOMOLOG. |
| | | PROTEIN SET. | | | INHIBITOR, CLADE F, | | | PRECURSOR | | | [MASS = 75487 |
| | | [MASS = 33406 | | | MEMBER 2. | | | (FRAGMENT). | | | |
| | | | | | [MASS = 54893 | | | [MASS = 70412 | | | |
| 5 | 5 | Rattus norvegicus (Rat) | 4 | 5 | Rattus norvegicus (Rat) | 4 | 5 | Rattus norvegicus (Rat) | 5 | 5 | Rattus norvegicus (Rat) |
| | | IRON-RESPONSIVE | | | SORTILIN PRECURSOR. | | | TRANSCOBALAMIN-2 | | | KALLISTATIN. [MASS = 48021 |
| | | ELEMENT-BINDING | | | [MASS = 91169 | | | PRECURSOR. | | | |
| | | PROTEIN 1. | | | | | | [MASS = 47420 | | | |
| | | [MASS = 98128 | | | | | | | | | |
| 5 | 6 | Rattus norvegicus (Rat) | 4 | 4 | Rattus norvegicus (Rat) | 4 | 4 | Rattus norvegicus (Rat) | 5 | 6 | Rattus norvegicus (Rat) |
| | | PREDICTED: SIMILAR | | | PREDICTED: | | | PREDICTED: SIMILAR | | | EXPORTIN-1. [MASS = 123335 |
| | | TO EUKARYOTIC | | | AMINOPEPTIDASE | | | TO VINCULIN. | | | |
| | | TRANSLATION | | | PUROMYCIN | | | [MASS = 116615 | | | |
| | | INITIATION FACTOR 3, | | | SENSITIVE. | | | | | | |
| | | SUBUNIT 10 THETA, | | | [MASS = 103344 | | | | | | |
| | | 150/170 KDA. | | | | | | | | | |
| | | [MASS = 192616 | | | | | | | | | |
| 5 | 9 | Rattus norvegicus (Rat) | 4 | 4 | Rattus norvegicus (Rat) | 4 | 4 | Rattus norvegicus (Rat) | 5 | 8 | Rattus norvegicus (Rat) |
| | | SERINE PEPTIDASE | | | PREDICTED: SIMILAR | | | PREDICTED: SIMILAR | | | PREDICTED-INHIBIN BINDING |
| | | INHIBITOR, CLADE F, | | | TO PROTOCADHERIN 1 | | | TO PLEXIN-B2 | | | PROTEIN LONG ISOFORM. |
| | | MEMBER 2. | | | ISOFORM 2 | | | PRECURSOR. | | | [MASS = 153224 |
| | | [MASS = 54893 | | | PRECURSOR. | | | [MASS = 216119 | | | |
| | | | | | [MASS = 138914 | | | | | | |
| 5 | 5 | Rattus norvegicus (Rat) | 4 | 4 | Rattus norvegicus (Rat) | 3 | 3 | Rattus norvegicus (Rat) | 5 | 10 | Rattus norvegicus (Rat) |
| | | PREDICTED: SIMILAR | | | EXPORTIN-1. | | | 14-3-3 PROTEIN | | | CATHEPSIN B PRECURSOR. |
| | | TO SEROTRANSFERRIN | | | [MASS = 123335 | | | ZETA/DELTA. | | | [MASS = 37470 |
| | | PRECURSOR. | | | | | | [MASS = 27771 | | | |
| | | [MASS = 76607 | | | | | | | | | |
| 5 | 7 | Rattus norvegicus (Rat) | 4 | 6 | Rattus norvegicus (Rat) | 3 | 3 | Rattus norvegicus (Rat) | 5 | 8 | Rattus norvegicus (Rat) |
| | | COLLAGEN ALPHA-1(III) | | | SPLICE ISOFORM HMW | | | COLLAGEN TYPE | | | PREDICTED: SIMILAR TO |
| | | CHAIN PRECURSOR. | | | OF KININOGEN-1 | | | A1(XI)7-8. [MASS = 45691 | | | RAN BINDING PROTEIN 5. |
| | | [MASS = 138936 | | | PRECURSOR. | | | | | | [MASS = 133476 |
| | | | | | [MASS = 70933 | | | | | | |
| 5 | 8 | Rattus norvegicus (Rat) | 4 | 7 | Rattus norvegicus (Rat) | 3 | 3 | Rattus norvegicus (Rat) | 5 | 16 | Rattus norvegicus (Rat) |
| | | TENASCIN | | | GLUTATHIONE | | | PROTECTIVE PROTEIN | | | CYSTATIN C PRECURSOR. |
| | | (FRAGMENT). | | | PEROXIDASE 3 | | | FOR BETA- | | | [MASS = 15437 |
| | | [MASS = 62473 | | | PRECURSOR. | | | GALACTOSIDASE. | | | |
| | | | | | [MASS = 25393 | | | [MASS = 51216 | | | |
| 5 | 7 | Rattus norvegicus (Rat) | 4 | 4 | Rattus norvegicus (Rat) | 3 | 4 | Rattus norvegicus (Rat) | 5 | 7 | Rattus norvegicus (Rat) |
| | | NUCLEOSOME | | | PREDICTED: SIMILAR | | | CREATINE KINASE B- | | | LACTADHERIN PRECURSOR. |
| | | ASSEMBLY PROTEIN 1- | | | TO PTK7 PROTEIN | | | TYPE. [MASS = 42712 | | | [MASS = 47413 |
| | | LIKE 1. [MASS = 45373 | | | TYROSINE KINASE 7. | | | | | | |
| | | | | | [MASS = 139818 | | | | | | |
| 5 | 5 | Rattus norvegicus (Rat) | 4 | 4 | Rattus norvegicus (Rat) | 3 | 3 | Rattus norvegicus (Rat) | 5 | 6 | Rattus norvegicus (Rat) |
| | | PREDICTED: VON | | | SPLICE ISOFORM 1 OF | | | PEROXIREDOXIN-2. | | | SPLICE ISOFORM GAMMA-B |
| | | WILLEBRAND FACTOR. | | | SEX HORMONE- | | | [MASS = 21652 | | | OF FIBRINOGEN GAMMA |
| | | [MASS = 308474 | | | BINDING GLOBULIN | | | | | | CHAIN PRECURSOR. |
| | | | | | PRECURSOR. | | | | | | [MASS = 50633 |
| | | | | | [MASS = 44533 | | | | | | |
| 5 | 5 | Rattus norvegicus (Rat) | 4 | 6 | Rattus norvegicus (Rat) | 3 | 3 | Rattus norvegicus (Rat) | 5 | 6 | Rattus norvegicus (Rat) |
| | | PREDICTED: SIMILAR | | | PEPTIDYLPROLYL | | | PREDICTED: SIMILAR | | | EPSILON 1 GLOBIN. |
| | | TO HEPATIC MULTIPLE | | | ISOMERASE C. | | | TO PROTOCADHERIN 1 | | | [MASS = 16105 |
| | | INOSITOL | | | [MASS = 23009 | | | ISOFORM 2 | | | |
| | | POLYPHOSPHATE | | | | | | PRECURSOR. | | | |
| | | PHOSPHATASE. | | | | | | [MASS = 138914 | | | |
| | | [MASS = 54619 | | | | | | | | | |
| 5 | 6 | Rattus norvegicus (Rat) | 4 | 5 | Rattus norvegicus (Rat) | 3 | 3 | Rattus norvegicus (Rat) | 5 | 5 | Rattus norvegicus (Rat) |
| | | 40S RIBOSOMAL | | | QUIESCIN Q6. | | | GAMMA-GLUTAMYL | | | TRIPEPTIDYL-PEPTIDASE 2. |
| | | PROTEIN SA. | | | [MASS = 82412 | | | HYDROLASE | | | [MASS = 138162 |
| | | [MASS = 32693 | | | | | | PRECURSOR. | | | |
| | | | | | | | | [MASS = 35830 | | | |
| 5 | 5 | Rattus norvegicus (Rat) | 4 | 6 | Rattus norvegicus (Rat) | 3 | 3 | Rattus norvegicus (Rat) | 5 | 7 | Rattus norvegicus (Rat) |
| | | TRIOSEPHOSPHATE | | | SPLICE ISOFORM 1 OF | | | PREDICTED: similar to | | | PREDICTED: SIMILAR TO |
| | | ISOMERASE. | | | HETEROGENEOUS | | | Slit-like 2. [MASS = 72321 | | | CRB2 PROTEIN. |
| | | [MASS = 26790 | | | NUCLEAR | | | | | | [MASS = 138781 |
| | | | | | RIBONUCLEOPROTEIN | | | | | | |
| | | | | | D0. [MASS = 38192 | | | | | | |
| 5 | 6 | Rattus norvegicus (Rat) | 4 | 6 | Rattus norvegicus (Rat) | 3 | 3 | Rattus norvegicus (Rat) | 5 | 5 | Rattus norvegicus (Rat) |
| | | HEMOPEXIN | | | PREDICTED: SIMILAR | | | PREDICTED: SIMILAR | | | PREDICTED: SIMILAR TO |
| | | PRECURSOR. | | | TO PHOSPHOLIPID | | | TO CRB2 PROTEIN. | | | ALANYL-TRNA SYNTHETASE. |
| | | [MASS = 51291 | | | TRANSFER PROTEIN. | | | [MASS = 138781 | | | [MASS = 106811 |
| | | | | | [MASS = 65430 | | | | | | |
| 5 | 5 | Rattus norvegicus (Rat) | 4 | 4 | Rattus norvegicus (Rat) | 3 | 4 | Rattus norvegicus (Rat) | 5 | 6 | Rattus norvegicus (Rat) |
| | | PREDICTED: SIMILAR | | | HEAT SHOCK PROTEIN | | | ACTIN, CYTOPLASMIC | | | PREDICTED: MINI |
| | | TO CCTETA, ETA | | | HSP 90-BETA. | | | 1. [MASS = 41737 | | | CHROMOSOME |
| | | SUBUNIT OF THE | | | [MASS = 83185 | | | | | | MAINTENANCE DEFICIENT 4 |
| | | CHAPERONIN | | | | | | | | | HOMOLOG. [MASS = 96685 |
| | | CONTAINING TCP-1. | | | | | | | | | |
| | | [MASS = 75684 | | | | | | | | | |
| 5 | 7 | Rattus norvegicus (Rat) | 4 | 6 | Rattus norvegicus (Rat) | 3 | 4 | Rattus norvegicus (Rat) | 5 | 5 | Rattus norvegicus (Rat) |
| | | PROFILIN-1. | | | LOW-DENSITY | | | LOW-DENSITY | | | PREDICTED: SIMILAR TO |
| | | [MASS = 14826 | | | LIPOPROTEIN | | | LIPOPROTEIN | | | VINCULIN. [MASS = 116615 |
| | | | | | RECEPTOR | | | RECEPTOR | | | |
| | | | | | PRECURSOR. | | | PRECURSOR. | | | |
| | | | | | [MASS = 96622 | | | [MASS = 96622 | | | |
| 5 | 6 | Rattus norvegicus (Rat) | 4 | 5 | Rattus norvegicus (Rat) | 3 | 3 | Rattus norvegicus (Rat) | 5 | 6 | Rattus norvegicus (Rat) |
| | | SPLICE ISOFORM 1 OF | | | BETA-2- | | | PREDICTED: SIMILAR | | | SPLICE ISOFORM 1 OF |
| | | NEURONAL CELL | | | MICROGLOBULIN | | | TO LERK-5. | | | ATTRACTIN PRECURSOR. |
| | | ADHESION MOLECULE | | | PRECURSOR. | | | [MASS = 37282 | | | [MASS = 163296 |
| | | PRECURSOR. | | | [MASS = 13720 | | | | | | |
| | | [MASS = 133912 | | | | | | | | | |
| 5 | 5 | Rattus norvegicus (Rat) | 4 | 5 | Rattus norvegicus (Rat) | 3 | 4 | Rattus norvegicus (Rat) | 5 | 7 | Rattus norvegicus (Rat) |
| | | PREDICTED: SIMILAR | | | FETUB PROTEIN. | | | HEPATOCYTE GROWTH | | | GLUCOSE PHOSPHATE |
| | | TO FRAS1 RELATED | | | [MASS = 43169 | | | FACTOR ACTIVATOR. | | | ISOMERASE. [MASS = 62827 |
| | | EXTRACELLULAR | | | | | | [MASS = 70737 | | | |
| | | MATRIX PROTEIN 2. | | | | | | | | | |
| | | [MASS = 378709 | | | | | | | | | |
| 5 | 8 | Rattus norvegicus (Rat) T- | 4 | 4 | Rattus norvegicus (Rat) | 3 | 3 | Rattus norvegicus (Rat) | 5 | 6 | Rattus norvegicus (Rat) |
| | | COMPLEX PROTEIN 1 | | | PREDICTED: SIMILAR | | | MANNOSIDASE 2, | | | SPLICE ISOFORM 2 OF |
| | | SUBUNIT DELTA. | | | TO PROTOCADHERIN | | | ALPHA B1. | | | RECEPTOR-TYPE TYROSINE- |
| | | [MASS = 57968 | | | 18 PRECURSOR. | | | [MASS = 114327 | | | PROTEIN PHOSPHATASE |
| | | | | | [MASS = 123552 | | | | | | ZETA PRECURSOR. |
| | | | | | | | | | | | [MASS = 164596 |
| 5 | 6 | Rattus norvegicus (Rat) | 4 | 5 | Rattus norvegicus (Rat) | 3 | 3 | Rattus norvegicus (Rat) | 5 | 5 | Rattus norvegicus (Rat) HEAT- |
| | | SPLICE ISOFORM HMW | | | COMPLEMENT | | | NEUROPILIN-2 | | | SHOCK PROTEIN 105 KDA. |
| | | OF KININOGEN-1 | | | COMPONENT 2. | | | PRECURSOR. | | | [MASS = 96419 |
| | | PRECURSOR. | | | [MASS = 83699 | | | [MASS = 104473 | | | |
| | | [MASS = 70933 | | | | | | | | | |
| 5 | 8 | Rattus norvegicus (Rat) | 4 | 4 | Rattus norvegicus (Rat) | 3 | 3 | Rattus norvegicus (Rat) | 5 | 7 | Rattus norvegicus (Rat) |
| | | PREDICTED: SIMILAR | | | PREDICTED: SIMILAR | | | PROCOLLAGEN C- | | | PREDICTED similar to Nuclear |
| | | TO RIBOSOMAL | | | TO HEPATIC MULTIPLE | | | ENDOPEPTIDASE | | | autoantigenic sperm protein. |
| | | PROTEIN L6. | | | INOSITOL | | | ENHANCER 1 | | | [MASS = 45764 |
| | | [MASS = 32944 | | | POLYPHOSPHATE | | | PRECURSOR. | | | |
| | | | | | PHOSPHATASE. | | | [MASS = 50185 | | | |
| | | | | | [MASS = 54619 | | | | | | |
| 5 | 6 | Rattus norvegicus (Rat) | 4 | 4 | Rattus norvegicus (Rat) | 3 | 3 | Rattus norvegicus (Rat) | 5 | 6 | Rattus norvegicus (Rat) LAR |
| | | 60S RIBOSOMAL | | | NESTIN. [MASS = 208797 | | | FATTY ACID SYNTHASE. | | | RECEPTOR-LINKED |
| | | PROTEIN L4. | | | | | | [MASS = 272650 | | | TYROSINE PHOSPHATASE. |
| | | [MASS = 47126 | | | | | | | | | [MASS = 181130 |
| 4 | 4 | Rattus norvegicus (Rat) IG | 4 | 4 | Rattus norvegicus (Rat) | 3 | 4 | Rattus norvegicus (Rat) | 4 | 5 | Rattus norvegicus (Rat) IG |
| | | KAPPA CHAIN C | | | SPLICE ISOFORM 2 OF | | | PEPTIDYL-PROLYL CIS- | | | KAPPA CHAIN C REGION, B |
| | | REGION, B ALLELE. | | | RECEPTOR-TYPE | | | TRANS ISOMERASE A. | | | ALLELE. [MASS = 11601 |
| | | [MASS = 11601 | | | TYROSINE-PROTEIN | | | [MASS = 17743 | | | |
| | | | | | PHOSPHATASE ZETA | | | | | | |
| | | | | | PRECURSOR. | | | | | | |
| | | | | | [MASS = 164596 | | | | | | |
| 4 | 4 | Rattus norvegicus (Rat) | 4 | 4 | Rattus norvegicus (Rat) | 3 | 3 | Rattus norvegicus (Rat) | 4 | 5 | Rattus norvegicus (Rat) |
| | | EUKARYOTIC | | | CULLIN-ASSOCIATED | | | SPLICE ISOFORM 1 OF | | | CHAPERONIN CONTAINING |
| | | TRANSLATION | | | NEDD8-DISSOCIATED | | | SEX HORMONE- | | | TCP1, SUBUNIT 2. |
| | | INITIATION FACTOR | | | PROTEIN 1. | | | BINDING GLOBULIN | | | [MASS = 57458 |
| | | 4A2. [MASS = 46489 | | | [MASS = 136362 | | | PRECURSOR. | | | |
| | | | | | | | | [MASS = 44533 | | | |
| 4 | 7 | Rattus norvegicus (Rat) | 4 | 4 | Rattus norvegicus (Rat) | 3 | 4 | Rattus norvegicus (Rat) | 4 | 4 | Rattus norvegicus (Rat) |
| | | HNRPK PROTEIN. | | | PREDICTED: SIMILAR | | | CELL GROWTH | | | PREDICTED: TUMOR |
| | | [MASS = 51028 | | | TO PROTOCADHERIN | | | REGULATOR WITH EF | | | REJECTION ANTIGEN GP96. |
| | | | | | 19 PRECURSOR. | | | HAND DOMAIN 1. | | | [MASS = 92771 |
| | | | | | [MASS = 125989 | | | [MASS = 30835 | | | |
| 4 | 5 | Rattus norvegicus (Rat) | 4 | 9 | Rattus norvegicus (Rat) | 3 | 3 | Rattus norvegicus (Rat) | 4 | 4 | Rattus norvegicus (Rat) |
| | | PREDICTED: SIMILAR | | | COLLAGEN ALPHA-1(III) | | | ROUNDABOUT | | | DREBRIN 1. [MASS = 77472 |
| | | TO RAN BINDING | | | CHAIN PRECURSOR. | | | HOMOLOG 1 | | | |
| | | PROTEIN 5. | | | [MASS = 138936 | | | PRECURSOR. | | | |
| | | [MASS = 133476 | | | | | | [MASS = 180748 | | | |
| 4 | 5 | Rattus norvegicus (Rat) | 4 | 5 | Rattus norvegicus (Rat) | 3 | 4 | Rattus norvegicus (Rat) | 4 | 4 | Rattus norvegicus (Rat) |
| | | PREDICTED: SIMILAR | | | EPSILON 3 GLOBIN. | | | BETA-2- | | | PREDICTED: SIMILAR TO |
| | | TO RAN BINDING | | | [MASS = 16540 | | | MICROGLOBULIN | | | DNA REPLICATION |
| | | PROTEIN 5. | | | | | | PRECURSOR. | | | LICENSING FACTOR MCM3. |
| | | [MASS = 99947 | | | | | | [MASS = 13720 | | | [MASS = 83429 |
| 4 | 5 | Rattus norvegicus (Rat) | 4 | 5 | Rattus norvegicus (Rat) | 3 | 6 | Rattus norvegicus (Rat) | 4 | 5 | Rattus norvegicus (Rat) |
| | | RATSG1. [MASS = 49199 | | | MANNOSE 6- | | | FOLLISTATIN-RELATED | | | PREDICTED: SIMILAR TO |
| | | | | | PHOSPHATE/INSULIN- | | | PROTEIN 1 | | | IMPORTIN 7. [MASS = 119704 |
| | | | | | LIKE GROWTH FACTOR | | | PRECURSOR. | | | |
| | | | | | II RECEPTOR. | | | [MASS = 34622 | | | |
| | | | | | [MASS = 273608 | | | | | | |
| 4 | 9 | Rattus norvegicus (Rat) | 4 | 4 | Rattus norvegicus (Rat) | 3 | 3 | Rattus norvegicus (Rat) | 4 | 4 | Rattus norvegicus (Rat) |
| | | HEAT SHOCK PROTEIN | | | PREDICTED | | | CLUSTERIN | | | PREDICTED: SIMILAR TO |
| | | HSP 90-BETA. | | | NUCLEOLIN-RELATED | | | PRECURSOR. | | | RANBP21. [MASS = 136714 |
| | | [MASS = 83185 | | | PROTEIN NRP. | | | [MASS = 51375 | | | |
| | | | | | [MASS = 57036 | | | | | | |
| 4 | 6 | Rattus norvegicus (Rat) | 4 | 4 | Rattus norvegicus (Rat) | 3 | 3 | Rattus norvegicus (Rat) | 4 | 4 | Rattus norvegicus (Rat) |
| | | PEPTIDYL-PROLYL CIS- | | | DYNEIN HEAVY CHAIN, | | | PROTEIN DISULFIDE- | | | ALPHA-ACTININ-1. |
| | | TRANS ISOMERASE A. | | | CYTOSOLIC. | | | ISOMERASE | | | [MASS = 102960 |
| | | [MASS = 17743 | | | [MASS = 532252 | | | PRECURSOR. | | | |
| | | | | | | | | [MASS = 56951 | | | |
| 4 | 6 | Rattus norvegicus (Rat) | 4 | 4 | Rattus norvegicus (Rat) | 3 | 5 | Rattus norvegicus (Rat) | 4 | 4 | Rattus norvegicus (Rat) |
| | | PEROXIREDOXIN-2. | | | PEROXIREDOXIN-1. | | | PREDICTED: SIMILAR | | | ARCADLIN. [MASS = 103800 |
| | | [MASS = 21652 | | | [MASS = 22109 | | | TO INTER-ALPHA- | | | |
| | | | | | | | | INHIBITOR H2 CHAIN. | | | |
| | | | | | | | | [MASS = 105715 | | | |
| 4 | 7 | Rattus norvegicus (Rat) | 4 | 22 | Rattus norvegicus (Rat) | 3 | 3 | Rattus norvegicus (Rat) | 4 | 4 | Rattus norvegicus (Rat) |
| | | SPLICE ISOFORM 1 OF | | | EXTRACELLULAR | | | PREDICTED: SIMILAR | | | PROTOCADHERIN GAMMA |
| | | HETEROGENEOUS | | | SUPEROXIDE | | | TO ELASTIN | | | SUBFAMILY C, 3. |
| | | NUCLEAR | | | DISMUTASE [CU-ZN] | | | MICROFIBRIL | | | [MASS = 101038 |
| | | RIBONUCLEOPROTEIN | | | PRECURSOR. | | | INTERFACER 1. | | | |
| | | D0. [MASS = 38192 | | | [MASS = 26620 | | | [MASS = 107560 | | | |
| 4 | 4 | Rattus norvegicus (Rat) | 4 | 4 | Rattus norvegicus (Rat) | 3 | 3 | Rattus norvegicus (Rat) | 4 | 4 | Rattus norvegicus (Rat) |
| | | PREDICTED: similar to | | | PREDICTED: SIMILAR | | | PREDICTED: SIMILAR | | | EUKARYOTIC TRANSLATION |
| | | Heterogeneous nuclear | | | TO FILAMIN A. | | | TO ALPHA 2 TYPE VI | | | INITIATION FACTOR 4A, |
| | | ribonucleoproteins | | | [MASS = 290169 | | | COLLAGEN ISOFORM | | | ISOFORM 1. [MASS = 46154 |
| | | A2/B1. [MASS = 32468 | | | | | | 2C2A PRECURSOR. | | | |
| | | | | | | | | [MASS = 98272 | | | |
| 4 | 4 | Rattus norvegicus (Rat) | 4 | 5 | Rattus norvegicus (Rat) | 3 | 4 | Rattus norvegicus (Rat) | 4 | 4 | Rattus norvegicus (Rat) |
| | | 40S RIBOSOMAL | | | COMPLEMENT | | | PREDICTED: SIMILAR | | | RIBONUCLEOTIDE |
| | | PROTEIN S3A. | | | INHIBITORY FACTOR H. | | | TO COLLAGEN ALPHA | | | REDUCTASE M1. |
| | | [MASS = 29814 | | | [MASS = 140344 | | | 2(IV) CHAIN | | | [MASS = 90293 |
| | | | | | | | | PRECURSOR - MOUSE. | | | |
| | | | | | | | | [MASS = 192535 | | | |
| 4 | 4 | Rattus norvegicus (Rat) | 4 | 4 | Rattus norvegicus (Rat) | 3 | 3 | Rattus norvegicus (Rat) | 4 | 7 | Rattus norvegicus (Rat) |
| | | TXNRD1 PROTEIN. | | | PREDICTED: KINESIN | | | SPLICE ISOFORM 1 OF | | | PREDICTED: RETINOL |
| | | [MASS = 63002 | | | FAMILY MEMBER 4. | | | NEUROFASCIN | | | BINDING PROTEIN 4, |
| | | | | | [MASS = 139682 | | | PRECURSOR. | | | PLASMA. [MASS = 50139 |
| | | | | | | | | [MASS = 138004 | | | |
| 4 | 5 | Rattus norvegicus (Rat) | 4 | 4 | Rattus norvegicus (Rat) | 3 | 3 | Rattus norvegicus (Rat) | 4 | 4 | Rattus norvegicus (Rat) |
| | | EUKARYOTIC | | | CELL GROWTH | | | SERINE (OR CYSTEINE) | | | PREDICTED: COMPLEMENT |
| | | TRANSLATION | | | REGULATOR WITH EF | | | PROTEINASE | | | COMPONENT 7. |
| | | INITIATION FACTOR 4A, | | | HAND DOMAIN 1. | | | INHIBITOR, CLADE A | | | [MASS = 90661 |
| | | ISOFORM 1. | | | [MASS = 30835 | | | (ALPHA-1 | | | |
| | | [MASS = 46154 | | | | | | ANTIPROTEINASE, | | | |
| | | | | | | | | ANTITRYPSIN), | | | |
| | | | | | | | | MEMBER 6. | | | |
| | | | | | | | | [MASS = 44671 | | | |
| 4 | 4 | Rattus norvegicus (Rat) | 4 | 4 | Rattus norvegicus (Rat) | 3 | 6 | Rattus norvegicus (Rat) | 4 | 5 | Rattus norvegicus (Rat) |
| | | 14-3-3 PROTEIN THETA. | | | LEUKEMIA INHIBITORY | | | SP120-Heterogeneous | | | PREDICTED: SIMILAR TO |
| | | [MASS = 27778 | | | FACTOR RECEPTOR | | | nuclear ribonucleoprotein | | | CCTETA, ETA SUBUNIT OF |
| | | | | | PRECURSOR. | | | U. [MASS = 87748 | | | THE CHAPERONIN |
| | | | | | [MASS = 122394 | | | | | | CONTAINING TCP-1. |
| | | | | | | | | | | | [MASS = 75684 |
| 4 | 4 | Rattus norvegicus (Rat) | 4 | 4 | Rattus norvegicus (Rat) | 3 | 6 | Rattus norvegicus (Rat) | 4 | 4 | Rattus norvegicus (Rat) |
| | | ALPHA ISOFORM OF | | | PREDICTED: SIMILAR | | | RIBOSOMAL PROTEIN | | | PREDICTED: COMPLEMENT |
| | | REGULATORY SUBUNIT | | | TO PLEXIN-B2 | | | S27A. [MASS = 17951 | | | COMPONENT 5. |
| | | A, PROTEIN | | | PRECURSOR. | | | | | | [MASS = 152144 |
| | | PHOSPHATASE 2. | | | [MASS = 216119 | | | | | | |
| | | [MASS = 65323 | | | | | | | | | |
| 4 | 4 | Rattus norvegicus (Rat) | 4 | 5 | Rattus norvegicus (Rat) | 3 | 4 | Rattus norvegicus (Rat) | 4 | 6 | Rattus norvegicus (Rat) |
| | | PROTEIN KINASE C- | | | LOW-DENSITY | | | SPLICE ISOFORM 2 OF | | | NEUROCAN CORE PROTEIN |
| | | BINDING PROTEIN | | | LIPOPROTEIN | | | RECEPTOR-TYPE | | | PRECURSOR. [MASS = 135545 |
| | | NELL2. [MASS = 91334 | | | RECEPTOR-RELATED | | | TYROSINE-PROTEIN | | | |
| | | | | | PROTEIN 2 | | | PHOSPHATASE ZETA | | | |
| | | | | | PRECURSOR. | | | PRECURSOR. | | | |
| | | | | | [MASS = 519276 | | | [MASS = 164596 | | | |
| 4 | 4 | Rattus norvegicus (Rat) | 4 | 4 | Rattus norvegicus (Rat) | 3 | 3 | Rattus norvegicus (Rat) | 4 | 5 | Rattus norvegicus (Rat) |
| | | STRUCTURAL | | | LEUKOCYTE COMMON | | | 60S RIBOSOMAL | | | PREDICTED: SIMILAR TO |
| | | MAINTENANCE OF | | | ANTIGEN-RELATED | | | PROTEIN L13. | | | SEIZURE 6-LIKE PROTEIN |
| | | CHROMOSOME 3. | | | PHOSPHATASE | | | [MASS = 24178 | | | PRECURSOR. [MASS = 145870 |
| | | [MASS = 138448 | | | PRECURSOR. | | | | | | |
| | | | | | [MASS = 212954 | | | | | | |
| 4 | 4 | Rattus norvegicus (Rat) | 4 | 7 | Rattus norvegicus (Rat) | 3 | 3 | Rattus norvegicus (Rat) | 4 | 4 | Rattus norvegicus (Rat) |
| | | PREDICTED: SIMILAR | | | PROTOCADHERIN | | | PREDICTED: | | | HEPARIN COFACTOR 2 |
| | | TO IMPORTIN 7. | | | GAMMA SUBFAMILY C, | | | COMPLEMENT | | | PRECURSOR. [MASS = 54552 |
| | | [MASS = 119704 | | | 3. [MASS = 101038 | | | COMPONENT 7. | | | |
| | | | | | | | | [MASS = 90661 | | | |
| 4 | 4 | Rattus norvegicus (Rat) | 4 | 4 | Rattus norvegicus (Rat) | 3 | 3 | Rattus norvegicus (Rat) | 4 | 4 | Rattus norvegicus (Rat) |
| | | CADHERIN-6 | | | MYOSIN-9. | | | VIMENTIN. | | | PREDICTED: SIMILAR TO |
| | | PRECURSOR. | | | [MASS = 226207 | | | [MASS = 53602 | | | DNA REPLICATION |
| | | [MASS = 88341 | | | | | | | | | LICENSING FACTOR MCM2. |
| | | | | | | | | | | | [MASS = 102272 |
| 4 | 7 | Rattus norvegicus (Rat) | 4 | 4 | Rattus norvegicus (Rat) | 3 | 3 | Rattus norvegicus (Rat) | 4 | 5 | Rattus norvegicus (Rat) |
| | | VITAMIN D-BINDING | | | 14-3-3 PROTEIN | | | LEUKOCYTE COMMON | | | PREDICTED: NIDOGEN. |
| | | PROTEIN PRECURSOR. | | | EPSILON. [MASS = 29174 | | | ANTIGEN-RELATED | | | [MASS = 138365 |
| | | [MASS = 53544 | | | | | | PHOSPHATASE | | | |
| | | | | | | | | PRECURSOR. | | | |
| | | | | | | | | [MASS = 207012 | | | |
| 4 | 4 | Rattus norvegicus (Rat) | 4 | 4 | Rattus norvegicus (Rat) | 3 | 3 | Rattus norvegicus (Rat) | 4 | 7 | Rattus norvegicus (Rat) |
| | | PREDICTED: SIMILAR | | | THROMBOSPONDIN 1. | | | NEUROSERPIN | | | PEPTIDYLPROLYL |
| | | TO ALANYL-TRNA | | | [MASS = 129671 | | | PRECURSOR. | | | ISOMERASE C. [MASS = 23009 |
| | | SYNTHETASE. | | | | | | [MASS = 46278 | | | |
| | | [MASS = 106811 | | | | | | | | | |
| 4 | 4 | Rattus norvegicus (Rat) | 4 | 7 | Rattus norvegicus (Rat) | 3 | 4 | Rattus norvegicus (Rat) | 4 | 4 | Rattus norvegicus (Rat) |
| | | PREDICTED: | | | HISTONE H1.2. | | | NEUROCAN CORE | | | SPLICE ISOFORM 2 OF |
| | | TRANSFORMING | | | [MASS = 21856 | | | PROTEIN PRECURSOR. | | | POLYPYRIMIDINE TRACT- |
| | | GROWTH FACTOR, | | | | | | [MASS = 135545 | | | BINDING PROTEIN 2. |
| | | BETA INDUCED, 68 KDA. | | | | | | | | | [MASS = 57645 |
| | | [MASS = 74369 | | | | | | | | | |
| 4 | 4 | Rattus norvegicus (Rat) | 3 | 3 | Rattus norvegicus (Rat) | 3 | 3 | Rattus norvegicus (Rat) | 4 | 4 | Rattus norvegicus (Rat) |
| | | PREDICTED: similar to | | | RATSG1. [MASS = 49199 | | | PREDICTED: SIMILAR | | | TXNRD1 PROTEIN. |
| | | phosphoribosylformyl- | | | | | | TO AMYLOID BETA (A4) | | | [MASS = 63002 |
| | | glycinamidine | | | | | | PRECURSOR-LIKE | | | |
| | | synthase. | | | | | | PROTEIN 1. | | | |
| | | [MASS = 146178 | | | | | | [MASS = 68777 | | | |
| 4 | 6 | Rattus norvegicus (Rat) | 3 | 3 | Rattus norvegicus (Rat) | 3 | 3 | Rattus norvegicus (Rat) | 4 | 5 | Rattus norvegicus (Rat) |
| | | PREDICTED: SIMILAR | | | CC2-27. [MASS = 120523 | | | HEPARIN COFACTOR 2 | | | TENASCIN (FRAGMENT). |
| | | TO CRB2 PROTEIN. | | | | | | PRECURSOR. | | | [MASS = 62473 |
| | | [MASS = 138781 | | | | | | [MASS = 54552 | | | |
| 4 | 4 | Rattus norvegicus (Rat) | 3 | 4 | Rattus norvegicus (Rat) | 3 | 3 | Rattus norvegicus (Rat) | 4 | 5 | Rattus norvegicus (Rat) |
| | | AFAMIN PRECURSOR. | | | PREDICTED: SIMILAR | | | NETRIN RECEPTOR | | | QUIESCIN Q6. [MASS = 82412 |
| | | [MASS = 69335 | | | TO ALPHA ENOLASE. | | | UNC5C PRECURSOR. | | | |
| | | | | | [MASS = 46489 | | | [MASS = 103135 | | | |
| 4 | 4 | Rattus norvegicus (Rat) | 3 | 4 | Rattus norvegicus (Rat) | 3 | 3 | Rattus norvegicus (Rat) | 4 | 6 | Rattus norvegicus (Rat) POLY |
| | | PREDICTED: SIMILAR | | | HYRAC. [MASS = 31353 | | | CULLIN-ASSOCIATED | | | [ADP-RIBOSE] POLYMERASE |
| | | TO RANBP21. | | | | | | NEDD8-DISSOCIATED | | | 1. [MASS = 112529 |
| | | [MASS = 136714 | | | | | | PROTEIN 1. | | | |
| | | | | | | | | [MASS = 136362 | | | |
| 4 | 6 | Rattus norvegicus (Rat) | 3 | 5 | Rattus norvegicus (Rat) | 3 | 3 | Rattus norvegicus (Rat) | 4 | 4 | Rattus norvegicus (Rat) |
| | | BETA-2- | | | VITAMIN D-BINDING | | | LEUKEMIA INHIBITORY | | | SPLICE ISOFORM 1 OF |
| | | MICROGLOBULIN | | | PROTEIN PRECURSOR. | | | FACTOR RECEPTOR | | | CULLIN-ASSOCIATED |
| | | PRECURSOR. | | | [MASS = 53544 | | | PRECURSOR. | | | NEDD8-DISSOCIATED |
| | | [MASS = 13720 | | | | | | [MASS = 122394 | | | PROTEIN 2. [MASS = 139673 |
| 4 | 6 | Rattus norvegicus (Rat) | 3 | 5 | Rattus norvegicus (Rat) | 3 | 3 | Rattus norvegicus (Rat) | 4 | 4 | Rattus norvegicus (Rat) |
| | | 60S RIBOSOMAL | | | EUKARYOTIC | | | CLATHRIN HEAVY | | | PREDICTED: SIMILAR TO |
| | | PROTEIN L13. | | | TRANSLATION | | | CHAIN. [MASS = 191599 | | | INTER-ALPHA TRYPSIN |
| | | [MASS = 24178 | | | INITIATION FACTOR | | | | | | INHIBITOR, HEAVY CHAIN 1. |
| | | | | | 4A2. [MASS = 46489 | | | | | | [MASS = 104581 |
| 4 | 6 | Rattus norvegicus (Rat) | 3 | 3 | Rattus norvegicus (Rat) | 3 | 3 | Rattus norvegicus (Rat) | 4 | 4 | Rattus norvegicus (Rat) |
| | | 40S RIBOSOMAL | | | PREDICTED: similar to | | | FAM3C-LIKE PROTEIN. | | | MANNOSIDASE 2, ALPHA B1. |
| | | PROTEIN S6. | | | phosphoribosylformylglycinamidine | | | [MASS = 24714 | | | [MASS = 114327 |
| | | [MASS = 28681 | | | synthase. | | | | | | |
| | | | | | [MASS = 146178 | | | | | | |
| 4 | 5 | Rattus norvegicus (Rat) | 3 | 4 | Rattus norvegicus (Rat) | 3 | 3 | Rattus norvegicus (Rat) | 4 | 4 | Rattus norvegicus (Rat) |
| | | 14-3-3 PROTEIN | | | FOLLISTATIN-RELATED | | | PREDICTED: SIMILAR | | | STRUCTURAL |
| | | GAMMA. [MASS = 28171 | | | PROTEIN 1 | | | TO LAMININ ALPHA-1 | | | MAINTENANCE OF |
| | | | | | PRECURSOR. | | | CHAIN PRECURSOR - | | | CHROMOSOME 3. |
| | | | | | [MASS = 34622 | | | MOUSE. [MASS = 338692 | | | [MASS = 138448 |
| 4 | 6 | Rattus norvegicus (Rat) | 3 | 3 | Rattus norvegicus (Rat) | 3 | 3 | Rattus norvegicus (Rat) | 4 | 4 | Rattus norvegicus (Rat) |
| | | PREDICTED: SIMILAR | | | 40S RIBOSOMAL | | | THROMBOSPONDIN 1. | | | DIHYDROPYRIMIDINASE- |
| | | TO PROGRAMMED CELL | | | PROTEIN SA. | | | [MASS = 129671 | | | RELATED PROTEIN 2. |
| | | DEATH 6 INTERACTING | | | [MASS = 32693 | | | | | | [MASS = 62278 |
| | | PROTEIN. [MASS = 75806 | | | | | | | | | |
| 4 | 4 | Rattus norvegicus (Rat) | 3 | 3 | Rattus norvegicus (Rat) | 2 | 2 | Rattus norvegicus (Rat) | 4 | 4 | Rattus norvegicus (Rat) |
| | | PREDICTED: SIMILAR | | | 14-3-3 PROTEIN THETA. | | | MANNOSIDASE, ALPHA, | | | PREDICTED: SIMILAR TO P30 |
| | | TO PUTATIVE PRE- | | | [MASS = 27778 | | | CLASS A1, MEMBER 1. | | | DBC PROTEIN. |
| | | MRNA SPLICING | | | | | | [MASS = 73125 | | | [MASS = 114440 |
| | | FACTOR RNA | | | | | | | | | |
| | | HELICASE. | | | | | | | | | |
| | | [MASS = 90977 | | | | | | | | | |
| 4 | 15 | Rattus norvegicus (Rat) | 3 | 3 | Rattus norvegicus (Rat) | 2 | 3 | Rattus norvegicus (Rat) | 4 | 4 | Rattus norvegicus (Rat) 26S |
| | | EXTRACELLULAR | | | 60S RIBOSOMAL | | | HYRAC. [MASS = 31353 | | | PROTEASOME NON-ATPASE |
| | | SUPEROXIDE | | | PROTEIN L18. | | | | | | REGULATORY SUBUNIT 1. |
| | | DISMUTASE [CU—ZN] | | | [MASS = 21527 | | | | | | [MASS = 105748 |
| | | PRECURSOR. | | | | | | | | | |
| | | [MASS = 26620 | | | | | | | | | |
| 4 | 4 | Rattus norvegicus (Rat) | 3 | 4 | Rattus norvegicus (Rat) | 2 | 2 | Rattus norvegicus (Rat) | 4 | 4 | Rattus norvegicus (Rat) |
| | | SMC4L1 PROTEIN. | | | ADENOSYLHOMOCYSTEINASE. | | | SEZ6B. [MASS = 105607 | | | FAM3C-LIME PROTEIN. |
| | | [MASS = 146806 | | | [MASS = 47407 | | | | | | [MASS = 24714 |
| 4 | 4 | Rattus norvegicus (Rat) | 3 | 3 | Rattus norvegicus (Rat) | 2 | 3 | Rattus norvegicus (Rat) | 4 | 4 | Rattus norvegicus (Rat) |
| | | SPLICEOSOME RNA | | | SPARC PRECURSOR. | | | IG KAPPA CHAIN C | | | LEUKEMIA INHIBITORY |
| | | HELICASE BAT1. | | | [MASS = 34384 | | | REGION, B ALLELE. | | | FACTOR RECEPTOR |
| | | [MASS = 49035 | | | | | | [MASS = 11601 | | | PRECURSOR. [MASS = 122394 |
| 4 | 4 | Rattus norvegicus (Rat) | 3 | 3 | Rattus norvegicus (Rat) | 2 | 2 | Rattus norvegicus (Rat) | 4 | 4 | Rattus norvegicus (Rat) |
| | | PREDICTED: SIMILAR | | | PROCOLLAGEN- | | | EUKARYOTIC | | | HAUSP. [MASS = 128431 |
| | | TO 40S RIBOSOMAL | | | LYSINE,2- | | | TRANSLATION | | | |
| | | PROTEIN S9. | | | OXOGLUTARATE 5- | | | INITIATION FACTOR | | | |
| | | [MASS = 22648 | | | DIOXYGENASE 3 | | | 4A2. [MASS = 46489 | | | |
| | | | | | PRECURSOR. | | | | | | |
| | | | | | [MASS = 85060 | | | | | | |
| 4 | 4 | Rattus norvegicus (Rat) | 3 | 3 | Rattus norvegicus (Rat) | 2 | 2 | Rattus norvegicus (Rat) | 4 | 4 | Rattus norvegicus (Rat) |
| | | 60S ACIDIC RIBOSOMAL | | | PREDICTED similar to C- | | | PREDICTED: SIMILAR | | | COLLAGEN ALPHA-1(I) |
| | | PROTEIN P0. | | | 1-TETRAHYDROFOLATE | | | TO CG1841-PA, | | | CHAIN PRECURSOR. |
| | | [MASS = 34215 | | | SYNTHASE, | | | ISOFORM A. | | | [MASS = 137886 |
| | | | | | CYTOPLASMIC. | | | [MASS = 52522 | | | |
| | | | | | [MASS = 100351 | | | | | | |
| 4 | 4 | Rattus norvegicus (Rat) | 3 | 3 | Rattus norvegicus (Rat) | 2 | 2 | Rattus norvegicus (Rat) | 4 | 6 | Rattus norvegicus (Rat) FETUB |
| | | PREDICTED: SIMILAR | | | M-CADHERIN. | | | ALPHA-2-HS- | | | PROTEIN. [MASS = 43169 |
| | | TO P30 DBC PROTEIN. | | | [MASS = 85753 | | | GLYCOPROTEIN | | | |
| | | [MASS = 11440 | | | | | | PRECURSOR. | | | |
| | | | | | | | | [MASS = 37982 | | | |
| 4 | 4 | Rattus norvegicus (Rat) | 3 | 11 | Rattus norvegicus (Rat) | 2 | 3 | Rattus norvegicus (Rat) | 3 | 3 | Rattus norvegicus (Rat) |
| | | PREDICTED: SIMILAR | | | RIBOSOMAL PROTEIN | | | VITAMIN D-BINDING | | | PROTEASOME SUBUNIT |
| | | TO ISOLEUCINE-TRNA | | | S27A. [MASS = 17951 | | | PROTEIN PRECURSOR. | | | ALPHA TYPE 2. [MASS = 25795 |
| | | SYNTHETASE. | | | | | | [MASS = 53544 | | | |
| | | [MASS = 144169 | | | | | | | | | |
| 4 | 5 | Rattus norvegicus (Rat) | 3 | 3 | Rattus norvegicus (Rat) | 2 | 2 | Rattus norvegicus (Rat) | 3 | 3 | Rattus norvegicus (Rat) |
| | | 40S RIBOSOMAL | | | PREDICTED: SIMILAR | | | VACUOLAR ATP | | | RATSG1. [MASS = 49199 |
| | | PROTEIN S10. | | | TO ALPHA NAC/1.9.2. | | | SYNTHASE SUBUNIT S1 | | | |
| | | [MASS = 18916 | | | PROTEIN. [MASS = 23384 | | | PRECURSOR. | | | |
| | | | | | | | | [MASS = 51123 | | | |
| 4 | 4 | Rattus norvegicus (Rat) | 3 | 5 | Rattus norvegicus (Rat) | 2 | 2 | Rattus norvegicus (Rat) | 3 | 4 | Rattus norvegicus (Rat) |
| | | PREDICTED: SIMILAR | | | SPLICE ISOFORM 1 OF | | | PREDICTED: ATPASE, | | | COFILIN-1. [MASS = 24588 |
| | | TO VINCULIN. | | | ALPHA-1B- | | | H+ TRANSPORTING, | | | |
| | | [MASS = 116615 | | | GLYCOPROTEIN | | | LYSOSOMAL | | | |
| | | | | | PRECURSOR. | | | ACCESSORY PROTEIN | | | |
| | | | | | [MASS = 56479 | | | 2. [MASS = 66094 | | | |
| 4 | 4 | Rattus norvegicus (Rat) | 3 | 3 | Rattus norvegicus (Rat) | 2 | 2 | Rattus norvegicus (Rat) | 3 | 5 | Rattus norvegicus (Rat) |
| | | PREDICTED: | | | SUPEROXIDE | | | PREDICTED: SIMILAR | | | PEPTIDYL-PROLYL CIS- |
| | | CALSYNTENIN 1. | | | DISMUTASE. | | | TO PROCOLLAGEN, | | | TRANS ISOMERASE A. |
| | | [MASS = 109351 | | | [MASS = 15780 | | | TYPE IX, ALPHA 2. | | | [MASS = 17743 |
| | | | | | | | | [MASS = 71422 | | | |
| 4 | 4 | Rattus norvegicus (Rat) | 3 | 4 | Rattus norvegicus (Rat) | 2 | 3 | Rattus norvegicus (Rat) | 3 | 4 | Rattus norvegicus (Rat) RAB |
| | | FAR UPSTREAM | | | EUKARYOTIC | | | PREDICTED: SIMILAR | | | GDP DISSOCIATION |
| | | ELEMENT-BINDING | | | TRANSLATION | | | TO ALPHA ENOLASE. | | | INHIBITOR BETA. |
| | | PROTEIN 2. | | | INITIATION FACTOR 4A, | | | [MASS = 46489 | | | [MASS = 50685 |
| | | [MASS = 74226 | | | ISOFORM 1. | | | | | | |
| | | | | | [MASS = 46154 | | | | | | |
| 4 | 5 | Rattus norvegicus (Rat) L- | 3 | 5 | Rattus norvegicus (Rat) | 2 | 3 | Rattus norvegicus (Rat) | 3 | 3 | Rattus norvegicus (Rat) 40S |
| | | LACTATE | | | COLLAGEN ALPHA-1(V) | | | SOLUBLE CALCIUM- | | | RIBOSOMAL PROTEIN SA. |
| | | DEHYDROGENASE A | | | CHAIN PRECURSOR. | | | ACTIVATED | | | [MASS = 32693 |
| | | CHAIN. [MASS = 36451 | | | [MASS = 184610 | | | NUCLEOTIDASE 1. | | | |
| | | | | | | | | [MASS = 45659 | | | |
| 4 | 4 | Rattus norvegicus (Rat) | 3 | 3 | Rattus norvegicus (Rat) | 2 | 2 | Rattus norvegicus (Rat) | 3 | 3 | Rattus norvegicus (Rat) |
| | | SPLICE ISOFORM 1 OF | | | NONO/P54NRB | | | VASCULAR CELL | | | PHOSPHATIDY- |
| | | ACETYL-COA | | | HOMOLOG. | | | ADHESION PROTEIN 1 | | | LETHANOLAMINE- |
| | | CARBOXYLASE 1. | | | [MASS = 75487 | | | PRECURSOR. | | | BINDING PROTEIN. |
| | | [MASS = 265421 | | | | | | [MASS = 81246 | | | [MASS = 20670 |
| 4 | 4 | Rattus norvegicus (Rat) | 3 | 3 | Rattus norvegicus (Rat) | 2 | 2 | Rattus norvegicus (Rat) | 3 | 3 | Rattus norvegicus (Rat) |
| | | MICROTUBULE- | | | PREDICTED: | | | PREDICTED: similar to | | | SECRETOGRANIN-3 |
| | | ASSOCIATED PROTEIN | | | CALSYNTENIN 1. | | | alpha 1 type II collagen. | | | PRECURSOR. [MASS = 53183 |
| | | 4. [MASS = 110301 | | | [MASS = 109351 | | | [MASS = 138706 | | | |
| 4 | 4 | Rattus norvegicus (Rat) | 3 | 3 | Rattus norvegicus (Rat) | 2 | 2 | Rattus norvegicus (Rat) | 3 | 5 | Rattus norvegicus (Rat) |
| | | COLD SHOCK DOMAIN- | | | RUVB-LIKE 1. | | | C-REACTIVE PROTEIN | | | PEROXIREDOXIN-2 |
| | | CONTAINING PROTEIN | | | [MASS = 50214 | | | PRECURSOR. | | | [MASS = 21652 |
| | | E1. [MASS = 88895 | | | | | | [MASS = 25468 | | | |
| 4 | 4 | Rattus norvegicus (Rat) | 3 | 4 | Rattus norvegicus (Rat) | 2 | 3 | Rattus norvegicus (Rat) | 3 | 5 | Rattus norvegicus (Rat) |
| | | 26S PROTEASOME | | | PREDICTED similar to | | | EPITHELIAL-CADHERIN | | | SERINE/CYSTEINE |
| | | NON-ATPASE | | | POSTSYNAPTIC | | | PRECURSOR. | | | PROTEINASE INHIBITOR, |
| | | REGULATORY SUBUNIT | | | DENSITY PROTEIN. | | | [MASS = 98715 | | | CLADE C, MEMBER 1. |
| | | 1. [MASS = 105748 | | | [MASS = 186848 | | | | | | [MASS = 52234 |
| 4 | 4 | Rattus norvegicus (Rat) | 3 | 3 | Rattus norvegicus (Rat) | 2 | 2 | Rattus norvegicus (Rat) | 3 | 3 | Rattus norvegicus (Rat) |
| | | PREDICTED: SIMILAR | | | 60S RIBOSOMAL | | | BIFUNCTIONAL | | | PREDICTED: SIMILAR TO |
| | | TO RAS GTPASE- | | | PROTEIN L4. | | | HEPARAN SULFATE N- | | | AMYLOID BETA (A4) |
| | | ACTIVATING-LIKE | | | [MASS = 47126 | | | DEACETYLASE/N- | | | PRECURSOR-LIKE PROTEIN |
| | | PROTEIN IQGAP1. | | | | | | SULFOTRANSFERASE 1 | | | 1. [MASS = 68777 |
| | | [MASS = 196522 | | | | | | (EC 2.8.2.8) | | | |
| | | | | | | | | (GLUCOSAMINYL N- | | | |
| | | | | | | | | DEACETYLASE/N- | | | |
| | | | | | | | | SULFOTRANSFERASE | | | |
| | | | | | | | | 1) (NDST-1) (. | | | |
| | | | | | | | | [MASS = 101202 | | | |
| 4 | 5 | Rattus norvegicus (Rat) | 3 | 3 | Rattus norvegicus (Rat) | 2 | 2 | Rattus norvegicus (Rat) | 3 | 5 | Rattus norvegicus (Rat) |
| | | GTP-BINDING NUCLEAR | | | PROTEIN DISULFIDE- | | | SPARC PRECURSOR. | | | PREDICTED: SIMILAR TO |
| | | PROTEIN RAN, TESTIS- | | | ISOMERASE | | | [MASS = 34384 | | | RAN BINDING PROTEIN 5. |
| | | SPECIFIC ISOFORM. | | | PRECURSOR. | | | | | | [MASS = 99947 |
| | | [MASS = 24451 | | | [MASS = 56951 | | | | | | |
| 3 | 3 | Rattus norvegicus (Rat) | 3 | 3 | Rattus norvegicus (Rat) | 2 | 2 | Rattus norvegicus (Rat) | 3 | 3 | Rattus norvegicus (Rat) |
| | | 14-3-3 PROTEIN | | | PREDICTED: SIMILAR | | | PREDICTED: | | | PREDICTED: SIMILAR TO |
| | | ZETA/DELTA. | | | TO LAMININ-2 ALPHA2 | | | GLYCOPROTEIN-4- | | | PROGRAMMED CELL DEATH |
| | | [MASS = 27771 | | | CHAIN PRECURSOR. | | | BETA- | | | 6 INTERACTING PROTEIN. |
| | | | | | [MASS = 359007 | | | GALACTOSYL- | | | [MASS = 75806 |
| | | | | | | | | TRANSFERASE | | | |
| | | | | | | | | 2. [MASS = 44484 | | | |
| 3 | 5 | Rattus norvegicus (Rat) | 3 | 6 | Rattus norvegicus (Rat) | 2 | 2 | Rattus norvegicus (Rat) | 3 | 6 | Rattus norvegicus (Rat) |
| | | PREDICTED: SIMILAR | | | PREDICTED: SIMILAR | | | PREDICTED: SIMILAR | | | PREDICTED: |
| | | TO ALPHA ENOLASE. | | | TO INTER-ALPHA- | | | TO PAPPALYSIN-2 | | | DYSTROGLYCAN 1. |
| | | [MASS = 46489 | | | INHIBITOR H2 CHAIN. | | | PRECURSOR. | | | [MASS = 96706 |
| | | | | | [MASS = 105715 | | | [MASS = 204770 | | | |
| 3 | 5 | Rattus norvegicus (Rat) | 3 | 3 | Rattus norvegicus (Rat) | 2 | 2 | Rattus norvegicus (Rat) | 3 | 3 | Rattus norvegicus (Rat) |
| | | GLUTATHIONE | | | SHEN-DAN. | | | MASP-3 PROTEIN. | | | PREDICTED: similar to Slit-like |
| | | PEROXIDASE 3 | | | [MASS = 131080 | | | [MASS = 82497 | | | 2. [MASS = 72321 |
| | | PRECURSOR. | | | | | | | | | |
| | | [MASS = 25393 | | | | | | | | | |
| 3 | 6 | Rattus norvegicus (Rat) | 3 | 3 | Rattus norvegicus (Rat) | 2 | 2 | Rattus norvegicus (Rat) | 3 | 3 | Rattus norvegicus (Rat) |
| | | PREDICTED: | | | FAR UPSTREAM | | | PREDICTED: similar to T- | | | SPLICE ISOFORM 1 OF |
| | | DYSTROGLYCAN 1. | | | ELEMENT-BINDING | | | complex protein 1 subunit | | | HETEROGENEOUS |
| | | [MASS = 96706 | | | PROTEIN 2. | | | theta. [MASS = 59745 | | | NUCLEAR |
| | | | | | [MASS = 74226 | | | | | | RIBONUCLEOPROTEIN D0. |
| | | | | | | | | | | | [MASS = 38192 |
| 3 | 3 | Rattus norvegicus (Rat) | 3 | 3 | Rattus norvegicus (Rat) | 2 | 2 | Rattus norvegicus (Rat) | 3 | 3 | Rattus norvegicus (Rat) |
| | | COFILIN-1. | | | PREDICTED: SIMILAR | | | NUCLEAR | | | NEURAL CELL ADHESION |
| | | [MASS = 24588 | | | TO IMPORTIN 9. | | | AUTOANTIGENIC | | | MOLECULE 1, 140 KDA |
| | | | | | [MASS = 131739 | | | SPERM PROTEIN. | | | ISOFORM PRECURSOR. |
| | | | | | | | | [MASS = 84200 | | | [MASS = 94658 |
| 3 | 3 | Rattus norvegicus (Rat) | 3 | 3 | Rattus norvegicus (Rat) | 2 | 2 | Rattus norvegicus (Rat) | 3 | 3 | Rattus norvegicus (Rat) |
| | | HEAT-SHOCK PROTEIN | | | PREDICTED: SIMILAR | | | SUPEROXIDE | | | PREDICTED: SIMILAR TO |
| | | 105 KDA. [MASS = 96419 | | | TO VINCULIN. | | | DISMUTASE. | | | PUTATIVE PRE-MRNA |
| | | | | | [MASS = 116615 | | | [MASS = 15780 | | | SPLICING FACTOR RNA |
| | | | | | | | | | | | HELICASE. [MASS = 90977 |
| 3 | 4 | Rattus norvegicus (Rat) | 3 | 4 | Rattus norvegicus (Rat) | 2 | 3 | Rattus norvegicus (Rat) | 3 | 3 | Rattus norvegicus (Rat) DNA |
| | | PREDICTED: SIMILAR | | | PREDICTED similar to | | | APOLIPOPROTEIN D | | | POLYMERASE ALPHA |
| | | TO ALPHA NAC/1.9.2. | | | Nuclear autoantigenic | | | PRECURSOR. | | | CATALYTIC SUBUNIT |
| | | PROTEIN. [MASS = 23384 | | | sperm protein. | | | [MASS = 21635 | | | (FRAGMENT). [MASS = 165306 |
| | | | | | [MASS = 45764 | | | | | | |
| 3 | 7 | Rattus norvegicus (Rat) | 3 | 3 | Rattus norvegicus (Rat) | 2 | 2 | Rattus norvegicus (Rat) | 3 | 3 | Rattus norvegicus (Rat) |
| | | TUMOR NECROSIS | | | FAM3C-LIKE PROTEIN. | | | PREDICTED: SIMILAR | | | PREDICTED: SIMILAR TO |
| | | FACTOR TYPE 1 | | | [MASS = 24714 | | | TO PROGRAMMED | | | ARX. [MASS = 121446 |
| | | RECEPTOR | | | | | | CELL DEATH 6 | | | |
| | | ASSOCIATED PROTEIN. | | | | | | INTERACTING | | | |
| | | [MASS = 80461 | | | | | | PROTEIN. [MASS = 75806 | | | |
| 3 | 4 | Rattus norvegicus (Rat) | 3 | 3 | Rattus norvegicus (Rat) | 2 | 2 | Rattus norvegicus (Rat) | 3 | 4 | Rattus norvegicus (Rat) |
| | | FRUCTOSE- | | | D-3- | | | PREDICTED: SIMILAR | | | FOLLISTATIN-RELATED |
| | | BISPHOSPHATE | | | PHOSPHOGLYCERATE | | | TO SEMAPHORIN 6D-4. | | | PROTEIN 1 PRECURSOR. |
| | | ALDOLASE A. | | | DEHYDROGENASE. | | | [MASS = 159473 | | | [MASS = 34622 |
| | | [MASS = 39221 | | | [MASS = 56362 | | | | | | |
| 3 | 3 | Rattus norvegicus (Rat) | 3 | 5 | Rattus norvegicus (Rat) | 2 | 4 | Rattus norvegicus (Rat) | 3 | 4 | Rattus norvegicus (Rat) |
| | | PREDICTED: BRAIN | | | PREDICTED: SIMILAR | | | IGH-1A PROTEIN. | | | SPLICE ISOFORM PYBP1 OF |
| | | GLYCOGEN | | | TO COLLAGEN ALPHA | | | [MASS = 51403 | | | POLYPYRIMIDINE TRACT- |
| | | PHOSPHORYLASE. | | | 2(IV) CHAIN | | | | | | BINDING PROTEIN 1. |
| | | [MASS = 96738 | | | PRECURSOR - MOUSE. | | | | | | [MASS = 56937 |
| | | | | | [MASS = 192535 | | | | | | |
| 3 | 7 | Rattus norvegicus (Rat) | 3 | 4 | Rattus norvegicus (Rat) | 2 | 2 | Rattus norvegicus (Rat) L- | 3 | 4 | Rattus norvegicus (Rat) |
| | | CYSTATIN C | | | PROLIFERATION- | | | LACTATE | | | PREDICTED: SIMILAR TO |
| | | PRECURSOR. | | | ASSOCIATED 2G4, | | | DEHYDROGENASE A | | | HEAT SHOCK PROTEIN HSP |
| | | [MASS = 15437 | | | 38 KDA. [MASS = 43657 | | | CHAIN. [MASS = 36451 | | | 90-BETA. [MASS = 80701 |
| 3 | 3 | Rattus norvegicus (Rat) | 3 | 6 | Rattus norvegicus (Rat) | 2 | 3 | Rattus norvegicus (Rat) | 3 | 3 | Rattus norvegicus (Rat) |
| | | PREDICTED: SIMILAR | | | 14-3-3 PROTEIN | | | RECEPTOR-LIKE | | | PREDICTED: SIMILAR TO |
| | | TO PTK7 PROTEIN | | | GAMMA. [MASS = 28171 | | | PROTEIN TYROSINE | | | DNA REPLICATION |
| | | TYROSINE KINASE 7. | | | | | | PHOSPHATASE KAPPA | | | LICENSING FACTOR MCM5. |
| | | [MASS = 139818 | | | | | | EXTRACELLULAR | | | [MASS = 91577 |
| | | | | | | | | REGION. [MASS = 56159 | | | |
| 3 | 10 | Rattus norvegicus (Rat) | 3 | 3 | Rattus norvegicus (Rat) | 2 | 2 | Rattus norvegicus (Rat) | 3 | 5 | Rattus norvegicus (Rat) |
| | | RIBOSOMAL PROTEIN | | | PREDICTED: SIMILAR | | | PREDICTED: SIMILAR | | | PROFILIN-1. [MASS = 14826 |
| | | S27A. [MASS = 17951 | | | TO RAS GTPASE- | | | TO SEROTRANSFERRIN | | | |
| | | | | | ACTIVATING-LIKE | | | PRECURSOR. | | | |
| | | | | | PROTEIN IQGAP1. | | | [MASS = 76607 | | | |
| | | | | | [MASS = 196522 | | | | | | |
| 3 | 3 | Rattus norvegicus (Rat) | 3 | 3 | Rattus norvegicus (Rat) | 2 | 2 | Rattus norvegicus (Rat) | 3 | 3 | Rattus norvegicus (Rat) |
| | | COMPLEMENT C4 | | | PROTOCADHERIN. | | | PREDICTED: SIMILAR | | | PREDICTED: CALSYNTENIN |
| | | PRECURSOR. | | | [MASS = 505997 | | | TO RAN BINDING | | | 1. [MASS = 109351 |
| | | [MASS = 192163 | | | | | | PROTEIN 5. | | | |
| | | | | | | | | [MASS = 133476 | | | |
| 3 | 3 | Rattus norvegicus (Rat) | 3 | 3 | Rattus norvegicus (Rat) | 2 | 3 | Rattus norvegicus (Rat) | 3 | 5 | Rattus norvegicus (Rat) 60S |
| | | POLY [ADP-RIBOSE] | | | CHAPERONIN SUBUNIT | | | APOLIPOPROTEIN M | | | RIBOSOMAL PROTEIN L18. |
| | | POLYMERASE 1. | | | 6A. [MASS = 58017 | | | PRECURSOR. | | | [MASS = 21527 |
| | | [MASS = 112529 | | | | | | [MASS = 21513 | | | |
| 3 | 4 | Rattus norvegicus (Rat) | 3 | 3 | Rattus norvegicus (Rat) | 2 | 2 | Rattus norvegicus (Rat) | 3 | 3 | Rattus norvegicus (Rat) |
| | | 40S RIBOSOMAL | | | PREDICTED: SIMILAR | | | HEAT SHOCK PROTEIN | | | PREDICTED: SIMILAR TO |
| | | PROTEIN S4, X | | | TO EUKARYOTIC | | | HSP 90-BETA. | | | SPLICING FACTOR 3B, |
| | | ISOFORM. [MASS = 29467 | | | TRANSLATION | | | [MASS = 83185 | | | SUBUNIT 3, 130 KDA. |
| | | | | | INITIATION FACTOR 3, | | | | | | [MASS = 174174 |
| | | | | | SUBUNIT 10 THETA, | | | | | | |
| | | | | | 150/170 KDA. | | | | | | |
| | | | | | [MASS = 192616 | | | | | | |
| 3 | 4 | Rattus norvegicus (Rat) | 3 | 3 | Rattus norvegicus (Rat) | 2 | 2 | Rattus norvegicus (Rat) | 3 | 3 | Rattus norvegicus (Rat) |
| | | 40S RIBOSOMAL | | | PREDICTED: SIMILAR | | | CATHEPSIN D | | | SPARC-LIKE PROTEIN 1 |
| | | PROTEIN S8. | | | TO VERY LARGE G | | | PRECURSOR. | | | PRECURSOR. [MASS = 70634 |
| | | [MASS = 24074 | | | PROTEIN-COUPLED | | | [MASS = 44681 | | | |
| | | | | | RECEPTOR 1. | | | | | | |
| | | | | | [MASS = 413840 | | | | | | |
| 3 | 3 | Rattus norvegicus (Rat) | 3 | 3 | Rattus norvegicus (Rat) | 2 | 3 | Rattus norvegicus (Rat) | 3 | 3 | Rattus norvegicus (Rat) |
| | | EPSILON 3 GLOBIN. | | | CADHERIN EGF LAG | | | MICROFIBRILLAR- | | | EUKARYOTIC TRANSLATION |
| | | [MASS = 16540 | | | SEVEN-PASS G-TYPE | | | ASSOCIATED PROTEIN | | | INITIATION FACTOR 4A2. |
| | | | | | RECEPTOR 2. | | | 4. [MASS = 29050 | | | [MASS = 46489 |
| | | | | | [MASS = 317122 | | | | | | |
| 3 | 3 | Rattus norvegicus (Rat) | 3 | 4 | Rattus norvegicus (Rat) | 2 | 2 | Rattus norvegicus (Rat) | 3 | 5 | Rattus norvegicus (Rat) |
| | | PREDICTED: | | | STATHMIN. | | | FRUCTOSE- | | | EXTRACELLULAR |
| | | PHOSPHORIBOSYL- | | | [MASS = 17157 | | | BISPHOSPHATE | | | SUPEROXIDE DISMUTASE |
| | | GLYCINAMIDE | | | | | | ALDOLASE A. | | | [CU—ZN] PRECURSOR. |
| | | FORMYLTRANSFERASE. | | | | | | [MASS = 39221 | | | [MASS = 26620 |
| | | [MASS = 107580 | | | | | | | | | |
| 3 | 6 | Rattus norvegicus (Rat) | 3 | 3 | Rattus norvegicus (Rat) | 2 | 3 | Rattus norvegicus (Rat) | 3 | 3 | Rattus norvegicus (Rat) |
| | | PREDICTED: SIMILAR | | | GTP-BINDING NUCLEAR | | | ADAMTS-1 | | | CADHERIN-6 PRECURSOR. |
| | | TO HEAT SHOCK | | | PROTEIN RAN, TESTIS- | | | PRECURSOR. | | | [MASS = 88341 |
| | | PROTEIN HSP 90-BETA. | | | SPECIFIC ISOFORM. | | | [MASS = 105719 | | | |
| | | [MASS = 80701 | | | [MASS = 24451 | | | | | | |
| 3 | 3 | Rattus norvegicus (Rat) | 3 | 4 | Rattus norvegicus (Rat) | 2 | 2 | Rattus norvegicus (Rat) | 3 | 5 | Rattus norvegicus (Rat) |
| | | SPLICE ISOFORM 1 OF | | | PREDICTED: SIMILAR | | | BETA-2- | | | RIBOSOMAL PROTEIN S27A. |
| | | ATTRACTIN | | | TO ISOLEUCINE-TRNA | | | GLYCOPROTEIN 1 | | | [MASS = 17951 |
| | | PRECURSOR. | | | SYNTHETASE. | | | PRECURSOR. | | | |
| | | [MASS = 163296 | | | [MASS = 144169 | | | [MASS = 33197 | | | |
| 3 | 3 | Rattus norvegicus (Rat) | 3 | 3 | Rattus norvegicus (Rat) | 2 | 2 | Rattus norvegicus (Rat) | 3 | 3 | Rattus norvegicus (Rat) |
| | | 40S RIBOSOMAL | | | HISTONE H1.0. | | | BONE | | | PREDICTED: NIDOGEN 2. |
| | | PROTEIN S3. | | | [MASS = 20754 | | | MORPHOGENETIC | | | [MASS = 173960 |
| | | [MASS = 26674 | | | | | | PROTEIN 1. | | | |
| | | | | | | | | [MASS = 111332 | | | |
| 3 | 7 | Rattus norvegicus (Rat) | 2 | 2 | Rattus norvegicus (Rat) | 2 | 2 | Rattus norvegicus (Rat) | 3 | 3 | Rattus norvegicus (Rat) |
| | | PREDICTED similar to | | | COFILIN-1. | | | RECEPTOR-LIKE | | | PREDICTED: SIMILAR TO |
| | | HEAT SHOCK PROTEIN | | | [MASS = 24588 | | | PROTEIN TYROSINE | | | UBIQUITIN SPECIFIC |
| | | 86. [MASS = 56953 | | | | | | PHOSPHATASE GAMMA | | | PROTEASE 9, X-LINKED. |
| | | | | | | | | B-TYPE ISOFORM. | | | [MASS = 290681 |
| | | | | | | | | [MASS = 156024 | | | |
| 3 | 3 | Rattus norvegicus (Rat) | 2 | 4 | Rattus norvegicus (Rat) | 2 | 6 | Rattus norvegicus (Rat) | 3 | 3 | Rattus norvegicus (Rat) |
| | | GAMMA-GLUTAMYL | | | ALPHA-2-HS- | | | DERMCIDIN. | | | SMC4L1 PROTEIN. |
| | | HYDROLASE | | | GLYCOPROTEIN | | | [MASS = 11284 | | | [MASS = 146806 |
| | | PRECURSOR. | | | PRECURSOR. | | | | | | |
| | | [MASS = 35830 | | | [MASS = 37982 | | | | | | |
| 3 | 3 | Rattus norvegicus (Rat) | 2 | 2 | Rattus norvegicus (Rat) | 2 | 2 | Rattus norvegicus (Rat) | 3 | 3 | Rattus norvegicus (Rat) |
| | | QUIESCIN Q6. | | | PREDICTED: SIMILAR | | | CALUMENIN | | | PREDICTED: similar to |
| | | [MASS = 82412 | | | TO LERK-5. | | | PRECURSOR. | | | Heterogeneous nuclear |
| | | | | | [MASS = 37282 | | | [MASS = 36997 | | | ribonucleoproteins |
| | | | | | | | | | | | A2/B1. [MASS = 32468 |
| 3 | 3 | Rattus norvegicus (Rat) | 2 | 3 | Rattus norvegicus (Rat) L- | 2 | 2 | Rattus norvegicus (Rat) | 3 | 3 | Rattus norvegicus (Rat) |
| | | PREDICTED: SIMILAR | | | LACTATE | | | PREDICTED: similar to | | | KINESIN-1 HEAVY CHAIN. |
| | | TO DNA REPLICATION | | | DEHYDROGENASE B | | | Periostin precursor (PN) | | | [MASS = 109531 |
| | | LICENSING FACTOR | | | CHAIN. [MASS = 36481 | | | (Osteoblast-specific | | | |
| | | MCM3. [MASS = 83429 | | | | | | factor 2) (OSF-2). | | | |
| | | | | | | | | [MASS = 90252 | | | |
| 3 | 3 | Rattus norvegicus (Rat) | 2 | 2 | Rattus norvegicus (Rat) | 2 | 8 | Rattus norvegicus (Rat) | 3 | 3 | Rattus norvegicus (Rat) |
| | | DELETED IN | | | PREDICTED: | | | AMBP PROTEIN | | | ADAMTS-1 PRECURSOR. |
| | | COLORECTAL CANCER. | | | PROTOCADHERIN 12. | | | PRECURSOR. | | | [MASS = 105719 |
| | | [MASS = 158142 | | | [MASS = 127964 | | | [MASS = 38851 | | | |
| 3 | 3 | Rattus norvegicus (Rat) | 2 | 2 | Rattus norvegicus (Rat) | 2 | 2 | Rattus norvegicus (Rat) | 3 | 3 | Rattus norvegicus (Rat) |
| | | ALPHA-ACTININ-1. | | | PREDICTED: ATPASE, | | | PROBABLE G-PROTEIN | | | PREDICTED: SIMILAR TO |
| | | [MASS = 102960 | | | H+ TRANSPORTING, | | | COUPLED RECEPTOR | | | PLEXIN-B2 PRECURSOR. |
| | | | | | LYSOSOMAL | | | 116 PRECURSOR. | | | [MASS = 216119 |
| | | | | | ACCESSORY PROTEIN | | | [MASS = 149446 | | | |
| | | | | | 2. [MASS = 66094 | | | | | | |
| 3 | 3 | Rattus norvegicus (Rat) | 2 | 2 | Rattus norvegicus (Rat) | 2 | 2 | Rattus norvegicus (Rat) | 3 | 3 | Rattus norvegicus (Rat) |
| | | COATOMER SUBUNIT | | | PREDICTED: SIMILAR | | | PLATELET | | | PREDICTED: KINESIN FAMILY |
| | | BETA′. [MASS = 102420 | | | TO HEAT SHOCK | | | ENDOTHELIAL CELL | | | MEMBER 4. [MASS = 139682 |
| | | | | | PROTEIN HSP 90-BETA. | | | ADHESION MOLECULE | | | |
| | | | | | [MASS = 80701 | | | PRECURSOR. | | | |
| | | | | | | | | [MASS = 76189 | | | |
| 3 | 4 | Rattus norvegicus (Rat) | 2 | 2 | Rattus norvegicus (Rat) | 2 | 3 | Rattus norvegicus (Rat) | 3 | 3 | Rattus norvegicus (Rat) NON- |
| | | PREDICTED: SIMILAR | | | IGH-1A PROTEIN. | | | PREDICTED similar to | | | ERYTHROCYTE BETA- |
| | | TO P59 IMMUNOPHILIN. | | | [MASS = 51403 | | | FIBRINOGEN, GAMMA | | | SPECTRIN. [MASS = 251205 |
| | | [MASS = 80396 | | | | | | POLYPEPTIDE. | | | |
| | | | | | | | | [MASS = 49121 | | | |
| 3 | 3 | Rattus norvegicus (Rat) | 2 | 2 | Rattus norvegicus (Rat) | 2 | 2 | Rattus norvegicus (Rat) | 3 | 3 | Rattus norvegicus (Rat) D-3- |
| | | FIBRINOGEN BETA | | | FIBRILLIN-2. | | | PREDICTED: | | | PHOSPHOGLYCERATE |
| | | CHAIN PRECURSOR. | | | [MASS = 313374 | | | AMINOPEPTIDASE | | | DEHYDROGENASE. |
| | | [MASS = 54303 | | | | | | PUROMYCIN | | | [MASS = 56362 |
| | | | | | | | | SENSITIVE. | | | |
| | | | | | | | | [MASS = 103344 | | | |
| 3 | 3 | Rattus norvegicus (Rat) | 2 | 2 | Rattus norvegicus (Rat) | 2 | 2 | Rattus norvegicus (Rat) | 3 | 3 | Rattus norvegicus (Rat) |
| | | PREDICTED: SIMILAR | | | NUCLEOSOME | | | SPLICE ISOFORM PAM- | | | SPLICE ISOFORM 1 OF |
| | | TO 40S RIBOSOMAL | | | ASSEMBLY PROTEIN 1- | | | 1 OF PEPTIDYL- | | | ACETYL-COA CARBOXYLASE |
| | | PROTEIN S3. | | | LIKE 1. [MASS = 45373 | | | GLYCINE ALPHA- | | | 1. [MASS = 265421 |
| | | [MASS = 26630 | | | | | | AMIDATING | | | |
| | | | | | | | | MONOOXYGENASE | | | |
| | | | | | | | | PRECURSOR. | | | |
| | | | | | | | | [MASS = 108819 | | | |
| 3 | 3 | Rattus norvegicus (Rat) | 2 | 2 | Rattus norvegicus (Rat) | 2 | 2 | Rattus norvegicus (Rat) | 3 | 3 | Rattus norvegicus (Rat) |
| | | ALPHA-1-ACID | | | PREDICTED: SIMILAR | | | PREDICTED: SIMILAR | | | NUCLEOLIN. [MASS = 77276 |
| | | GLYCOPROTEIN | | | TO UBIQUITIN- | | | TO FILAMIN A. | | | |
| | | PRECURSOR. | | | CONJUGATING ENZYME | | | [MASS = 290169 | | | |
| | | [MASS = 23575 | | | E2 L3. [MASS = 17862 | | | | | | |
| 3 | 3 | Rattus norvegicus (Rat) | 2 | 2 | Rattus norvegicus (Rat) | 2 | 2 | Rattus norvegicus (Rat) | 3 | 3 | Rattus norvegicus (Rat) |
| | | ALPHA-1- | | | FRUCTOSE- | | | PREDICTED: SIMILAR | | | PREDICTED: SIMILAR TO |
| | | MACROGLOBULIN. | | | BISPHOSPHATE | | | TO PROTOCADHERIN | | | SEROTRANSFERRIN |
| | | [MASS = 167125 | | | ALDOLASE A. | | | 19 PRECURSOR. | | | PRECURSOR. [MASS = 76607 |
| | | | | | [MASS = 39221 | | | [MASS = 125989 | | | |
| 3 | 3 | Rattus norvegicus (Rat) | 2 | 2 | Rattus norvegicus (Rat) | 2 | 2 | Rattus norvegicus (Rat) | 3 | 4 | Rattus norvegicus (Rat) |
| | | LIVER | | | APOLIPOPROTEIN M | | | VITAMIN K-DEPENDENT | | | PREDICTED: HYPOTHETICAL |
| | | CARBOXYLESTERASE 1 | | | PRECURSOR. | | | PROTEIN S | | | PROTEIN XP_579585. |
| | | PRECURSOR. | | | [MASS = 21513 | | | PRECURSOR. | | | [MASS = 275729 |
| | | [MASS = 60175 | | | | | | [MASS = 74627 | | | |
| 3 | 3 | Rattus norvegicus (Rat) | 2 | 2 | Rattus norvegicus (Rat) | 2 | 2 | Rattus norvegicus (Rat) | 3 | 3 | Rattus norvegicus (Rat) |
| | | SIMILAR TO RIKEN | | | HETEROGENEOUS | | | SEMA4B PROTEIN | | | PREDICTED: SIMILAR TO |
| | | CDNA 2810409H07. | | | NUCLEAR | | | (FRAGMENT). | | | COLONIC AND HEPATIC |
| | | [MASS = 44535 | | | RIBONUCLEOPROTEIN | | | [MASS = 79477 | | | TUMOR OVER-EXPRESSED |
| | | | | | C. [MASS = 34385 | | | | | | PROTEIN ISOFORM A. |
| | | | | | | | | | | | [MASS = 198456 |
| 3 | 4 | Rattus norvegicus (Rat) | 2 | 2 | Rattus norvegicus (Rat) | 2 | 2 | Rattus norvegicus (Rat) | 3 | 3 | Rattus norvegicus (Rat) |
| | | PREDICTED: SIMILAR | | | IG KAPPA CHAIN C | | | RHO GDP | | | PREDICTED: SIMILAR TO |
| | | TO PHOSPHOLIPID | | | REGION, B ALLELE. | | | DISSOCIATION | | | FILAMIN B. [MASS = 291469 |
| | | TRANSFER PROTEIN. | | | [MASS = 11601 | | | INHIBITOR (GDI) ALPHA. | | | |
| | | [MASS = 65430 | | | | | | [MASS = 23407 | | | |
| 3 | 3 | Rattus norvegicus (Rat) | 2 | 4 | Rattus norvegicus (Rat) | 2 | 2 | Rattus norvegicus (Rat) | 3 | 3 | Rattus norvegicus (Rat) |
| | | 26S PROTEASE | | | PYRUVATE KINASE, | | | PREDICTED: SIMILAR | | | PREDICTED: SIMILAR TO |
| | | REGULATORY SUBUNIT | | | MUSCLE. [MASS = 57976 | | | TO EXPRESSED | | | CHROMOSOME |
| | | 8. [MASS = 45626 | | | | | | SEQUENCE C79407. | | | CONDENSATION PROTEIN G. |
| | | | | | | | | [MASS = 113188 | | | [MASS = 113137 |
| 3 | 5 | Rattus norvegicus (Rat) | 2 | 2 | Rattus norvegicus (Rat) | 2 | 2 | Rattus norvegicus (Rat) | 3 | 13 | Rattus norvegicus (Rat) AMBP |
| | | PREDICTED: SIMILAR | | | NUCLEOSIDE | | | MYOSIN-10. | | | PROTEIN PRECURSOR. |
| | | TO HEAT SHOCK | | | DIPHOSPHATE KINASE | | | [MASS = 228965 | | | [MASS = 38851 |
| | | PROTEIN HSP 90-BETA. | | | A. [MASS = 17193 | | | | | | |
| | | [MASS = 54660 | | | | | | | | | |
| 3 | 3 | Rattus norvegicus (Rat) | 2 | 2 | Rattus norvegicus (Rat) | 2 | 2 | Rattus norvegicus (Rat) | 3 | 3 | Rattus norvegicus (Rat) |
| | | SUPEROXIDE | | | TUBULIN, BETA, 2. | | | GM2 GANGLIOSIDE | | | Neogenin precursor. |
| | | DISMUTASE. | | | [MASS = 49801 | | | ACTIVATOR PROTEIN. | | | [MASS = 156144 |
| | | [MASS = 15780 | | | | | | [MASS = 21493 | | | |
| 3 | 3 | Rattus norvegicus (Rat) | 2 | 2 | Rattus norvegicus (Rat) | 2 | 2 | Rattus norvegicus (Rat) | 3 | 3 | Rattus norvegicus (Rat) |
| | | PREDICTED: | | | PREDICTED: | | | PREDICTED: | | | LEUCYL-TRNA SYNTHETASE. |
| | | HYPOTHETICAL | | | GLYCOPROTEIN-4- | | | PROCOLLAGEN, TYPE | | | [MASS = 134279 |
| | | PROTEIN XP_579585. | | | BETA- | | | XII, ALPHA 1. | | | |
| | | [MASS = 275729 | | | GALACTOSYLTRANSFERASE | | | [MASS = 367709 | | | |
| | | | | | 2. [MASS = 44484 | | | | | | |
| 3 | 3 | Rattus norvegicus (Rat) | 2 | 2 | Rattus norvegicus (Rat) | 2 | 2 | Rattus norvegicus (Rat) | 3 | 3 | Rattus norvegicus (Rat) |
| | | LOW-DENSITY | | | ALPHA-1-ACID | | | EPSILON 2 GLOBIN. | | | PREDICTED: |
| | | LIPOPROTEIN | | | GLYCOPROTEIN | | | [MASS = 16388 | | | THROMBOSPONDIN 4. |
| | | RECEPTOR | | | PRECURSOR. | | | | | | [MASS = 121361 |
| | | PRECURSOR. | | | [MASS = 23575 | | | | | | |
| | | [MASS = 96622 | | | | | | | | | |
| 3 | 3 | Rattus norvegicus (Rat) | 2 | 2 | Rattus norvegicus (Rat) | 2 | 4 | Rattus norvegicus (Rat) | 3 | 3 | Rattus norvegicus (Rat) |
| | | PREDICTED: SIMILAR | | | BETA-1,3-N- | | | HISTONE H1.2. | | | PREDICTED: SIMILAR TO |
| | | TO PROTOCADHERIN 18 | | | ACETYLGLUCOSAMINYL- | | | [MASS = 21856 | | | TALIN 2. [MASS = 273281 |
| | | PRECURSOR. | | | TRANSFERASE | | | | | | |
| | | | | | LUNATIC FRINGE. | | | | | | |
| | | [MASS = 123552 | | | [MASS = 41958 | | | | | | |
| 3 | 3 | Rattus norvegicus (Rat) | 2 | 2 | Rattus norvegicus (Rat) | 2 | 2 | Rattus norvegicus (Rat) | 3 | 3 | Rattus norvegicus (Rat) |
| | | SPLICE ISOFORM B OF | | | PREDICTED: SIMILAR | | | 60S RIBOSOMAL | | | PREDICTED: SIMILAR TO |
| | | AP-1 COMPLEX | | | TO ELASTIN | | | PROTEIN L8. | | | OLLISTATIN-LIKE 5. |
| | | SUBUNIT BETA-1. | | | MICROFIBRIL | | | [MASS = 27893 | | | [MASS = 95918 |
| | | [MASS = 103873 | | | INTERFACER 1. | | | | | | |
| | | | | | [MASS = 107560 | | | | | | |
| 3 | 3 | Rattus norvegicus (Rat) | 2 | 2 | Rattus norvegicus (Rat) | 2 | 3 | Rattus norvegicus (Rat) | 3 | 3 | Rattus norvegicus (Rat) |
| | | COLLAGEN ALPHA-2(I) | | | PREDICTED: SIMILAR | | | PREDICTED: SIMILAR | | | THROMBOSPONDIN 1. |
| | | CHAIN PRECURSOR. | | | TO HETEROGENEOUS | | | TO LIPOPROTEIN | | | [MASS = 129671 |
| | | [MASS = 129564 | | | NUCLEAR | | | RECEPTOR-RELATED | | | |
| | | | | | RIBONUCLEOPROTEIN | | | PROTEIN. | | | |
| | | | | | A2/B1. [MASS = 38284 | | | [MASS = 504889 | | | |
| 3 | 3 | Rattus norvegicus (Rat) | 2 | 2 | Rattus norvegicus (Rat) | 2 | 2 | Rattus norvegicus (Rat) | 3 | 4 | Rattus norvegicus (Rat) |
| | | SHEN-DAN. | | | TPA: proteasome subunit | | | PREDICTED: SIMILAR | | | STATHMIN. [MASS = 17157 |
| | | [MASS = 131080 | | | beta type 6-like. | | | TO SUSHI, VON | | | |
| | | | | | [MASS = 25304 | | | WILLEBRAND FACTOR | | | |
| | | | | | | | | TYPE A, EGF AND | | | |
| | | | | | | | | PENTRAXIN DOMAIN | | | |
| | | | | | | | | CONTAINING 1. | | | |
| | | | | | | | | [MASS = 383558 | | | |
| 3 | 3 | Rattus norvegicus (Rat) | 2 | 2 | Rattus norvegicus (Rat) | 2 | 2 | Rattus norvegicus (Rat) | 3 | 4 | Rattus norvegicus (Rat) |
| | | SPARC-LIKE PROTEIN 1 | | | BETA-2- | | | GTP-BINDING NUCLEAR | | | PREDICTED: |
| | | PRECURSOR. | | | GLYCOPROTEIN 1 | | | PROTEIN RAN, TESTIS- | | | GLYCOPROTEIN-4-BETA- |
| | | [MASS = 70634 | | | PRECURSOR. | | | SPECIFIC ISOFORM. | | | GALACTOSYLTRANSFERASE |
| | | | | | [MASS = 33197 | | | [MASS = 24451 | | | 2. [MASS = 44484 |
| 3 | 3 | Rattus norvegicus (Rat) | 2 | 2 | Rattus norvegicus (Rat) | 2 | 2 | Rattus norvegicus (Rat) | 3 | 4 | Rattus norvegicus (Rat) |
| | | NUCLEOSIDE | | | GRP78 BINDING | | | 14-3-3 PROTEIN | | | HISTONE H1.2. [MASS = 21856 |
| | | DIPHOSPHATE KINASE | | | PROTEIN. | | | EPSILON. [MASS = 29174 | | | |
| | | B. [MASS = 17283 | | | [MASS = 110574 | | | | | | |
| 3 | 3 | Rattus norvegicus (Rat) | 2 | 2 | Rattus norvegicus (Rat) | 2 | 2 | Rattus norvegicus (Rat) | 3 | 3 | Rattus norvegicus (Rat) |
| | | D-3- | | | HAPTOGLOBIN | | | INSULIN-LIKE GROWTH | | | PEROXIREDOXIN-1. |
| | | PHOSPHOGLYCERATE | | | PRECURSOR. | | | FACTOR-BINDING | | | [MASS = 22109 |
| | | DEHYDROGENASE. | | | [MASS = 38549 | | | PROTEIN COMPLEX | | | |
| | | [MASS = 56362 | | | | | | ACID LABILE CHAIN | | | |
| | | | | | | | | PRECURSOR. | | | |
| | | | | | | | | [MASS = 66812 | | | |
| 3 | 3 | Rattus norvegicus (Rat) | 2 | 3 | Rattus norvegicus (Rat) | | | | 2 | 3 | Rattus norvegicus (Rat) |
| | | PROTEIN DISULFIDE- | | | PREDICTED: similar to | | | | | | ALPHA-ENOLASE. |
| | | ISOMERASE A3 | | | alpha 1 type II collagen. | | | | | | [MASS = 46997 |
| | | PRECURSOR. | | | [MASS = 138706 | | | | | | |
| | | [MASS = 57079 | | | | | | | | | |
| 3 | 3 | Rattus norvegicus (Rat) | 2 | 2 | Rattus norvegicus (Rat) | | | | 2 | 4 | Rattus norvegicus (Rat) RAB |
| | | LARGE PROLINE-RICH | | | COLLAGEN TYPE | | | | | | GDP DISSOCIATION |
| | | PROTEIN BAT3. | | | A1(XI)7-8. [MASS = 45691 | | | | | | INHIBITOR ALPHA. |
| | | [MASS = 114647 | | | | | | | | | [MASS = 50537 |
| 3 | 5 | Rattus norvegicus (Rat) | 2 | 2 | Rattus norvegicus (Rat) | | | | 2 | 2 | Rattus norvegicus (Rat) |
| | | PREDICTED: SIMILAR | | | PROPROTEIN | | | | | | ANGIOTENSIN-CONVERTING |
| | | TO IMPORTIN 9. | | | CONVERTASE | | | | | | ENZYME, SOMATIC ISOFORM |
| | | [MASS = 131739 | | | SUBTILISIN/KEXIN TYPE | | | | | | PRECURSOR. [MASS = 150908 |
| | | | | | 9 PRECURSOR. | | | | | | |
| | | | | | [MASS = 74709 | | | | | | |
| 3 | 3 | Rattus norvegicus (Rat) | 2 | 2 | Rattus norvegicus (Rat) | | | | 2 | 2 | Rattus norvegicus (Rat) |
| | | PREDICTED: SIMILAR | | | NUCLEIC ACID BINDING | | | | | | PREDICTED: SIMILAR TO |
| | | TO 25 KDA FK506- | | | FACTOR PRM10. | | | | | | ALPHA ENOLASE. |
| | | BINDING PROTEIN. | | | [MASS = 33815 | | | | | | [MASS = 46489 |
| | | [MASS = 25179 | | | | | | | | | |
| 3 | 3 | Rattus norvegicus (Rat) | 2 | 2 | Rattus norvegicus (Rat) | | | | 2 | 4 | Rattus norvegicus (Rat) |
| | | DNA POLYMERASE | | | PREDICTED: SIMILAR | | | | | | PREDICTED similar to HEAT |
| | | DELTA CATALYTIC | | | TO ALPHA 3 TYPE VI | | | | | | SHOCK PROTEIN 86. |
| | | SUBUNIT. | | | COLLAGEN ISOFORM 1 | | | | | | [MASS = 56953 |
| | | [MASS = 123601 | | | PRECURSOR. | | | | | | |
| | | | | | [MASS = 369017 | | | | | | |
| 3 | 3 | Rattus norvegicus (Rat) | 2 | 2 | Rattus norvegicus (Rat) | | | | 2 | 2 | Rattus norvegicus (Rat) ALPHA |
| | | CHAPERONIN SUBUNIT | | | MANNOSIDASE 2, | | | | | | ACTININ 4. [MASS = 104915 |
| | | 6A. [MASS = 58017 | | | ALPHA B1. | | | | | | |
| | | | | | [MASS = 114327 | | | | | | |
| 3 | 3 | Rattus norvegicus (Rat) | 2 | 3 | Rattus norvegicus (Rat) | | | | 2 | 2 | Rattus norvegicus (Rat) |
| | | SPLICE ISOFORM 2 OF | | | METLLOPROTEINASE | | | | | | SPLICE ISOFORM APP770 OF |
| | | INTERLEUKIN | | | INHIBITOR 1 | | | | | | AMYLOID BETA A4 PROTEIN |
| | | ENHANCER-BINDING | | | PRECURSOR. | | | | | | PRECURSOR (FRAGMENT). |
| | | FACTOR 3. | | | [MASS = 23794 | | | | | | [MASS = 86704 |
| | | [MASS = 97680 | | | | | | | | | |
| 3 | 3 | Rattus norvegicus (Rat) | 2 | 2 | Rattus norvegicus (Rat) | | | | 2 | 2 | Rattus norvegicus (Rat) |
| | | SPLICE ISOFORM 1 OF | | | CHAPERONIN | | | | | | PREDICTED similar to T- |
| | | REELIN PRECURSOR. | | | CONTAINING TCP1, | | | | | | KININOGEN 2 PRECURSOR |
| | | [MASS = 387531 | | | SUBUNIT 5. | | | | | | (Fragment). [MASS = 72419 |
| | | | | | [MASS = 59537 | | | | | | |
| 3 | 3 | Rattus norvegicus (Rat) | 2 | 2 | Rattus norvegicus (Rat) | | | | 2 | 2 | Rattus norvegicus (Rat) |
| | | PREDICTED: SIMILAR | | | PREDICTED: SIMILAR | | | | | | PREDICTED: SIMILAR TO |
| | | TO DNA REPLICATION | | | TO VESICULAR | | | | | | HEAT SHOCK 70 KDA |
| | | LICENSING FACTOR | | | INTEGRAL-MEMBRANE | | | | | | PROTEIN 4 LIKE. |
| | | MCM2. [MASS = 102272 | | | PROTEIN VIP36 | | | | | | [MASS = 136266 |
| | | | | | PRECURSOR. | | | | | | |
| | | | | | [MASS = 40393 | | | | | | |
| 3 | 3 | Rattus norvegicus (Rat) | 2 | 2 | Rattus norvegicus (Rat) | | | | 2 | 4 | Rattus norvegicus (Rat) |
| | | 40S RIBOSOMAL | | | IRON-RESPONSIVE | | | | | | TUBULIN BETA-3 CHAIN. |
| | | PROTEIN S18. | | | ELEMENT-BINDING | | | | | | [MASS = 50419 |
| | | [MASS = 17719 | | | PROTEIN 1. | | | | | | |
| | | | | | [MASS = 98128 | | | | | | |
| 3 | 4 | Rattus norvegicus (Rat) | 2 | 2 | Rattus norvegicus (Rat) | | | | 2 | 2 | Rattus norvegicus (Rat) |
| | | HISTIDINE-RICH | | | RIBONUCLEOTIDE | | | | | | EUKARYOTIC TRANSLATION |
| | | GLYCOPROTEIN. | | | REDUCTASE M1. | | | | | | INITIATION FACTOR 5A-1. |
| | | [MASS = 57581 | | | [MASS = 90293 | | | | | | [MASS = 16701 |
| 3 | 4 | Rattus norvegicus (Rat) | 2 | 2 | Rattus norvegicus (Rat) | | | | 2 | 4 | Rattus norvegicus (Rat) |
| | | HISTONE H1.2. | | | 60S ACIDIC RIBOSOMAL | | | | | | PYRUVATE KINASE, |
| | | [MASS = 21856 | | | PROTEIN P0. | | | | | | MUSCLE. [MASS = 57976 |
| | | | | | [MASS = 34215 | | | | | | |
| 3 | 3 | Rattus norvegicus (Rat) | 2 | 4 | Rattus norvegicus (Rat) | | | | 2 | 2 | Rattus norvegicus (Rat) |
| | | DYNACTIN-1. | | | AMBP PROTEIN | | | | | | PREDICTED: SIMILAR TO |
| | | [MASS = 141930 | | | PRECURSOR. | | | | | | PEPTIDOGLYCAN |
| | | | | | [MASS = 38851 | | | | | | RECOGNITION PROTEIN 2. |
| | | | | | | | | | | | [MASS = 39775 |
| 3 | 3 | Rattus norvegicus (Rat) | 2 | 2 | Rattus norvegicus (Rat) | | | | 2 | 2 | Rattus norvegicus (Rat) L- |
| | | PREDICTED: SIMILAR | | | SYNTENIN-1. | | | | | | LACTATE DEHYDROGENASE |
| | | TO REGULATOR OF | | | [MASS = 32423 | | | | | | B CHAIN. [MASS = 36481 |
| | | NONSENSE | | | | | | | | | |
| | | TRANSCRIPTS 1. | | | | | | | | | |
| | | [MASS = 88226 | | | | | | | | | |
| 3 | 3 | Rattus norvegicus (Rat) | 2 | 3 | Rattus norvegicus (Rat) | | | | 2 | 2 | Rattus norvegicus (Rat) IGH-1A |
| | | VIGILIN. [MASS = 141584 | | | RECEPTOR-LIKE | | | | | | PROTEIN. [MASS = 51403 |
| | | | | | PROTEIN TYROSINE | | | | | | |
| | | | | | PHOSPHATASE KAPPA | | | | | | |
| | | | | | EXTRACELLULAR | | | | | | |
| | | | | | REGION. [MASS = 56159 | | | | | | |
| 3 | 4 | Rattus norvegicus (Rat) | 2 | 2 | Rattus norvegicus (Rat) | | | | 2 | 4 | Rattus norvegicus (Rat) |
| | | 60S RIBOSOMAL | | | PREDICTED: SIMILAR | | | | | | ALPHA-2-HS-GLYCOPROTEIN |
| | | PROTEIN L3. | | | TO HEMICENTIN 1. | | | | | | PRECURSOR. [MASS = 37982 |
| | | [MASS = 46005 | | | [MASS = 639647 | | | | | | |
| 3 | 3 | Rattus norvegicus (Rat) | 2 | 3 | Rattus norvegicus (Rat) | | | | 2 | 2 | Rattus norvegicus (Rat) |
| | | ALPHA-MANNOSIDASE | | | EPITHELIAL-CADHERIN | | | | | | SPLICE ISOFORM IIBA OF |
| | | 2. [MASS = 131242 | | | PRECURSOR. | | | | | | DYNAMIN-2. [MASS = 98246 |
| | | | | | [MASS = 98715 | | | | | | |
| 3 | 3 | Rattus norvegicus (Rat) | 2 | 2 | Rattus norvegicus (Rat) | | | | 2 | 2 | Rattus norvegicus (Rat) |
| | | PROTOCADHERIN. | | | VASCULAR CELL | | | | | | SORTILIN PRECURSOR. |
| | | [MASS = 505997 | | | ADHESION PROTEIN 1 | | | | | | [MASS = 91169 |
| | | | | | PRECURSOR. | | | | | | |
| | | | | | [MASS = 81246 | | | | | | |
| 3 | 3 | Rattus norvegicus (Rat) | 2 | 2 | Rattus norvegicus (Rat) | | | | 2 | 3 | Rattus norvegicus (Rat) |
| | | PROLIFERATION- | | | PREDICTED: SIMILAR | | | | | | HEMOGLOBIN BETA-1 |
| | | ASSOCIATED 2G4, | | | TO CG1841-PA, | | | | | | SUBUNIT. [MASS = 15848 |
| | | 38 KDA. [MASS = 43657 | | | ISOFORM A. | | | | | | |
| | | | | | [MASS = 52522 | | | | | | |
| 3 | 5 | Rattus norvegicus (Rat) | 2 | 2 | Rattus norvegicus (Rat) | | | | 2 | 2 | Rattus norvegicus (Rat) |
| | | STATHMIN. | | | CLUSTERIN | | | | | | TRANSLATIONALLY- |
| | | [MASS = 17157 | | | PRECURSOR. | | | | | | CONTROLLED TUMOR |
| | | | | | [MASS = 51375 | | | | | | PROTEIN. [MASS = 19462 |
| 3 | 3 | Rattus norvegicus (Rat) | 2 | 2 | Rattus norvegicus (Rat) | | | | 2 | 4 | Rattus norvegicus (Rat) ALPHA |
| | | PREDICTED: similar to | | | PREDICTED: SIMILAR | | | | | | 2 MACROGLOBULIN |
| | | 60S ribosomal protein | | | TO 40S RIBOSOMAL | | | | | | CARDIAC ISOFORM. |
| | | L38. [MASS = 8215 | | | PROTEIN S3. | | | | | | [MASS = 163218 |
| | | | | | [MASS = 26630 | | | | | | |
| 3 | 3 | Rattus norvegicus (Rat) | 2 | 2 | Rattus norvegicus (Rat) | | | | 2 | 2 | Rattus norvegicus (Rat) RAT |
| | | 40S RIBOSOMAL | | | PREDICTED to similar to | | | | | | ALPHA(1)-INHIBITOR 3, |
| | | PROTEIN S7. | | | HEAT SHOCK PROTEIN | | | | | | VARIANT I PRECURSOR. |
| | | [MASS = 22127 | | | HSP 90-BETA. | | | | | | [MASS = 165326 |
| | | | | | [MASS = 50669 | | | | | | |
| 3 | 3 | Rattus norvegicus (Rat) | 2 | 2 | Rattus norvegicus (Rat) | | | | 2 | 2 | Rattus norvegicus (Rat) |
| | | COLLAGEN ALPHA-1(V) | | | 60S RIBOSOMAL | | | | | | PROTEASOME SUBUNIT |
| | | CHAIN PRECURSOR. | | | PROTEIN L13. | | | | | | BETA TYPE 1. [MASS = 26479 |
| | | [MASS = 184610 | | | [MASS = 24178 | | | | | | |
| 3 | 3 | Rattus norvegicus (Rat) | 2 | 2 | Rattus norvegicus (Rat) | | | | 2 | 2 | Rattus norvegicus (Rat) |
| | | HAUSP. [MASS = 128431 | | | ARCADLIN. | | | | | | PREDICTED: SIMILAR TO |
| | | | | | [MASS = 103800 | | | | | | IMMUNOGLOBULIN HEAVY |
| | | | | | | | | | | | CHAIN. [MASS = 120447 |
| 3 | 3 | Rattus norvegicus (Rat) | 2 | 2 | Rattus norvegicus (Rat) | | | | 2 | 2 | Rattus norvegicus (Rat) |
| | | PROTOCADHERIN | | | PREDICTED: SIMILAR | | | | | | SPLICE ISOFORM B OF AP-1 |
| | | GAMMA SUBFAMILY C, | | | TO CELLULAR | | | | | | COMPLEX SUBUNIT BETA-1. |
| | | 3. [MASS = 101038 | | | APOPTOSIS | | | | | | [MASS = 103873 |
| | | | | | SUSCEPTIBILITY | | | | | | |
| | | | | | PROTEIN. | | | | | | |
| | | | | | [MASS = 110214 | | | | | | |
| 3 | 5 | Rattus norvegicus (Rat) | 2 | 2 | Rattus norvegicus (Rat) | | | | 2 | 3 | Rattus norvegicus (Rat) GPI- |
| | | NUCLEOLIN. | | | PREDICTED: SIMILAR | | | | | | ANCHORED MEMBRANE |
| | | [MASS = 77276 | | | TO SEMAPHORIN 6D-4. | | | | | | PROTEIN 1. [MASS = 75707 |
| | | | | | [MASS = 159473 | | | | | | |
| 3 | 3 | Rattus norvegicus (Rat) | 2 | 2 | Rattus norvegicus (Rat) | | | | 2 | 2 | Rattus norvegicus (Rat) T- |
| | | PREDICTED: SIMILAR | | | 60S RIBOSOMAL | | | | | | COMPLEX PROTEIN 1 |
| | | TO UBIQUITIN SPECIFIC | | | PROTEIN L3. | | | | | | SUBUNIT DELTA. |
| | | PROTEASE 9, X-LINKED. | | | [MASS = 46005 | | | | | | [MASS = 57968 |
| | | [MASS = 290681 | | | | | | | | | |
| 3 | 3 | Rattus norvegicus (Rat) | 2 | 2 | Rattus norvegicus (Rat) | | | | 2 | 2 | Rattus norvegicus (Rat) |
| | | PREDICTED: SIMILAR | | | TENASCIN | | | | | | PROTECTIVE PROTEIN FOR |
| | | TO ENHANCER-TRAP- | | | (FRAGMENT). | | | | | | BETA-GALACTOSIDASE. |
| | | LOCUS-1. | | | [MASS = 67815 | | | | | | [MASS = 51216 |
| | | [MASS = 159154 | | | | | | | | | |
| 3 | 6 | Rattus norvegicus (Rat) | 2 | 2 | Rattus norvegicus (Rat) | | | | 2 | 2 | Rattus norvegicus (Rat) |
| | | PHOSPHOGLYCERATE | | | HEAT SHOCK 70 KDA | | | | | | PREDICTED similar to |
| | | MUTASE 2. | | | PROTEIN 1A/1B. | | | | | | Proteasome 26S subunit, |
| | | [MASS = 28624 | | | [MASS = 70185 | | | | | | ATPase 3. [MASS = 50509 |
| 3 | 5 | Rattus norvegicus (Rat) | 2 | 2 | Rattus norvegicus (Rat) | | | | 2 | 2 | Rattus norvegicus (Rat) |
| | | DAMAGE-SPECIFIC DNA | | | PREDICTED: similar to | | | | | | PREDICTED: ADAPTOR- |
| | | BINDING PROTEIN 1. | | | Slit-like 2. [MASS = 72321 | | | | | | RELATED PROTEIN |
| | | [MASS = 127059 | | | | | | | | | COMPLEX 1, GAMMA 1 |
| | | | | | | | | | | | SUBUNIT. [MASS = 91693 |
| 3 | 3 | Rattus norvegicus (Rat) | 2 | 2 | Rattus norvegicus (Rat) | | | | 2 | 2 | Rattus norvegicus (Rat) |
| | | PREDICTED: SIMILAR | | | PREDICTED: SIMILAR | | | | | | PREDICTED: SIMILAR TO |
| | | TO SIDEKICK 2. | | | TO POLY(RC)-BINDING | | | | | | HSPC263. [MASS = 37041 |
| | | [MASS = 466498 | | | PROTEIN 1. | | | | | | |
| | | | | | [MASS = 37498 | | | | | | |
| 3 | 3 | Rattus norvegicus (Rat) | 2 | 2 | Rattus norvegicus (Rat) | | | | 2 | 3 | Rattus norvegicus (Rat) |
| | | PEPTIDYLPROLYL | | | C-REACTIVE PROTEIN | | | | | | PREDICTED: SIMILAR TO |
| | | ISOMERASE C. | | | PRECURSOR. | | | | | | ALPHA NAC/1.9.2. PROTEIN. |
| | | [MASS = 23009 | | | [MASS = 25468 | | | | | | [MASS = 23384 |
| 3 | 3 | Rattus norvegicus (Rat) | 2 | 3 | Rattus norvegicus (Rat) | | | | 2 | 3 | Rattus norvegicus (Rat) |
| | | TENASCIN | | | PREDICTED similar to | | | | | | ALPHA-ACTININ-4. |
| | | (FRAGMENT). | | | FIBRINOGEN, GAMMA | | | | | | [MASS = 104786 |
| | | [MASS = 67815 | | | POLYPEPTIDE. | | | | | | |
| | | | | | [MASS = 49121 | | | | | | |
| 3 | 3 | Rattus norvegicus (Rat) | 2 | 2 | Rattus norvegicus (Rat) | | | | 2 | 2 | Rattus norvegicus (Rat) |
| | | PREDICTED: SIMILAR | | | NEUROSERPIN | | | | | | PREDICTED: VON |
| | | TO RIBOSOMAL | | | PRECURSOR. | | | | | | WILLEBRAND FACTOR. |
| | | PROTEIN L28. | | | [MASS = 46278 | | | | | | [MASS = 308474 |
| | | [MASS = 16313 | | | | | | | | | |
| 2 | 2 | Rattus norvegicus (Rat) | 2 | 2 | Rattus norvegicus (Rat) | | | | 2 | 2 | Rattus norvegicus (Rat) FATTY |
| | | ALPHA-ENOLASE. | | | PREDICTED: SIMILAR | | | | | | ACID-BINDING PROTEIN, |
| | | [MASS = 46997 | | | TO PUTATIVE PRE- | | | | | | BRAIN. [MASS = 14733 |
| | | | | | MRNA SPLICING | | | | | | |
| | | | | | FACTOR RNA | | | | | | |
| | | | | | HELICASE. | | | | | | |
| | | | | | [MASS = 90977 | | | | | | |
| 2 | 2 | Rattus norvegicus (Rat) | 2 | 3 | Rattus norvegicus (Rat) | | | | 2 | 2 | Rattus norvegicus (Rat) GM2 |
| | | PREDICTED similar to T- | | | SERINE (OR CYSTEINE | | | | | | GANGLIOSIDE ACTIVATOR |
| | | KININOGEN 2 | | | PROTEINASE | | | | | | PROTEIN. [MASS = 21493 |
| | | PRECURSOR | | | INHIBITOR, CLADE A | | | | | | |
| | | (Fragment) | | | (ALPHA-1 | | | | | | |
| | | [MASS = 72419 | | | ANTIPROTEINASE, | | | | | | |
| | | | | | ANTITRYPSIN), | | | | | | |
| | | | | | MEMBER 6. | | | | | | |
| | | | | | [MASS = 44671 | | | | | | |
| 2 | 3 | Rattus norvegicus (Rat) | 2 | 3 | Rattus norvegicus (Rat) | | | | 2 | 2 | Rattus norvegicus (Rat) SSB |
| | | HYRAC. [MASS = 31353 | | | HEMOPEXIN | | | | | | PROTEIN. [MASS = 43926 |
| | | | | | PRECURSOR. | | | | | | |
| | | | | | [MASS = 51291 | | | | | | |
| 2 | 2 | Rattus norvegicus (Rat) | 2 | 2 | Rattus norvegicus (Rat) | | | | 2 | 2 | Rattus norvegicus (Rat) |
| | | HEAT SHOCK-RELATED | | | PREDICTED: BRAIN | | | | | | MANNOSIDASE, ALPHA, |
| | | 70 KDA PROTEIN 2. | | | GLYCOGEN | | | | | | CLASS 1A, MEMBER 1. |
| | | [MASS = 69528 | | | PHOSPHORYLASE. | | | | | | [MASS = 73125 |
| | | | | | [MASS = 96738 | | | | | | |
| 2 | 3 | Rattus norvegicus (Rat) | 2 | 2 | Rattus norvegicus (Rat) | | | | 2 | 2 | Rattus norvegicus (Rat) |
| | | PROLIFERATING CELL | | | PREDICTED: SIMILAR | | | | | | VIGILIN. [MASS = 141584 |
| | | NUCLEAR ANTIGEN. | | | TO 40S RIBOSOMAL | | | | | | |
| | | [MASS = 28749 | | | PROTEIN S9. | | | | | | |
| | | | | | [MASS = 22648 | | | | | | |
| 2 | 2 | Rattus norvegicus (Rat) | 2 | 2 | Rattus norvegicus (Rat) | | | | 2 | 2 | Rattus norvegicus (Rat) |
| | | PREDICTED: SIMILAR | | | PREDICTED: SIMILAR | | | | | | PREDICTED-MATRIN-3. |
| | | TO ALPHA ENOLASE. | | | TO 60S RIBOSOMAL | | | | | | [MASS = 44733 |
| | | [MASS = 27286 | | | PROTEIN L12. | | | | | | |
| | | | | | [MASS = 17847 | | | | | | |
| 2 | 3 | Rattus norvegicus (Rat) | 2 | 2 | Rattus norvegicus (Rat) | | | | 2 | 2 | Rattus norvegicus (Rat) |
| | | HYDROXYMETHYL- | | | SEZ6B. [MASS = 105607 | | | | | | SPLICE ISOFORM 1 OF |
| | | GLUTARYL- | | | | | | | | | FIBRINOGEN ALPHA CHAIN |
| | | COA SYNTHASE, | | | | | | | | | PRECURSOR. [MASS = 86686 |
| | | CYTOPLASMIC. | | | | | | | | | |
| | | [MASS = 57434 | | | | | | | | | |
| 2 | 3 | Rattus norvegicus (Rat) | 2 | 2 | Rattus norvegicus (Rat) | | | | 2 | 2 | Rattus norvegicus (Rat) |
| | | PREDICTED: SIMILAR | | | SEMA4B PROTEIN | | | | | | EPITHELIAL-CADHERIN |
| | | TO HEAT SHOCK 70 KDA | | | (FRAGMENT). | | | | | | PRECURSOR. [MASS = 98715 |
| | | PROTEIN 4 LIKE. | | | [MASS = 79477 | | | | | | |
| | | [MASS = 136266 | | | | | | | | | |
| 2 | 2 | Rattus norvegicus (Rat) | 2 | 2 | Rattus norvegicus (Rat) | | | | 2 | 2 | Rattus norvegicus (Rat) |
| | | RUVB-LIKE 2. | | | SPLICE ISOFORM 1 OF | | | | | | FRUCTOSE-BISPHOSPHATE |
| | | [MASS = 51147 | | | NEUROFASCIN | | | | | | ALDOLASE C. [MASS = 39153 |
| | | | | | PRECURSOR. | | | | | | |
| | | | | | [MASS = 138004 | | | | | | |
| 2 | 3 | Rattus norvegicus (Rat) | 2 | 3 | Rattus norvegicus (Rat) | | | | 2 | 2 | Rattus norvegicus (Rat) |
| | | SPLICE ISOFORM 2 OF | | | PREDICTED: SIMILAR | | | | | | TUBULIN BETA CHAIN. |
| | | PLASMINOGEN | | | TO COLLAGEN ALPHA1 | | | | | | [MASS = 49963 |
| | | ACTIVATOR INHIBITOR | | | TYPE VI-PRECURSOR. | | | | | | |
| | | 1 RNA-BINDING | | | [MASS = 130760 | | | | | | |
| | | PROTEIN. [MASS = 42984 | | | | | | | | | |
| 2 | 2 | Rattus norvegicus (Rat) | 2 | 2 | Rattus norvegicus (Rat) | | | | 2 | 2 | Rattus norvegicus (Rat) |
| | | RUVB-LIKE 1. | | | 60S ACIDIC RIBOSOMAL | | | | | | ISOCITRATE |
| | | [MASS = 50214 | | | PROTEIN P2. | | | | | | DEHYDROGENASE [NADP] |
| | | | | | [MASS = 11692 | | | | | | CYTOPLASMIC. [MASS = 46734 |
| 2 | 2 | Rattus norvegicus (Rat) | 2 | 5 | Rattus norvegicus (Rat) | | | | 2 | 2 | Rattus norvegicus (Rat) |
| | | GLUTATHIONE S- | | | DERMCIDIN. | | | | | | PREDICTED: SIMILAR TO |
| | | TRANSFERASE P. | | | [MASS = 11284 | | | | | | EUKARYOTIC TRANSLATION |
| | | [MASS = 23308 | | | | | | | | | INITIATION FACTOR 4, |
| | | | | | | | | | | | GAMMA 1 ISOFORM A. |
| | | | | | | | | | | | [MASS = 93472 |
| 2 | 2 | Rattus norvegicus (Rat) | 2 | 2 | Rattus norvegicus (Rat) | | | | 2 | 2 | Rattus norvegicus (Rat) HEAT |
| | | MANNOSIDASE, ALPHA, | | | EPSILON 2 GLOBIN. | | | | | | SHOCK-RELATED 70 KDA |
| | | CLASS 1A, MEMBER 1. | | | [MASS = 16388 | | | | | | PROTEIN 2. [MASS = 69528 |
| | | [MASS = 73125 | | | | | | | | | |
| 2 | 2 | Rattus norvegicus (Rat) | 2 | 2 | Rattus norvegicus (Rat) | | | | 2 | 2 | Rattus norvegicus (Rat) |
| | | RHO GDP | | | TUBULIN BETA CHAIN. | | | | | | KINESIN-LIKE PROTEIN |
| | | DISSOCIATION | | | [MASS = 49963 | | | | | | KIF15. [MASS = 159554 |
| | | INHIBITOR (GDI) ALPHA. | | | | | | | | | |
| | | [MASS = 23407 | | | | | | | | | |
| 2 | 2 | Rattus norvegicus (Rat) | 2 | 2 | Rattus norvegicus (Rat) | | | | 2 | 2 | Rattus norvegicus (Rat) |
| | | SPLICE ISOFORM | | | PREDICTED: SIMILAR | | | | | | DAMAGE-SPECIFIC DNA |
| | | PYBP1 OF | | | TO SERINE PROTEASE | | | | | | BINDING PROTEIN 1. |
| | | POLYPYRIMIDINE | | | INHIBITOR 2.4. | | | | | | [MASS = 127059 |
| | | TRACT-BINDING | | | [MASS = 46841 | | | | | | |
| | | PROTEIN 1. | | | | | | | | | |
| | | [MASS = 56937 | | | | | | | | | |
| 2 | 3 | Rattus norvegicus (Rat) | 2 | 2 | Rattus norvegicus (Rat) | | | | 2 | 2 | Rattus norvegicus (Rat) HEAT |
| | | PREDICTED-HEAT | | | PREDICTED: SIMILAR | | | | | | SHOCK 70 KDA PROTEIN |
| | | SHOCK PROTEIN HSP | | | TO COATOMER | | | | | | 1A/1B. [MASS = 70185 |
| | | 90-BETA (Frgament). | | | PROTEIN COMPLEX | | | | | | |
| | | [MASS = 50669 | | | SUBUNIT ALPHA. | | | | | | |
| | | | | | [MASS = 138360 | | | | | | |
| 2 | 2 | Rattus norvegicus (Rat) | 2 | 2 | Rattus norvegicus (Rat) | | | | 2 | 2 | Rattus norvegicus (Rat) |
| | | METALLOPROTEINASE | | | PREDICTED: similar to | | | | | | PREDICTED: SIMILAR TO |
| | | INHIBITOR 1 | | | Periostin precursor (PN) | | | | | | GLYCERALDEHYDE-3- |
| | | PRECURSOR. | | | (Osteoblast-specific | | | | | | PHOSPHATE |
| | | [MASS = 23794 | | | factor 2) (OSF-2). | | | | | | DEHYDROGENASE. |
| | | | | | [MASS = 90252 | | | | | | [MASS = 35200 |
| 2 | 2 | Rattus norvegicus (Rat) | 2 | 2 | Rattus norvegicus (Rat) | | | | 2 | 3 | Rattus norvegicus (Rat) |
| | | PREDICTED: NIDOGEN | | | SPLICE ISOFORM 1 OF | | | | | | COLLAGEN ALPHA-2(I) |
| | | 2. [MASS = 173960 | | | MYOSIN-11 | | | | | | CHAIN PRECURSOR. |
| | | | | | [FRAGMENT). | | | | | | [MASS = 129564 |
| | | | | | [MASS = 152492 | | | | | | |
| 2 | 2 | Rattus norvegicus (Rat) | 2 | 2 | Rattus norvegicus (Rat) | | | | 2 | 2 | Rattus norvegicus (Rat) |
| | | PREDICTED: SIMILAR | | | 40S RIBOSOMAL | | | | | | EPSILON 3 GLOBIN. |
| | | TO ENO1 PROTEIN. | | | PROTEIN S10. | | | | | | [MASS = 16540 |
| | | [MASS = 47532 | | | [MASS = 18916 | | | | | | |
| 2 | 2 | Rattus norvegicus (Rat) | 2 | 3 | Rattus norvegicus (Rat) | | | | 2 | 2 | Rattus norvegicus (Rat) |
| | | SORTILIN PRECURSOR. | | | HISTIDINE-RICH | | | | | | HEMOPEXIN PRECURSOR. |
| | | [MASS = 91169 | | | GLYCOPROTEIN. | | | | | | [MASS = 51291 |
| | | | | | [MASS = 57581 | | | | | | |
| 2 | 3 | Rattus norvegicus (Rat) | 2 | 3 | Rattus norvegicus (Rat) | | | | 2 | 6 | Rattus norvegicus (Rat) |
| | | 60S ACIDIC RIBOSOMAL | | | RAB GDP | | | | | | COLLAGEN ALPHA-1(V) |
| | | PROTEIN P2. | | | DISSOCIATION | | | | | | CHAIN PRECURSOR. |
| | | [MASS = 11692 | | | INHIBITOR BETA. | | | | | | [MASS = 184610 |
| | | | | | [MASS = 50685 | | | | | | |
| 2 | 3 | Rattus norvegicus (Rat) | 2 | 2 | Rattus norvegicus (Rat) | | | | 2 | 2 | Rattus norvegicus (Rat) 1- |
| | | FOLLISTATIN-RELATED | | | LIVER | | | | | | PHOSPHATIDYLINOSITOL- |
| | | PROTEIN 1 | | | CARBOXYLESTERASE 1 | | | | | | 4,5-BISPHOSPHATE |
| | | PRECURSOR. | | | PRECURSOR. | | | | | | PHOSPHODIESTERASE |
| | | [MASS = 34622 | | | [MASS = 60175 | | | | | | GAMMA 1. [MASS = 148548 |
| 2 | 2 | Rattus norvegicus (Rat) | 2 | 2 | Rattus norvegicus (Rat) | | | | 2 | 5 | Rattus norvegicus (Rat) BETA- |
| | | PREDICTED: SIMILAR | | | PREDICTED: TUMOR | | | | | | 2-MICROGLOBULIN |
| | | TO PSMC6 PROTEIN. | | | REJECTION ANTIGEN | | | | | | PRECURSOR. [MASS = 13720 |
| | | [MASS = 45797 | | | GP96. [MASS = 92771 | | | | | | |
| 2 | 2 | Rattus norvegicus (Rat) | 2 | 2 | Rattus norvegicus (Rat) | | | | 2 | 3 | Rattus norvegicus (Rat) |
| | | SYNTENIN-1. | | | LOC362795 PROTEIN. | | | | | | STRUCTURAL |
| | | [MASS = 32423 | | | [MASS = 52392 | | | | | | MAINTENANCE OF |
| | | | | | | | | | | | CHROMOSOME 1-LIKE 1 |
| | | | | | | | | | | | PROTEIN. [MASS = 143205 |
| 2 | 2 | Rattus norvegicus (Rat) | 2 | 2 | Rattus norvegicus (Rat) | | | | 2 | 3 | Rattus norvegicus (Rat) T- |
| | | INOSINE | | | PREDICTED: SIMILAR | | | | | | CADHERIN. [MASS = 78086 |
| | | MONOPHOSPHATE | | | TO UBIQUITIN | | | | | | |
| | | DEHYDROGENASE 2. | | | CARBOXYL-TERMINAL | | | | | | |
| | | [MASS = 55799 | | | HYDROLASE 5. | | | | | | |
| | | | | | [MASS = 95779 | | | | | | |
| 2 | 2 | Rattus norvegicus (Rat) | 2 | 2 | Rattus norvegicus (Rat) | | | | 2 | 2 | Rattus norvegicus (Rat) BETA- |
| | | PREDICTED: ATPASE, | | | INOSINE | | | | | | 2-GLYCOPROTEIN 1 |
| | | H+ TRANSPORTING, | | | MONOPHOSPHATE | | | | | | PRECURSOR. [MASS = 33197 |
| | | LYSOSOMAL | | | DEHYDROGENASE 2. | | | | | | |
| | | ACCESSORY PROTEIN | | | [MASS = 55799 | | | | | | |
| | | 2. [MASS = 66094 | | | | | | | | | |
| 2 | 2 | Rattus norvegicus (Rat) | 2 | 2 | Rattus norvegicus (Rat) | | | | 2 | 2 | Rattus norvegicus (Rat) |
| | | DNA PRIMASE LARGE | | | HYPOTHETICAL | | | | | | CALCIUM-DEPENDENT |
| | | SUBUNIT. [MASS = 58603 | | | PROTEIN RGD1305890. | | | | | | SECRETION ACTIVATOR 1. |
| | | | | | [MASS = 31776 | | | | | | [MASS = 146266 |
| 2 | 2 | Rattus norvegicus (Rat) | 2 | 2 | Rattus norvegicus (Rat) | | | | 2 | 2 | Rattus norvegicus (Rat) |
| | | EUKARYOTIC | | | PREDICTED: SIMILAR | | | | | | EUKARYOTIC TRANSLATION |
| | | TRANSLATION | | | TO BETA- | | | | | | INITIATION FACTOR 3 |
| | | INITIATION FACTOR 3 | | | GALACTOSIDASE | | | | | | SUBUNIT 9. [MASS = 107985 |
| | | SUBUNIT 9. | | | PRECURSOR. | | | | | | |
| | | [MASS = 107985 | | | [MASS = 73228 | | | | | | |
| 2 | 2 | Rattus norvegicus (Rat) | 2 | 2 | Rattus norvegicus (Rat) | | | | 2 | 2 | Rattus norvegicus (Rat) |
| | | SPLICE ISOFORM 1 OF | | | MICROFIBRILLAR- | | | | | | ADAPTOR PROTEIN |
| | | SEX HORMONE- | | | ASSOCIATED PROTEIN | | | | | | COMPLEX AP-2, ALPHA 2 |
| | | BINDING GLOBULIN | | | 4. [MASS = 29050 | | | | | | SUBUNIT. [MASS = 104174 |
| | | PRECURSOR. | | | | | | | | | |
| | | [MASS = 44533 | | | | | | | | | |
| 2 | 2 | Rattus norvegicus (Rat) | 2 | 2 | Rattus norvegicus (Rat) | | | | 2 | 4 | Rattus norvegicus (Rat) |
| | | PREDICTED: SIMILAR | | | PREDICTED: SIMILAR | | | | | | NUCLEOSOME ASSEMBLY |
| | | TO COLLAGEN ALPHA1 | | | TO LAMININ ALPHA-1 | | | | | | PROTEIN 1-LIKE 1. |
| | | TYPE VI-PRECURSOR. | | | CHAIN PRECURSOR- | | | | | | [MASS = 45373 |
| | | [MASS = 130760 | | | MOUSE. [MASS = 338692 | | | | | | |
| 2 | 2 | Rattus norvegicus (Rat) | 2 | 2 | Rattus norvegicus (Rat) | | | | 2 | 2 | Rattus norvegicus (Rat) |
| | | PREDICTED: SIMILAR | | | SPLICEOSOME RNA | | | | | | PROTHROMBIN PRECURSOR |
| | | TO 26S PROTEASOME | | | HELICASE BAT1. | | | | | | (FRAGMENT). [MASS = 70412 |
| | | NON-ATPASE | | | [MASS = 49035 | | | | | | |
| | | REGULATORY SUBUNIT | | | | | | | | | |
| | | 11. [MASS = 54019 | | | | | | | | | |
| 2 | 2 | Rattus norvegicus (Rat) | 2 | 2 | Rattus norvegicus (Rat) | | | | 2 | 2 | Rattus norvegicus (Rat) |
| | | PREDICTED- | | | 60S RIBOSOMAL | | | | | | PREDICTED: CADHERIN 11. |
| | | HETEROGENEOUS | | | PROTEIN L24. | | | | | | [MASS = 88036 |
| | | NUCLEAR | | | [MASS = 17779 | | | | | | |
| | | RIBONUCLEOPROTEIN | | | | | | | | | |
| | | A1. [MASS = 33656 | | | | | | | | | |
| 2 | 2 | Rattus norvegicus (Rat) | 2 | 2 | Rattus norvegicus (Rat) | | | | 2 | 2 | Rattus norvegicus (Rat) M- |
| | | GUANINE NUCLEOTIDE- | | | PREDICTED: SIMILAR | | | | | | CADHERIN. [MASS = 85753 |
| | | BINDING PROTEIN BETA | | | TO STABILIN-1. | | | | | | |
| | | SUBUNIT 2-LIKE 1. | | | [MASS = 288663 | | | | | | |
| | | [MASS = 35419 | | | | | | | | | |
| 2 | 3 | Rattus norvegicus (Rat) | 2 | 2 | Rattus norvegicus (Rat) | | | | 2 | 2 | Rattus norvegicus (Rat) |
| | | PREDICTED: | | | PREDICTED: SIMILAR | | | | | | PREDICTED: similar to |
| | | EUKARYOTIC | | | TO 26S PROTEASOME | | | | | | ubiquitin-activating enzyme E1. |
| | | TRANSLATION | | | NON-ATPASE | | | | | | [MASS = 117931 |
| | | ELONGATION FACTOR 1 | | | REGULATORY SUBUNIT | | | | | | |
| | | GAMMA. [MASS = 72445 | | | 11. [MASS = 54019 | | | | | | |
| 2 | 2 | Rattus norvegicus (Rat) | 2 | 2 | Rattus norvegicus (Rat) | | | | 2 | 2 | Rattus norvegicus (Rat) |
| | | PREDICTED: SIMILAR | | | PREDICTED: similar to | | | | | | SPLICEOSOME RNA |
| | | TO DEAD/H BOX | | | 60S ribosomal protein | | | | | | HELICASE BAT1. |
| | | POLYPEPTIDE 36 | | | L29. [MASS = 16961 | | | | | | [MASS = 49035 |
| | | PROTEIN. | | | | | | | | | |
| | | [MASS = 113843 | | | | | | | | | |
| 2 | 2 | Rattus norvegicus (Rat) | | | | | | | 2 | 2 | Rattus norvegicus (Rat) |
| | | PREDICTED: SIMILAR | | | | | | | | | PREDICTED: SIMILAR TO |
| | | TO PYRUVATE KINASE | | | | | | | | | RANBP4. [MASS = 118926 |
| | | 3. [MASS = 84928 | | | | | | | | | |
| 2 | 3 | Rattus norvegicus (Rat) | | | | | | | 2 | 2 | Rattus norvegicus (Rat) |
| | | PREDICTED: SIMILAR | | | | | | | | | PREDICTED: PROCOLLAGEN, |
| | | TO HSPC263. | | | | | | | | | TYPE XII, ALPHA 1. |
| | | [MASS = 37041 | | | | | | | | | [MASS = 367709 |
| 2 | 2 | Rattus norvegicus (Rat) | | | | | | | 2 | 2 | Rattus norvegicus (Rat) |
| | | FIBRILLIN-2. | | | | | | | | | PREDICTED: SIMILAR TO |
| | | [MASS = 313374 | | | | | | | | | SLIT-ROBO RHO GTPASE- |
| | | | | | | | | | | | ACTIVATING PROTEIN 1. |
| | | | | | | | | | | | [MASS = 133329 |
| 2 | 2 | Rattus norvegicus (Rat) | | | | | | | 2 | 2 | Rattus norvegicus (Rat) |
| | | 15 KDA PROTEIN. | | | | | | | | | PREDICTED: HISTONE |
| | | [MASS = 14671 | | | | | | | | | DEACETYLASE 6. |
| | | | | | | | | | | | [MASS = 168631 |
| 2 | 2 | Rattus norvegicus (Rat) | | | | | | | 2 | 5 | Rattus norvegicus (Rat) |
| | | SPLICE ISOFORM 1 OF | | | | | | | | | DERMCIDIN. [MASS = 11284 |
| | | FIBRINOGEN ALPHA | | | | | | | | | |
| | | CHAIN PRECURSOR. | | | | | | | | | |
| | | [MASS = 86686 | | | | | | | | | |
| 2 | 2 | Rattus norvegicus (Rat) | | | | | | | 2 | 2 | Rattus norvegicus (Rat) |
| | | 40S RIBOSOMAL | | | | | | | | | TRIOSEPHOSPHATE |
| | | PROTEIN S13. | | | | | | | | | ISOMERASE. [MASS = 26790 |
| | | [MASS = 17091 | | | | | | | | | |
| 2 | 3 | Rattus norvegicus (Rat) | | | | | | | 2 | 2 | Rattus norvegicus (Rat) |
| | | SPLICE ISOFORM 1 OF | | | | | | | | | GLUCOSAMINE. |
| | | DNA-BINDING PROTEIN | | | | | | | | | [MASS = 60914 |
| | | A. [MASS = 38852 | | | | | | | | | |
| 2 | 2 | Rattus norvegicus (Rat) | | | | | | | 2 | 2 | Rattus norvegicus (Rat) |
| | | RETINOL-BINDING | | | | | | | | | PREDICTED: SIMILAR TO N- |
| | | PROTEIN I, CELLULAR. | | | | | | | | | TERMINAL |
| | | [MASS = 15703 | | | | | | | | | ACEYLTRANSFERASE 1. |
| | | | | | | | | | | | [MASS = 100994 |
| 2 | 2 | Rattus norvegicus (Rat) | | | | | | | 2 | 2 | Rattus norvegicus (Rat) |
| | | TPA: proteasome subunit | | | | | | | | | SPLICE ISOFORM 2 OF DNA. |
| | | beta type 6-like. | | | | | | | | | [MASS = 173853 |
| | | [MASS = 25304 | | | | | | | | | |
| 2 | 2 | Rattus norvegicus (Rat) | | | | | | | 2 | 2 | Rattus norvegicus (Rat) |
| | | PROTEIN ARGININE N- | | | | | | | | | PROTEASOME SUBUNIT |
| | | METHYLTRANSFERASE | | | | | | | | | BETA TYPE 2. [MASS = 22912 |
| | | 1. [MASS = 42436 | | | | | | | | | |
| 2 | 2 | Rattus norvegicus (Rat) | | | | | | | 2 | 4 | Rattus norvegicus (Rat) 14-3-3 |
| | | TRANSLATIONALLY- | | | | | | | | | PROTEIN THETA. |
| | | CONTROLLED TUMOR | | | | | | | | | [MASS = 27778 |
| | | PROTEIN. [MASS = 19462 | | | | | | | | | |
| 2 | 3 | Rattus norvegicus (Rat) | | | | | | | 2 | 2 | Rattus norvegicus (Rat) 14-3-3 |
| | | PREDICTED: SIMILAR | | | | | | | | | PROTEIN BETA/ALPHA. |
| | | TO STABILIN-1. | | | | | | | | | [MASS = 27923 |
| | | [MASS = 288663 | | | | | | | | | |
| 2 | 2 | Rattus norvegicus (Rat) | | | | | | | 2 | 2 | Rattus norvegicus (Rat) |
| | | 60S RIBOSOMAL | | | | | | | | | CALMODULIN. [MASS = 16706 |
| | | PROTEIN L7. | | | | | | | | | |
| | | [MASS = 30329 | | | | | | | | | |
| 2 | 2 | Rattus norvegicus (Rat) | | | | | | | 2 | 2 | Rattus norvegicus (Rat) |
| | | PREDICTED: SIMILAR | | | | | | | | | INSULIN-LIKE GROWTH |
| | | TO SERINE PROTEASE | | | | | | | | | FACTOR-BINDING PROTEIN |
| | | INHIBITOR 2.4. | | | | | | | | | COMPLEX ACID LABILE |
| | | [MASS = 46841 | | | | | | | | | CHAIN PRECURSOR. |
| | | | | | | | | | | | [MASS = 66812 |
| 2 | 2 | Rattus norvegicus (Rat) | | | | | | | 2 | 2 | Rattus norvegicus (Rat) |
| | | NUCLEOSIDE | | | | | | | | | PREDICTED: |
| | | DIPHOSPHATE KINASE | | | | | | | | | CHROMODOMAIN HELICASE |
| | | A. [MASS = 17193 | | | | | | | | | DNA BINDING PROTEIN 4. |
| | | | | | | | | | | | [MASS = 222452 |
| 2 | 2 | Rattus norvegicus (Rat) | | | | | | | 2 | 2 | Rattus norvegicus (Rat) |
| | | EUKARYOTIC | | | | | | | | | PREDICTED: SIMILAR TO |
| | | TRANSLATION | | | | | | | | | RIBOSOMAL PROTEIN L6. |
| | | INITIATION FACTOR 5A- | | | | | | | | | [MASS = 32944 |
| | | 1. [MASS = 16701 | | | | | | | | | |
| 2 | 2 | Rattus norvegicus (Rat) | | | | | | | 2 | 2 | Rattus norvegicus (Rat) |
| | | PREDICTED-40S | | | | | | | | | PREDICTED: SIMILAR TO |
| | | ribosomal protein S17. | | | | | | | | | STABILIN-1. [MASS = 288663 |
| | | [MASS = 16340 | | | | | | | | | |
| 2 | 3 | Rattus norvegicus (Rat) | | | | | | | 2 | 2 | Rattus norvegicus (Rat) |
| | | SPLICE ISOFORM 2 OF | | | | | | | | | TENASCIN (FRAGMENT). |
| | | RECEPTOR-TYPE | | | | | | | | | [MASS = 67815 |
| | | TYROSINE-PROTEIN | | | | | | | | | |
| | | PHOSPHATASE ZETA | | | | | | | | | |
| | | PRECURSOR. | | | | | | | | | |
| | | [MASS = 164596 | | | | | | | | | |
| 2 | 2 | Rattus norvegicus (Rat) | | | | | | | 2 | 2 | Rattus norvegicus (Rat) |
| | | PREDICTED: SIMILAR | | | | | | | | | PREDICTED: SIMILAR TO |
| | | TO CCR4-NOT | | | | | | | | | LIPOPROTEIN RECEPTOR- |
| | | TRANSCRIPTION | | | | | | | | | RELATED PROTEIN. |
| | | COMPLEX, SUBUNIT 1 | | | | | | | | | [MASS = 504889 |
| | | ISOFORM A. | | | | | | | | | |
| | | [MASS = 93683 | | | | | | | | | |
| 2 | 2 | Rattus norvegicus (Rat) | | | | | | | 2 | 2 | Rattus norvegicus (Rat) ZERO |
| | | HEAT SHOCK 70 KDA | | | | | | | | | BETA-1 GLOBIN. |
| | | PROTEIN 1A/1B. | | | | | | | | | [MASS = 16023 |
| | | [MASS = 70185 | | | | | | | | | |
| 2 | 3 | Rattus norvegicus (Rat) | | | | | | | 2 | 2 | Rattus norvegicus (Rat) GTP- |
| | | RECEPTOR-LIKE | | | | | | | | | BINDING NUCLEAR PROTEIN |
| | | PROTEIN TYROSINE | | | | | | | | | RAN, TESTIS-SPECIFIC |
| | | PHOSPHATASE KAPPA | | | | | | | | | ISOFORM. [MASS = 24451 |
| | | EXTRACELLULAR | | | | | | | | | |
| | | REGION. [MASS = 56159 | | | | | | | | | |
| 2 | 3 | Rattus norvegicus (Rat) | | | | | | | 2 | 2 | Rattus norvegicus (Rat) |
| | | NUCLEIC ACID BINDING | | | | | | | | | COLLAGEN ALPHA-1(III) |
| | | FACTOR PRM10. | | | | | | | | | CHAIN PRECURSOR. |
| | | [MASS = 33815 | | | | | | | | | [MASS = 138936 |
| 2 | 2 | Rattus norvegicus (Rat) | | | | | | | 2 | 2 | Rattus norvegicus (Rat) |
| | | SPLICE ISOFORM | | | | | | | | | PREDICTED: SIMILAR TO |
| | | APP770 OF AMYLOID | | | | | | | | | HEPATIC MULTIPLE |
| | | BETA A4 PROTEIN | | | | | | | | | INOSITOL POLYPHOSPHATE |
| | | PRECURSOR | | | | | | | | | PHOSPHATASE. |
| | | (FRAGMENT). | | | | | | | | | [MASS = 54619 |
| | | [MASS = 86704 | | | | | | | | | |
| 2 | 5 | Rattus norvegicus (Rat) | | | | | | | 2 | 2 | Rattus norvegicus (Rat) |
| | | PREDICTED: similar to | | | | | | | | | PREDICTED: SIMILAR TO |
| | | Fibulin-1 precursor. | | | | | | | | | MUCIN 17. [MASS = 189893 |
| | | [MASS = 75381 | | | | | | | | | |
| 2 | 2 | Rattus norvegicus (Rat) | | | | | | | 2 | 9 | Rattus norvegicus (Rat) |
| | | CADHERIN EGF LAG | | | | | | | | | SERINE (OR CYSTEINE) |
| | | SEVEN-PASS G-TYPE | | | | | | | | | PROTEINASE INHIBITOR, |
| | | RECEPTOR 2. | | | | | | | | | CLADE A (ALPHA-1 |
| | | [MASS = 317122 | | | | | | | | | ANTIPROTEINASE, |
| | | | | | | | | | | | ANTITRYPSIN), MEMBER 6. |
| | | | | | | | | | | | [MASS = 44671 |
| 2 | 2 | Rattus norvegicus (Rat) | | | | | | | 2 | 2 | Rattus norvegicus (Rat) |
| | | PREDICTED: SIMILAR | | | | | | | | | FARNESYL |
| | | TO ARX. [MASS = 121446 | | | | | | | | | PYROPHOSPHATE |
| | | | | | | | | | | | SYNTHETASE. [MASS = 40830 |
| 2 | 4 | Rattus norvegicus (Rat) | | | | | | | 2 | 3 | Rattus norvegicus (Rat) |
| | | HETEROGENEOUS | | | | | | | | | PREDICTED: SIMILAR TO |
| | | NUCLEAR | | | | | | | | | HEPARAN SULFATE |
| | | RIBONUCLEOPROTEIN | | | | | | | | | PROTEOGLYCAN 2. |
| | | C. [MASS = 34385 | | | | | | | | | [MASS = 377284 |
| 2 | 2 | Rattus norvegicus (Rat) | | | | | | | 2 | 2 | Rattus norvegicus (Rat) |
| | | PREDICTED: | | | | | | | | | PREDICTED: SIMILAR TO |
| | | PROTEASOME | | | | | | | | | ELAV. [MASS = 49528 |
| | | (PROSOME, | | | | | | | | | |
| | | MACROPAIN) SUBUNIT, | | | | | | | | | |
| | | BETA TYPE 5. | | | | | | | | | |
| | | [MASS = 37128 | | | | | | | | | |
| 2 | 2 | Rattus norvegicus (Rat) | | | | | | | 2 | 2 | Rattus norvegicus (Rat) |
| | | 109 KDA PROTEIN. | | | | | | | | | PREDICTED: SIMILAR TO |
| | | [MASS = 108509 | | | | | | | | | TUBULIN-SPECIFIC |
| | | | | | | | | | | | CHAPERONE D. |
| | | | | | | | | | | | [MASS = 60160 |
| 2 | 2 | Rattus norvegicus (Rat) | | | | | | | 2 | 2 | Rattus norvegicus (Rat) |
| | | AMBP PROTEIN | | | | | | | | | COATOMER SUBUNIT BETA′. |
| | | PRECURSOR. | | | | | | | | | [MASS = 102420 |
| | | [MASS = 38851 | | | | | | | | | |
| 2 | 2 | Rattus norvegicus (Rat) | | | | | | | 2 | 2 | Rattus norvegicus (Rat) |
| | | PROTEASOME. | | | | | | | | | PREDICTED: SIMILAR TO |
| | | [MASS = 60688 | | | | | | | | | EXPRESSED SEQUENCE |
| | | | | | | | | | | | AI314180. [MASS = 203921 |
| 2 | 2 | Rattus norvegicus (Rat) | | | | | | | 2 | 2 | Rattus norvegicus (Rat) |
| | | PREDICTED: SIMILAR | | | | | | | | | PREDICTED: SIMILAR TO |
| | | TO RIBOSOMAL | | | | | | | | | REGULATOR OF NONSENSE |
| | | PROTEIN L14. | | | | | | | | | TRANSCRIPTS 1. |
| | | [MASS = 18408 | | | | | | | | | [MASS = 88226 |
| 2 | 2 | Rattus norvegicus (Rat) | | | | | | | 2 | 2 | Rattus norvegicus (Rat) |
| | | LOW-DENSITY | | | | | | | | | PREDICTED: SIMILAR TO |
| | | LIPOPROTEIN | | | | | | | | | SORCSB SPLICE VARIANT |
| | | RECEPTOR-RELATED | | | | | | | | | OF THE VPS10 DOMAIN |
| | | PROTEIN 2 | | | | | | | | | RECEPTOR SORCS. |
| | | PRECURSOR. | | | | | | | | | [MASS = 129969 |
| | | [MASS = 519276 | | | | | | | | | |
| 2 | 2 | Rattus norvegicus (Rat) | | | | | | | 2 | 2 | Rattus norvegicus (Rat) |
| | | GRP78 BINDING | | | | | | | | | PREDICTED: SIMILAR TO 40S |
| | | PROTEIN. | | | | | | | | | RIBOSOMAL PROTEIN S19. |
| | | [MASS = 110574 | | | | | | | | | [MASS = 16172 |
| 2 | 3 | Rattus norvegicus (Rat) | | | | | | | 2 | 2 | Rattus norvegicus (Rat) |
| | | PREDICTED: SIMILAR | | | | | | | | | PREDICTED: SIMILAR TO |
| | | TO COLLAGEN ALPHA | | | | | | | | | CYFIP1 PROTEIN. |
| | | 2(IV) CHAIN | | | | | | | | | [MASS = 144933 |
| | | PRECURSOR - MOUSE. | | | | | | | | | |
| | | [MASS = 192535 | | | | | | | | | |
| 2 | 2 | Rattus norvegicus (Rat) | | | | | | | 2 | 2 | Rattus norvegicus (Rat) |
| | | 60S RIBOSOMAL | | | | | | | | | PREDICTED similar to |
| | | PROTEIN L7A. | | | | | | | | | FIBRINOGEN, GAMMA |
| | | [MASS = 29864 | | | | | | | | | POLYPEPTIDE. [MASS = 49121 |
| 2 | 3 | Rattus norvegicus (Rat) | | | | | | | 2 | 2 | Rattus norvegicus (Rat) |
| | | PREDICTED-Proteasome | | | | | | | | | PREDICTED: SIMILAR TO |
| | | 26S subunit, ATPase 3. | | | | | | | | | LAMININ B1. [MASS = 228429 |
| | | [MASS = 50509 | | | | | | | | | |
| 2 | 2 | Rattus norvegicus (Rat) | | | | | | | 2 | 2 | Rattus norvegicus (Rat) |
| | | PREDICTED: SIMILAR | | | | | | | | | URIDINE MONOPHOSPHATE |
| | | TO N-TERMINAL | | | | | | | | | SYNTHETASE. [MASS = 52379 |
| | | ACEYLTRANSFERASE 1. | | | | | | | | | |
| | | [MASS = 100994 | | | | | | | | | |
| 2 | 2 | Rattus norvegicus (Rat) | | | | | | | 2 | 2 | Rattus norvegicus (Rat) |
| | | O-GLCNACASE. | | | | | | | | | PREDICTED: SIMILAR TO |
| | | [MASS = 102918 | | | | | | | | | PROTOCADHERIN 18 |
| | | | | | | | | | | | PRECURSOR. [MASS = 123552 |
| 2 | 2 | Rattus norvegicus (Rat) | | | | | | | 2 | 2 | Rattus norvegicus (Rat) |
| | | 40S RIBOSOMAL | | | | | | | | | ASCC3L1 PROTEIN. |
| | | PROTEIN S21. | | | | | | | | | [MASS = 244875 |
| | | [MASS = 9127 | | | | | | | | | |
| 2 | 2 | Rattus norvegicus (Rat) | | | | | | | 2 | 2 | Rattus norvegicus (Rat) |
| | | PREDICTED: SIMILAR | | | | | | | | | PROLIFERATION- |
| | | TO CYFIP1 PROTEIN. | | | | | | | | | ASSOCIATED 2G4, 38 KDA. |
| | | [MASS = 144933 | | | | | | | | | [MASS = 43657 |
| 2 | 4 | Rattus norvegicus (Rat) | | | | | | | 2 | 2 | Rattus norvegicus (Rat) T- |
| | | SPLICE ISOFORM 1 OF | | | | | | | | | COMPLEX PROTEIN 1 |
| | | 40S RIBOSOMAL | | | | | | | | | SUBUNIT ALPHA. |
| | | PROTEIN S24. | | | | | | | | | [MASS = 60360 |
| | | [MASS = 15423 | | | | | | | | | |
| 2 | 7 | Rattus norvegicus (Rat) | | | | | | | 2 | 2 | Rattus norvegicus (Rat) |
| | | DERMCIDIN. | | | | | | | | | INOSINE MONOPHOSPHATE |
| | | [MASS = 11284 | | | | | | | | | DEHYDROGENASE 2. |
| | | | | | | | | | | | [MASS = 55799 |
| 2 | 2 | Rattus norvegicus (Rat) | | | | | | | 2 | 2 | Rattus norvegicus (Rat) 60S |
| | | PREDICTED: SIMILAR | | | | | | | | | RIBOSOMAL PROTEIN L7A. |
| | | TO 60S RIBOSOMAL | | | | | | | | | [MASS = 29864 |
| | | PROTEIN L26. | | | | | | | | | |
| | | [MASS = 24366 | | | | | | | | | |
| 2 | 2 | Rattus norvegicus (Rat) | | | | | | | 2 | 2 | Rattus norvegicus (Rat) LOW- |
| | | PREDICTED: SPLICING | | | | | | | | | DENSITY LIPOPROTEIN |
| | | FACTOR 3B, SUBUNIT 1. | | | | | | | | | RECEPTOR PRECURSOR. |
| | | [MASS = 152445 | | | | | | | | | [MASS = 96622 |
| 2 | 2 | Rattus norvegicus (Rat) | | | | | | | 2 | 3 | Rattus norvegicus (Rat) |
| | | HAPTOGLOBIN | | | | | | | | | PREDICTED: SIMILAR TO |
| | | PRECURSOR. | | | | | | | | | NISCHARIN. [MASS = 148481 |
| | | [MASS = 38549 | | | | | | | | | |
| 2 | 2 | Rattus norvegicus (Rat) | | | | | | | 2 | 2 | Rattus norvegicus (Rat) |
| | | PREDICTED: SIMILAR | | | | | | | | | PREDICTED: SIMILAR TO |
| | | TO EXPRESSED | | | | | | | | | GTPASE ACTIVATING |
| | | SEQUENCE AI314180. | | | | | | | | | PROTEIN AND VPS9 |
| | | [MASS = 203921 | | | | | | | | | DOMAINS 1. [MASS = 160359 |
| 2 | 2 | Rattus norvegicus (Rat) | | | | | | | 2 | 4 | Rattus norvegicus (Rat) |
| | | ARCADLIN. | | | | | | | | | PREDICTED: SIMILAR TO |
| | | [MASS = 103800 | | | | | | | | | ALDEHYDE |
| | | | | | | | | | | | DEHYDROGENASE FAMILY 7, |
| | | | | | | | | | | | MEMBER A1. [MASS = 58749 |
| 2 | 2 | Rattus norvegicus (Rat) | | | | | | | 2 | 2 | Rattus norvegicus (Rat) |
| | | EPITHELIAL-CADHERIN | | | | | | | | | NUCLEOSIDE DIPHOSPHATE |
| | | PRECURSOR. | | | | | | | | | KINASE B. [MASS = 17283 |
| | | [MASS = 98715 | | | | | | | | | |
| 2 | 3 | Rattus norvegicus (Rat) | | | | | | | 2 | 2 | Rattus norvegicus (Rat) |
| | | PREDICTED: SIMILAR | | | | | | | | | COMPLEMENT COMPONENT |
| | | TO 60S RIBOSOMAL | | | | | | | | | C6 PRECURSOR. |
| | | PROTEIN L7A. | | | | | | | | | [MASS = 105114 |
| | | [MASS = 13842 | | | | | | | | | |
| 2 | 2 | Rattus norvegicus (Rat) | | | | | | | 2 | 2 | Rattus norvegicus (Rat) |
| | | PREDICTED: SIMILAR | | | | | | | | | PREDICTED: TYROSINE |
| | | TO NISCHARIN. | | | | | | | | | KINASE RECEPTOR 1. |
| | | [MASS = 148481 | | | | | | | | | [MASS = 125210 |
| 2 | 2 | Rattus norvegicus (Rat) | | | | | | | 2 | 4 | Rattus norvegicus (Rat) |
| | | SPARC PRECURSOR. | | | | | | | | | HEMOGLOBIN ALPHA-1/2 |
| | | [MASS = 34384 | | | | | | | | | SUBUNIT. [MASS = 15197 |
| 2 | 2 | Rattus norvegicus (Rat) | | | | | | | 2 | 2 | Rattus norvegicus (Rat) 40S |
| | | SERINE (OR CYSTEINE) | | | | | | | | | RIBOSOMAL PROTEIN S18. |
| | | PROTEINASE | | | | | | | | | [MASS = 17719 |
| | | INHIBITOR, CLADE A | | | | | | | | | |
| | | (ALPHA-1 | | | | | | | | | |
| | | ANTIPROTEINASE, | | | | | | | | | |
| | | ANTITRYPSIN), MEMBER | | | | | | | | | |
| | | 6. [MASS = 44671 | | | | | | | | | |
| 2 | 2 | Rattus norvegicus (Rat) | | | | | | | 2 | 2 | Rattus norvegicus (Rat) |
| | | VESICLE ASSOCIATED | | | | | | | | | SPLICE ISOFORM V0 OF |
| | | PROTEIN. | | | | | | | | | VERSICAN CORE PROTEIN |
| | | [MASS = 135350 | | | | | | | | | PRECURSOR (FRAGMENT). |
| | | | | | | | | | | | [MASS = 300008 |
| 2 | 3 | Rattus norvegicus (Rat) | | | | | | | 2 | 3 | Rattus norvegicus (Rat) |
| | | HYPOTHETICAL | | | | | | | | | PREDICTED: SIMILAR TO |
| | | PROTEIN ALDOAL1. | | | | | | | | | MAM DOMAIN CONTAINING |
| | | [MASS = 39492 | | | | | | | | | 2. [MASS = 68019 |
| 2 | 2 | Rattus norvegicus (Rat) | | | | | | | 2 | 3 | Rattus norvegicus (Rat) |
| | | PREDICTED: SIMILAR | | | | | | | | | PREDICTED: SIMILAR TO |
| | | TO EUKARYOTIC | | | | | | | | | PUTATIVE E3 LIGASE. |
| | | TRANSLATION | | | | | | | | | [MASS = 526904 |
| | | INITIATION FACTOR 4, | | | | | | | | | |
| | | GAMMA 1 ISOFORM A. | | | | | | | | | |
| | | [MASS = 93472 | | | | | | | | | |
| 2 | 2 | Rattus norvegicus (Rat) | | | | | | | 2 | 2 | Rattus norvegicus (Rat) |
| | | SPLICE ISOFORM CDK2- | | | | | | | | | INSULIN-LIKE GROWTH |
| | | ALPHA OF CELL | | | | | | | | | FACTOR 1 RECEPTOR |
| | | DIVISION PROTEIN | | | | | | | | | PRECURSOR. [MASS = 155524 |
| | | KINASE 2. [MASS = 33887 | | | | | | | | | |
| 2 | 2 | Rattus norvegicus (Rat) | | | | | | | 2 | 2 | Rattus norvegicus (Rat) |
| | | COMPLEMENT | | | | | | | | | GLUCOSIDASE, ALPHA; |
| | | COMPONENT 2. | | | | | | | | | ACID. [MASS = 106207 |
| | | [MASS = 83699 | | | | | | | | | |
| 2 | 2 | Rattus norvegicus (Rat) | | | | | | | 2 | 2 | Rattus norvegicus (Rat) |
| | | ZINC | | | | | | | | | COMPLEMENT COMPONENT |
| | | PHOSPHODIESTERASE | | | | | | | | | 1, S SUBCOMPONENT. |
| | | ELAC PROTEIN 2. | | | | | | | | | [MASS = 77713 |
| | | [MASS = 92340 | | | | | | | | | |
| 2 | 2 | Rattus norvegicus (Rat) | | | | | | | 2 | 3 | Rattus norvegicus (Rat) |
| | | PREDICTED: SIMILAR | | | | | | | | | SPLICE ISOFORM 2 OF |
| | | TO 116 KDA U5 SMALL | | | | | | | | | TROPOMYOSIN BETA CHAIN. |
| | | NUCLEAR | | | | | | | | | [MASS = 32958 |
| | | RIBONUCLEOPROTEIN | | | | | | | | | |
| | | COMPONENT. | | | | | | | | | |
| | | [MASS = 109478 | | | | | | | | | |
| 2 | 2 | Rattus norvegicus (Rat) | | | | | | | 2 | 2 | Rattus norvegicus (Rat) |
| | | PREDICTED: SIMILAR | | | | | | | | | HYPOTHETICAL PROTEIN |
| | | TO 40S RIBOSOMAL | | | | | | | | | ALDOAL1. [MASS = 39492 |
| | | PROTEIN S16. | | | | | | | | | |
| | | [MASS = 20192 | | | | | | | | | |
| 2 | 2 | Rattus norvegicus (Rat) | | | | | | | 2 | 2 | Rattus norvegicus (Rat) DNA |
| | | SERINE/THREONINE- | | | | | | | | | POLYMERASE DELTA |
| | | PROTEIN | | | | | | | | | CATALYTIC SUBUNIT. |
| | | PHOSPHATASE 2A | | | | | | | | | [MASS = 123601 |
| | | CATALYTIC SUBUNIT | | | | | | | | | |
| | | BETA ISOFORM. | | | | | | | | | |
| | | [MASS = 35575 | | | | | | | | | |
| 2 | 2 | Rattus norvegicus (Rat) | | | | | | | 2 | 4 | Rattus norvegicus (Rat) AC2- |
| | | MATRIN-3. [MASS = 94447 | | | | | | | | | 008. [MASS = 26204 |
| 2 | 2 | Rattus norvegicus (Rat) | | | | | | | 2 | 2 | Rattus norvegicus (Rat) PRX |
| | | LEUKEMIA INHIBITORY | | | | | | | | | IV. [MASS = 31007 |
| | | FACTOR RECEPTOR | | | | | | | | | |
| | | PRECURSOR. | | | | | | | | | |
| | | [MASS = 122394 | | | | | | | | | |
| 2 | 3 | Rattus norvegicus (Rat) | | | | | | | | | |
| | | PREDICTED: SIMILAR | | | | | | | | | |
| | | TO POLY(RC)-BINDING | | | | | | | | | |
| | | PROTEIN 1. | | | | | | | | | |
| | | [MASS = 37498 | | | | | | | | | |
| 2 | 2 | Rattus norvegicus (Rat) | | | | | | | | | |
| | | LRRGT00164. | | | | | | | | | |
| | | [MASS = 111258 | | | | | | | | | |
| 2 | 2 | Rattus norvegicus (Rat) | | | | | | | | | |
| | | DNA LIGASE 1. | | | | | | | | | |
| | | [MASS = 102482 | | | | | | | | | |
| 2 | 2 | Rattus norvegicus (Rat) | | | | | | | | | |
| | | HEMOGLOBIN ALPHA- | | | | | | | | | |
| | | 1/2 SUBUNIT. | | | | | | | | | |
| | | [MASS = 15197 | | | | | | | | | |
| 2 | 2 | Rattus norvegicus (Rat) | | | | | | | | | |
| | | PREDICTED: SIMILAR | | | | | | | | | |
| | | TO RIBOSOMAL | | | | | | | | | |
| | | PROTEIN L34. | | | | | | | | | |
| | | [MASS = 13582 | | | | | | | | | |
| 2 | 2 | Rattus norvegicus (Rat) | | | | | | | | | |
| | | TRANSKETOLASE. | | | | | | | | | |
| | | [MASS = 71159 | | | | | | | | | |
| 2 | 2 | Rattus norvegicus (Rat) | | | | | | | | | |
| | | SPLICE ISOFORM 1 OF | | | | | | | | | |
| | | HETEROGENEOUS | | | | | | | | | |
| | | NUCLEAR | | | | | | | | | |
| | | RIBONUCLEOPROTEIN | | | | | | | | | |
| | | M. [MASS = 74350 | | | | | | | | | |
| 2 | 3 | Rattus norvegicus (Rat) | | | | | | | | | |
| | | HISTONE H2A. | | | | | | | | | |
| | | [MASS = 14189 | | | | | | | | | |
| 2 | 2 | Rattus norvegicus (Rat) | | | | | | | | | |
| | | 40S RIBOSOMAL | | | | | | | | | |
| | | PROTEIN S25. | | | | | | | | | |
| | | [MASS = 13742 | | | | | | | | | |
| 2 | 2 | Rattus norvegicus (Rat) | | | | | | | | | |
| | | HYPOTHETICAL | | | | | | | | | |
| | | PROTEIN RGD1305890. | | | | | | | | | |
| | | [MASS = 31776 | | | | | | | | | |
| 2 | 2 | Rattus norvegicus (Rat) | | | | | | | | | |
| | | SSB PROTEIN. | | | | | | | | | |
| | | [MASS = 43926 | | | | | | | | | |
| 2 | 2 | Rattus norvegicus (Rat) | | | | | | | | | |
| | | C-REACTIVE PROTEIN | | | | | | | | | |
| | | PRECURSOR. | | | | | | | | | |
| | | [MASS = 25468 | | | | | | | | | |
| 2 | 2 | Rattus norvegicus (Rat) | | | | | | | | | |
| | | GLUCOSAMINE. | | | | | | | | | |
| | | [MASS = 60914 | | | | | | | | | |
| 2 | 2 | Rattus norvegicus (Rat) | | | | | | | | | |
| | | TUBULIN BETA CHAIN. | | | | | | | | | |
| | | [MASS = 49963 | | | | | | | | | |
| 2 | 2 | Rattus norvegicus (Rat) | | | | | | | | | |
| | | LEUKOCYTE COMMON | | | | | | | | | |
| | | ANTIGEN-RELATED | | | | | | | | | |
| | | PHOSPHATASE | | | | | | | | | |
| | | PRECURSOR. | | | | | | | | | |
| | | [MASS = 212954 | | | | | | | | | |
| 2 | 2 | Rattus norvegicus (Rat) | | | | | | | | | |
| | | PREDICTED similar to | | | | | | | | | |
| | | POSTSYNAPTIC | | | | | | | | | |
| | | DENSITY PROTEIN. | | | | | | | | | |
| | | [MASS = 186848 | | | | | | | | | |
| 2 | 2 | Rattus norvegicus (Rat) | | | | | | | | | |
| | | COMPLEMENT | | | | | | | | | |
| | | INHIBITORY FACTOR H. | | | | | | | | | |
| | | [MASS = 140344 | | | | | | | | | |
| 2 | 2 | Rattus norvegicus (Rat) | | | | | | | | | |
| | | PROTEASOME SUBUNIT | | | | | | | | | |
| | | BETA TYPE 2. | | | | | | | | | |
| | | [MASS = 22912 | | | | | | | | | |
| 2 | 2 | Rattus norvegicus (Rat) | | | | | | | | | |
| | | 40S RIBOSOMAL | | | | | | | | | |
| | | PROTEIN S11. | | | | | | | | | |
| | | [MASS = 18431 | | | | | | | | | |
| 2 | 2 | Rattus norvegicus (Rat) | | | | | | | | | |
| | | PREDICTED: SIMILAR | | | | | | | | | |
| | | TO EIF4G1 PROTEIN. | | | | | | | | | |
| | | [MASS = 175705 | | | | | | | | | |
| 2 | 2 | Rattus norvegicus (Rat) | | | | | | | | | |
| | | 92 KDA PROTEIN. | | | | | | | | | |
| | | [MASS = 91785 | | | | | | | | | |
| 2 | 3 | Rattus norvegicus (Rat) | | | | | | | | | |
| | | PREDICTED: SIMILAR | | | | | | | | | |
| | | TO RANBP4. | | | | | | | | | |
| | | [MASS = 118926 | | | | | | | | | |
| 2 | 7 | Rattus norvegicus (Rat) | | | | | | | | | |
| | | BETA-ENOLASE. | | | | | | | | | |
| | | [MASS = 46830 | | | | | | | | | |
| 2 | 2 | Rattus norvegicus (Rat) | | | | | | | | | |
| | | PEROXIREDOXIN-1. | | | | | | | | | |
| | | [MASS = 22109 | | | | | | | | | |
| 2 | 3 | Rattus norvegicus (Rat) | | | | | | | | | |
| | | PREDICTED: SIMILAR | | | | | | | | | |
| | | TO APOLIPOPROTEIN | | | | | | | | | |
| | | C2. [MASS = 10695 | | | | | | | | | |
| 2 | 2 | Rattus norvegicus (Rat) | | | | | | | | | |
| | | UDP-N- | | | | | | | | | |
| | | ACETYLGLUCOSAMINE-- | | | | | | | | | |
| | | PEPTIDE N- | | | | | | | | | |
| | | ACETYLGLUCOSAMINYL | | | | | | | | | |
| | | TRANSFERASE 110 KDA | | | | | | | | | |
| | | SUBUNIT. | | | | | | | | | |
| | | [MASS = 116954 | | | | | | | | | |
| 2 | 2 | Rattus norvegicus (Rat) | | | | | | | | | |
| | | PREDICTED: SIMILAR | | | | | | | | | |
| | | TO NUCLEAR PORE | | | | | | | | | |
| | | COMPLEX-ASSOCIATED | | | | | | | | | |
| | | INTRANUCLEAR | | | | | | | | | |
| | | COILED-COIL PROTEIN | | | | | | | | | |
| | | TPR. [MASS = 279947 | | | | | | | | | |
| 2 | 2 | Rattus norvegicus (Rat) | | | | | | | | | |
| | | PROTEASOME SUBUNIT | | | | | | | | | |
| | | ALPHA TYPE 6. | | | | | | | | | |
| | | [MASS = 27399 | | | | | | | | | |
| 2 | 2 | Rattus norvegicus (Rat) | | | | | | | | | |
| | | PREDICTED: SIMILAR | | | | | | | | | |
| | | TO HCF. [MASS = 215082 | | | | | | | | | |
| 2 | 2 | Rattus norvegicus (Rat) | | | | | | | | | |
| | | PREDICTED: SIMILAR | | | | | | | | | |
| | | TO SHPRH PROTEIN. | | | | | | | | | |
| | | [MASS = 192457 | | | | | | | | | |
| 2 | 2 | Rattus norvegicus (Rat) | | | | | | | | | |
| | | PREDICTED: SIMILAR | | | | | | | | | |
| | | TO 60S RIBOSOMAL | | | | | | | | | |
| | | PROTEIN L12. | | | | | | | | | |
| | | [MASS = 17847 | | | | | | | | | |
| 2 | 2 | Rattus norvegicus (Rat) | | | | | | | | | |
| | | PREDICTED: SIMILAR | | | | | | | | | |
| | | TO TRANSLIN- | | | | | | | | | |
| | | ASSOCIATED FACTOR X | | | | | | | | | |
| | | (TSNAX) INTERACTING | | | | | | | | | |
| | | PROTEIN 1. | | | | | | | | | |
| | | [MASS = 85228 | | | | | | | | | |
| 2 | 2 | Rattus norvegicus (Rat) | | | | | | | | | |
| | | JUNCTION | | | | | | | | | |
| | | PLAKOGLOBIN. | | | | | | | | | |
| | | [MASS = 81801 | | | | | | | | | |
| 2 | 5 | Rattus norvegicus (Rat) | | | | | | | | | |
| | | PREDICTED: SIMILAR | | | | | | | | | |
| | | TO GLYCERALDEHYDE- | | | | | | | | | |
| | | 3-PHOSPHATE | | | | | | | | | |
| | | DEHYDROGENASE. | | | | | | | | | |
| | | [MASS = 35856 | | | | | | | | | |
| 2 | 2 | Rattus norvegicus (Rat) | | | | | | | | | |
| | | PREDICTED: SIMILAR | | | | | | | | | |
| | | TO DESMOPLAKIN | | | | | | | | | |
| | | ISOFORM II. | | | | | | | | | |
| | | [MASS = 264186 | | | | | | | | | |
| 2 | 2 | Rattus norvegicus (Rat) | | | | | | | | | |
| | | GM2 GANGLIOSIDE | | | | | | | | | |
| | | ACTIVATOR PROTEIN. | | | | | | | | | |
| | | [MASS = 21493 | | | | | | | | | |
| 2 | 2 | Rattus norvegicus (Rat) | | | | | | | | | |
| | | KINESIN-LIKE PROTEIN | | | | | | | | | |
| | | KIF15. [MASS = 159554 | | | | | | | | | |
| 2 | 2 | Rattus norvegicus (Rat) | | | | | | | | | |
| | | 29 KDA PROTEIN. | | | | | | | | | |
| | | [MASS = 29242 | | | | | | | | | |
| 2 | 2 | Rattus norvegicus (Rat) | | | | | | | | | |
| | | RIBOSOMAL PROTEIN | | | | | | | | | |
| | | L13A. [MASS = 23446 | | | | | | | | | |
| 2 | 2 | Rattus norvegicus (Rat) | | | | | | | | | |
| | | 60S RIBOSOMAL | | | | | | | | | |
| | | PROTEIN L8. | | | | | | | | | |
| | | [MASS = 27893 | | | | | | | | | |
| 2 | 2 | Rattus norvegicus (Rat) | | | | | | | | | |
| | | PREDICTED: TYROSINE | | | | | | | | | |
| | | KINASE RECEPTOR 1. | | | | | | | | | |
| | | [MASS = 125210 | | | | | | | | | |

Parada et al (J Proteome Res, 2005. 4:2420-8) identified 31 proteins within the rat e-CSF finding an abundance of extracellular matrix proteins, enzymes, and enzyme regulators, consistent with our study. We identified a much larger number of proteins within the CSF while identifying 24 of the 31 previously identified proteins. The 7 proteins that we did not find are the following: calreticulin, DJ-1, EEf1 g, laminin receptor 1, malate dehydrogenase 1, set beta isoform, and tyrosine 3-monooxygenase/tryptophan 5-monooxygenase activation protein theta polypeptide. The differences between our study and the study by Parada et al appear a consequence of methodology rather than sample differences. Parada et al chose for mass spectrometry the most prominent silver-stained e-CSF proteins resolved by two-dimensional electrophoresis, whereas we performed an analysis of the entire e-CSF separated by one-dimensional electrophoresis. Our one-dimensional approach enabled a more comprehensive analysis (one which would be unwieldy for an entire two dimensional gel), the study by Parada et al is complementary with this one as some proteins resolved in two dimensions would have a reduced likelihood of becoming suppressed due to co-migration in one dimension with abundant protein species such as albumin.

Our analyses are semi-quantitative, and we identified interesting differences between our various rat e-CSF samples. Apolipoprotein M is found in both E14.5 LV and E14.5 4thV but our analysis did not identify it in either E12.5 LV or E17.5 LV, phosphatidylethanolamine binding protein was found only in the E17.5 LV, collagen alpha I (XI) was identified in E14.5 and E17.5 LV, and phosphatase 2 (alpha isoform of regulatory subunit A) was found in E12.5 LV. Also, apolipoprotein D, an apolipoprotein that was not identified by Parada et al was identified only in the E14.5 4^{th}V.

### Comparison of Human and Rat CSF

In a comparison of proteins found in the human e-CSF to the proteins found in the rat e-CSF, we found that of the 188 proteins identified in the human e-CSF, 135 human proteins were identified in any one of the four samples of embryonic rat CSF. 83 of those proteins were present in all four samples of embryonic rat CSF. Table 1 includes the human proteins found common to rat CSF. We have indicated the specific rat samples in which each protein was identified. Out of the top 50 proteins found in the human CSF, 45 were also found in the rat CSF.

Proteins common to human and rat CSF presumably represent proteins related to fundamental CSF functions. For example, e-CSF contains many transport and carrier proteins including transferrin, albumin, alpha-fetoprotein, transthyretin, ceruloplasmin, and plasma retinol-binding protein that are all involved in either metal ion or vitamin transport through fluid or across cell membranes. There are a number of apolipoproteins involved in the transport and metabolism of lipids and fatty acids in the CSF as reported in this paper and by Parada et al (supra). There are also a large number of enzymes and protease inhibitors in the CSF that are involved in regulating immune response and maintaining homeostasis.

Other proteins common to rat and human CSF may play more specific roles in neurogenesis. One factor in the e-CSF is Amyloid Beta A4 Protein Precursor (APP), which we identified in rat CSF at E12.5, E14.5, and E17.5 and human CSF at CS20. This protein is normally present in brain and a soluble form is known to circulate in adult CSF (Palmert et al., Proc Natl Acad Sci USA, 1989. 86:6338-42). The soluble form of APP has been shown to stimulate proliferation of embryonic neural stem cells as well as adult neural progenitor cells from the subventricular zone (Caille et al., Development, 2004. 131:2173-81; Hayashi et al., Biochem Biophys Res Commun, 1994. 205:936-43; Ohsawa et al., Eur J Neurosci, 1999. 11:1907-13). APP may play a role during neurogenesis not only within the cell but may be released in the extracellular space and taken up in the CSF in order to diffuse throughout the CSF a play a function at more distant sites. Similarly, Tenascin, which we found in all CSF samples from rat and human from CS 20, is a secreted extracellular matrix glycoprotein implicated in axon guidance during development and regeneration (von Hoist et al., J Biol Chem, 2007. 282: 9172-81), which was recently shown to be expressed in progenitor cells in the ventricular zone of the developing brain. CSF contains multiple critical extracellular matrix factors including fibronectin, laminin, tenascin, fibulin, versican, and neurocan core protein. Because many of these factors can support or orient neuronal migration, they may be acting in the CSF as external cues for proliferating and differentiating neuronal progenitor cells.

Few proteins were identified that may be exclusive to rat or human e-CSF. The protein Pigment Epithelium Derived Factor (PEDF) was only found in the human e-CSF and is known to circulate in the adult CSF and is significantly decreased in CSF of patients with frontotemporal dementia (Davidsson et al., Brain Res Mol Brain Res, 2002. 109:128-33). This secreted serine protease inhibitor, known to be released by retinal pigment cell into the matrix, is a known neurotrophic protein involved in survival and potentially differentiation of specific neurons (Houenou et al., J Comp Neurol, 1999. 412:506-14). PEDF is known to act on photoreceptor cells but also may play a role in spinal motor neuronal survival. It is likely that PEDF is released by the photoreceptor cells into the matrix and taken up by the CSF and may act on cell types and neurons by diffusion through the CSF. Similarly, the Neuronal Cell Adhesion Molecule L1-Like Protein, also found only in the human e-CSF is known to play important roles in neurite outgrowth and neuronal survival (Hillenbrand et al., Eur J Neurosci, 1999. 11:813-26; Montag-Sallaz et al., Mol Cell Biol, 2002. 22:7967-81; Nishimune et al., J Neurosci Res, 2005. 80:593-9).

Conversely, we only observed the Extracellular Superoxide Dismutase, a protein known to remove free radicals that can be toxic to cells in rat e-CSF. One of the functions of the e-CSF may be the removal of toxins and toxin metabolic byproducts, and therefore it would important to have proteins within the CSF that help neutralize some of the toxic products released into the CSF. Additionally, we found in the rat e-CSF Mannose 6-phosphate/Insulin-like Growth Factor II Receptor (IGF2R), a soluble form of the receptor has been found in the serum, amniotic fluid and urine of both rodents and humans, affecting organ size based on its interaction with IGF2 and other factors (Causin et al., Biochem J, 1988. 252:795-9; Kiess et al., Proc Natl Acad Sci USA, 1987. 84:7720-4; MacDonald et al., J Biol Chem, 1989. 264:3256-61; Xu et al., J Clin Endocrinol Metab, 1998. 83:437-42; Zaina et al., J Biol Chem, 1998. 273:28610-6). Confirmation of these apparent differences would require Western blotting, and may lead to studies of their intriguing biological potential in the e-CSF.

Subcellular Localization of e-CSF Proteins

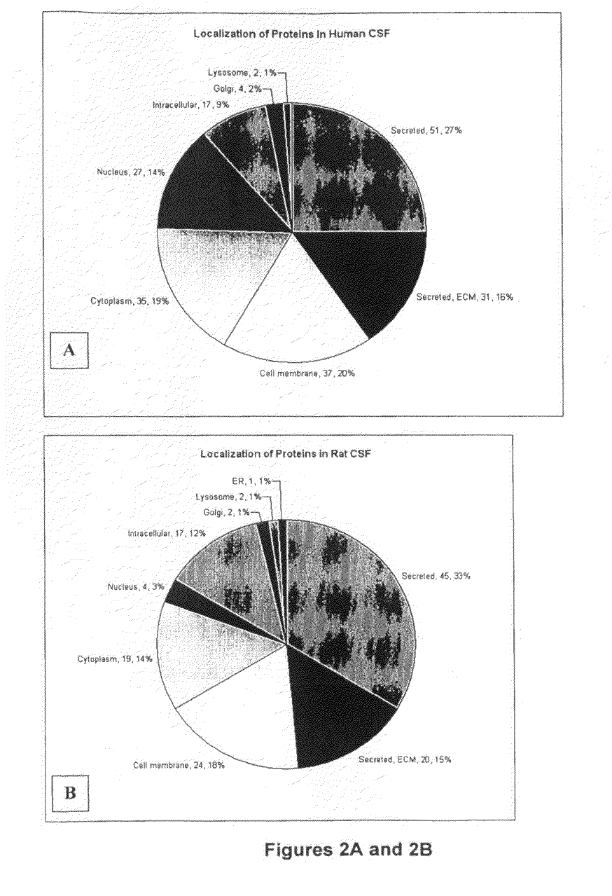

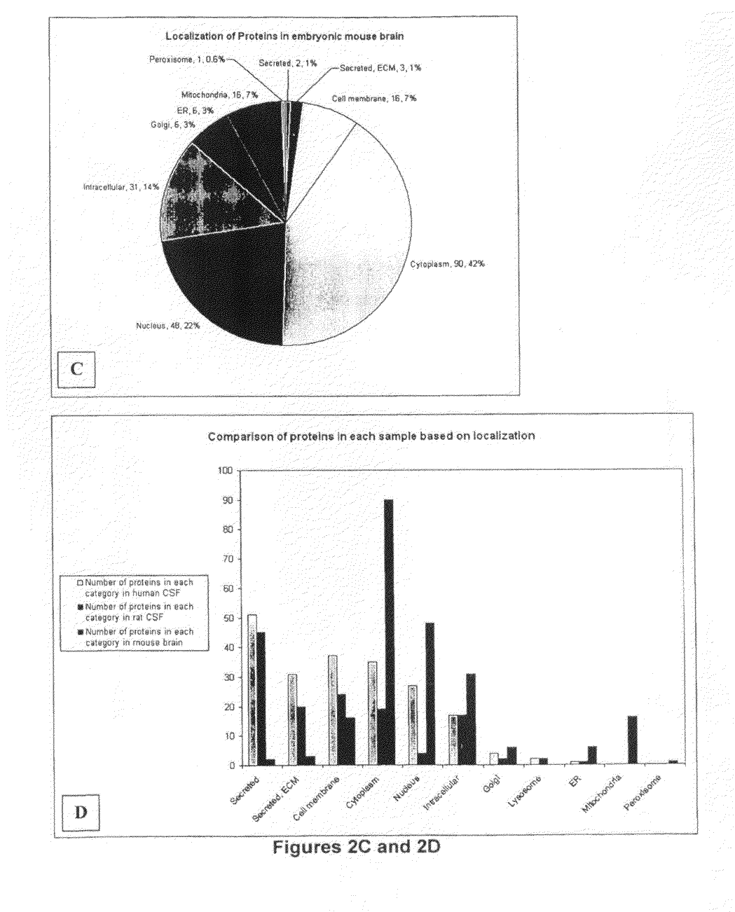

To compare the e-CSF of human and rat further we analyzed the 188 proteins found in the human e-CSF and the 137 proteins in the rat e-CSF present in all samples based on subcellular localization, molecular function, and biological process. The subcellular localization of each protein in the CSF is shown in Tables 1 and 3. The majority of proteins found in the human (FIG. 2A) and rat (FIG. 2B) e-CSF are secreted proteins which compose 27% and 33% of the total proteins found within the CSF respectively. The second most common localization of proteins found in the e-CSF of both humans and rats are cell membrane proteins, composing 20% and 18% respectively. The relatively high percentage of membrane proteins is consistent with the recent discovery of membrane bound particles in the CSF (Marzesco et al., J Cell Sci, 2005. 118:2849-58). Out of 188 proteins found in the human e-CSF, 19% are cytoplasmic proteins, 16% are secreted proteins found in the extracellular space or extracellular matrix (ECM), 14% are nuclear proteins, and 9% are intracellular proteins that could not be specifically localized to one compartment. Out of 137 proteins present in all rat e-CSF samples 14% are cytoplasmic proteins, 15% are ECM proteins, 3% are nuclear proteins, and 12% are intracellular proteins. As a control to assess subcellular localization in a protein population of embryonic brain, we chose to analyze the top 200 proteins identified from E16.5 mouse forebrain and midbrain in a previous study (Ballif et al., Mol Cell Proteomics, 2004. 3:1093-101). FIG. 2C shows that 42% of these proteins are in the cytoplasm, 22% nuclear, 14% intracellular, 7% at the cell membrane, and 7% mitochondrial. Strikingly no mitochondrial proteins were found in the CSF. Out of the 200 proteins analyzed from embryonic mouse brain, two are secreted and three are found in the extracellular space or matrix. FIG. 2D shows a graphical representation of the comparison of embryonic human CSF, rat CSF and mouse brain based on localization. The e-CSF as compared to brain tissue clearly has an abundant number of secreted proteins, extracellular matrix proteins, and cell membrane proteins as opposed to an overwhelming majority of cytoplasmic, nuclear, and mitochondrial proteins found in the brain tissue.

### Molecular Function and Biological Process

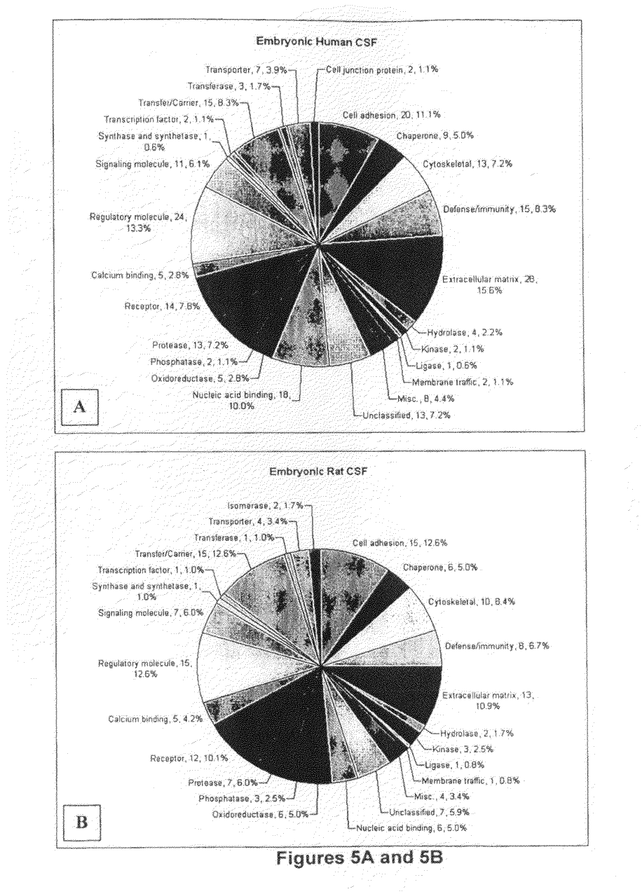

We used the PANTHER protein ontology database to classify the proteins into distinct categories of molecular function and biological process. Panther identified 180 out of 188 proteins with a total number of 237 functional hits for the human e-CSF, 119 out of 137 proteins with a total number of 155 functional hits for the rat e-CSF, and 191 out of 200 proteins with a total number of 234 functional hits for embryonic mouse brain. Table 1 shows the percentage of proteins assigned to each functional category in the embryonic human CSF, embryonic rat CSF, and E16.5 embryonic mouse brain. FIGS. 5A-5C represent functional classification of the samples as individual pie charts including the absolute number of proteins assigned to each function group.

TABLE 5
List of protein categories based on molecular function for embryonic
human CSF, rat CSF and mouse brain.

| | Percent | | Percent | | Percent |
|---|---|---|---|---|---|
| | proteins | | proteins | | proteins |
| | in each | | in each | | in each |
| Human CSF | category | Rat CSF | category | Mouse brain | category |
| Cell adhesion | 11.1% | Cell adhesion | 12.6% | Cell adhesion | 2.60% |
| Chaperone | 5.0% | Chaperone | 5.0% | Chaperone | 8.40% |
| Cytoskeletal | 7.2% | Cytoskeletal | 8.4% | Cytoskeletal | 11.50% |
| Defense/Immunity | 8.3% | Defense/Immunity | 6.7% | Defense/Immunity | 0.00% |
| Extracellular matrix | 15.6% | Extracellular matrix | 10.9% | Extracellular matrix | 0.50% |
| Hydrolase | 2.2% | Hydrolase | 1.7% | Hydrolase | 6.30% |
| Kinase | 1.1% | Kinase | 2.5% | Kinase | 2.60% |
| Ligase | 0.6% | Ligase | 0.8% | Ligase | 3.70% |
| Membrane traffic | 1.1% | Membrane traffic | 0.8% | Membrane traffic | 2.60% |
| Miscellaneous | 4.4% | Miscellaneous | 3.4% | Miscellaneous | 2.60% |
| Unclassified | 7.2% | Unclassified | 5.9% | Unclassified | 14.70% |
| Nucleic acid | 10.0% | Nucleic acid binding | 5.0% | Nucleic acid binding | 18.30% |
| binding | | | | | |
| Oxidoreductase | 2.8% | Oxidoreductase | 5.0% | Oxidoreductase | 4.70% |
| Phosphatase | 1.1% | Phosphatase | 2.5% | Phosphatase | 1.60% |
| Protease | 7.2% | Protease | 6.0% | Protease | 1.60% |
| Receptor | 7.8% | Receptor | 10.1% | Receptor | 2.10% |
| Calcium binding | 2.8% | Calcium binding | 4.2% | Calcium binding | 3.70% |
| Regulatory | 13.3% | Regulatory molecule | 12.6% | Regulatory molecule | 8.40% |
| molecule | | | | | |
| Signaling molecule | 6.1% | Signaling molecule | 6.0% | Signaling molecule | 1.60% |
| Synthase and | 0.6% | Synthase and | 1.0% | Synthase and | 2.60% |
| synthetase | | synthetase | | synthetase | |
| Transcription factor | 1.1% | Transcription factor | 1.0% | Transcription factor | 3.70% |
| Transfer/Carrier | 8.3% | Transfer/Carrier | 12.6% | Transfer/Carrier | 3.70% |
| Transferase | 1.7% | Transferase | 1.0% | Transferase | 4.70% |
| Transporter | 3.9% | Transporter | 3.4% | Transporter | 4.70% |
| Cell junction | 1.1% | Cell junction protein | 0% | Cell junction protein | 0% |
| protein | | | | | |
| Lyase | 0% | Lyase | 0% | Lyase | 1.60% |
| Ion channel | 0% | Ion channel | 0% | Ion channel | 1.60% |
| Isomerase | 0% | Isomerase | 1.7% | Isomerase | 1.00% |

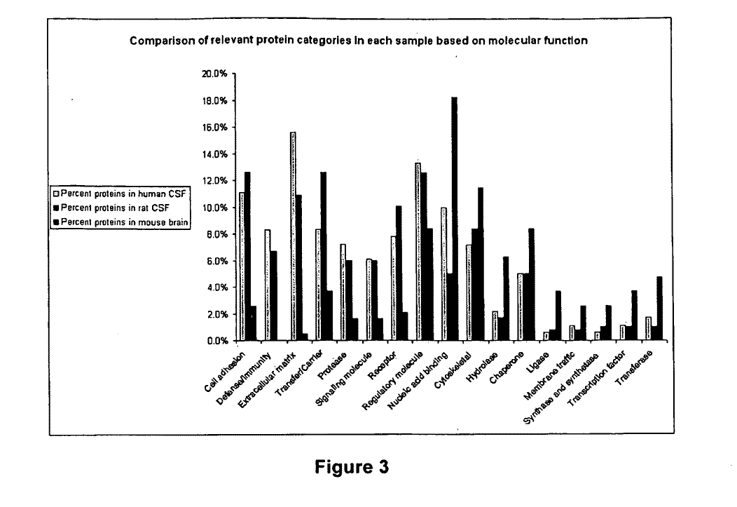

Panther analysis of molecular function reveals the majority of proteins found within the human and rat CSF share similar functional categories (Table 4, FIG. 3, and FIGS. 5A-5C). Proteins involved in extracellular matrix function make up, respectively, 16% and 11% of the majority of proteins found in the e-CSF of humans and rats. Other abundant categories of proteins found in the e-CSF include regulatory molecules such as protease inhibitors (human-13%, rat-13%), cell adhesion proteins (human-11%, rat-13%), nucleic acid binding proteins (human-10%, rat-5%), transfer/carrier proteins (human-8%, rat-13%), immune defense proteins (human-8%, rat-7%), and receptors (human-8%, rat-10%). The total number of enzymes also is a large component of the CSF. The embryonic human CSF has a total of 28 different functional enzymes (16%) and embryonic rat CSF has a total of 23 different functional enzymes (19%). Furthermore, the e-CSF is composed of a large number of different enzyme classes, and is particularly high in proteases (human-7%, rat-6%), and oxidoreductases (human-3%, rat-5%).

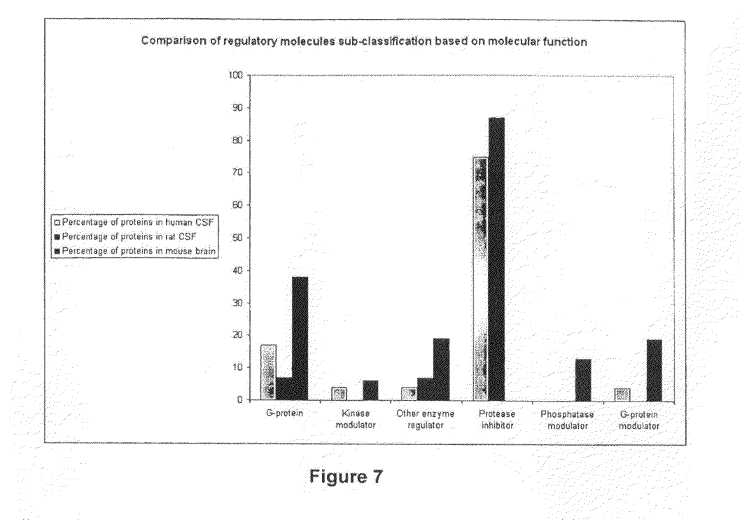

Panther analysis reveals distinct functional groups of proteins present in the CSF as compared to embryonic tissue. Protein categories in the embryonic human and rat CSF are quite similar and to control for random similarity in categorization based on molecular function we compared the CSF protein samples to a sample of 200 most abundant proteins in embryonic E16.5 mouse brain (Table 4). The comparison of relevant protein categories in each sample is shown in FIG. 3. The two largest categories of proteins in the embryonic mouse brain include nucleic acid binding proteins (18.3%) and cytoskeletal proteins (11.5%). Interestingly, proteins involved in defense and immunity which comprised 7-8% of e-CSF were completely absent from the top 200 proteins in the embryonic mouse brain sample. One category of proteins that appears to be similar in all three comparisons is the regulatory molecules (13.3% in human CSF, 12.6% in rat CSF, and 8.4% in mouse brain). We further classified the regulatory molecules into smaller categories and although the larger classification shows similar percentages of regulatory molecules, the sub-classification clearly distinguishes the e-CSF samples from the embryonic brain sample (FIG. 7). The majority of proteins in the e-CSF within the regulatory molecule class are sub-classified as protease inhibitors comprising 75% and 87% of proteins within the class in human and rat CSF respectively as compared to 0% in the mouse brain (FIG. 7). Based on molecular function the most abundant classes of protein present in the e-CSF are found to be proteins of the extracellular matrix, regulatory molecules, transfer/carrier proteins, cell adhesion proteins, and proteins involved in immunity and defense.

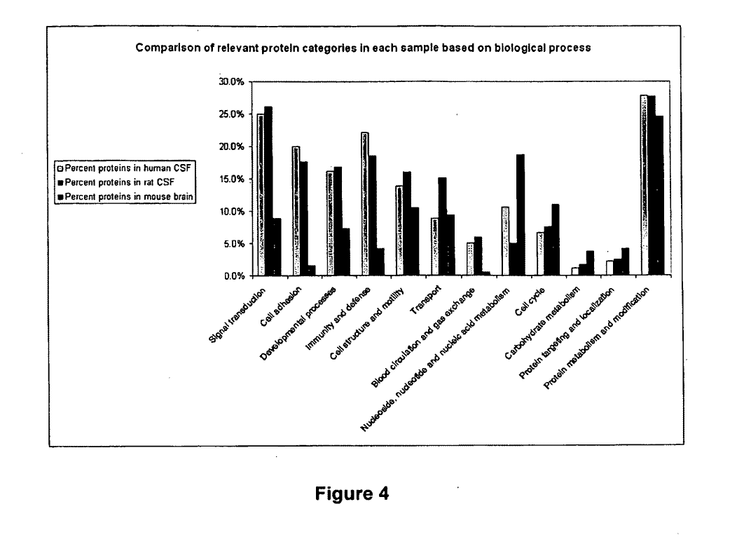

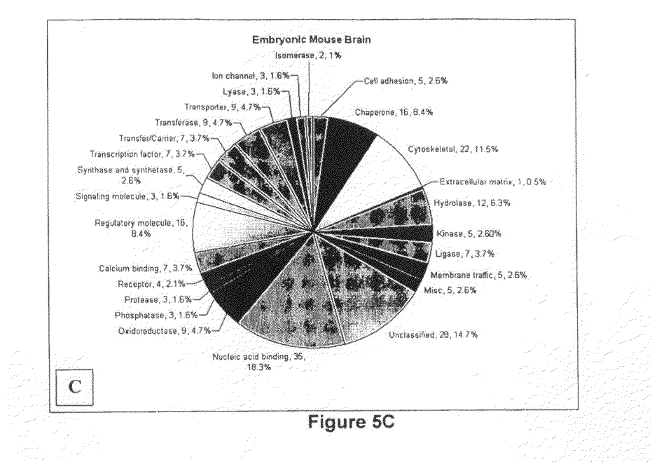

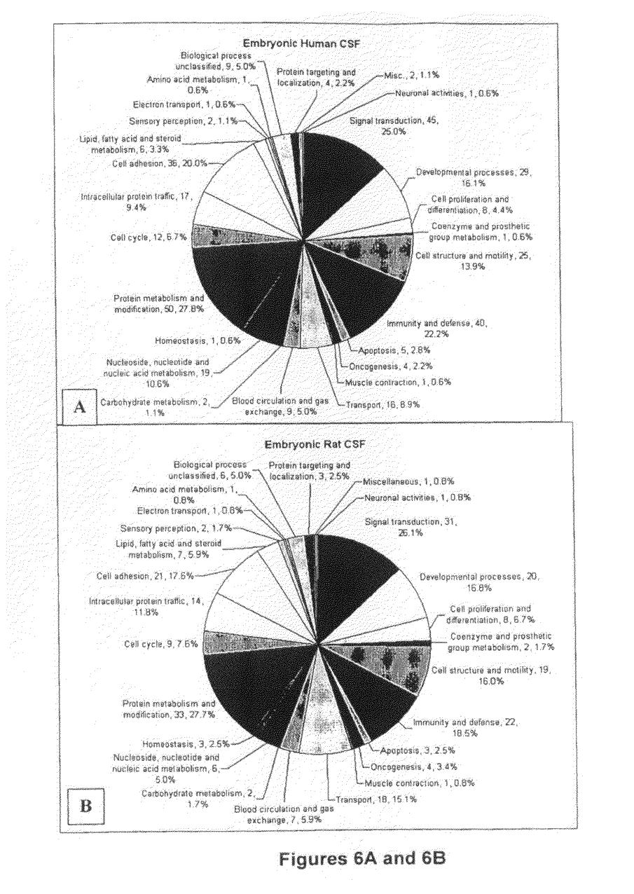

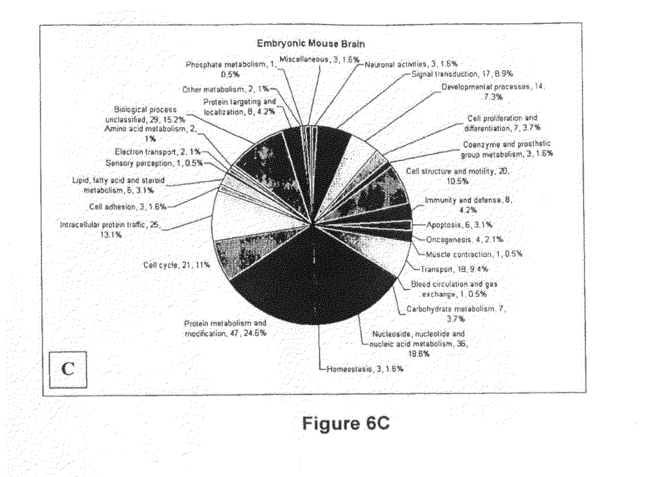

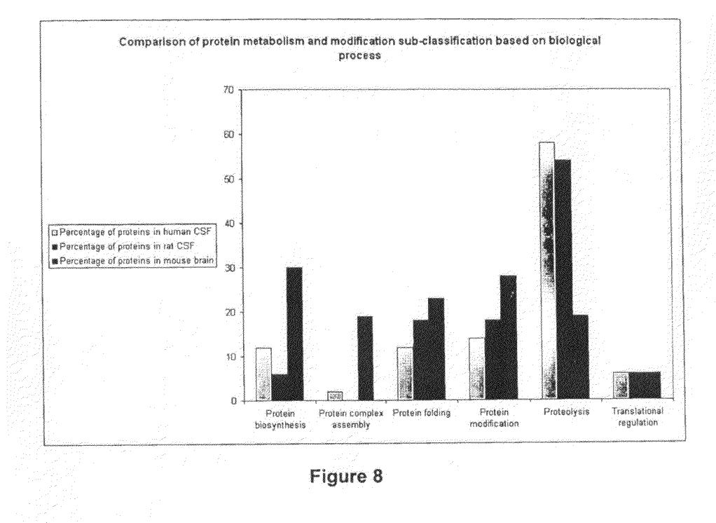

Panther analysis of proteins based on biological process reveals strong similarity between the embryonic human and rat CSF and differences between the CSF and the embryonic brain (Table 6, FIG. 4, and FIGS. 6A-6C). The five most abundant classes in both embryonic human and rat CSF are protein metabolism and modification, signal transduction, immunity and defense, cell adhesion, and developmental processes. The majority of proteins in the analysis of the embryonic mouse brain are involved in protein metabolism and modification, nucleic acid metabolism, intracellular protein traffic, cell cycle, and cell structure and motility. Comparing the analysis of the mouse brain with the e-CSF shows that the CSF samples contain proteins that are enriched for a number of various biological processes that are distinct from that of embryonic brain tissue (FIG. 4). Interestingly, all three samples are most abundant in proteins involved in protein metabolism and modification (FIG. 4). However, Panther analysis shows that CSF and brain show different types of proteins even among the same overall class (FIG. 8). Sub-classification of this category reveals the majority of proteins in the mouse brain involved in protein biosynthesis (30%) and protein modification (28%) with only 19% of proteins involved in proteolysis (FIG. 8). However in both the human and rat e-CSF the overwhelming majority of proteins in both samples are involved in proteolysis comprising 58% in humans and 54% in rats (FIG. 8). This class of biological processes includes the large number of protease inhibitors and proteases found within the CSF.

TABLE 6
List of protein categories based on biological process for
embryonic human CSF, rat CSF and mouse brain.

| | Percent | | Percent | | |
|---|---|---|---|---|---|
| | proteins in | | proteins in | | Percent |
| | each | | each | | proteins in each |
| Human CSF | category | Rat CSF | category | Mouse brain | category |
| Neuronal | 0.6% | Neuronal | 0.8% | Neuronal | 1.60% |
| activities | | activities | | activities | |
| Signal | 25.0% | Signal | 26.1% | Signal | 8.90% |
| transduction | | transduction | | transduction | |
| Developmental | 16.1% | Developmental | 16.8% | Developmental | 7.30% |
| processes | | processes | | processes | |
| Cell proliferation | 4.4% | Cell proliferation | 6.7% | Cell proliferation | 3.70% |
| and differentiation | | and differentiation | | and | |
| | | | | differentiation | |
| Coenzyme and | 0.6% | Coenzyme and | 1.7% | Coenzyme and | 1.60% |
| prosthetic group | | prosthetic group | | prosthetic group | |
| metabolism | | metabolism | | metabolism | |
| Cell structure and | 13.9% | Cell structure and | 16.0% | Cell structure and | 10.50% |
| motility | | motility | | motility | |
| Immunity and | 22.2% | Immunity and | 18.5% | Immunity and | 4.20% |
| defense | | defense | | defense | |
| Apoptosis | 2.8% | Apoptosis | 2.5% | Apoptosis | 3.10% |
| Oncogenesis | 2.2% | Oncogenesis | 3.4% | Oncogenesis | 2.10% |
| Muscle | 0.6% | Muscle | 0.8% | Muscle | 0.50% |
| contraction | | contraction | | contraction | |
| Transport | 8.9% | Transport | 15.1% | Transport | 9.40% |
| Blood circulation | 5.0% | Blood circulation | 5.9% | Blood circulation | 0.50% |
| and gas exchange | | and gas exchange | | and gas exchange | |
| Carbohydrate | 1.1% | Carbohydrate | 1.7% | Carbohydrate | 3.70% |
| metabolism | | metabolism | | metabolism | |
| Nucleoside, | 10.6% | Nucleoside, | 5.0% | Nucleoside, | 18.80% |
| nucleotide and | | nucleotide and | | nucleotide and | |
| nucleic acid | | nucleic acid | | nucleic acid | |
| metabolism | | metabolism | | metabolism | |
| Homeostasis | 0.6% | Homeostasis | 2.5% | Homeostasis | 1.60% |
| Protein | 27.8% | Protein | 27.7% | Protein | 24.60% |
| metabolism and | | metabolism and | | metabolism and | |
| modification | | modification | | modification | |
| Cell cycle | 6.7% | Cell cycle | 7.6% | Cell cycle | 11.00% |
| Intracellular | 9.4% | Intracellular | 11.8% | Intracellular | 13.10% |
| protein traffic | | protein traffic | | protein traffic | |
| Cell adhesion | 20.0% | Cell adhesion | 17.6% | Cell adhesion | 1.60% |
| Lipid, fatty acid | 3.3% | Lipid, fatty acid | 5.9% | Lipid, fatty acid | 3.10% |
| and steroid | | and steroid | | and steroid | |
| metabolism | | metabolism | | metabolism | |
| Sensory | 1.1% | Sensory | 1.7% | Sensory | 0.50% |
| perception | | perception | | perception | |
| Electron transport | 0.6% | Electron transport | 0.8% | Electron | 1.00% |
| | | | | transport | |
| Amino acid | 0.6% | Amino acid | 0.8% | Amino acid | 1.00% |
| metabolism | | metabolism | | metabolism | |
| Biological process | 5.0% | Biological process | 5.0% | Biological | 15.20% |
| unclassified | | unclassified | | process | |
| | | | | unclassified | |
| Protein targeting | 2.2% | Protein targeting | 2.5% | Protein targeting | 4.20% |
| and localization | | and localization | | and localization | |
| Miscellaneous | 1.1% | Miscellaneous | 0.8% | Miscellaneous | 1.60% |
| Phosphate | 0.0% | Phosphate | 0.0% | Phosphate | 0.50% |
| metabolism | | metabolism | | metabolism | |
| Other metabolism | 0.0% | Other metabolism | 0.0% | Other | 1.00% |
| | | | | metabolism | |

The similarities between the embryonic human and rat CSF are apparent when the proteins are classified into groups and analyzed on the basis of subcellular localization, molecular function, and biological process. Based on the functional characteristics of the proteins found in the e-CSF, the CSF is a heterogeneous mixture of many types of classes of proteins with varying functions. The e-CSF is far more complex than previously thought. This may be due to active secretion from the choroid plexus into the CSF, or from the contents within the extracellular membrane bound particles that are present in the rodent CSF during development, or potentially to aposomes budding from the choroid plexus and floating within the CSF that have been shown previously to support protein translation (Saunders et al., Cell Mol Neurobiol, 2000. 20:29-40; Agnew et al., Cell Tissue Res, 1980. 208:261-81; Gudeman et al., J Neurosci Res, 1989. 24:184-91).

Although we did not find the growth factor FGF-2 as reported by Martin et al (Dev Biol, 2006. 297:402-16), many growth factors are in low abundance and are of smaller molecular weight making them more challenging to identify by multiple peptide assignments using mass spectrometry on a complex mixture.

Cortical Explants in e-CSF

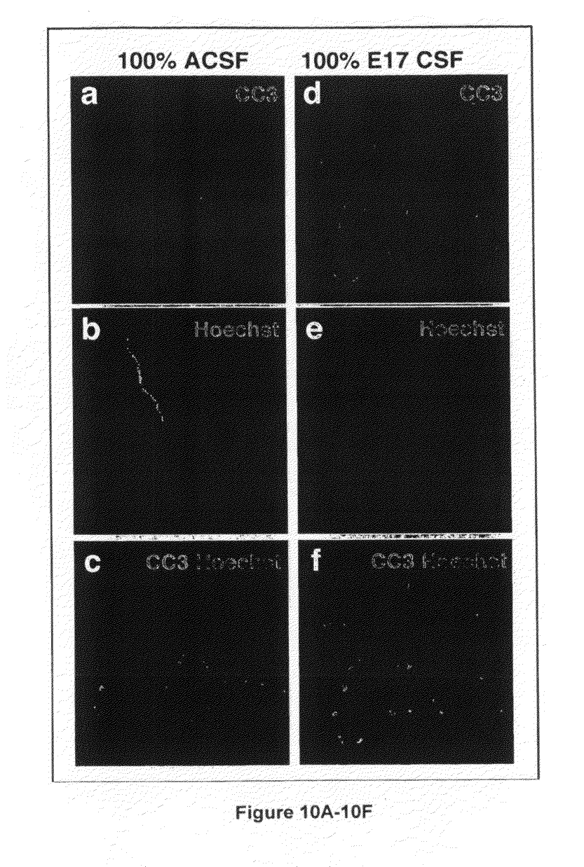

Cortical explants can survive and proliferate in the presence of e-CSF. Embryonic day 16 (E16) rat cortical explants cultured with 100% E17 CSF for 24 hours, without additional exogenous media or factors, retained tissue architecture, cell proliferation, and cell viability, approximating in vivo E17 rat cortex (FIGS. 9B, 9C, 9E, and 9F). In contrast, culturing E16 explants with 100% artificial CSF (ACSF) failed to maintain the integrity of the embryonic cortical tissue, as reflected by decreased proliferation and mitotic activity, disorganized neuronal morphology, and a striking increase in cell death (FIGS. 9D and 9G and FIGS. 10A-10F). Thus, the embryonic CSF proteome provides an endogenous signaling milieu of essential growth and survival factors for the developing cortex.

Comparison of E13 and E17 Rat e-CSF

The primary source of CSF is the choroid plexus, a highly vascularized secretory epithelial tissue that extends into the ventricles. To determine if the embryonic choroid-plexus-derived-CSF provides support and instructive cues to the developing cortex we compared CSF from E13 embryos (pre choroid plexus formation) with that from E17 embryos (post choroid plexus formation). E17 CSF increased the frequency of PH3-labeled proliferating cells in E16 cortical explants compared to explants cultured with E13 CSF (E17 mean: 44.1±±1.43; E13 mean: 25±4.2; n=4; p<0.05) (FIGS. 9H, 9I, and 9N) with a greater than 2.5-fold increase in PH3-positive-staining cells along the ventricular zone (VZ) (E17 mean: 32.3±0.79; E13 mean: 12.8±3.9; n=4, p<0.05) (FIG. 9O). To determine the identity of mitotic cells, explants were stained with an anti-phosphorylated Vimentin 4A4 antibody, an established marker of proliferating neural progenitor cells (Anthony et al., Neuron 41, 881-90 (2004); Weissman et al., Cereb Cortex 13, 550-9 (2003)). E16 explants cultured in E17 CSF revealed increased Vimentin 4A4-positive-staining cells per explant compared to explants grown in E13 CSF (E17 mean: 38.4±1.1; E13 mean: 13.9±2.2; n=3, p<0.05) (FIG. 1j, k, p). No difference in the number of Tbr2-positive-staining cells undergoing division was observed (data not shown). Taken together, these data suggest that age-dependent differences in embryonic CSF signals are both supportive and instructive for precursor proliferation in the developing cortex.

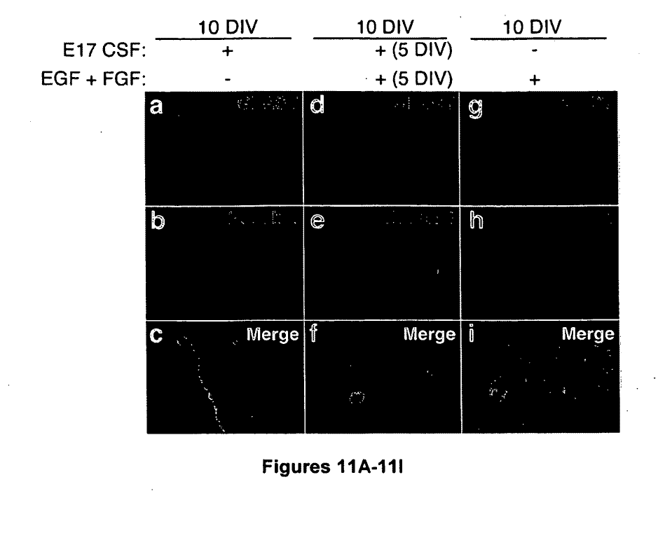

CSF was then determined to maintain and stimulate proliferation of primary dissociated cortical progenitors cultured as neurospheres, an in vitro experimental model for neural stem cells. Primary neurospheres derived from E14 rat embryos were dissociated, plated at clonal density, and cultured with CSF collected from E13 or E17 embryos. Both E13 and E17 CSF supported the generation of small neurospheres composed primarily of GLAST-positive-staining cells in the absence of supplemental FGF and EGF for 10 days in vitro (DIV) (FIGS. 9Q-9Y). Neurospheres failed to form in the presence of ACSF. Consistent with our explant experiments, cells cultured in E17 CSF generated not only increased numbers of neurospheres (FIG. 9Z), but also larger spheres (data not shown), indicating that E17 CSF contains instructive proliferative signals. In addition, neurospheres grown in CSF retained responsiveness to FGF and EGF, indicating that the CSF is maintaining the stem cells in an uncommitted fate (FIGS. 11A-11I).

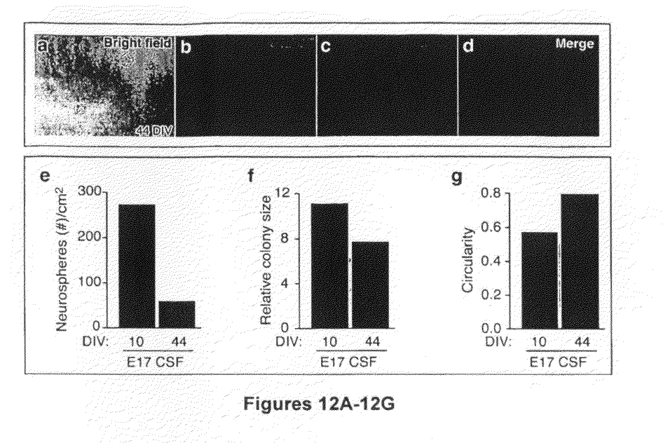

Both E13 and E17 CSF maintain viable GLAST-positive-staining neurospheres (FIGS. 12A-12G) after 44 DIV, while E17 CSF promoted the survival of an increased number of neurospheres compared to E13 CSF. Thus, embryonic CSF is sufficient for maintaining and stimulating proliferating cortical progenitor cells.

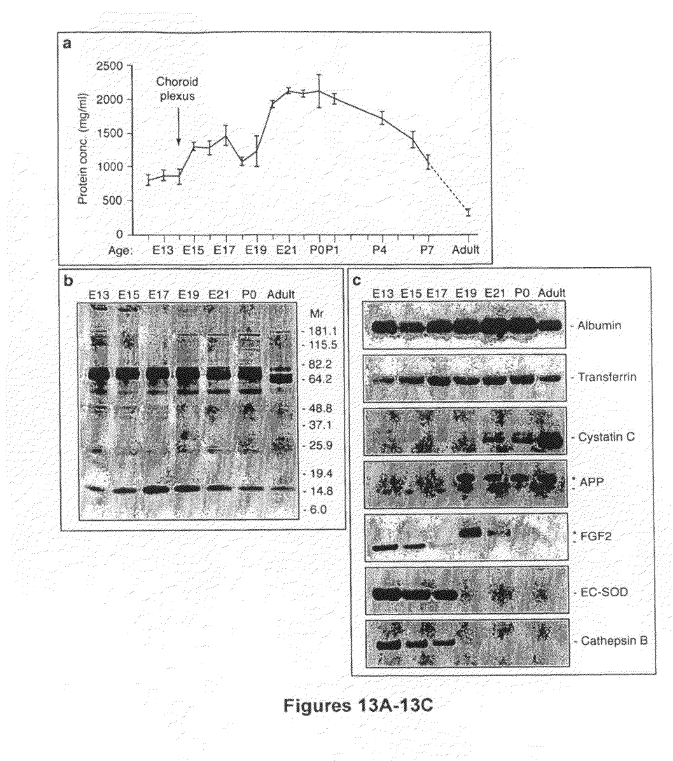

We next characterized the embryonic CSF proteome to determine how the CSF drives the proliferation of cortical progenitor cells. Total CSF protein concentration increased from E12 on, peaked at birth (P0) and declined into adulthood (FIG. 13A). We visualized the overall protein composition of CSF by silver staining, and observed a graded transition of CSF constituents from E13 to adulthood (FIG. 13B). Immunoblot analysis of proteins identified by tandem mass spectrometry (LC-MS/MS) (Zappaterra et al., J Proteome Res 6, 3537-48 (2007)) revealed dynamic changes in different classes of proteins in CSF during development (FIG. 13C and data not shown). For example, several proteins known to regulate proliferation of neural progenitors including transferrin, cystatin C, FGF2, and soluble isoforms of amyloid precursor protein (sAPP) were expressed throughout development and, in some cases, in the adult CSF (FIG. 13C). Other proteins involved in tissue homeostasis, such as the antioxidant and free radical scavenger extracellular super oxide dismutase (EC-SOD, Sod3), and the protease Cathepsin B were robustly expressed early in development and rapidly downregulated thereafter (FIG. 13C). Together with the CSF proteome, these proteins contribute to the role of CSF in development by providing essential growth-promoting cues to the developing cortex.

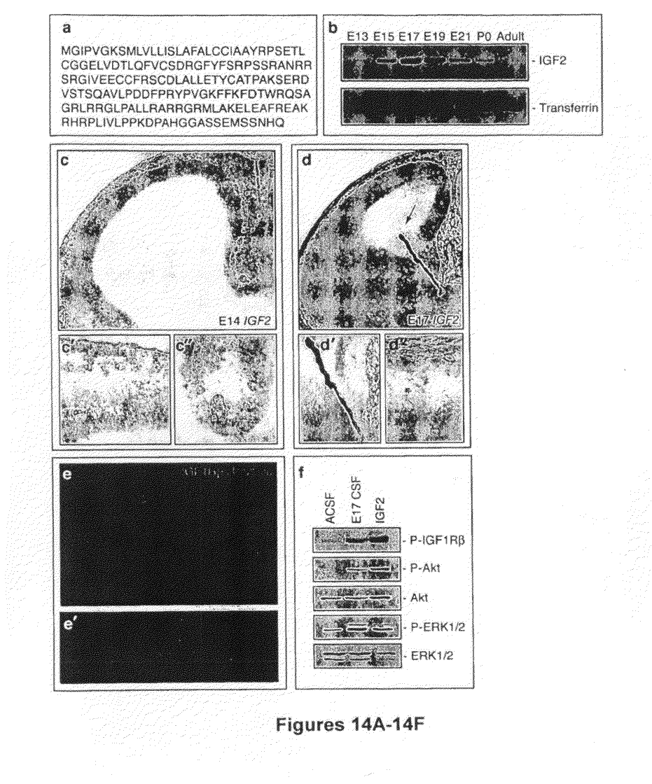

To investigate the distinct effects of embryonic CSF at different developmental stages, we performed extensive LC-MS/MS analyses on increased volumes of E17 CSF. From these E17 rat proteome analyses, we identified several peptides corresponding to Insulin-like growth factor 2 (IGF2) in the CSF (FIG. 14A and Table 7). IGF2 is a particularly compelling CSF resident protein given the crucial role of IGF signaling in prenatal growth and brain size, as well as in regulating neural progenitor cell division (Randhawa et al., Mol Genet Metab 86, 84-90 (2005); Hodge et al., J Neurosci 24, 10201-10 (2004); Baker et al., Cell 75, 73-82 (1993)). IGF2 is also essential in the embryonic stem (ES) cell niche (Bendall et al., Nature 448, 1015-21 (2007)). Interestingly, we found that IGF2 is transiently expressed in the CSF during development. IGF2 was first detected at E13 and maximally expressed during cortical neurogenesis (E15-E19), after which its expression declined postnatally (FIG. 14B). The dynamic availability of Igf2 in the embryonic CSF raised the possibility that IGF signaling may contribute to the differential capacity of embryonic CSF between E13 and E17 to support cortical neural progenitor proliferation (Bendall et al., supra). We therefore sought to characterize the role IGF2 in neural development, as outlined below.

TABLE 7

| Protein | MW |
|---|---|
| 14-3-3 protein | 28037 |
| beta/alpha | |
| 14-3-3 protein | 29345 |
| epsilon | |
| 14-3-3 protein | 28342 |
| gamma | |
| 14-3-3 protein | 28063 |
| theta | |
| 14-3-3 protein | 27942 |
| zeta/delta | |
| 18 kDa protein | 18663 |
| 20 kDa protein | 20033 |
| 21 kDa protein | 21585 |
| 26S protease | 45797 |
| regulatory | |
| subunit 8 | |
| 29 kDa protein | 29540 |
| 31 kDa protein | 31415 |
| 32 kDa protein | 32525 |
| 32 kDa protein | 32833 |
| 33 kDa protein | 32943 |
| 35 kDa protein | 35005 |
| 35 kDa protein | 35717 |
| 35 kDa protein | 35586 |
| 38 kDa protein | 38434 |
| 38 kDa protein | 38434 |
| 38 kDa protein | 38510 |
| 39 kDa protein | 39428 |
| 40S ribosomal | 18602 |
| protein S11 | |
| 40S ribosomal | 26845 |
| protein S3 | |
| 40S ribosomal | 30042 |
| protein S3a | |
| 40S ribosomal | 29694 |
| protein S4, X | |
| isoform | |
| 40S ribosomal | 22918 |
| protein S5 | |
| 40S ribosomal | 28851 |
| protein S6 | |
| 40S ribosomal | 22127 |
| protein S7 | |
| 40S ribosomal | 24359 |
| protein S8 | |
| 40S ribosomal | 32807 |
| protein SA | |
| 42 kDa protein | 42846 |
| 43 kDa protein | 45093 |
| 45 kDa protein | 44917 |
| 45 kDa protein | 45796 |
| 45 kDa protein | 45796 |
| 45 kDa protein | 45796 |
| 46 kDa protein | 46534 |
| 47 kDa heat | 46631 |
| shock protein | |
| precursor | |
| 50 kDa protein | 50675 |
| 50 kDa protein | 50450 |
| 51 kDa protein | 51503 |
| 60S acidic | 34386 |
| ribosomal | |
| protein P0 | |
| 60S ribosomal | 20349 |
| protein L11 | |
| 60S ribosomal | 24129 |
| protein L15 | |
| 60S ribosomal | 34555 |
| protein L5 | |
| 60S ribosomal | 30386 |
| protein L7 | |
| 90 kDa protein | 90936 |
| Aa1064 | 538071 |
| Ab2-076 | 104278 |
| Ab2-379 | 62360 |
| Ac1873 | 87398 |
| Actin, alpha | 42393 |
| skeletal muscle | |
| Actin, gamma- | 42276 |
| enteric smooth | |
| muscle | |
| Adenine | 20049 |
| phosphoribosyltransferase | |
| Adenosylhomocysteinase | 47920 |
| ADP-ribosylation | 20526 |
| factor 3 | |
| ADP-ribosylation | 20379 |
| factor 4 | |
| ADP-ribosylation | 20065 |
| factor 6 | |
| Alcohol | 36602 |
| dehydrogenase | |
| Aldose | 36122 |
| reductase | |
| Alpha-1- | 46306 |
| antiproteinase | |
| precursor | |
| Alpha-2 | 46522 |
| antiplasmin | |
| Alpha-2-globin | 15456 |
| chain | |
| Alpha-2-HS- | 38781 |
| glycoprotein | |
| precursor | |
| AMBP protein | 39763 |
| precursor | |
| AMBP protein | 39763 |
| precursor | |
| Angiotensin II | 57615 |
| type 1A receptor | |
| associated | |
| protein | |
| Angiotensinogen | 52209 |
| precursor | |
| APEX | 32752 |
| Apolipoprotein | 30119 |
| A-I | |
| Apolipoprotein | 11496 |
| A-II precursor | |
| Apolipoprotein | 44456 |
| A-IV precursor | |
| Apolipoprotein E | 35810 |
| precursor | |
| Apolipoprotein M | 21855 |
| precursor | |
| Ba1-667 | 109614 |
| Beta-1,3-N- | 42471 |
| acetylglucosaminyl- | |
| transferase lunatic | |
| fringe | |
| Beta-2-glycoprotein I | 34338 |
| precursor | |
| Beta-2-microglobulin | 13834 |
| precursor | |
| Calcyclin binding | 26655 |
| protein | |
| cAMP-dependent | 43191 |
| protein kinase type I- | |
| alpha regulatory | |
| subunit | |
| Carbonic anhydrase 2 | 29153 |
| Casein kinase II, | 45187 |
| alpha chain | |
| Cathepsin B, | 38457 |
| preproprotein | |
| Cathepsin L | 38231 |
| precursor | |
| Cathepsin Z | 34879 |
| precursor | |
| Cd81 protein | 26589 |
| Cell division control | 34191 |
| protein 2 homolog | |
| Cfh protein | 46732 |
| Cofilin-1 | 25102 |
| Collagen alpha 1(V) | 185292 |
| chain precursor | |
| Collagen alpha 1(XI) | 171893 |
| chain precursor | |
| Collagen alpha 2(I) | 130076 |
| chain precursor | |
| Collagen alpha 2(I) | 130076 |
| chain precursor | |
| Collagen alpha1 | 138912 |
| Complement C1q | 26817 |
| subcomponent, B | |
| chain precursor | |
| Complement C1q | 25971 |
| subcomponent, C | |
| chain precursor | |
| Connective tissue | 39981 |
| growth factor | |
| precursor | |
| Creatine kinase B- | 42997 |
| type | |
| Cystatin C | 15665 |
| Cytosolic malate | 36654 |
| dehydrogenase | |
| DJ-1 protein | 20202 |
| EGF-containing | 46847 |
| fibulin-like | |
| extracellular matrix | |
| protein 2 | |
| Elongation factor 1- | 50456 |
| alpha 1 | |
| Epsilon 1 globin | 16161 |
| Epsilon-sarcoglycan | 50239 |
| Eukaryotic | 36262 |
| translation initiation | |
| factor 2 subunit 1 | |
| Eukaryotic | 40076 |
| translation initiation | |
| factor 3, subunit 3 | |
| gamma, 40 kDa | |
| Extracellular | 27019 |
| superoxide | |
| dismutase [Cu—Zn] | |
| precursor | |
| FAM3C-like protein | 24999 |
| Fatty acid-binding | 15018 |
| protein, brain | |
| Fetuin-B precursor | 42388 |
| Fructose- | 39677 |
| bisphosphate | |
| aldolase A | |
| Fructose- | 39551 |
| bisphosphate | |
| aldolase C | |
| Galactokinase 1 | 42832 |
| Gelsolin | 86856 |
| Glutathione S- | 23536 |
| transferase P | |
| Glyceraldehyde-3- | 36079 |
| phosphate | |
| dehydrogenase | |
| GTP-binding nuclear | 24594 |
| protein Ran | |
| GTP-binding nuclear | 24622 |
| protein Ran, testis- | |
| specific isoform | |
| Guanine nucleotide- | 35875 |
| binding protein beta | |
| subunit 2-like 1 | |
| Guanine nucleotide- | 38044 |
| binding protein | |
| G(I)/G(S)/G(T) beta | |
| subunit 1 | |
| Hemoglobin alpha-1 | 15368 |
| and alpha-2 chains | |
| Hemoglobin beta | 15962 |
| chain, major-form | |
| Hemoglobin beta | 15965 |
| chain, minor-form | |
| Heterogeneous | 34252 |
| nuclear | |
| ribonucleoprotein A1 | |
| Heterogeneous | 32914 |
| nuclear | |
| ribonucleoprotein C | |
| Heterogeneous | 46072 |
| nuclear | |
| ribonucleoprotein F | |
| Hypothetical | 32346 |
| LOC315594 | |
| Hypothetical | 35039 |
| LOC316842 | |
| Hypothetical | 33553 |
| LOC363644 | |
| Hypothetical protein | 59591 |
| Hypothetical protein | 37353 |
| Hypothetical protein | 34736 |
| Hypothetical protein | 34748 |
| LOC311078 | |
| Hypothetical protein | 33918 |
| RGD1308228_predicted | |
| HYRAC | 32094 |
| Ig lambda-2 chain C | 25750 |
| region | |
| Insulin-like growth | 33966 |
| factor binding protein | |
| 2 precursor | |
| Insulin-like growth | 28886 |
| factor binding protein | |
| 4 precursor | |
| Insulin-like growth | 20542 |
| factor II precursor | |
| Isocitrate | 47076 |
| dehydrogenase | |
| [NADP] cytoplasmic | |
| Lactadherin | 48553 |
| precursor | |
| LIM and SH3 | 30369 |
| domain protein 1 | |
| L-lactate | 36735 |
| dehydrogenase A | |
| chain | |
| L-lactate | 36766 |
| dehydrogenase B | |
| chain | |
| LOC367586 | 51633 |
| protein | |
| LOC500183 | 26034 |
| protein | |
| LRRGT00147 | 108859 |
| Lysozyme | 17186 |
| Mannose-binding | 81703 |
| protein | |
| associated serine | |
| protease-1 | |
| Masp1 protein | 45168 |
| Matrix Gla-protein | 12208 |
| precursor | |
| Metalloproteinase | 24478 |
| inhibitor 1 | |
| precursor | |
| Metalloproteinase | 25041 |
| inhibitor 2 | |
| precursor | |
| Multifunctional | 47706 |
| protein ADE2 | |
| Neurexin-2-alpha | 187392 |
| precursor | |
| Neutrophil | 10452 |
| antibiotic peptide | |
| NP-4 precursor | |
| NG,NG- | 31694 |
| dimethylarginine | |
| dimethylaminohydrolase 1 | |
| Nuclear migration | 38412 |
| protein nudC | |
| Pancreatic | 52181 |
| triacylglycerol | |
| lipase precursor | |
| Peptidyl-prolyl cis- | 17971 |
| trans isomerase A | |
| Peptidyl-prolyl cis- | 23859 |
| trans isomerase | |
| B precursor | |
| Peptidylprolyl | 23066 |
| isomerase C | |
| Peroxiredoxin 1 | 22337 |
| Peroxiredoxin 2 | 21823 |
| Phosphatidylethanolamine- | 20784 |
| binding | |
| protein | |
| Phosphoglycerate | 44937 |
| kinase 1 | |
| Phosphoglycerate | 45409 |
| kinase 2 | |
| Phosphoglycerate | 28571 |
| mutase 1 | |
| Phosphoglycerate | 28795 |
| mutase 2 | |
| Phosphoserine | 40969 |
| aminotransferase 1 | |
| Plasma | 25678 |
| glutathione | |
| peroxidase | |
| precursor | |
| Platelet-activating | 47109 |
| factor | |
| acetylhydrolase | |
| IB alpha subunit | |
| PREDICTED: | 66492 |
| ATPase, H+ | |
| transporting, | |
| lysosomal | |
| accessory protein 2 | |
| PREDICTED: | 97048 |
| dystroglycan 1 | |
| PREDICTED: | 73357 |
| eukaryotic | |
| translation | |
| elongation factor | |
| 1 gamma | |
| PREDICTED: | 44883 |
| Glycoprotein-4- | |
| beta- | |
| galactosyltransferase 2 | |
| PREDICTED: | 73188 |
| multiple inositol | |
| polyphosphate | |
| histidine | |
| phosphatase 1 | |
| PREDICTED: | 176810 |
| nidogen 2 | |
| (predicted) | |
| PREDICTED: | 182884 |
| procollagen type | |
| XI alpha 1 | |
| PREDICTED: | 37413 |
| proteasome | |
| (prosome, | |
| macropain) | |
| subunit, beta type 5 | |
| PREDICTED: | 50766 |
| retinol binding | |
| protein 4, plasma | |
| PREDICTED: | 62084 |
| serine (or | |
| cysteine) | |
| proteinase | |
| inhibitor, clade F, | |
| member 2 | |
| (predicted) | |
| PREDICTED: | 25236 |
| similar to 25 kDa | |
| FK506-binding | |
| protein | |
| PREDICTED: | 22761 |
| similar to 40S | |
| ribosomal protein | |
| S9 | |
| PREDICTED: | 25560 |
| similar to 60S | |
| ribosomal protein | |
| L7 | |
| PREDICTED: | 28385 |
| similar to 60S | |
| ribosomal protein | |
| L8 | |
| PREDICTED: | 21971 |
| similar to 60S | |
| ribosomal protein | |
| L9 | |
| PREDICTED: | 79786 |
| similar to Ab2-076 | |
| PREDICTED: | 39999 |
| similar to actin | |
| related protein ⅔ | |
| complex subunit 2 | |
| PREDICTED: | 62610 |
| similar to Actin, | |
| cytoplasmic 2 | |
| (Gamma-actin) | |
| PREDICTED: | 370838 |
| similar to alpha 3 | |
| type VI collagen | |
| isoform 1 | |
| precursor | |
| PREDICTED: | 47602 |
| similar to Alpha | |
| enolase (2- | |
| phospho-D- | |
| glycerate hydro- | |
| lyase) (Non- | |
| neural enolase) | |
| (NNE) | |
| PREDICTED: | 46831 |
| similar to Alpha | |
| enolase (2- | |
| phospho-D- | |
| glycerate hydro- | |
| lyase) (Non- | |
| neural enolase) | |
| (NNE) | |
| PREDICTED: | 23384 |
| similar to alpha | |
| NAC/1.9.2. | |
| protein | |
| PREDICTED: | 51683 |
| similar to Alpha- | |
| centractin | |
| (Centractin) | |
| (Centrosome- | |
| associated actin | |
| homolog) (Actin- | |
| RP | |
| PREDICTED: | 10695 |
| similar to | |
| Apolipoprotein C2 | |
| PREDICTED: | 18823 |
| similar to cofilin | |
| PREDICTED: | 194644 |
| similar to collagen | |
| alpha 2(IV) chain | |
| precursor - | |
| mouse | |
| PREDICTED: | 49756 |
| similar to ELAV | |
| (embryonic lethal, | |
| abnormal vision, | |
| Drosophila)-like 1 | |
| (Hu antigen R) | |
| PREDICTED: | 38691 |
| similar to | |
| Glyceraldehyde- | |
| 3-phosphate | |
| dehydrogenase | |
| (GAPDH) | |
| PREDICTED: | 22732 |
| similar to | |
| glyceraldehyde-3- | |
| phosphate | |
| dehydrogenase | |
| (phosphorylating) | |
| (EC 1.2.1.12) - m | |
| PREDICTED: | 54235 |
| similar to | |
| glycosyltransferase | |
| 28 domain | |
| containing 1 | |
| PREDICTED: | 506188 |
| similar to heparan | |
| sulfate | |
| proteoglycan 2 | |
| (perlecan) | |
| PREDICTED: | 102890 |
| similar to | |
| heterogeneous | |
| nuclear | |
| ribonucleoprotein | |
| A0 | |
| PREDICTED: | 46478 |
| similar to | |
| heterogeneous | |
| nuclear | |
| ribonucleoprotein | |
| D-like | |
| PREDICTED: | 40077 |
| similar to | |
| heterogeneous | |
| nuclear | |
| ribonucleoprotein | |
| H3 isoform a | |
| PREDICTED: | 37486 |
| similar to | |
| Heterogeneous | |
| nuclear | |
| ribonucleoproteins | |
| A2/B1 (hnRNP | |
| A2/hnRNP B1) | |
| PREDICTED: | 25049 |
| similar to High | |
| mobility group | |
| protein 1 (HMG-1) | |
| (Amphoterin) | |
| (Heparin-binding | |
| protein p | |
| PREDICTED: | 29708 |
| similar to IGFBP- | |
| like protein | |
| PREDICTED: | 26598 |
| similar to | |
| immunoglobulin | |
| light chain | |
| PREDICTED: | 64111 |
| similar to keratin | |
| 6 alpha | |
| PREDICTED: | 151853 |
| similar to | |
| mKIAA1631 | |
| protein | |
| PREDICTED: | 307380 |
| similar to myosin- | |
| VIIb | |
| PREDICTED: | 298228 |
| similar to | |
| OTTHUMP00000060196 | |
| PREDICTED: | 650022 |
| similar to | |
| OTTHUMP00000065631 | |
| PREDICTED: | 43632 |
| similar to | |
| Phosphoglycerate | |
| kinase 1 | |
| PREDICTED: | 35269 |
| similar to | |
| poly(rC) binding | |
| protein 2 | |
| PREDICTED: | 38011 |
| similar to | |
| Poly(rC)-binding | |
| protein 1 (Alpha- | |
| CP1) (hnRNP- | |
| E1) | |
| PREDICTED: | 117067 |
| similar to | |
| procollagen, type | |
| IX, alpha 1 | |
| PREDICTED: | 34868 |
| similar to | |
| Proteasome | |
| subunit alpha | |
| type 7-like | |
| PREDICTED: | 32587 |
| similar to purine- | |
| nucleoside | |
| phosphorylase | |
| PREDICTED: | 33227 |
| similar to | |
| pyrophosphatase | |
| PREDICTED: | 35237 |
| similar to RAB5B | |
| protein | |
| PREDICTED: | 18522 |
| similar to | |
| ribosomal protein | |
| L14 | |
| PREDICTED: | 42305 |
| similar to RIKEN | |
| cDNA | |
| 4732495G21 | |
| gene | |
| PREDICTED: | 50964 |
| similar to Tubulin | |
| alpha-2 chain | |
| (Alpha-tubulin 2) | |
| PREDICTED: | 44993 |
| similar to tubulin, | |
| beta, 2 | |
| PREDICTED: | 40735 |
| similar to | |
| Vesicular | |
| integral- | |
| membrane | |
| protein VIP36 | |
| precursor (Lectin, | |
| mannose-binding | |
| PREDICTED: | 124213 |
| thrombospondin 4 | |
| Proliferating cell | 29091 |
| nuclear antigen | |
| Proliferation- | 43999 |
| associated 2G4, | |
| 38 kDa | |
| ProProtein | 82452 |
| convertase | |
| subtilisin/kexin | |
| tyPe 9 | |
| Proteasome | 25908 |
| (Prosome, | |
| macroPain) | |
| subunit, beta | |
| tyPe 4 | |
| Proteasome | 25909 |
| subunit alpha | |
| type 2 | |
| Proteasome | 28639 |
| subunit alpha | |
| type 3-like | |
| Proteasome | 29783 |
| subunit alpha | |
| type 4 | |
| Proteasome | 27856 |
| subunit alpha | |
| type 6 | |
| Proteasome | 26707 |
| subunit beta type 1 | |
| Proteasome | 23083 |
| subunit beta type 2 | |
| Proteasome | 25532 |
| subunit beta type | |
| 6-like | |
| Proteasome | 30269 |
| subunit beta type | |
| 7 precursor | |
| Protein arginine | 43063 |
| N- | |
| methyltransferase 1 | |
| Prothrombin | 71837 |
| precursor | |
| Ras-related | 23086 |
| protein Rab-10 | |
| Ras-related | 24471 |
| protein Rab-11B | |
| Ras-related | 24155 |
| protein Rab-14 | |
| Ras-related | 23774 |
| protein Rab-8B | |
| Rho GDP | 23464 |
| dissociation | |
| inhibitor (GDI) | |
| alpha | |
| Ribose- | 35319 |
| phosphate | |
| pyrophosphokinase | |
| I-like | |
| Serine/threonine | 36178 |
| protein | |
| phosphatase 2A, | |
| catalytic subunit, | |
| alpha isoform | |
| Serine/threonine | 38253 |
| protein | |
| phosphatase | |
| PP1-alpha | |
| catalytic subunit | |
| Serum albumin | 70715 |
| precursor | |
| Similar to Ras- | 22348 |
| related protein | |
| Rab-1B | |
| Soluble calcium- | 45715 |
| activated | |
| nucleotidase 1 | |
| SPARC | 35297 |
| precursor | |
| Splice Isoform 1 | 70211 |
| of Alpha- | |
| fetoprotein | |
| precursor | |
| Splice Isoform 1 | 276159 |
| of Fibronectin | |
| precursor | |
| Splice Isoform 1 | 38363 |
| of | |
| Heterogeneous | |
| nuclear | |
| ribonucleoprotein | |
| D0 | |
| Splice Isoform 1 | 44875 |
| of Sex hormone- | |
| binding globulin | |
| precursor | |
| Splice Isoform 3 | 214603 |
| of Agrin | |
| precursor | |
| Splice Isoform | 40889 |
| Long of | |
| Hyaluronan and | |
| proteoglycan link | |
| protein 1 | |
| precursor | |
| Splice Isoform | 28497 |
| RC6-IL of | |
| Proteasome | |
| subunit alpha | |
| type 7 | |
| Spliceosome | 49491 |
| RNA helicase | |
| Bat1 | |
| Spp-24 | 23455 |
| Stathmin | 17157 |
| Syntenin-1 | 32651 |
| TGF-beta | 95072 |
| receptor type III | |
| precursor | |
| Thrombospondin 1 | 133664 |
| Transcobalamin | 47876 |
| II precursor | |
| Transforming | 22124 |
| protein RhoA | |
| Translationally | 19576 |
| controlled tumor | |
| protein | |
| Transthyretin | 15834 |
| precursor | |
| Triosephosphate | 27303 |
| isomerase | |
| Tubb3 protein | 50875 |
| Tubulin alpha-1 | 50820 |
| chain | |
| Tubulin alpha-3 | 50644 |
| chain | |
| Tubulin beta-5 | 50127 |
| chain | |
| Tubulin, beta, 2 | 50257 |
| Type A/B hnRNP | 30967 |
| p38 | |
| Ubiquitin | 25180 |
| carboxyl-terminal | |
| hydrolase | |
| isozyme L1 | |
| Ubiquitin-like 1 | 38969 |
| Vascular | 41763 |
| endothelial cell | |
| specific protein | |
| 11 | |
| Vimentin | 53658 |
| Vitamin D- | 55141 |
| binding protein | |
| precursor | |
| Zero beta-1 | 16079 |
| globin | |

### IGF2 in the CSF

To test if CSF could serve as a vehicle for IGF signaling, we assessed the expression of IGF1 and IGF2 mRNA in the developing cortex. IGF2 mRNA was highly expressed in the choroid plexus of E17 rat embryos, as well as in vascular endothelial cells and the leptomeninges of both E14 and E17 rat brain (FIGS. 14C and 14D). IGF1 and IGF2 mRNA was not detected levels (data not shown) in developing neural progenitor cells or the cortical mantle, as has been previously reported (Ayer-le Lievre et al., Development 111, 105-15 (1991)). While vascular sources of signaling molecules are important for neural progenitor cell fate (Shen et al. Science 304, 1338-40 (2004); Palmer et al., J Comp Neurol 425, 479-94 (2000)), our IGF2 expression data suggests that the choroid plexus is the primary source of IGF2 in the CSF.

To determine if CSF-borne IGF2 has the capacity to stimulate IGF signaling in the developing cortex, we first examined the localization of the IGF1 receptor (IGF1R) in the developing cortex. IGF1R, which binds IGF2 and is essential for the proliferative response to IGF signaling (references), localized to the apical, ventricular surface of radial neuroepithelial cells that contacts the CSF (FIG. 14E). Further, embryonic CSF activated IGF signaling in primary cortical precursor cells and neurons via the IGF1 receptor, as reflected by induction of phosphorylated IGF1Rβ (p-IGF1Rβ) (FIG. 14F). Embryonic CSF also stimulated the activation of the AKT and MAPK signaling pathways (FIG. 14F), both downstream targets of IGF signaling as well as other growth-factor-stimulated signaling cascades. IGF2 treatment alone induced IGF signaling similar to embryonic CSF (FIG. 14F). Thus, cortical progenitor cells appropriately express cell surface receptors required to engage CSF-borne cues such as IGF2, and reciprocally, CSF-borne IGF2 is capable of inducing the activation of IGF signaling in cortical progenitor cells (Hodge et al., J Neurosci 24, 10201-10 (2004); Dudek et al., Science 275, 661-5 (1997); Hodge et al., Int J Dev Neurosci 25, 233-41 (2007)).

We then tested whether Igf2 could maintain GLAST-positive cortical progenitor cells in vitro by culturing primary neurosphere dissociated cells with Igf2 (FIG. 15A). Interestingly, cells cultured in IGF2 formed small GLAST-positive-staining neurospheres (FIGS. 15A-15C) indicating that IGF2 alone provides a modest proliferative signal and that cells retain their neural progenitor cell fate in the presence of IGF2. We then determined whether IGF2 is both necessary and sufficient to induce maintenance and proliferation of neural progenitor cells along the ventricular zone in cortical explants. E16 cortical explants grown in E17 CSF control conditions showed numerous Vimentin 4A4-labeled, proliferating cells along the ventricle (FIG. 15E). In contrast, E16 explants cultured in E17 CSF and in the presence of an IGF2 neutralizing antibody (IGF2 NAb) revealed a striking decrease of Vimentin 4A4-labeled-cells along the ventricle (FIGS. 15E-15G) (E17 control mean: 28.8±4.3; E17 Igf2 neutralizing antibody mean: 13.9±2.0; n=4, p<0.05). In addition, addition of IGF2 (2 ng/ml) to Neural Basal Media (NBM) plus 20% ACSF stimulated the proliferation of Vimentin 4A4 positive progenitor cells in E16 explants (Igf2 supplementation mean: 36.7±2.1; control mean: 20.4±4.46; n=8, p<0.05) (FIGS. 15H-15J) and in E13 explants (data not shown).

### Methods

The following methods were used to perform the above described experiments.

Cortical Explants

Rat embryos were removed from extra-embryonic membranes and placed in sterile Hanks Balanced Salt Solution (HBSS). The lateral wall of the developing cortex was dissected using a fine scalpel and demarcated in the rostral-caudal direction by the width of the lateral ganglionic eminence, in the dorsal direction by the in-fold of the medial cortical wall, and in the lateral direction by the border with the lateral ganglionic eminence. The dissected cortex was transferred to a polycarbonate membrane (Whatman; 13 mm, 8.0 um) using a platinum wire loop. Explants were then cultured for 24 hours in conditions described in text. Artificial CSF (ACSF) was made fresh for each use. ACSF consisted of NaCl 119 mM, KCl 2.5 mM, NaHCO₃ 26 mM, NaH₂PO₄ 1 mM, Glucose 11 mM, MgCl₂ 2 mM, CaCl₂ 2.8 mM. Supplemental IGF2 (US Biologicals) was added to ACSF at a final concentration of 2 ng/ml. 15 μg of IGF2 neutralization antibody in 15 μl of PBS (Millipore) was incubated with 100% E17 CSF for 1 hour rotating at 4° C. As a control, 15 μl of PBS was incubated with 100% E17 CSF. For BrdU labeling, explants were pulsed with BrdU for 30 minutes immediately prior to fixation. Explants were fixed (60% methanol, 30% chloroform, and 10% acetic acid) for 5-10 minutes, washed with 70% ethanol, embedded in paraffin, and sectioned at 5 um. Explant integrity was visualized by Hematoxylin and Eosin staining (data not shown).

Immunohistochemical and Immunoblot Analysis

The following antibodies were purchased: mouse anti-Tuj 11:250 (Covance), rat anti-BrdU 1:400 (AbD Serotec), rabbit anti-PH3 1:400 (Upstate), mouse anti-Vimentin 4A4 1:100 (Assay Designs), guinea pig anti-GLAST 1:100 (Company name), anti-phospho-Histone H3 1:400 (Upstate), rabbit P-AKT 1:100 (Cell Signaling), rabbit P-IGF1R, 1:100 (Cell Signaling), HRP conjugated anti-albumin 1:10,000 (Immunology Consultants Laboratory, Inc.), HRP conjugated anti-transferrin 1:1000 (Immunology Consultants Laboratory, Inc.), rabbit anti-Cystatin C 1:1000 (abeam), rabbit anti-Cathepsin B 1:1000 (abeam), rabbit anti-IGF2 1:100 (Santa Cruz Biotechnology), rabbit anti-FGF2 1:100 (Santa Cruz Biotechnology), rabbit anti-EC-SOD 1:1000 (Stressgen), mouse anti-APP 1:100 (Chemicon International).

Cortical Neurospheres

E14 rat cortex was dissected in sterile HBSS followed by gentle trituration. Primary spheres were generated in DMEM/F12 supplemented with heparin, N2, FGF (10 ng/ml), and EGF (20 ng/ml) and collected after 7-9 days in vitro (DIV). Primary spheres were then re-suspended in media without EGF or FGF, dissociated into single cells, plated at a final density of 2,500 cells/cm², and cultured in various media conditions. Fresh media was supplemented on day 4 of incubation. Cells were fixed in 4% Paraformaldehyde and stained for GLAST after 10 DIV.

Cortical Cell Cultures

Cultures of mixed cortical progenitor cells and neurons were prepared. Briefly, mouse embryonic E13.5 cortices were isolated and dissociated by Papain Dissociation System according to the manufacturer's instructions (Worthington Biochem. Corp). Cells were cultured in NBM supplemented with 1% penicillin-streptomycin, 1% glutamine, N2, and bFGF (10 ng/ml). The following day, cells were deprived of growth factors for 6 hours, followed by a 5 minute pulse of ACSF, embryonic CSF, or Igf2 (20 ng/ml).

In Situ Hybridization

Non-radioactive in situ hybridization was performed as described (Berger et al., J Comp Neurol 433, 101-14 (2001)), using a digoxigenin (DIG)-labelled cRNA probe generated from a TA vector (Invitrogen) clone of IGF1 or IGF2 cDNA and frozen rat brain sections.

### Other Embodiments

All patents, patent applications, and publications mentioned in this specification, including U.S. Provisional Application No. 60/963,211, filed Aug. 3, 2007, are hereby incorporated by reference to the same extent as if each independent patent, patent application, or publication was specifically and individually indicated to be incorporated by reference.

What is claimed is:

1. A composition comprising at least one component of e-CSF, wherein said component is present at an enhanced level relative to the level in e-CSF and said composition is capable of supporting proliferation, maintenance, or differentiation of a cultured cell.

2. The composition of claim 1 comprising at least two components of e-CSF.

3. The composition of claim 1, wherein said component is a polypeptide.

4. The composition of claim 3, wherein said polypeptide is 14-3-3 protein beta/alpha, 14-3-3 protein epsilon, 14-3-3 protein gamma, 14-3-3 protein theta, 14-3-3 protein zeta/delta, 15 kda protein, 170 kda protein-glutamyl-prolyl-trna synthetase, 1-phosphatidylinositol-4,5-bisphosphate phosphodiesterase gamma 1, 26s protease regulatory subunit 8, 26s proteasome non-ATPase regulatory subunit 1, 284 kda protein. 0, 29 kda protein, 40s ribosomal protein s10, 40s ribosomal protein s11, 40s ribosomal protein s13, 40s ribosomal protein s18, 40s ribosomal protein s21, 40s ribosomal protein s25, 40s ribosomal protein s3, 40s ribosomal protein s3a, 40s ribosomal protein s4, x isoform, 40s ribosomal protein s6, 40s ribosomal protein s7, 40s ribosomal protein s8, 40s ribosomal protein sa, 60s acidic ribosomal protein p0, 60s acidic ribosomal protein p2, 60s ribosomal protein l13, 60s ribosomal protein l18, 60s ribosomal protein l24, 60s ribosomal protein l3, 60s ribosomal protein l4, 60s ribosomal protein l7, 60s ribosomal protein 17a, 60s ribosomal protein l8, 92 kda protein, 109 kda protein, aa1064-apolipoprotein b, AC2-008, actin, alpha skeletal muscle, actin, cytoplasmic 1, adamts-1 precursor, adaptor protein complex AP-2 (alpha 2 subunit), adenosylhomocysteinase, afamin precursor, alpha 2 macroglobulin cardiac isoform, alpha actinin 4, alpha isoform of regulatory subunit a (protein phosphatase 2), alpha-1-acid glycoprotein precursor, alpha-1-antiproteinase precursor, alpha-1-inhibitor 3 precursor, alpha-1-macroglobulin, alpha-2 antiplasmin, alpha-2-globin chain, alpha-2-hs-glycoprotein precursor, alpha-2-macroglobulin precursor, alpha-actinin-1, alpha-actinin-4, alpha-enolase, alpha-mannosidase 2, ambp protein precursor, angiotensin-converting enzyme (somatic isoform precursor), angiotensinogen precursor, apolipoprotein A-I precursor, apolipoprotein a-iv precursor, apolipoprotein b-fragment, apolipoprotein d precursor, apolipoprotein e precursor, apolipoprotein m precursor, arcadlin, ASCC3L1 protein, ATP-citrate synthase, BA1-667-transferrin, beta-1,3-n-acetylglucosaminyltransferase lunatic fringe, beta-2-glycoprotein 1 precursor, beta-2-microglobulin precursor, beta-enolase, bifunctional heparan sulfate n-deacetylase/n-sulfotransferase 1 (ec 2.8.2.8)(glucosaminyl n-deacetylase/n-sulfotransferase 1) (ndst-1), bone morphogenetic protein 1, cadherin egf lag seven-pass g-type receptor 2, cadherin-6 precursor, calcium-dependent secretion activator 1, calmodulin, calumenin precursor, cathepsin b precursor, cathepsin d precursor, cc2-27, cell growth regulator with of hand domain 1, chaperonin containing tcp1, subunit 2, chaperonin containing tcp1, subunit 5, chaperonin subunit 6a, chloride intracellular channel 6, clathrin heavy chain, clip-associating protein 2, clusterin precursor, coatomer subunit beta′, coatomer subunit beta, cofilin-1, cold shock domain-containing protein e1, collagen alpha-1(i) chain precursor, collagen alpha-1(iii) chain precursor, collagen alpha-1(v) chain precursor, collagen alpha-2(i) chain precursor, collagen type a1(xi)7-8, complement c3 precursor, complement c4 precursor, complement component 1, s subcomponent, complement component 2, complement component c6 precursor, complement inhibitory factor h, contactin-1 precursor, contactin-2 precursor, contrapsin-like protease inhibitor 1 precursor, contrapsin-like protease inhibitor 3 precursor, contrapsin-like protease inhibitor 6 precursor, corticosteroid-binding globulin precursor, c-reactive protein precursor, creatine kinase b-type, cullin-associated nedd8-dissociated protein 1, cystatin c precursor, d-3-phosphoglycerate dehydrogenase, da1-24-complement factor b, damage-specific DNA binding protein 1, deleted in colorectal cancer, dermcidin, dihydropyrimidinase-related protein 2, DNA ligase 1, DNA polymerase alpha catalytic subunit (fragment), DNA polymerase delta catalytic subunit, DNA primase large subunit, drebrin 1, dynactin-1, dynein heavy chain, cytosolic, ectonucleotide pyrophosphatase/phosphodiesterase 2, elongation factor 1-alpha 1, elongation factor 2, epithelial-cadherin precursor, epsilon 1 globin, epsilon 2 globin, epsilon 3 globin, eukaryotic translation initiation factor 3 subunit 9, eukaryotic translation initiation factor 4a, isoform 1, eukaryotic translation initiation factor 4a2, eukaryotic translation initiation factor 5a-1, exportin-1, extracellular superoxide dismutase [cu-zn] precursor, fam3c-like protein, far upstream element-binding protein 2, farnesyl pyrophosphate synthetase, fatty acid synthase, fatty acid-binding protein, brain, fetub protein, fibrillin-2, fibrinogen beta chain precursor, fibulin-2 isoform a, follistatin-related protein 1 precursor, fructose-bisphosphate aldolase a, fructose-bisphosphate aldolase c, gamma-glutamyl hydrolase precursor, gelsolin, glucosamine, glucose phosphate isomerase, glucosidase, alpha; acid, glutamyl-prolyl-trna synthetase, glutathione peroxidase 3 precursor, glutathione s-transferase p, gm2 ganglioside activator protein, gpi-anchored ceruloplasmin, gpi-anchored membrane protein 1, grp78 binding protein, GTP-binding nuclear protein ran, testis-specific isoform, guanine nucleotide-binding protein beta subunit 2-like 1, haptoglobin precursor, hausp, heat shock 70 kda protein 1a/1b, heat shock cognate 71 kda protein, heat shock protein 86, heat shock protein hsp 90-beta, heat shock-related 70 kda protein 2, heat-shock protein 105 kda, hemoglobin alpha-1/2 subunit, hemoglobin beta-1 subunit, hemopexin precursor, heparin cofactor 2 precursor, hepatocyte growth factor activator, hephaestin precursor, heterogeneous nuclear ribonucleoprotein c, histidine-rich glycoprotein, histone H1.0, histone H1.2, histone H2A, hnrpk protein, hydroxymethylglutaryl-coa synthase, cytoplasmic, hypothetical protein aldoal1, hypothetical protein loc314432-similar to ubiquitin-protein ligase (ec 6.3.2.19)e1, hypothetical protein rgd1305887-tubulin beta chain, hypothetical protein rgd1305890, hyrac, ig kappa chain c region, b allele, igh-1a protein, ikap, importin beta-1 subunit, inosine monophosphate dehydrogenase 2, insulin-like growth factor 1 receptor precursor, insulin-like growth factor-binding protein complex acid labile chain precursor, inter-alpha trypsin inhibitor, heavy chain 3, inter-alpha-inhibitor h4 heavy chain, iron-responsive element-binding protein 1, ischemia responsive 94 kda protein, isocitrate dehydrogenase [nadp] cytoplasmic, junction plakoglobin, kallistatin, kinesin-1 heavy chain, kinesin-like protein kif15, lactadherin precursor, lar receptor-linked tyrosine phosphatase, large proline-rich protein bat3, leucyl-trna synthetase, leukemia inhibitory factor receptor precursor, leukocyte common antigen-related phosphatase precursor, liver carboxylesterase 1 precursor, l-lactate dehydrogenase a chain, l-lactate dehydrogenase b chain, loc362795 protein, loc367586 xprotein-immunoglobulin gamma heavy chain, low-density lipoprotein receptor precursor, low-density lipoprotein receptor-related protein 2 precursor, lrrgt00164, lumican precursor, mama, mannose 6-phosphate/insulin-like growth factor ii receptor, mannosidase 2, alpha b1, mannosidase, alpha, class 1a, member 1, masp-3 protein, matrin-3, m-cadherin, metalloproteinase inhibitor 1 precursor, microfibrillar-associated protein 4, microtubule-associated protein 4, myosin-10, myosin-9, neogenin precursor, neogenin precursor, nestin, netrin receptor unc5c precursor, neural cell adhesion molecule 1, 140 kda isoform precursor, neural-cadherin precursor, neurocan core protein precursor, neuropilin-2 precursor, neuroserpin precursor, non-erythrocyte beta-spectrin, NONO/P54NRB homolog, nuclear autoantigenic sperm protein, nucleic acid binding factor PRM10, nucleolin, nucleoside diphosphate kinase a, nucleoside diphosphate kinase b, nucleosome assembly protein 1-like 1, o-glcnacase, peptidyl-prolyl cis-trans isomerase a, peptidylprolyl isomerase c, peroxiredoxin-1, peroxiredoxin-2, phosphatidylethanolamine-binding protein, phosphoglycerate kinase 1, phosphoglycerate mutase 2, plasminogen precursor, platelet endothelial cell adhesion molecule precursor, poly [adp-ribose] polymerase 1, predicted c-1-tetrahydrofolate synthase, cytoplasmic, predicted nucleolin-related protein nrp, predicted similar to c-1-tetrahydrofolate synthase, cytoplasmic, predicted similar to fibrinogen, gamma polypeptide, predicted similar to heat shock protein 86, predicted similar to heat shock protein hsp 90-beta, predicted similar to nuclear autoantigenic sperm protein, predicted similar to postsynaptic density protein, predicted similar to proteasome 26s subunit, ATPase 3, predicted similar to t-kininogen 2 precursor (fragment), predicted: adaptor-related protein complex 1, gamma 1 subunit, predicted: aminopeptidase puromycin sensitive, predicted: ATPase, h+ transporting, lysosomal accessory protein 2, predicted: brain glycogen phosphorylase, predicted: cadherin 11, predicted: calsyntenin 1, predicted: chromodomain helicase DNA binding protein 4, predicted: complement component 5, predicted: complement component 7, predicted: dystroglycan 1, predicted: eukaryotic translation elongation factor 1 gamma, predicted: glycoprotein-4-beta-galactosyltransferase 2, predicted: histone deacetylase 6, predicted: hypothetical protein xp_{—}344107, predicted: hypothetical protein xp_{—}579585, predicted: kinesin family member 4, predicted: laminin, gamma 1, predicted: microtubule-associated protein 1b, predicted: mini chromosome maintenance deficient 4 homolog, predicted: mini chromosome maintenance deficient 6, predicted: neural precursor cell expressed, developmentally down-regulated gene 4a, predicted: nidogen 2, predicted: nidogen, predicted: phosphoribosylglycinamide formyltransferase, predicted: procollagen, type xii, alpha 1, predicted: proteasome (prosome, macropain) subunit, beta type 5, predicted: protocadherin 12, predicted: retinol binding protein 4, plasma, predicted: similar to 116 kda u5 small nuclear ribonucleoprotein component, predicted: similar to 25 kda fk506-binding protein, predicted: similar to 26s proteasome non-ATPase regulatory subunit 11, predicted: similar to 40s ribosomal protein s16, predicted: similar to 40s ribosomal protein s19, predicted: similar to 40s ribosomal protein s3, predicted: similar to 40s ribosomal protein s9, predicted: similar to 60s ribosomal protein l12, predicted: similar to 60s ribosomal protein l26, predicted: similar to 60s ribosomal protein l29, predicted: similar to 60s ribosomal protein l38, predicted: similar to 60s ribosomal protein 17a, predicted: similar to alanyl-trna synthetase, predicted: similar to aldehyde dehydrogenase family 7, member a1, predicted: similar to alpha 1 type ii collagen, predicted: similar to alpha 2 type vi collagen isoform 2c2a precursor, predicted: similar to alpha 3 type vi collagen isoform 1 precursor, predicted: similar to alpha enolase, predicted: similar to alpha nac/1.9.2. protein, predicted: similar to amyloid beta (a4) precursor-like protein 1, predicted: similar to apolipoprotein c2, predicted: similar to arx, predicted: similar to beta-galactosidase precursor, predicted: similar to cad protein, predicted: similar to cadherin-5, predicted: similar to ccr4-not transcription complex, subunit 1 isoform a, predicted: similar to ccteta, eta subunit of the chaperonin containing tcp-1, predicted: similar to cellular apoptosis susceptibility protein, predicted: similar to cg1841-pa, isoform a, predicted: similar to chromatin-specific transcription elongation factor, 140 kda subunit, predicted: similar to chromosome condensation protein G, predicted: similar to coatomer protein complex subunit alpha, predicted: similar to collagen alpha 2(iv) chain precursor-mouse, predicted: similar to collagen alpha1 type vi-precursor, predicted: similar to colonic and hepatic tumor over-expressed protein isoform a, predicted: similar to crb2 protein, predicted: similar to cyfip1 protein, predicted: similar to dead/h box polypeptide 36 protein, predicted: similar to desmoplakin isoform ii, predicted: similar to DNA replication licensing factor mcm2, predicted: similar to DNA replication licensing factor mcm3, predicted: similar to DNA replication licensing factor mcm5, predicted: similar to eif4g1 protein, predicted: similar to elastin microfibril interfacer 1, predicted: similar to elav, predicted: similar to enhancer-trap-locus-1, predicted: similar to eno1 protein, predicted: similar to eukaryotic translation initiation factor 3, subunit 10 theta, 150/170 kda, predicted: similar to eukaryotic translation initiation factor 4, gamma 1 isoform a, predicted: similar to expressed sequence ai314180, predicted: similar to expressed sequence c79407, predicted: similar to fibulin-1 precursor, predicted: similar to filamin a, predicted: similar to filamin b, predicted: similar to fras1 related extracellular matrix protein 2, predicted: similar to gamma-filamin, predicted: similar to gcn1 general control of amino-acid synthesis 1-like 1, predicted: similar to glyceraldehyde-3-phosphate dehydrogenase, predicted: similar to gtpase activating protein and vps9 domains 1, predicted: similar to hcf, predicted: similar to heat shock 70 kda protein 4 like, predicted: similar to heat shock protein hsp 90-beta, predicted: similar to hemicentin 1, predicted: similar to heparan sulfate proteoglycan 2, predicted: similar to hepatic multiple inositol polyphosphate phosphatase, predicted: similar to heterogeneous nuclear ribonucleoprotein a2/b1, predicted: similar to hspc263, predicted: similar to immunoglobulin heavy chain, predicted: similar to importin 7, predicted: similar to importin 9, predicted: similar to inter-alpha trypsin inhibitor, heavy chain 1, predicted: similar to inter-alpha-inhibitor h2 chain, predicted: similar to isoleucine-trna synthetase, predicted: similar to kinesin family member 23, predicted: similar to laminin alpha-1 chain precursor-mouse, predicted: similar to laminin b1, predicted: similar to laminin-2 alpha2 chain precursor, predicted: similar to lerk-5, predicted: similar to lipoprotein receptor-related protein, predicted: similar to mam domain containing 2, predicted: similar to methionine-trna synthetase, predicted: similar to mucin 17, predicted: similar to nischarin, predicted: similar to n-terminal aceyltransferase 1, predicted: similar to nuclear pore complex-associated intranuclear coiled-coil protein tpr, predicted: similar to ollistatin-like 5, predicted: similar to p30 dbc protein, predicted: similar to p59 immunophilin, predicted: similar to pappalysin-2 precursor, predicted: similar to peptidoglycan recognition protein 2, predicted: similar to periostin precursor (pn) (osteoblast-specific factor 2) (OSF-2), predicted: similar to phospholipid transfer protein, predicted: similar to phosphoribosylformylglycinamidine synthase, predicted: similar to plexin-b2 precursor, predicted: similar to poly(rc)-binding protein 1, predicted: similar to procollagen, type ix, alpha 2, predicted: similar to programmed cell death 6 interacting protein, predicted: similar to protocadherin 1 isoform 2 precursor, predicted: similar to protocadherin 18 precursor, predicted: similar to protocadherin 19 precursor, predicted: similar to psmc6 protein, predicted: similar to ptk7 protein tyrosine kinase 7, predicted: similar to putative E3 ligase, predicted: similar to putative pre-mrna splicing factor ma helicase, predicted: similar to pyruvate kinase (ec 2.7.1.40) isozyme m2-rat, predicted: similar to pyruvate kinase 3, predicted: similar to ran binding protein 5, predicted: similar to ranbp21, predicted: similar to ranbp4, predicted: similar to ras gtpase-activating-like protein iqgap1, predicted: similar to regulator of nonsense transcripts 1, predicted: similar to ribosomal protein L14, predicted: similar to ribosomal protein L28, predicted: similar to ribosomal protein L34, predicted: similar to ribosomal protein L6, predicted: similar to riken cDNA b430218107 gene, predicted: similar to ma helicase a, predicted: similar to seizure 6-like protein precursor, predicted: similar to sema6a protein, predicted: similar to semaphorin 6d-4, predicted: similar to serine protease inhibitor 2.4, predicted: similar to serotransferrin precursor, predicted: similar to shprh protein, predicted: similar to sidekick 2, predicted: similar to slit-like 2, predicted: similar to slit-robo rho gtpase-activating protein 1, predicted: similar to smc2 protein, predicted: similar to sorcsb splice variant of the vps 10 domain receptor sores, predicted: similar to splicing factor 3b, subunit 3, 130 kda, predicted: similar to stabilin-1, predicted: similar to sushi, von willebrand factor type a, egf and pentraxin domain containing 1, predicted: similar to talin 2, predicted: similar to t-complex protein 1 subunit theta, predicted: similar to translin-associated factor x (tsnax) interacting protein 1, predicted: similar to tubulin-specific chaperone d, predicted: similar to ubiquitin carboxyl-terminal hydrolase 5, predicted: similar to ubiquitin specific protease 9, x-linked, predicted: similar to ubiquitin-activating enzyme e1, predicted: similar to ubiquitin-conjugating enzyme e2 13, predicted: similar to very large g protein-coupled receptor 1, predicted: similar to vesicular integral-membrane protein vip36 precursor, predicted: similar to vinculin, predicted: splicing factor 3b, subunit 1, predicted: thrombospondin 4, predicted: transforming growth factor, beta induced, 68 kda, predicted: tripartite motif protein 28, predicted: tumor rejection antigen gp96, predicted: tyrosine kinase receptor 1, predicted: von willebrand factor, predicted-40s ribosomal protein s17, predicted-heat shock protein hsp 90-beta (fragment), predicted-heterogeneous nuclear ribonucleoprotein a1, predicted-inhibin binding protein long isoform, predicted-matrin-3, predicted-proteasome 26s subunit, ATPase 3, probable g-protein coupled receptor 116 precursor, procollagen c-endopeptidase enhancer 1 precursor, procollagen-lysine, 2-oxoglutarate 5-dioxygenase 3 precursor, procollagen-lysine, 2-oxoglutarate 5-dioxygenase 3 precursor, profilin-1, proliferating cell nuclear antigen, proliferation-associated 2g4, 38 kda, prominin-1s1 splice variant, proprotein convertase subtilisin/kexin type 9 precursor, proteasome (prosome, macropain) 26s subunit, non-ATPase, 2, proteasome subunit alpha type 2, proteasome subunit alpha type 6, proteasome subunit beta type 1, proteasome subunit beta type 2, protective protein for beta-galactosidase, protein arginine n-methyltransferase 1, protein disulfide-isomerase a3 precursor, protein disulfide-isomerase precursor, protein kinase c-binding protein nell2, prothrombin precursor (fragment), protocadherin gamma subfamily c, 3, protocadherin, PRX IV, pyruvate kinase, muscle, quiescin q6, rab gdp dissociation inhibitor alpha, rab gdp dissociation inhibitor beta, rat alpha(1)-inhibitor 3, variant i precursor, rat t-kininogen, ratsg1, receptor-like protein tyrosine phosphatase gamma b-type isoform, receptor-like protein tyrosine phosphatase kappa extracellular region, retinol-binding protein i, cellular, rho gdp dissociation inhibitor (gdi) alpha, ribonucleotide reductase m1, ribosomal protein l13a, ribosomal protein s27a, roundabout homolog 1 precursor, ruvb-like 1, ruvb-like 2, secretogranin-3 precursor, sema4b protein (fragment), serine (or cysteine) proteinase inhibitor, Glade a (alpha-1 antiproteinase, antitrypsin), member 6, serine peptidase inhibitor, Glade f, member 2, serine peptidase inhibitor, Glade g, member 1, serine/cysteine proteinase inhibitor, Glade c, member 1, serine/threonine-protein phosphatase 2a catalytic subunit beta isoform, serum albumin precursor, sez6b, shen-dan, similar to riken cDNA 2810409h07, smc4l1 protein, soluble calcium-activated nucleotidase 1, sortilin precursor, sp120-heterogeneous nuclear ribonucleoprotein u, sparc precursor, sparc-like protein 1 precursor, spectrin alpha chain, brain, splice isoform 1 of 40s ribosomal protein s24, splice isoform 1 of acetyl-coa carboxylase 1, splice isoform 1 of agrin precursor, splice isoform 1 of alpha-1b-glycoprotein precursor, splice isoform 1 of alpha-fetoprotein precursor, splice isoform 1 of attractin precursor, splice isoform 1 of cullin-associated nedd8-dissociated protein 2, splice isoform 1 of DNA-binding protein a, splice isoform 1 of fibrinogen alpha chain precursor, splice isoform 1 of fibronectin precursor, splice isoform 1 of heterogeneous nuclear ribonucleoprotein d0, splice isoform 1 of heterogeneous nuclear ribonucleoprotein m, splice isoform 1 of myosin-11 (fragment), splice isoform 1 of neurofascin precursor, splice isoform 1 of neuronal cell adhesion molecule precursor, splice isoform 1 of protein set, splice isoform 1 of reelin precursor, splice isoform 1 of sex hormone-binding globulin precursor, splice isoform 2 of DNA, splice isoform 2 of interleukin enhancer-binding factor 3, splice isoform 2 of plasminogen activator inhibitor 1 rna-binding protein, splice isoform 2 of polypyrimidine tract-binding protein 2, splice isoform 2 of receptor-type tyrosine-protein phosphatase zeta precursor, splice isoform 2 of tropomyosin beta chain, splice isoform app770 of amyloid beta a4 protein precursor (fragment), splice isoform b of ap-1 complex subunit beta-1, splice isoform cdk2-alpha of cell division protein kinase 2, splice isoform gamma-b of fibrinogen gamma chain precursor, splice isoform hmw of kininogen-1 precursor, splice isoform iiba of dynamin-2, splice isoform long of hyaluronan and proteoglycan link protein 1 precursor, splice isoform pam-1 of peptidyl-glycine alpha-amidating monooxygenase precursor, splice isoform pybp1 of polypyrimidine tract-binding protein 1, splice isoform v0 of versican core protein precursor (fragment), spliceosome rna helicase bat1, ssb protein, staphylococcal nuclease domain-containing protein 1, stathmin, structural maintenance of chromosome 1-like 1 protein, structural maintenance of chromosome 3, superoxide dismutase, syntenin-1, t-cadherin, t-complex protein 1 subunit alpha, t-complex protein 1 subunit delta, tenascin (fragment), thrombospondin 1, tln protein, TPA: proteasome subunit beta type 6-like, transcobalamin-2 precursor, transitional endoplasmic reticulum ATPase, transketolase, translationally-controlled tumor protein, transthyretin precursor, triosephosphate isomerase, tripeptidyl-peptidase 2, tubulin alpha-1 chain, tubulin beta chain, tubulin beta-3 chain, tubulin beta-5 chain, tubulin, beta, 2, tumor necrosis factor type 1 receptor associated protein, txnrd1 protein, udp-n-acetylglucosamine-peptide n-acetylglucosaminyltransferase 110 kda subunit, uridine monophosphate synthetase, vacuolar ATP synthase subunit s1 precursor, valyl-trna synthetase, vascular cell adhesion protein 1 precursor, vesicle associated protein, vigilin, vimentin, vitamin d-binding protein precursor, vitamin k-dependent protein s precursor, zero beta-1 globin, zinc phosphodiesterase elac protein 2, or a functional fragment thereof.

5. The composition of claim 3, wherein said polypeptide is 114 kda protein, 116 kda u5 small nuclear ribonucleoprotein component, 120 kda protein-importin 7, 127 kda protein-ran binding protein 5, 14-3-3 protein epsilon, 14-3-3 protein eta, 14-3-3 protein gamma, 14-3-3 protein theta, 14-3-3 protein zeta/delta, 150 kda oxygen-regulated protein precursor, 16 kda protein, 182 kda tankyrase 1-binding protein, 1-phosphatidylinositol-4,5-bisphosphate phosphodiesterase gamma 1, 22 kda protein, 26s protease regulatory subunit 4, 26s protease regulatory subunit 6a, 26s protease regulatory subunit 7, 26s protease regulatory subunit 8, 26s protease regulatory subunit s10b, 26s proteasome non-ATPase regulatory subunit 12, 26s proteasome non-ATPase regulatory subunit 14, 26s proteasome non-ATPase regulatory subunit 2, 26s proteasome non-ATPase regulatory subunit 3, 26s proteasome non-ATPase regulatory subunit 6, 26s proteasome non-ATPase regulatory subunit 7, 3-mercaptopyruvate sulfurtransferase, 40s ribosomal protein s10, 40s ribosomal protein s13, 40s ribosomal protein s14, 40s ribosomal protein s15, 40s ribosomal protein s16, 40s ribosomal protein s17, 40s ribosomal protein s18, 40s ribosomal protein s19, 40s ribosomal protein s2, 40s ribosomal protein s21, 40s ribosomal protein s23, 40s ribosomal protein s25, 40s ribosomal protein s3, 40s ribosomal protein s4, x isoform, 40s ribosomal protein s7, 40s ribosomal protein s8, 40s ribosomal protein s9, 45 kda protein-homologous to phospholipid transfer protein, 47 kda heat shock protein precursor, 55 kda protein, 60 kda heat shock protein, mitochondrial precursor, 60s acidic ribosomal protein p0, 60s acidic ribosomal protein p2, 60s ribosomal protein l10a, 60s ribosomal protein l12, 60s ribosomal protein l18, 60s ribosomal protein l18a, 60s ribosomal protein l19, 60s ribosomal protein l21, 60s ribosomal protein l23a, 60s ribosomal protein l28, 60s ribosomal protein l3, 60s ribosomal protein l38, 60s ribosomal protein l4, 60s ribosomal protein l4, 60s ribosomal protein l5, 60s ribosomal protein 17a, 60s ribosomal protein 17a, 60s ribosomal protein l8, 60s ribosomal protein l8, 6-phosphogluconate dehydrogenase, decarboxylating, 6-phosphogluconolactonase, 72 kda type iv collagenase precursor, acetyl-coa acetyltransferase, cytosolic, acetyl-coa carboxylase 1, acidic leucine-rich nuclear phosphoprotein 32 family member a, aconitate hydratase, mitochondrial precursor, actin, aortic smooth muscle, actin, cytoplasmic 1, actin-like protein 2, actin-related protein 2/3 complex subunit 1a, acylamino-acid-releasing enzyme, adenosylhomocysteinase, adenylate kinase isoenzyme 1, adenylosuccinate synthetase isozyme 2, adenylyl cyclase-associated protein 1, adp-ribosylation factor 1, adp-ribosylation factor-like protein 3, adp-sugar pyrophosphatase, aflatoxin b 1 aldehyde reductase member 2, agrin precursor, a-kinase anchor protein 12 isoform 2, alanyl-tRNA synthetase, alb (albumin) protein, alb protein, alcadein beta, alcohol dehydrogenase, aldehyde dehydrogenase 16 family, member a1, alpha 3 type vi collagen isoform 1 precursor, alpha isoform of regulatory subunit a, protein phosphatase 2, alpha-1-acid glycoprotein 2 precursor, alpha-1-antitrypsin precursor, alpha-1b-glycoprotein precursor, alpha-2-antiplasmin precursor, alpha-2-hs-glycoprotein precursor, alpha-2-macroglobulin precursor, alpha-actinin-1, alpha-actinin-4, alpha-centractin, alpha-enolase, lung specific, alpha-fetoprotein precursor, alpha-internexin, alpha-mannosidase 2, alpha-soluble nsf attachment protein, ambp protein precursor, amyloid-like protein 1 precursor, angiotensinogen precursor, angiotensinogen precursor, ankyrin repeat and fyve domain containing 1 isoform 1, annexin a5, antithrombin iii variant, ap-1 complex subunit mu-1, ap-2 complex subunit alpha-2, apolipoprotein a-i precursor, apolipoprotein a-ii precursor, apolipoprotein a-iv precursor, apolipoprotein b-100 precursor, apolipoprotein e precursor, apolipoprotein m, aspartate aminotransferase, cytoplasmic, aspartyl-tRNA synthetase, astrocytic phosphoprotein pea-15, ataxin-10, ATP synthase subunit alpha, mitochondrial precursor, ATP-citrate synthase, ATP-dependent DNA helicase 2 subunit 1, ATP-dependent DNA helicase 2 subunit 2, ATP-dependent RNA helicase a, ATP-dependent RNA helicase ddxl, ATP-dependent RNA helicase ddx3x, ba395114.12, basement membrane-specific heparan sulfate proteoglycan core protein precursor (perlecan), bifunctional purine biosynthesis protein purh, biliverdin reductase a precursor, bleomycin hydrolase, bm-010, brain acid soluble protein 1, c-1-tetrahydrofolate synthase, cytoplasmic, cad protein, cadherin egf lag seven-pass g-type receptor 2 precursor, cadherin-2 precursor (neuronal cadherin), cadherin-2 precursor, cadherin-5 precursor, calcium-binding protein 39, calmodulin, calnexin precursor, calpain-1 catalytic subunit, calponin-3, calreticulin precursor, calsyntenin 1 isoform 2, carboxypeptidase n subunit 2 precursor, ccr4-not transcription complex, subunit 1 isoform a, cDNA flj33352 fis, clone brace2005087, weakly similar to pre-mRNA splicing helicase brr2, cDNA flj45525 fis, clone brtha2026311, highly similar to protein disulfide isomerase a6, cDNA flj45706 fis, clone febra2028457, highly similar to nucleolin, cell division cycle 5-like protein, cellular retinoic acid-binding protein 1, centrosomal protein 170 kda isoform alpha, ceruloplasmin precursor, cgi-150 protein, chaperonin containing tcp1, subunit 3 isoform b, chaperonin containing tcp1, subunit 8, class iii alcohol dehydrogenase 5 chi subunit, clathrin heavy chain 1, cleavage and polyadenylation specificity factor 73 kda subunit, clusterin precursor, coatomer subunit alpha, coatomer subunit beta, coatomer subunit beta′, coatomer subunit gamma-2, cofilin-1, cold-inducible RNA-binding protein, collagen alpha-1(i) chain precursor, collagen alpha-1(iii) chain precursor, collagen alpha-1(v) chain precursor, collagen alpha-2(i) chain precursor, collagen alpha-2(iv) chain precursor, complement c1r subcomponent precursor, complement c1s subcomponent precursor, complement c2 precursor (fragment), complement c-4-a precursor, complement c5 precursor, complement component 3 precursor, complement component c6 precursor, condensin complex subunit 1, condensin complex subunit 2, condensin complex subunit 3, contactin-2 precursor, cop9 signalosome complex subunit 5, cop9 signalosome complex subunit 6, coronin-1c, corticosteroid-binding globulin precursor, creatine kinase b-type, crk-like protein, csnk2a1 protein, ctp synthase 1, cttn protein, cystatin b, cystatin c precursor, cysteinyl-tRNA synthetase isoform c, cytochrome b5 reductase isoform 1, cytoplasmic dynein 1 light intermediate chain 2, cytoplasmic fmr1 interacting protein 1 isoform a, cytoskeleton-associated protein 5, cytosolic aminopeptidase p, cytosolic purine 5′-nucleotidase, d-3-phosphoglycerate dehydrogenase, d-dopachrome decarboxylase, dead (asp-glu-ala-asp) box polypeptide 39, isoform 2, dead box polypeptide 42 protein, deah (asp-glu-ala-his) box polypeptide 15, debranching enzyme homolog 1, desmoglein 2, developmentally-regulated gtp-binding protein 1, dihydropyrimidinase-like 2, dihydropyrimidinase-related protein 1, dihydropyrimidinase-related protein 2, dihydropyrimidinase-related protein 4, dihydropyrimidinase-related protein 5, DNA damage-binding protein 1, DNA ligase 1, DNA mismatch repair protein msh2, DNA polymerase delta catalytic subunit, DNA replication licensing factor mcm2, DNA replication licensing factor mcm3, DNA replication licensing factor mcm4, DNA replication licensing factor mcm5, DNA replication licensing factor mcm6, DNA-(apurinic or apyrimidinic site) lyase, DNA-binding protein taxreb107, DNA-directed RNA polymerase ii 140 kda polypeptide, DNA-directed RNA polymerase ii largest subunit, DNAj homolog subfamily a member 1, DNAj homolog subfamily c member 7, dolichyl-diphosphooligosaccharide-protein glycosyltransferase 67 kda subunit precursor, doublecortex\; lissencephaly, x-linked, dpysl3 protein, drebrin, dynactin 2, dync1h1 protein, dynein heavy chain, cytosolic, early endosome antigen 1, echinoderm microtubule-associated protein-like 4, eef1a1 protein, clay, clay-like protein 1, elongation factor 1-alpha 2, elongation factor 1-delta, elongation factor 1-gamma, elongation factor 2, endoplasmin precursor, eno1p protein, enolase 1, esterase d, eukaryotic initiation factor 4a-i, eukaryotic initiation factor 5a isoform i variant a, eukaryotic translation initiation factor 2 subunit 1, eukaryotic translation initiation factor 2c 1, eukaryotic translation initiation factor 3 subunit 10, eukaryotic translation initiation factor 3 subunit 2, eukaryotic translation initiation factor 3 subunit 6, eukaryotic translation initiation factor 3 subunit 8, eukaryotic translation initiation factor 4 gamma 2, eukaryotic translation initiation factor 4 gamma, 1 isoform 2, eukaryotic translation initiation factor 4 gamma, 1 isoform 4, eukaryotic translation initiation factor 5, eukaryotic translation initiation factor 5b, exosome complex exonuclease rrp42, exportin-1, exportin-7, exportin-t, extracellular matrix protein 1 precursor, fact complex subunit spt16, fact complex subunit ssrp1, f-actin capping protein alpha-1 subunit, f-actin capping protein alpha-2 subunit, factor vii active site mutant immunoconjugate, far upstream element-binding protein 2, farnesyl diphosphate synthase, fascin, fatty acid synthase, fibrillarin, fibrinogen beta chain precursor, filamin a, alpha, fk506-binding protein 3, flap endonuclease 1, flj00385 protein (fragment), fructose-bisphosphate aldolase a, fructose-bisphosphate aldolase c, fuse-binding protein-interacting repressor isoform a, galectin-3-binding protein precursor, gamma-enolase, gamma-g globin (fragment), gars protein, gcn1-like protein 1, glucosamine-6-phosphate isomerase, glucose-6-phosphate isomerase, glucosidase 2 subunit beta precursor, glutaminyl-tRNA synthetase, glutamyl-prolyl tRNA synthetase, glutathione s-transferase p, glyceraldehyde-3-phosphate dehydrogenase, glycogen phosphorylase, brain form, glyoxylate reductase/hydroxypyruvate reductase, gmp synthase, golgi phosphoprotein 2, gpi-anchored protein p137, gtp binding protein 1, gtp-binding nuclear protein ran, guanine nucleotide-binding protein g(i)/g(s)/g(t) subunit beta 2, heat shock 70 kda protein 1, heat shock 70 kda protein 4, heat shock 70 kda protein 41, heat shock 70 kda protein 5, heat shock protein 86 (fragment), heat shock protein hsp 90-alpha 2, hemoglobin subunit alpha, hemoglobin subunit beta, hemoglobin subunit epsilon, hemoglobin subunit gamma-1, hemoglobin subunit zeta, heparin cofactor 2 precursor, hepatoma-derived growth factor, heterogeneous nuclear ribonucleoprotein a0, heterogeneous nuclear ribonucleoprotein a1 isoform b, heterogeneous nuclear ribonucleoprotein c-like 1, heterogeneous nuclear ribonucleoprotein d-like, heterogeneous nuclear ribonucleoprotein f, heterogeneous nuclear ribonucleoprotein g, heterogeneous nuclear ribonucleoprotein h1, heterogeneous nuclear ribonucleoprotein 1 isoform a, heterogeneous nuclear ribonucleoprotein m isoform a, heterogeneous nuclear ribonucleoprotein r, heterogeneous nuclear ribonucleoprotein u isoform a, heterogeneous nuclear ribonucleoprotein u, high mobility group protein 1-like 10, high mobility group protein b2, high-mobility group box 1, histidine-rich glycoprotein precursor, histone acetyltransferase type b catalytic subunit, histone h1.2, histone h1x, histone h2b type 2-e, histone h4, histone-binding protein rbbp4, hiv tat specific factor 1, hnrpa2b1 protein, hsc70-interacting protein, hsp90 co-chaperone cdc37, hspc117 protein, hspc121, hydroxymethylglutaryl-coa synthase, cytoplasmic, hypothetical protein dkfzp451d234, hypothetical protein dkfzp451p021, hypothetical protein dkfzp547j2313, hypothetical protein dkfzp564e242, hypothetical protein dkfzp686i0180 (fragment), hypothetical protein dkfzp686m09245, hypothetical protein dkfzp761k0511-heat shock 90 kda protein 1, beta, hypothetical protein dkfzp761k0511, hypothetical protein dkfzp781k0743, hypothetical protein loc345651, hypothetical protein loc387104, Tars protein, igkv1-5 (immunoglobulin kappa variable 1-5) protein, iglcl protein, importin alpha-4 subunit, importin beta-1 subunit, importin-7, importin-9, inorganic pyrophosphatase, inosine-5′-monophosphate dehydrogenase 2, insulin-like growth factor 2 mRNA binding protein 1, inter-alpha-trypsin inhibitor heavy chain h1 precursor, inter-alpha-trypsin inhibitor heavy chain h2 precursor, interleukin enhancer-binding factor 2, iron-responsive element-binding protein 1, isocitrate dehydrogenase [nadp] cytoplasmic, isocitrate dehydrogenase [nadp], mitochondrial precursor, isoform 1 of 26s protease regulatory subunit 6b, isoform 1 of 26s proteasome non-ATPase regulatory subunit 1, isoform 1 of 40s ribosomal protein s24, isoform 1 of acidic leucine-rich nuclear phosphoprotein 32 family member b, isoform 1 of actin-like protein 6a, isoform 1 of alpha-1-antichymotrypsin precursor, isoform 1 of alpha-adducin, isoform 1 of apoptosis inhibitor 5, isoform 1 of ATP-dependent RNA helicase ddx19b, isoform 1 of attractin precursor, isoform 1 of beta-catenin, isoform 1 of cadherin-6 precursor, isoform 1 of chromodomain helicase-DNA-binding protein 4, isoform 1 of clathrin heavy chain 2, isoform 1 of clathrin heavy chain 2, isoform 1 of cleavage and polyadenylation specificity factor 6, isoform 1 of collagen alpha-1(ix) chain precursor, isoform 1 of complement factor b precursor (fragment), isoform 1 of complement factor b precursor (fragment), isoform 1 of complement factor h precursor, isoform 1 of contactin-1 precursor, isoform 1 of cullin-3, isoform 1 of cullin-associated nedd8-dissociated protein 1, isoform 1 of cullin-associated nedd8-dissociated protein 1, isoform 1 of cytoplasmic linker protein 2, isoform 1 of cytosolic acyl coenzyme a thioester hydrolase, isoform 1 of daz-associated protein 1, isoform 1 of dipeptidyl-peptidase 3, isoform 1 of DNA replication licensing factor mcm7, isoform 1 of DNA, isoform 1 of DNA-binding protein a, isoform 1 of DNA-dependent protein kinase catalytic subunit, isoform 1 of double-strand break repair protein mre11a, isoform 1 of dynamin-2, isoform 1 of ectonucleotide pyrophosphatase/phosphodiesterase 2, isoform 1 of elav-like protein 3, isoform 1 of eukaryotic translation initiation factor 3 subunit 9, isoform 1 of exosome complex exonuclease rrp44, isoform 1 of exportin-2, isoform 1 of exportin-5, isoform 1 of fibrinogen alpha chain precursor, isoform 1 of fibronectin precursor, isoform 1 of filamin-b, isoform 1 of filamin-c, isoform 1 of focal adhesion kinase 1, isoform 1 of gelsolin precursor, isoform 1 of general transcription factor ii-i, isoform 1 of glucosamine-fructose-6-phosphate aminotransferase [isomerizing] 1, isoform 1 of heat shock cognate 71 kda protein, isoform 1 of heterogeneous nuclear ribonucleoprotein a3, isoform 1 of heterogeneous nuclear ribonucleoprotein d0, isoform 1 of heterogeneous nuclear ribonucleoprotein h3, isoform 1 of heterogeneous nuclear ribonucleoprotein k, isoform 1 of heterogeneous nuclear ribonucleoprotein k, isoform 1 of heterogeneous nuclear ribonucleoprotein q, isoform 1 of heterogeneous nuclear ribonucleoprotein u-like protein 1, isoform 1 of host cell factor, isoform 1 of jmjc domain-containing histone demethylation protein 2b, isoform 1 of kh domain-containing, RNA-binding, signal transduction-associated protein 1, isoform 1 of lira and sh3 domain protein 1, isoform 1 of melanoma-associated antigen d2, isoform 1 of microtubule-associated protein 2, isoform 1 of microtubule-associated protein rp/eb family member 2, isoform 1 of multiple epidermal growth factor-like domains 8, isoform 1 of neogenin precursor, isoform 1 of neuronal cell adhesion molecule precursor, isoform 1 of nuclear autoantigenic sperm protein, isoform 1 of periostin precursor, isoform 1 of phospholipid transfer protein precursor, isoform 1 of phosphoserine aminotransferase, isoform 1 of plasminogen activator inhibitor 1 RNA-binding protein, isoform 1 of plexin domain-containing protein 2 precursor, isoform 1 of polyadenylate-binding protein 1, isoform 1 of polyadenylate-binding protein 4, isoform 1 of polypyrimidine tract-binding protein 1, isoform 1 of probable ATP-dependent RNA helicase ddx17, isoform 1 of proteasome subunit alpha type 7, isoform 1 of protein 4.1, isoform 1 of protein arginine n-methyltransferase 1, isoform 1 of protein phosphatase 1 regulatory subunit 7, isoform 1 of protein set, isoform 1 of ras gtpase-activating protein 1, isoform 1 of regulator of nonsense transcripts 1, isoform 1 of reticulon-4, isoform 1 of RNA-binding protein nova-1, isoform 1 of roundabout homolog 1 precursor, isoform 1 of slit-robo rho gtpase-activating protein 3, isoform 1 of spectrin alpha chain, brain, isoform 1 of spectrin beta chain, brain 2, isoform 1 of squamous cell carcinoma antigen recognized by t-cells 3, isoform 1 of structural maintenance of chromosome 2-like 1 protein, isoform 1 of symplekin, isoform 1 of tenascin precursor, isoform 1 of transcription elongation factor spt5, isoform 1 of ubiquitin-protein ligase bre1b, isoform 1 of uridine 5′-monophosphate synthase, isoform 1 of vinculin, isoform 2 of at-rich interactive domain-containing protein 1a, isoform 2 of cadherin-11 precursor, isoform 2 of DNA replication licensing factor mcm7, isoform 2 of far upstream element-binding protein 1, isoform 2 of guanine nucleotide-binding protein g(i), alpha-2 subunit, isoform 2 of hect, uba and wwe domain-containing protein 1, isoform 2 of inter-alpha-trypsin inhibitor heavy chain h4 precursor, isoform 2 of microtubule-actin crosslinking factor 1, isoforms 1/2/3/5, isoform 2 of microtubule-associated protein 4, isoform 2 of neural cell adhesion molecule l1-like protein precursor, isoform 2 of neutral alpha-glucosidase ab precursor, isoform 2 of nmda receptor-regulated protein 1, isoform 2 of nsfl1 cofactor p47, isoform 2 of nuclear mitotic apparatus protein 1, isoform 2 of nucleophosmin, isoform 2 of proteasome subunit alpha type 3, isoform 2 of protein enabled homolog, isoform 2 of protein kiaa1967, isoform 2 of putative gtp-binding protein ptd004, isoform 2 of serine/threonine-protein kinase dcamkl1, isoform 2 of serine/threonine-protein kinase pak 1, isoform 2 of splicing factor 1, isoform 2 of structural maintenance of chromosomes 4-like 1 protein, isoform 2 of suppressor of g2 allele of skp1 homolog, isoform 2 of swi/snf-related matrix-associated actin-dependent regulator of chromatin subfamily c member 2, isoform 2 of transcription factor btf3, isoform 2 of ubiquitin carboxyl-terminal hydrolase 47, isoform 2a of desmocollin-2 precursor, isoform 2c of cytoplasmic dynein 1 intermediate chain 2, isoform 3 of anamorsin, isoform 3 of DNA repair protein rad50, isoform 3 of drebrin-like protein, isoform 3 of polypyrimidine tract-binding protein 2, isoform 3 of udp-n-acetylglucosamine-peptide n-acetylglucosaminyltransferase 110 kda subunit, isoform 4 of afadin, isoform 4 of heterogeneous nuclear ribonucleoprotein a/b, isoform 4 of saps domain family member 3, isoform 4 of tubulin-specific chaperone d, isoform 5 of dynamin-1-like protein, isoform 5 of interleukin enhancer-binding factor 3, isoform a22 of neuropilin-2 precursor, isoform app770 of amyloid beta a4 protein precursor (fragment), isoform b of arsenite-resistance protein 2, isoform b of fibulin-1 precursor, isoform b of mannose-6-phosphate receptor-binding protein 1, isoform b of neuronal-specific septin-3, isoform b1 of heterogeneous nuclear ribonucleoproteins a2/b1, isoform beta of heat-shock protein 105 kda, isoform beta-2 of DNA topoisomerase 2-beta, isoform c of fibulin-1 precursor, isoform c of neural cell adhesion molecule 1, 120 kda isoform precursor, isoform c1 of heterogeneous nuclear ribonucleoproteins c1/c2, isoform delta-1 of serine/threonine-protein phosphatase 2a 56 kda regulatory subunit delta isoform, isoform dpi of desmoplakin, isoform dut-m of deoxyuridine 5′-triphosphate nucleotidohydrolase, mitochondrial precursor, isoform ews-b of RNA-binding protein ews, isoform gamma-1 of serine/threonine-protein phosphatase pp1-gamma catalytic subunit, isoform gamma-b of fibrinogen gamma chain precursor, isoform gtbp-alt of DNA mismatch repair protein msh6, isoform gtbp-n of DNA mismatch repair protein msh6, isoform hmw of kininogen-1 precursor, isoform ii of ubiquitin-protein ligase e3a, isoform long of 60 kda ss-a/ro ribonucleoprotein, isoform long of cold shock domain-containing protein e1, isoform long of collagen alpha-1(xii) chain precursor, isoform long of spectrin beta chain, brain 1, isoform long of splicing factor, proline- and glutamine-rich, isoform long of trifunctional purine biosynthetic protein adenosine-3, isoform long of ubiquitin carboxyl-terminal hydrolase 5, isoform m1 of pyruvate kinase isozymes m1/m2, isoform p150 of dynactin-1, isoform short of heterogeneous nuclear ribonucleoprotein u, isoform short of proteasome subunit alpha type 1, isoform short of receptor-type tyrosine-protein phosphatase zeta precursor, isoform short of RNA-binding protein fus, isoform short of tata-binding protein-associated factor 2n, isoform v0 of versican core protein precursor, isopentenyl-diphosphate delta isomerase, kh-type splicing regulatory protein, kinesin heavy chain, kinesin heavy chain isoform 5c, kinesin light chain 1 isoform 2, lactate dehydrogenase a, lamina-associated polypeptide 2 isoform alpha, laminin alpha 2 subunit precursor, laminin beta-1 chain precursor, laminin gamma-1 chain precursor (laminin b2 chain), lethal giant larvae homolog 1, leucine zipper transcription factor-like 1, leucine-rich repeat-containing protein 15 precursor, leucine-rich repeat-containing protein 47, leucyl-tRNA synthetase, cytoplasmic, liver phosphofructokinase isoform a, l-lactate dehydrogenase b chain, lumican precursor, lung cancer oncogene 7, lupus 1a protein, lysyl-tRNA synthetase, malate dehydrogenase, cytoplasmic, malate dehydrogenase, mitochondrial precursor, marcks-related protein, matrin-3, meprin a subunit alpha precursor, metastasis-associated protein mta2, methionine adenosyltransferase ii, beta isoform 1, methionyl-tRNA synthetase, mgea5 protein, microsomal triglyceride transfer protein large subunit precursor, microtubule-associated protein 1b, microtubule-associated protein rp/eb family member 1, mitogen-activated protein kinase 1, moesin, multifunctional protein ade2, myosin-10, myosin-11, myosin-9, myristoylated alanine-rich c-kinase substrate, nascent polypeptide-associated complex subunit alpha, ncl (nucleolin) protein, ncl protein, nestin, netrin receptor dcc precursor, neurocan core protein precursor, neuronal protein np25, ng,ng-dimethylarginine dimethylaminohydrolase 2, nidogen-2 precursor, non-pou domain-containing octamer-binding protein, nuclear cap-binding protein subunit 1, nuclear migration protein nudc, nuclease sensitive element-binding protein 1, nucleoside diphosphate kinase a, nucleoside diphosphate kinase b, nucleosome assembly protein 1-like 1, nucleosome assembly protein 1-like 4, pdcd6ip protein, pentraxin-related protein ptx3 precursor, peptidyl-prolyl cis-trans isomerase a, peptidylprolyl isomerase b precursor, peripherin, peroxiredoxin-1, peroxiredoxin-2, peroxiredoxin-6, peroxisomal multifunctional enzyme type 2, phenylalanyl-tRNA synthetase beta chain, phosphatidylethanolamine-binding protein 1, phosphatidylinositol transfer protein, beta, phosphofructokinase, muscle, phosphoglucomutase-2-like 1, phosphoglycerate kinase 1, phosphoglycerate mutase 2, phospholipase a-2-activating protein, phosphoribosyl pyrophosphate synthetase-associated protein 2, phosphoribosylformylglycinamidine synthase, phytanoyl-coa hydroxylase interacting protein-like, pigment epithelium-derived factor precursor (pedf), pigment epithelium-derived factor precursor, plasma protease c1 inhibitor precursor, plasma retinol-binding protein precursor, plasminogen precursor, platelet-activating factor acetylhydrolase, isoform ib, alpha subunit, pnas-125, poly [adp-ribose] polymerase 1, poly(rc)-binding protein 1, poly(rc)-binding protein 2 isoform b, pp856, predicted: similar to ATP-dependent DNA helicase ii, 70 kda subunit (lupus ku autoantigen protein p70) (ku70) (70 kda subunit of ku antigen) (thyroid-lupus autoantigen) (tlaa) (ctc box binding factor 75 kda subunit) (ctcbf) (ctc75) isoform 1, predicted: similar to basic leucine zipper and w2 domains 1, predicted: similar to chloride intracellular channel protein 4, predicted: similar to heterogeneous nuclear ribonucleoprotein a1, predicted: similar to heterogeneous nuclear ribonucleoprotein a3 isoform 1, predicted: similar to heterogeneous nuclear ribonucleoprotein k isoform a isoform 2, predicted: similar to heterogeneous nuclear ribonucleoprotein u, predicted: similar to peptidylprolyl isomerase a isoform 1, predicted: similar to phosphoglycerate mutase 1 (phosphoglycerate mutase isozyme b) (pgam-b) (bpg-dependent pgam 1) isoform 1, predicted: similar to ran-specific gtpase-activating protein, predicted: similar to ribosomal protein l13 isoform 1, predicted: similar to ribosomal protein s3a isoform 1, predicted: structural maintenance of chromosomes flexible hinge domain containing 1, pregnancy zone protein precursor, pre-mRNA-processing factor 6 homolog, pre-mRNA-processing-splicing factor 8, pre-mRNA-splicing factor 19, pro2275-serpin peptidase inhibitor, Glade a (alpha-1 antiproteinase, antitrypsin), member 1, pro2275, probable ATP-dependent RNA helicase ddx23, probable ATP-dependent RNA helicase ddx46, probable ATP-dependent RNA helicase ddx48, probable ATP-dependent RNA helicase ddx5, profilin 2 isoform a, profilin-1, proliferating cell nuclear antigen, proliferation-associated protein 2g4, prolyl endopeptidase, proteasome 26s non-ATPase subunit 11 variant (fragment), proteasome 26s non-ATPase subunit 13 isoform 2, proteasome activator complex subunit 1, proteasome subunit alpha type 2, proteasome subunit alpha type 6, proteasome subunit beta type 1, proteasome subunit beta type 4 precursor, protein c14orf166, protein disulfide-isomerase a3 precursor, protein disulfide-isomerase a4 precursor, protein disulfide-isomerase precursor, protein dj-1, protein fam49b, protein fam98b, protein kinase c-binding protein nell2 precursor, protein phosphatase 2c isoform gamma, protein rcc2, protein transport protein sec23a, protein transport protein sec24c, protein tyrosine phosphatase, receptor-type, zeta1 precursor, prothrombin precursor (fragment), prothymosin alpha, puromycin-sensitive aminopeptidase, quiescin q6 isoform a, quinone oxidoreductase, rab gdp dissociation inhibitor alpha, rab gdp dissociation inhibitor beta, rab1a, member ras oncogene family, radixin, ran binding protein 5, ras gtpase-activating-like protein iqgap1, ras-gtpase-activating protein-binding protein 1, ras-related protein rab-14, ras-related protein rab-2a, ras-related protein rab-5c, ras-related protein rab-7, rcc1 protein, rctpi1 (fragment), receptor-type tyrosine-protein phosphatase f precursor, replication protein a 70 kda DNA-binding subunit, reticulocalbin-1 precursor, retinoblastoma-associated factor 600, rho gdp-dissociation inhibitor 1, ribonucleoside-diphosphate reductase large subunit, RNA binding motif protein, x-linked-like 1, RNA binding protein (fragment), RNA-binding protein 12, RNA-binding protein musashi homolog 1, ruvb-like 1, ruvb-like 2, s-adenosylmethionine synthetase isoform type-2, scc-112 protein, sec31l1 protein, selenide, water dikinase 1, septin 9, septin-11, septin-2, septin-7, serine/threonine-protein kinase mrck beta, serine/threonine-protein phosphatase 2a catalytic subunit alpha isoform, serine/threonine-protein phosphatase 4 catalytic subunit, serine-threonine kinase receptor-associated protein, serotransferrin precursor, seryl-tRNA synthetase, sf3b3 protein, signal recognition particle 14 kda protein, similar to annexin a2 isoform 1, similar to nestin, small glutamine-rich tetratricopeptide repeat-containing protein a, small nuclear ribonucleoprotein sm d1, small nuclear ribonucleoprotein sm d2, smarca4 isoform 2, sorting nexin 1 isoform c, spermatid perinuclear RNA-binding protein, spermidine synthase, spliceosome RNA helicase bat1, splicing factor 3 subunit 1, splicing factor 3a subunit 3, splicing factor 3b subunit 1, splicing factor 3b subunit 2, splicing factor 3b subunit 3, splicing factor u2af 65 kda subunit, splicing factor, arginine/serine-rich 1, splicing factor, arginine/serine-rich 2, splicing factor, arginine/serine-rich 4, staphylococcal nuclease domain-containing protein 1, stathmin, stress-70 protein, mitochondrial precursor, stress-induced-phosphoprotein 1, structural maintenance of chromosome 1-like 1 protein, structural maintenance of chromosome 3, superkiller viralicidic activity 2-like 2, swi/snf-related matrix-associated actin-dependent regulator of chromatin subfamily a member 5, synaptic vesicle membrane protein vat-1 homolog, taldo 1 protein, talin-1, tar DNA-binding protein 43, t-complex protein 1 subunit alpha, t-complex protein 1 subunit beta, t-complex protein 1 subunit delta, t-complex protein 1 subunit epsilon, t-complex protein 1 subunit eta, t-complex protein 1 subunit zeta, thimet oligopeptidase, thioredoxin reductase 1, cytoplasmic precursor, thioredoxin, thioredoxin-like protein 1, thioredoxin-like protein 2, tho complex subunit 4, threonyl-tRNA synthetase, cytoplasmic, thymidylate synthase, thymopoietin isoform beta, transitional endoplasmic reticulum ATPase, transketolase, transmembrane protein 132a isoform b, transportin 1, transthyretin precursor, tripartite motif-containing 28 protein, tripartite motif-containing protein 2, tripeptidyl-peptidase 2, tropomyosin 1 alpha chain isoform 2, tropomyosin 4, TRYPSIN PRECURSOR (EC 3.4.21.4)>PIR1:TRPGTR trypsin (EC 3.4.21.4), trypsin precursor (ec 3.4.21.4)>pir1:trpgtr trypsin (ec 3.4.21.4), tryptophanyl-tRNA synthetase, tuba6 protein, tubulin alpha-1 chain, tubulin beta-1 chain, tubulin beta-2 chain, tubulin beta-2c chain, tubulin beta-3 chain, tubulin beta-4 chain, tubulin, beta 2, tubulin-specific chaperone a, tubulin-specific chaperone b, tubulin-tyrosine ligase-like protein 12, tumor protein, translationally-controlled 1, twinfilin isoform 1, type 1 tumor necrosis factor receptor shedding aminopeptidase regulator isoform a, tyrosine 3-monooxygenase/tryptophan 5-monooxygenase activation protein, beta polypeptide, tyrosyl-tRNA synthetase, cytoplasmic, u1 small nuclear ribonucleoprotein a, u4/u6.u5 tri-snrnp-associated protein 1, u5 small nuclear ribonucleoprotein 200 kda helicase, ubiquitin and ribosomal protein s27a precursor, ubiquitin carboxyl-terminal hydrolase 10, ubiquitin carboxyl-terminal hydrolase 7, ubiquitin carboxyl-terminal hydrolase isozyme 11, ubiquitin specific protease 9, x-linked isoform 4, ubiquitin-activating enzyme e1, ubiquitin-conjugating enzyme e2 n, ubiquitin-like 1-activating enzyme e1a, ubiquitin-like 1-activating enzyme e1b, udp-glucose 6-dehydrogenase, udp-glucose ceramide glucosyltransferase-like 1 isoform 1, uncharacterized protein c20orf77, uroporphyrinogen decarboxylase, uv excision repair protein rad23 homolog b, vacuolar ATP synthase catalytic subunit a, ubiquitous isoform, vacuolar protein sorting 26a, vacuolar protein sorting 35, valyl-tRNA synthetase, vasorin precursor, vesicle-fusing ATPase, villin 2, vimentin, vitronectin precursor, von hippel-lindau binding protein 1, von willebrand factor precursor, wd repeat protein 61, wd40 protein, wugsc:h_rg054d04.1 protein, ww domain-binding protein 11, or zyxin, or a functional fragment thereof.

6. The composition of claim 3, wherein said polypeptide is produced recombinantly.

7. The composition of claim 1, wherein said component is purified.

8. The composition of claim 1, wherein said component is present at a level sufficient to enhance cell proliferation, maintenance, or differentiation.

9. The composition of claim 1, wherein said cultured cell is a stem cell or progenitor cell.

10. The composition of claim 1, wherein said cultured cell is neural cell.

11. The composition of claim 1, wherein said component is not found in adult CSF.

12. The composition of claim 1, wherein said e-CSF is rat or human.

13. A cell culture composition comprising a cell and a composition of claim 1.

14. A kit comprising:
(a) a composition comprising at least one component of e-CSF, wherein the component is present at an enhanced level relative to naturally occurring e-CSF; and
(b) instructions for using (a) for cell culture.

15. A method of culturing a stem cell or a progenitor cell, comprising incubating said cell in culture media containing at least one isolated component of rat or human e-CSF.

16. The method of claim 15, wherein said component is a polypeptide.

17. The method of claim 16, wherein said polypeptide is listed in Tables 1-4, or a functional fragment thereof.

18. The method of claim 16, wherein said polypeptide is produced recombinantly.

19. The method of claim 15, wherein said component is purified.

20. The method of claim 15, wherein said cell is a neural stem cell or a neural progenitor cell.

21. The method of claim 15, wherein said component is not found in adult CSF.

22. A method of isolating embryonic cerebrospinal fluid (e-CSF) comprising:
(a) providing an embryo;
(b) inserting a capillary needle into a ventricle of the central nervous system of said embryo such that the tip of said needle contacts CSF; and
(c) extracting CSF from said embryo through said needle, thereby isolating e-CSF.

23. The method of claim 22, further comprising:
(d) removing intact contaminating cells.

24. The method of claim 23, wherein step (d) removing is performed by centrifugation or filtration.

25. The method of claim 22, wherein said step (c) is performed such that said needle tip does not contact the neuroepithelium during said extraction.

26. The method of claim 22, wherein said e-CSF is removed from a lateral ventricle or from the third or fourth ventricle of said embryo, or a combination thereof.

27. The method of claim 26, wherein e-CSF is removed from said lateral ventricle.

28. The method of claim 22, further comprising storing the e-CSF at less than about −20° C. to about −80° C.

29. The composition of claim 1, wherein said component is insulin-like growth factor 2 (IGF2).
